(19) United States
(12) Patent Application Publication
(10) Pub. No.: US 2010/0093675 A1
(43) Pub. Date: Apr. 15, 2010

(54) NOVEL METHOD

(75) Inventors: Enrico Colli, Milan (IT); Margherita Mariani, Milan (IT); Paola Panina, Milano (IT)

Correspondence Address: EDWARDS ANGELL PALMER & DODGE LLP, P.O. BOX 55874, BOSTON, MA, 02205, US

(73) Assignee: BIOXELL S.P.A., Milan (IT)

(21) Appl. No.: 12/442,239

(22) PCT Filed: Sep. 21, 2007
(86) PCT No.: PCT/EP2007/060068

§ 371 Date: Nov. 30, 2009

Related U.S. Application Data
(60) Provisional application No. 60/846,832, filed on Sep. 22, 2006.

(30) Foreign Application Priority Data
Sep. 22, 2006 (GB) GB 0618700.9

(51) Int. Cl. A61K 31/59; A61P 41/00

(52) U.S. Cl. 514/167

(57) ABSTRACT

There is provided a method of prevention of adhesions, eg surgical adhesions, which comprises using a vitamin D compound.

### FIELD OF THE INVENTION

The present invention relates to novel uses and methods, and compounds for use therein, specifically the use of vitamin D compounds for preventing adhesions.

### BACKGROUND OF THE INVENTION

A major clinical problem relating to repair of the peritoneum and other tissues is the formation of adhesions. Adhesions form when closely apposed surfaces are damaged e.g. due to surgical trauma, mechanical injury, ischemic injury, inflammation, chemical insult, radiotherapy, infection or other foreign body reaction.

A particularly significant cause of adhesions is due to surgery. Intra-abdominal adhesion formation and reformation after surgery is a significant cause of morbidity. Post-surgical adhesions can result in intestinal obstruction. The presence of adhesions from prior surgery can increase operating times and increase intraoperative complications, including damage to the bowel, bladder, ureters and bleeding.

Pelvic adhesions can cause infertility and pain. Adhesions can cause infertility by causing mechanical blockage of the Fallopian tubes thus preventing oocyte retrieval.

Certain surgical procedures tend to be particularly associated with the formation of adhesions such as cholecystectomies, appendectomies, colon surgery and pelvic surgery.

Adhesions are particularly associated with the peritoneum, however, they may also be associated with the tissues of the thorax, heart, spine, joints, eye and nose amongst others.

When adhesions are associated with inflammation or infective processes, they may be associated with, for example, peritonitis, pericarditis, pleuritis, cholecystitis and pelvic inflammatory disease.

The incidence of adhesions following surgical procedures ranges from 55-94%, with a higher incidence following gynaecological surgery. These percentages can be imprecise due to inability to diagnose adhesions by imaging modalities as well as the absence of blood tests which allow their diagnosis. Today, the only way to identify post-surgical adhesion formation is observation at another surgery.

The incidence of post-surgical adhesions is increased by the introduction into the body of foreign matter, such as talc from surgical gloves.

The development of post-surgical adhesions is thought to occur within the immediate 3-5 days following the surgical procedure. Thus, modulation of the healing process during this time period is critical to minimize (and hopefully prevent), post-surgical adhesion development.

The initiation of adhesion formation begins with formation of a fibrin matrix which typically occurs during coagulation in the presence of suppressed fibrinolysis. Surgical injury of tissues reduces or eliminates blood flow thereby producing ischemia which leads to local persistence of fibrin matrix. This matrix is gradually replaced by vascular granular tissue. Eventually the adhesion matures into a fibrous band, often containing small nodules of calcification. The adhesions are often covered by a mesothelium layer and contain blood vessels and connective tissue fibres. Nerve tissue has been found in pelvic adhesions, including those with a history of pelvic pain.

Currently there is no ideal method of preventing adhesion formation or reformation. In terms of surgical technique, gentle tissue handling, meticulous hemostatis, copious irrigation, prevention of infection and avoidance of foreign body response (e.g. due to use of powdered gloves) is recommended. Agents that prevent inflammation (such as steroids and COX-2 inhibitors) and agents that degrade fibrin (such as tissue plasminogen activator) or prevent coagulation (such as heparin) have been evaluated. Current methods commercially available include physical barriers, which act by effectively separating the traumatised tissues. This separation can be achieved by using solid mechanical barriers (film and gels like Interceed or Seprafilm) or by the use of fluids by hydroflotation (icodextrin). However, most of barrier methods may be inappropriate because difficult to handle, not suitable for certain types of surgery (for instance they might not be suited for laparoscopic surgery) and because may increase anastomotic leaks and even become ineffective in the presence of blood. Since icodextrin solutions support bacterial growth, their use is contraindicated where there is risk of bacterial infection, which is therefore a major drawback.

US2005/0281883A (Daniloff) discloses compositions which cross-link in situ and are said to be of use in the reduction of adhesions. According to this disclosure, the compositions may be combined with a range of anti-fibrotic agents including inosine monophosphate dehydrogenase inhibitors such as 1-alpha 25-dihydroxy vitamin D3 or an analogue or derivative thereof.

WO2006/094064 (Avocet Polymer Technologies Inc) concerns methods and compositions for improving the appearance and/or reducing the size of a closed wound, involving the administration of vitamin D or active vitamin D analogues.

U.S. Pat. No. 5,194,248 (Boston University) relates to methods for the provision of vitamin D analogues to the skin through the administration of agents which are converted into vitamin D precursors by low energy UV light. The methods are said to be of use in wound healing and the inhibition of scar formation.

In general current methods for treating adhesions are associated with limited success. Therefore a strong need exists for more selective and specific methods for the prevention of adhesions which are free of the well recognised disadvantages of the current approaches.

### SUMMARY OF THE INVENTION

The present inventors have developed a new method of preventing adhesions, in particular post-surgical adhesions, with a view to mitigating or alleviating the aforementioned disadvantages. The method is based on the use of calcitriol and analogues thereof, collectively referred to herein as “vitamin D compounds”.

The importance of vitamin D (cholecalciferol) in the biological systems of higher animals has been recognized since its discovery by Mellanby in 1920 (Mellanby, E. (1921) Spec. Rep. Ser. Med. Res. Council (GB) SRS 61:4). It was in the interval of 1920-1930 that vitamin D officially became classified as a “vitamin” that was essential for the normal development of the skeleton and maintenance of calcium and phosphorus homeostasis.

Studies involving the metabolism of vitamin D₃ were initiated with the discovery and chemical characterization of the plasma metabolite, 25-hydroxyvitamin D₃ [25(OH) D₃] (Blunt, J. W. et al. (1968) Biochemistry 6:3317-3322) and the hormonally active form, 1-alpha,25(OH)₂D₃ (Myrtle, J. F. et al. (1970) J. Biol. Chem. 245:1190-1196; Norman, A. W. et al. (1971) Science 173:51-54; Lawson, D. E. M. et al. (1971) Nature 230:228-230; Holick, M. F. (1971) Proc. Natl. Acad. Sci. USA 68:803-804). The formulation of the concept of a vitamin D endocrine system was dependent both upon appreciation of the key role of the kidney in producing 1-alpha,25(OH)₂D₃ in a carefully regulated fashion (Fraser, D. R. and Kodicek, E (1970) Nature 288:764-766; Wong, R. G. et al. (1972) J. Clin. Invest. 51:1287-1291), and the discovery of a nuclear receptor for 1-alpha,25(OH)₂D₃ (VD₃R) in the intestine (Haussler, M. R. et al. (1969) Exp. Cell Res. 58:234-242; Tsai, H. C. and Norman, A. W. (1972) J. Biol. Chem. 248:5967-5975).

The operation of the vitamin D endocrine system depends on the following: first, on the presence of cytochrome P450 enzymes in the liver (Bergman, T. and Postlind, H. (1991) Biochem. J. 276:427-432; Ohyama, Y. and Okuda, K. (1991) J. Biol. Chem. 266:8690-8695) and kidney (Henry, H. L. and Norman, A. W. (1974) J. Biol. Chem. 249:7529-7535; Gray, R. W. and Ghazarian, J. G. (1989) Biochem. J. 259:561-568), and in a variety of other tissues to effect the conversion of vitamin D₃ into biologically active metabolites such as 1-alpha,25(OH)₂D₃ and 24R,25(OH)₂D₃; second, on the existence of the plasma vitamin D binding protein (DBP) to effect the selective transport and delivery of these hydrophobic molecules to the various tissue components of the vitamin D endocrine system (Van Baelen, H. et al. (1988) Ann NY Acad. Sci. 538:60-68; Cooke, N. E. and Haddad, J. G. (1989) Endocr. Rev. 10:294-307; Bikle, D. D. et al. (1986) J. Clin. Endocrinol. Metab. 63:954-959); and third, upon the existence of stereoselective receptors in a wide variety of target tissues that interact with the agonist 1-alpha,25(OH)₂D₃ to generate the requisite specific biological responses for this secosteroid hormone (Pike, J. W. (1991) Annu Rev. Nutr. 11:189-216). To date, there is evidence that nuclear receptors for 1-alpha,25(OH)₂D₃ (VD₃R) exist in more than 30 tissues and cancer cell lines (Reichel, H. and Norman, A. W. (1989) Annu Rev. Med. 40:71-78), including the normal eye (Johnson J A et al. Curr Eye Res. 1995 February; 14(2): 101-8).

Vitamin D₃ and its hormonally active forms are well-known regulators of calcium and phosphorus homeostasis. These compounds are known to stimulate, at least one of, intestinal absorption of calcium and phosphate, mobilization of bone mineral, and retention of calcium in the kidneys. Furthermore, the discovery of the presence of specific vitamin D receptors in more than 30 tissues has led to the identification of vitamin D₃ as a pluripotent regulator outside its classical role in calcium/bone homeostasis. A paracrine role for 1-alpha,25(OH)₂ D₃ has been suggested by the combined presence of enzymes capable of oxidizing vitamin D₃ into its active forms, e.g., 25-OHD-1-alpha-hydroxylase, and specific receptors in several tissues such as bone, keratinocytes, placenta, and immune cells. Moreover, vitamin D₃ hormone and active metabolites have been found to be capable of regulating cell proliferation and differentiation of both normal and malignant cells (Reichel, H. et al. (1989) Ann. Rev. Med. 40: 71-78).

Given the activities of vitamin D₃ and its metabolites, much attention has focused on the development of synthetic analogues of these compounds. A large number of these analogues involve structural modifications in the A ring, B ring, C/D rings, and, primarily, the side chain (Bouillon, R. et al. (1995) Endocrine Reviews 16(2):201-204). Although a vast majority of the vitamin D₃ analogues developed to date involve structural modifications in the side chain, a few studies have reported the biological profile of A-ring diastereomers (Norman, A. W. et al. (1993) J. Biol. Chem. 268 (27): 20022-20030). Furthermore, biological esterification of steroids has been studied (Hochberg, R. B., (1998) Endocr. Rev. 19(3): 331-348), and esters of vitamin D₃ are known (WO 97/11053).

Moreover, despite much effort in developing synthetic analogues, clinical applications of vitamin D and its structural analogues have been limited by the undesired side effects elicited by these compounds after administration to a subject for known indications/applications of vitamin D compounds.

The activated form of vitamin D, vitamin D₃, and some of its analogues have been described as potent regulators of cell growth and differentiation. It has previously been found that vitamin D₃ as well as an analogue (analogue V), inhibited BPH cell proliferation and counteracted the mitogenic activity of potent growth factors for BPH cells, such as keratinocyte growth factor (KGF) and insulin-like growth factor (IGF1). Moreover, the analogue induced bcl-2 protein expression, intracellular calcium mobilization, and apoptosis in both unstimulated and KGF-stimulated BPH cells.

As described in the Examples herein, the inventors have found that vitamin D compounds such as calcitriol and analogues of calcitriol (“Compound X” and “Compound Y” in the Examples) can prevent adhesions in animal models, such as mice and rabbit models.

Without being limited by theory, it is believed that vitamin D compounds exert their beneficial effect through a modulation of the fibrinolytic pathway in conjunction with having an antiinflammatory effect. As demonstrated by the Examples, the beneficial effect may be achieved without adverse impact on wound healing. In theory, once a fibrous adhesion is formed, it is not expected that an anti-fibrotic agent would be active in elimination the adhesion, without compromising the healing process. Nevertheless, vitamin D compounds have been found to be effective. Moreover unlike certain prior art methods, there does not seem to be any adverse impact on mortality due to potentiation of infection.

Thus, in one aspect, the invention provides the use of a vitamin D compound in the prevention of adhesions. Also provided is a method for preventing adhesions in a subject by administering an effective amount of a vitamin D compound. Further provided is the use of a vitamin D compound in the manufacture of a medicament for the prevention of adhesions. Further provided is a vitamin D compound for use in the prevention of adhesions. Also provided is a kit containing a vitamin D compound together with instructions directing administration of said compound to a patient in need of the prevention of adhesions thereby to prevent adhesions in said patient.

In one aspect, the invention provides a method of prevention of adhesions using a vitamin D compound.

In another aspect, the invention provides a method for preventing adhesions in a subject, comprising administering to a subject in need thereof an effective amount of a vitamin D compound, such that adhesions are prevented in the subject.

In one embodiment, the invention provides a method as described above, further comprising identifying a subject in need of prevention of adhesions. In another embodiment, the invention provides a method as described above, further comprising the step of obtaining the vitamin D compound. In one embodiment of the methods described herein, the subject is a mammal. In a further embodiment, the subject is a human.

In another embodiment, the invention provides a method as described herein wherein the vitamin D compound is formulated in a pharmaceutical composition together with a pharmaceutically acceptable diluent or carrier.

In another aspect, the invention provides a use of a vitamin D compound in the manufacture of a medicament for the prevention of adhesions.

In another aspect, the invention provides a pharmaceutical formulation comprising a vitamin D compound and a pharmaceutically acceptable carrier for use in the prevention of adhesions.

In yet another aspect, the invention provides a pharmaceutical formulation comprising a vitamin D compound and a pharmaceutically acceptable carrier packaged with instructions for use in the prevention of adhesions.

In another aspect, the invention provides a vitamin D compound for use in the prevention of adhesions.

The invention provides for a kit containing a vitamin D compound together with instructions directing administration of said compound to a patient in need of the prevention of adhesions thereby to prevent adhesions in said patient.

In one embodiment, the invention provides for the method formulation, compound or kit, wherein the vitamin D compound is administered separately, sequentially or simultaneously in separate or combined pharmaceutical formulations with a second medicament for the prevention and/or treatment of adhesions. In another embodiment, the invention provides for the method formulation, compound or kit, wherein said vitamin D compound is calcitriol, Compound X or Compound Y as defined below.

### BRIEF DESCRIPTION OF THE DRAWINGS

FIG. 1 shows a comparison of adhesion scores following the administration of Compound X or vehicle.

FIG. 2 shows a comparison of adhesion scores following the administration of Compound Y or vehicle.

FIG. 3 shows (upper figure) a comparison of adhesion scores following the administration of Compound X, Compound Y, calcitriol or vehicle and (lower figure) the corresponding adhesion lengths.

FIG. 4 shows blood serum calcium levels for animals of the experiment following the administration of Compound X, Compound Y, calcitriol or vehicle.

FIG. 5 shows blood serum calcium levels for animals of the experiment following the administration of Compound Y at different doses: the upper trace shows the results following i.p. administration (single dose) and the lower trace shows the results following i.p. administration (single dose) together with oral administration for 3 days at MTD (3 ug/kg).

FIG. 6 shows a comparion of adhesion scores following following the administration of leuprolide acetate (positive control) or vehicle.

FIG. 7 shows the in vitro effect of Compound Y on the fibrinolysis pathway as evidenced by lysis of fibrin clots by mouse fibroblasts 3T3.L1 conditioned supernatants.

FIG. 8 shows the in vitro effect of Compound Y on fibrinolysis pathway as evidenced by the tPA/PAI ratio in human mesothelial cells.

FIG. 9 shows the effect of intraperitoneal single administration of Compound Y to mice on serum calcium levels.

FIG. 10 shows a dose response efficacy study of Compound Y in a mouse model of post surgical adhesion (CPSS).

FIG. 11 shows a dose response efficacy study of Compound Y in a rabbit model of post surgical adhesion (DUH).

FIG. 12 shows the effect of intraperitoneal single administration of Compound Y to DUH rabbits on serum calcium levels.

FIG. 13 shows the effect of Compound Y in a mouse model of tissue healing.

FIG. 14 shows the effect of Compound Y on VEGF and TGF-B levels in mice

FIG. 15 shows the effect of Compound Y in a mouse model of mortality due to infection potentiation.

FIG. 16 shows the efficacy of Compound Y as compared with icodextrin in the DUH rabbit model

FIG. 17 shows the efficacy of Compound Y as compared with lazaroids in the DUH rabbit model

### DETAILED DESCRIPTION OF THE INVENTION

### 1. Definitions

Before further description of the present invention, and in order that the invention may be more readily understood, certain terms are first defined and collected here for convenience.

By “adhesions” is meant unwanted fibrous connections (specifically connections formed of a persistent fibrin matrix) between tissues, for example tissues of the peritoneum, thorax, heart, spine, joints, eye, nose and the like. Such adhesions may result from a number of causative circumstances such as surgical procedures or underlying disease conditions. In particular, circumstances in which adhesions may form include gynecological surgery and caesarean delivery. It will be recognised that a number of underlying chronic conditions such as inflammatory bowel disease, Crohn's disease, endometriosis and ulcerative cholitis may sometimes give rise to the formation of adhesions, such underlying chronic conditions are not themselves included within the term “adhesions”. In one embodiment the subject to which the vitamin D compound is administered for the prevention of adhesions is not suffering from endometriosis. In a second embodiment, the subject to which the vitamin D compound is administered for the prevention of adhesions is not suffering from inflammatory bowel disease, Crohn's disease, endometriosis or ulcerative cholitis.

By “prevention” or “prophylaxis” when used herein in respect of the prevention or prophylaxis of adhesions is meant a reduction in the number of adhesions formed and/or the size/severity of adhesions formed. As adhesion formation involves a range of molecular components and progresses through a number of stages, it will be understood that “prevention” or “prophylaxis” encompasses both the administration of vitamin D compounds before adhesion formation begins and also at any stage where the vitamin D compound acts to reverse the process of adhesion formation. Prevention or “prophylaxis” does not extend to the treatment of pre-existing adhesions or to the administration of a vitamin D compound at a stage in adhesion formation where the vitamin D compound is unable to reverse the process of adhesion formation.

By “peritoneal adhesions” it is meant adhesions of the peritoneum, for example abdominal and pelvic adhesions, typically as a result of surgery, ischemic injury, inflammation, bacterial and chemical peritonitis, radiotherapy or foreign body reaction.

By “post-surgical adhesions” is meant adhesions formed between tissues subsequent to surgery, including (but not limited to) traditional surgery and laparoscopy, for example following cholecystectomies, appendectomies, colon surgery, heart surgery, lung surgery and pelvic surgery.

Those skilled in the art will recognise that the vitamin D compounds may be used in human or veterinary medicine. Thus, in accordance with the invention, the terms “subject” and “patient” are used interchangeably, and are intended to include mammals, for example, humans. It is preferred that the vitamin D compound be used in the prevention of adhesions in human patients.

The term “administration” or “administering” includes all routes of introducing the vitamin D compound(s) to a subject to perform their intended function. Examples of routes of administration which can be used include injection (subcutaneous, intravenous, parenterally, intraperitoneally), or administration by oral, inhalation, rectal or transdermal routes or via instillation e.g. bladder or intra-peritoneal instillation. The pharmaceutical preparations are, of course, given by forms suitable for each administration route. For example, these preparations are administered in tablets or capsule form, by injection, infusion, inhalation, lotion, ointment, suppository, etc. Oral administration or administration directly to the peritoneum (i.e. i.p. route) is preferred. The injection can be bolus or can be continuous infusion. Depending on the route of administration, the vitamin D compound can be coated with or disposed in a selected material to protect it from natural conditions which may detrimentally affect its ability to perform its intended function. The vitamin D compound can be administered alone, or in conjunction with either another agent useful in the prevention and/or treatment of adhesions (for example anti-inflammatory agents such as corticosteroids and fibrinolytic agents such as tissue plasminogen activator), or with a pharmaceutically-acceptable carrier, or both. The vitamin D compound can be administered prior to the administration of the other agent, simultaneously with the agent, or after the administration of the agent. Furthermore, the vitamin D compound can also be administered in a pro-form which is converted into its active metabolite, or more active metabolite in vivo.

The term “effective amount” includes an amount effective, at dosages and for periods of time necessary, to achieve the desired result, i.e. sufficient to prevent adhesions. An effective amount of vitamin D compound may vary according to factors such as the causative background (i.e. underlying disease state or particular surgical procedure involved), age and weight of the subject, and the ability of the vitamin D compound to elicit a desired response in the subject. Dosage regimens may be adjusted to provide the optimum prophylactic response. An effective amount is also one in which any toxic or detrimental effects (e.g., side effects) of the vitamin D compound are outweighed by the prophylactically beneficial effects.

A prophylactically effective amount of vitamin D compound (i.e., an effective dosage) may range from about 0.001 to 30 ug/kg body weight, preferably about 0.01 to 25 ug/kg body weight, more preferably about 0.1 to 20 ug/kg body weight, and even more preferably about 1 to 10 ug/kg, 2 to 9 ug/kg, 3 to 8 ug/kg, 4 to 7 ug/kg, or 5 to 6 ug/kg body weight per day. Higher concentrations eg up to 600 ug/kg may also be tolerated. The skilled artisan will appreciate that certain factors may influence the dosage required to effectively prevent adhesions in a subject, including but not limited to the severity of the disease or disorder, previous treatments, the general health and/or age of the subject, and other diseases present. In addition, the dose administered will also depend on the particular vitamin D compound used, the effective amount of each compounds can be determined by titration methods known in the art. Moreover, prevention of adhesion formation in a subject with a prophylactically effective amount of a vitamin D compound can include a single administration or, preferably, can include a series of administrations. In one example, a subject is administered a vitamin D compound in the range of between about 0.1 to 20 ug/kg body weight, one time per day for one or two weeks, for example in the case of elective surgery starting just before and concluding shortly after surgery.

An “on-off” or intermittent administration regime can also be considered. It will be appreciated that the effective dosage of a vitamin D compound used for the prevention of adhesions may increase or decrease over the course of a particular period of administration.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can optionally further include (for example, in one embodiment alkyl groups do not include) oxygen, nitrogen, sulfur or phosphorus atoms replacing one or more carbons of the hydrocarbon backbone, e.g., oxygen, nitrogen, sulfur or phosphorus atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer, especially 6 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above.

An “alkylaryl” moiety is an alkyl substituted with an aryl (e.g., phenylmethyl (benzyl)). Unsubstituted alkyl (including cycloalkyl) groups or groups substituted by halogen, especially fluorine, are generally preferred over other substituted groups. The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, propyl (n-propyl and i-propyl), butyl (tert-butyl, n-butyl and sec-butyl), pentyl, hexyl, heptyl, octyl and so forth. In preferred embodiment, the term “lower alkyl” includes a straight chain alkyl having 4 or fewer carbon atoms in its backbone, e.g., C₁-C₄ alkyl.

Thus specific examples of alkyl include C₁₋₆ alkyl or C₁₋₄alkyl (such as methyl or ethyl). Specific examples of hydroxyalkyl include C₁₋₆hydroxyalkyl or C₁₋₄hydroalkyl (such as hydroxymethyl).

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like.

Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The terms “alkenyl” and “alkynyl” refer to unsaturated aliphatic groups analogueous in length and possible substitution to the alkyls described above, but that contain at least one double or triple bond, respectively. For example, the invention contemplates cyano and propargyl groups.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

As used herein, the term “halogen” designates —F, —Cl, —Br or —I; the term “sulfhydryl” or “thiol” means —SH; the term “hydroxyl” means —OH.

The term “haloalkyl” is intended to include alkyl groups as defined above that are mono-, di- or polysubstituted by halogen, e.g., C₁₋₆haloalkyl or C₁₋₄haloalkyl such as fluoromethyl and trifluoromethyl.

The term “heteroatom” as used herein means an atom of any element other than carbon or hydrogen. Preferred heteroatoms are nitrogen, oxygen, sulfur and phosphorus.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “isomers” or “stereoisomers” refers to compounds which have identical chemical constitution, but differ with regard to the arrangement of the atoms or groups in space.

The terms “isolated” or “substantially purified” are used interchangeably herein and refer to vitamin D₃ compounds in a non-naturally occurring state. The compounds can be substantially free of cellular material or culture medium when naturally produced, or chemical precursors or other chemicals when chemically synthesized. In one embodiment of the invention an isolated vitamin D compound is at least 75% pure, especially at least 85% pure, in particular at least 95% pure and preferably at least 99% pure on a w/w basis, said purity being by reference to compounds with which the vitamin D compound is naturally associated or else chemically associated in the course of chemical synthesis.

In certain preferred embodiments, the terms “isolated” or “substantially purified” also refer to preparations of a chiral compound which substantially lack one of the enantiomers; i.e., enantiomerically enriched or non-racemic preparations of a molecule.

Similarly, the terms “isolated epimers” or “isolated diastereomers” refer to preparations of chiral compounds which are substantially free of other stereochemical forms. For instance, isolated or substantially purified vitamin D₃ compounds include synthetic or natural preparations of a vitamin D₃ enriched for the stereoisomers having a substituent attached to the chiral carbon at position 3 of the A-ring in an alpha-configuration, and thus substantially lacking other isomers having a beta-configuration. Unless otherwise specified, such terms refer to vitamin D₃ compositions in which the ratio of alpha to beta forms is greater than 1:1 by weight. For instance, an isolated preparation of an a epimer means a preparation having greater than 50% by weight of the alpha-epimer relative to the beta stereoisomer, more preferably at least 75% by weight, and even more preferably at least 85% by weight. Of course the enrichment can be much greater than 85%, providing “substantially epimer-enriched” preparations, i.e., preparations of a compound which have greater than 90% of the alpha-epimer relative to the beta stereoisomer, and even more preferably greater than 95%. The term “substantially free of the beta stereoisomer” will be understood to have similar purity ranges.

As used herein, the term “vitamin D compound” includes any compound being an analogue of vitamin D that is capable of preventing adhesions. Generally, compounds which are ligands for the Vitamin D receptor (VDR ligands) and which are capable of preventing adhesions are considered to be within the scope of the invention. Vitamin D compounds are preferably agonists of the vitamin D receptor. Thus, vitamin D compounds are intended to include secosteroids. Examples of specific vitamin D compounds suitable for use in the methods of the present invention are further described herein. A vitamin D compound includes vitamin D₂ compounds, vitamin D₃ compounds, isomers thereof, or derivatives/analogues thereof. Preferred vitamin D compounds are vitamin D₃ compounds which are ligands of (more preferably are agonists of) the vitamin D receptor. Preferably the vitamin D compound (e.g., the vitamin D₃ compound) is a more potent agonist of the vitamin D receptor than the native ligand (i.e., the vitamin D, e.g., vitamin D₃). Vitamin D₁ compounds, vitamin D₂ compounds and vitamin D₃ compounds include, respectively, vitamin D₁, D₂, D₃ and analogues thereof. In certain embodiments, the vitamin D compound may be a steroid, such as a secosteroid, e.g., calciol, calcidiol or calcitriol. Non-limiting examples of certain preferred vitamin D compounds in accordance with the invention include those described in U.S. Pat. No. 6,492,353 and published international applications WO 2005/030222.

As used herein, the term “obtaining” includes purchasing, synthesizing, isolating or otherwise acquiring one or more of the vitamin D compounds used in practicing the invention.

The term “secosteroid” is art-recognized and includes compounds in which one of the cyclopentanoperhydro-phenanthrene rings of the steroid ring structure is broken. For example, 1-alpha,25(OH)₂D₃ and analogues thereof are hormonally active secosteroids. In the case of vitamin D₃, the 9-10 carbon-carbon bond of the B-ring is broken, generating a seco-B-steroid. The official IUPAC name for vitamin D₃ is 9,10-secocholesta-5,7,10(19)-trien-3B-ol. For convenience, a 6-s-trans conformer of 1-alpha,25(OH)₂D₃ is illustrated herein having all carbon atoms numbered using standard steroid notation.

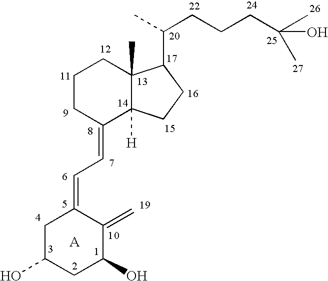

In the formulas presented herein, the various substituents on ring A are illustrated as joined to the steroid nucleus by one of these notations: a dotted line [symbol] indicating a substituent which is in the beta-orientation (i.e., above the plane of the ring), a wedged solid line [symbol] indicating a substituent which is in the alpha-orientation (i.e., below the plane of the molecule), or a wavy line [symbol] indicating that a substituent may be either above or below the plane of the ring. In regard to ring A, it should be understood that the stereochemical convention in the vitamin D field is opposite from the general chemical field, wherein a dotted line indicates a substituent on Ring A which is in an alpha-orientation (i.e., below the plane of the molecule), and a wedged solid line indicates a substituent on ring A which is in the beta-orientation (i.e. above the plane of the ring).

Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. Regardless, both configurations, cis/trans and/or Z/E are contemplated for the compounds for use in the present invention.

As shown, the A ring of the hormone 1-alpha,25(OH)₂D₃ contains two asymmetric centers at carbons 1 and 3, each one containing a hydroxyl group in well-characterized configurations, namely the 1-alpha- and 3-beta-hydroxyl groups. In other words, carbons 1 and 3 of the A ring are said to be “chiral carbons” or “carbon centers.”

With respect to the nomenclature of a chiral center, terms “d” and “l” configuration are as defined by the IUPAC Recommendations. As to the use of the terms, diastereomer, racemate, epimer and enantiomer will be used in their normal context to describe the stereochemistry of preparations.

Also, throughout the patent literature, the A ring of a vitamin D compound is often depicted in generic formulae as any one of the following structures:

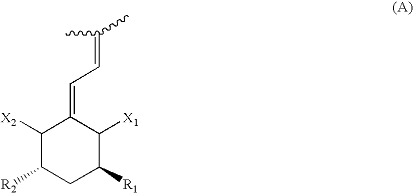

wherein X₁ and X₂ are defined as H or ═CH₂; or

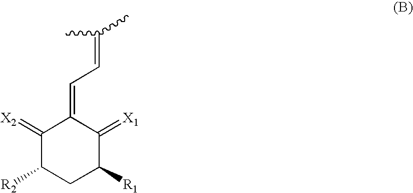

wherein X₁ and X₂ are defined as H₂ or CH₂.

Although there does not appear to be any set convention, it is clear that one of ordinary skill in the art understands either formula (A) or (B) to represent an A ring in which, for example, X₁ is ═CH₂ and X₂ is defined as H₂, as follows:

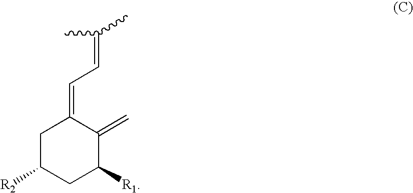

For purposes of the instant invention, formula (B) will be used in all generic structures.

In one embodiment of the invention, the vitamin D compound is a compound of formula (I):

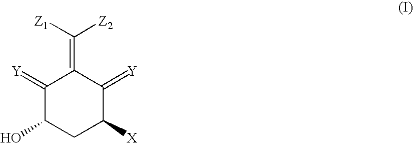

wherein:

X is hydroxyl or fluoro;

Y is H₂ or CH₂;

Z₁ and Z₂ are H or a substituent represented by formula (II), provided Z₁ and Z₂ are different (preferably Z₁ and Z₂ do not both represent formula (II)):

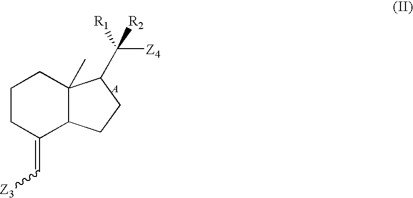

wherein:

Z₃ represents the above-described formula (I);

A is a single bond or a double bond;
R₁, R₂, and Z₄, are each, independently, hydrogen, alkyl, or a saturated or unsaturated carbon chain represented by formula (III), provided that at least one of R₁, R₂, and Z₄ is the saturated or unsaturated carbon chain represented by formula (III) and provided that all of R₁, R₂, and Z₄ are not saturated or unsaturated carbon chain represented by formula (III):

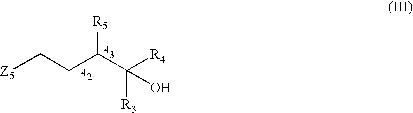

wherein:

Z₅ represents the above-described formula (II);

A₂ is a single bond, a double bond, or a triple bond; and

A₃ is a single bond or a double bond; and

R₃, and R₄, are each, independently, hydrogen, alkyl, haloalkyl, hydroxyalkyl; and R₅ is H₂ or oxygen. R₅ may also represent hydrogen or may be absent.

Thus, in the above structure of formula (III) (and in corresponding structures below), when A₂ represents a triple bond R₅ is absent. When A₂ represents a double bond R₅ represents hydrogen. When A₂ represents a single bond R₅ represents a carbonyl group or two hydrogen atoms.

In another embodiment of the invention, the vitamin D compound is a compound of formula (IV):

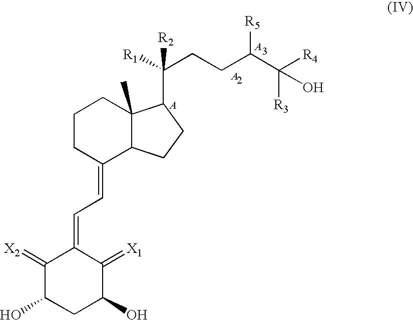

wherein:

X₁ and X₂ are H₂ or CH₂, wherein X₁ and X₂ are not CH₂ at the same time;

A is a single or double bond;

A₂ is a single, double or triple bond;

A₃ is a single or double bond;

R₁ and R₂ are hydrogen, C₁-C₄ alkyl or 4-hydroxy-4-methylpentyl, wherein R₁ and R₂ are not both hydrogen;

R₅ is H₂ or oxygen, R₅ may also represent hydrogen or may be absent;

R₃ is C₁-C₄ alkyl, hydroxyalkyl or haloalkyl, eg., fluoroalkyl, e.g., fluoromethyl and trifluoromethyl; and

R₄ is C₁-C₄ alkyl, hydroxyalkyl or haloalkyl, eg., fluoroalkyl, e.g., fluoromethyl and trifluoromethyl.

In yet another embodiment of the invention, the vitamin D compound is a compound of formula (V):

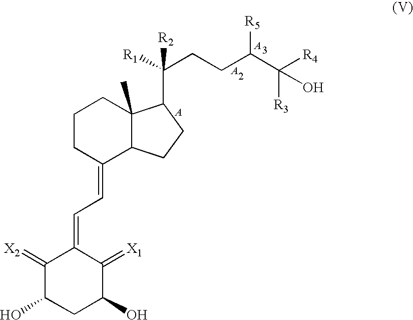

wherein:

X₁ and X₂ are H₂ or CH₂, wherein X₁ and X₂ are not CH₂ at the same time;

A is a single or double bond;

A₂ is a single, double or triple bond;

A₃ is a single or double bond;

R₁ and R₂ are hydrogen, C₁-C₄ alkyl, wherein R₁ and R₂ are not both hydrogen;

R₅ is H₂ or oxygen, R₅ may also represent hydrogen or may be absent;

R₃ is C₁-C₄ alkyl, hydroxyalkyl or haloalkyl, e.g., fluoroalkyl, e.g., fluoromethyl and trifluoromethyl; and

R₄ is C₁-C₄ alkyl, hydroxyalkyl haloalkyl, e.g., or fluoroalkyl, e.g., fluoromethyl and trifluoromethyl.

An example of the above structure of formula (V) is 1,25-dihydroxy-16-ene-23-yne cholecalciferol.

In yet another embodiment, the vitamin D compound is a “geminal” compound of formula (VI):

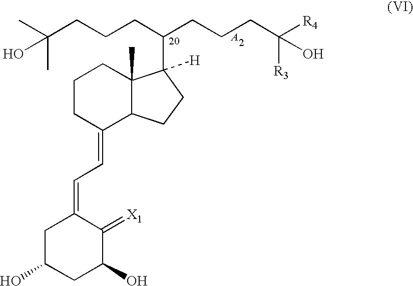

wherein:

X₁ is H₂ or CH₂;

A₂ is a single, a double or a triple bond;

R₃ is C₁-C₄ alkyl, hydroxyalkyl, or haloalkyl, e.g., fluoroalkyl, e.g., fluoromethyl and trifluoromethyl;

R₄ is C₁-C₄ alkyl, hydroxyalkyl or haloalkyl, e.g., fluoroalkyl, e.g., fluoromethyl and trifluoromethyl;

and the configuration at C₂₀ is R or S.

Compounds of this type may be referred to as “geminal” or “gemini” vitamin D₃ compounds due to the presence of two alkyl chains at C20.

An example geminal compound of formula (VI) is 1,25-dihydroxy-21-(3-hydroxy-3-methylbutyl)-19-nor-cholecalciferol (elsewherein herein referred to as “Compound Y”):

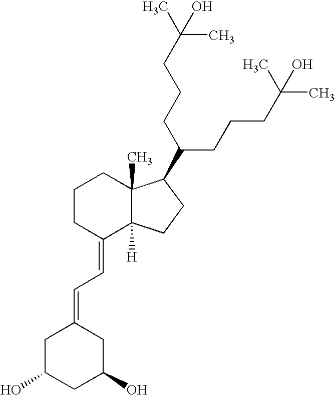

The synthesis of 1,25-dihydroxy-21-(3-hydroxy-3-methylbutyl)-19-nor-cholecalciferol is described in WO 98/49138 and U.S. Pat. No. 6,030,962, the disclosures of which are incorporated herein by reference. The synthesis is described below in Example 2.

In another embodiment, the vitamin D compound is a compound of formula (VII):

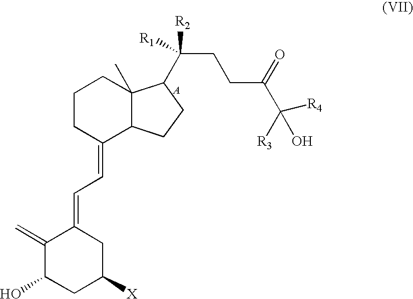

wherein:

A is a single or double bond;

R₁ and R₂ are each, independently, hydrogen, alkyl (for example methyl);

R₃, and R₄, are each, independently, alkyl, and

X is hydroxyl or fluoro.

In a further embodiment, the vitamin D compound is a compound having formula (VIII):

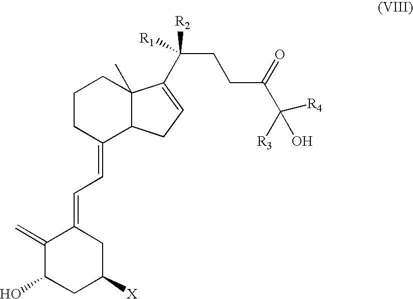

wherein:

R₁ and R₂, are each, independently, hydrogen, or alkyl, e.g., methyl;

R₃ is alkyl, e.g., methyl,

R₄ is alkyl, e.g., methyl; and

X is hydroxyl or fluoro.

In specific embodiments of the invention, the vitamin D compound is selected from the group consisting of:

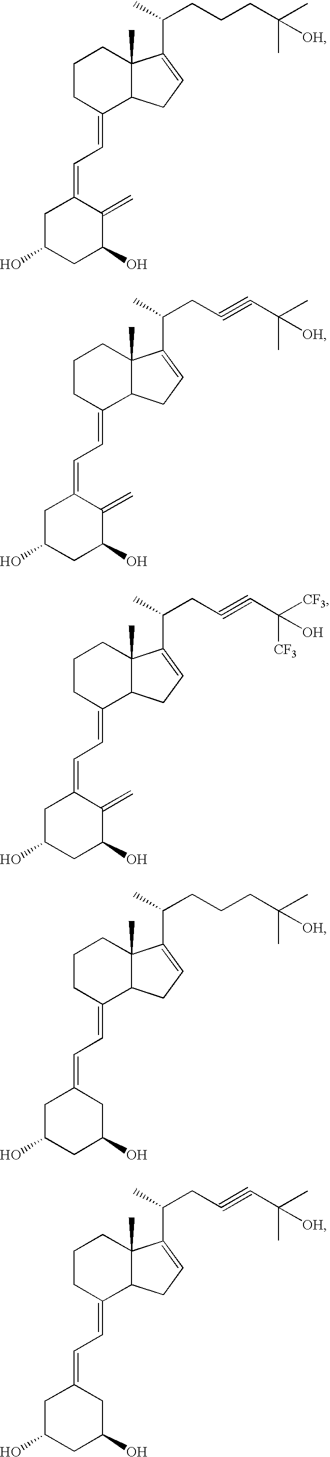

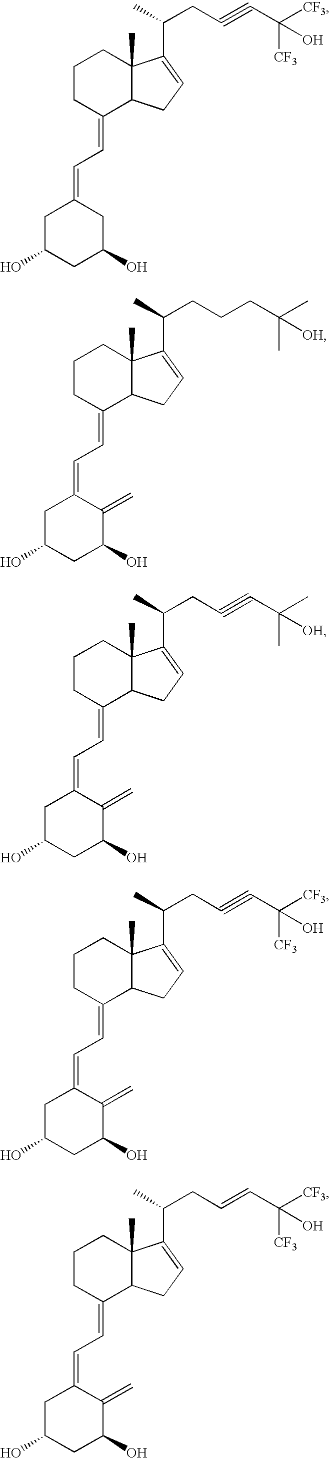

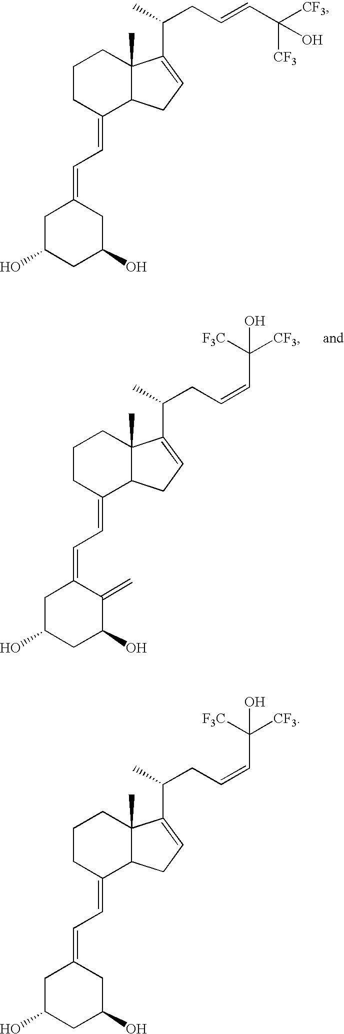

In other specific embodiments of the invention, the vitamin D compound is selected from the group consisting of:

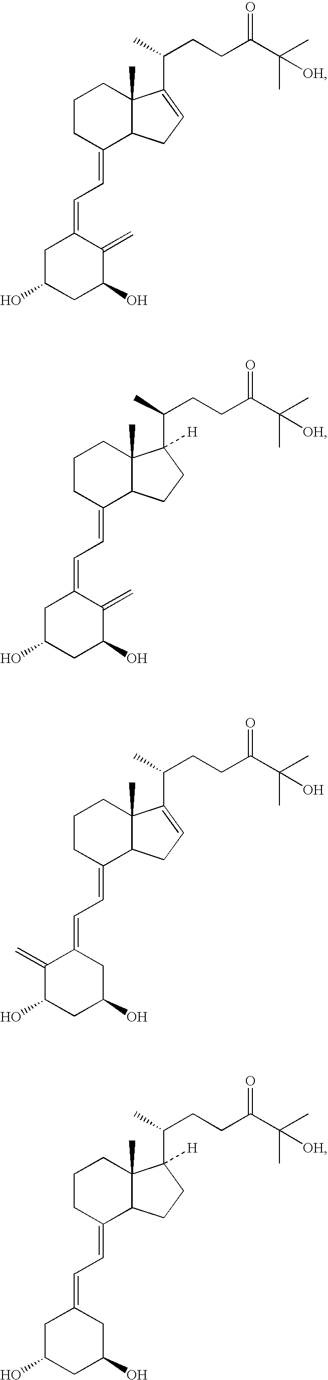

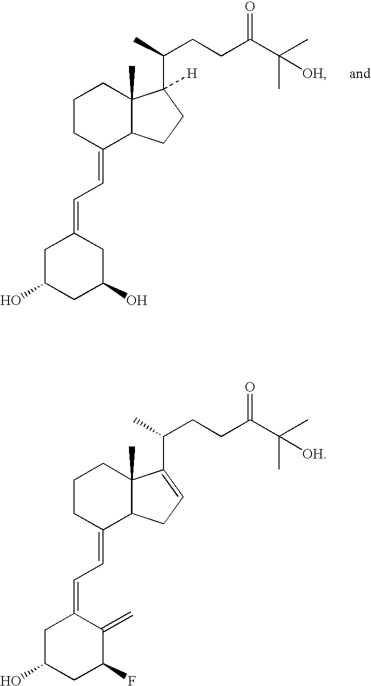

In further specific embodiments of the invention, the vitamin D compound is selected from the group of geminal compounds consisting of:

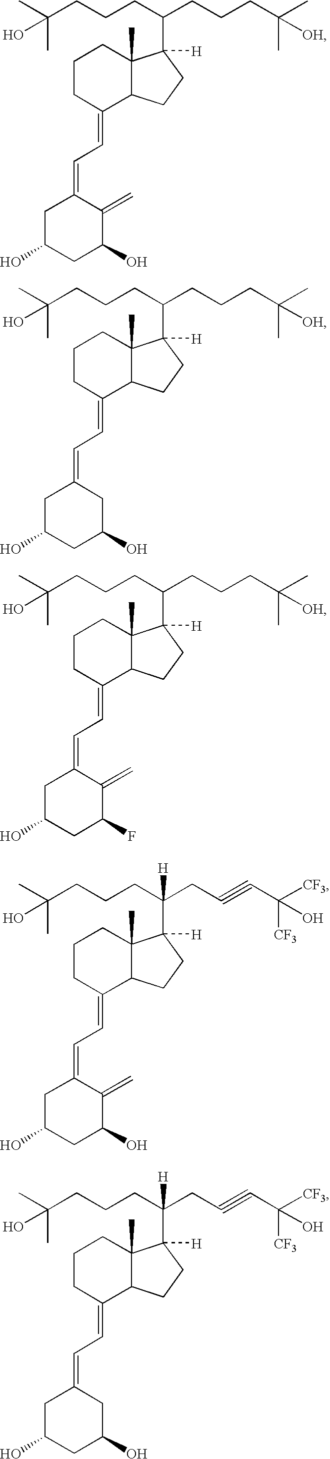

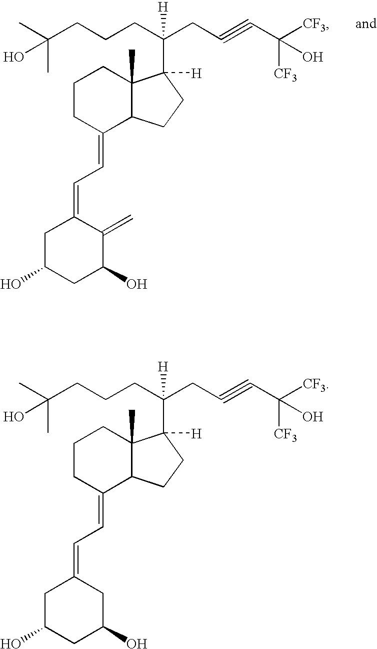

In yet another aspect, the invention provides Gemini vitamin D₃ compounds of formula (IX):

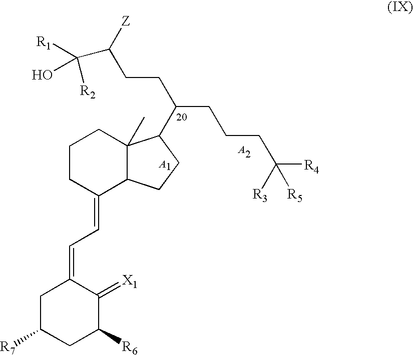

wherein:

A₁ is a single or double bond;

A₂ is a single, a double or a triple bond;

R₁, R₂, R₃ and R₄ are each independently C₁-C₄ alkyl, C₁-C₄ deuteroalkyl, hydroxyalkyl, or haloalkyl;

R₅, R₆ and R₇ are each independently hydroxyl, OC(O)C₁-C₄ alkyl, OC(O)hydroxyalkyl, or OC(O)haloalkyl;

the configuration at C₂₀ is R or S;

X₁ is H₂ or CH₂;

Z is hydrogen when at least one of R₁ and R₂ is C₁-C₄ deuteroalkyl and at least one of R₃ and R₄ is haloalkyl or when at least one of R₁ and R₂ is haloalkyl and at least one of R₃ and R₄ is C₁-C₄ deuteroalkyl; or Z is —OH, ═O, —SH, or —NH₂;

and pharmaceutically acceptable esters, salts, and prodrugs thereof.

Various embodiments of this aspect of the invention include individual compounds of formula I wherein: A₁ is a single bond; A₂ is a single bond; A₂ is a triple bond; R₁, R₂, R₃, and R₄ are each independently methyl or ethyl; R₁, R₂, R₃, and R₄ are each independently C₁-C₄ deuteroalkyl or haloalkyl; R₅ is hydroxyl; R₆ and R₇ are hydroxyl; R₆ and R₇ are each OC(O)C₁-C₄ alkyl; X₁ is H₂; X₁ is CH₂; Z is hydrogen; or Z is ═O.

In certain embodiments, R₅, R₆ and R₇ are hydroxyl. In other embodiments, R₆ and R₇ are each acetyloxy.

In yet other embodiments, Z is hydrogen when at least one of R₁ and R₂ is C₁-C₄ deuteroalkyl and at least one of R₃ and R₄ is haloalkyl or when at least one of R₁ and R₂ is haloalkyl and at least one of R₃ and R₄ is Cᵢ⁻C₄ deuteroalkyl; Z is

—OH, ═O, —SH, or —NH₂ when X₁ is CH₂; Z is —OH, ═O, —SH, or —NH₂ when X₁ is H₂ and the configuration at C₂₀ is S; or Z is ═O, —SH, or —NH₂ when X₁ is H₂ and the configuration at C₂₀ is R. In one embodiment, Z is —OH.

Still other embodiments of this aspect of invention include those wherein X₁ is CH₂; A₂ is a single bond; R₁, R₂, R₃, and R₄ are each independently methyl or ethyl; and Z is —OH. In one embodiment, X₁ is CH₂; A₂ is a single bond; R₁, R₂, R₃, and R₄ are each independently methyl or ethyl; and Z is ═O. In one embodiment, X₁ is H₂; A₂ is a single bond; R₁, R₂, R₃, and R₄ are each independently methyl or ethyl; the configuration at C₂₀ is S; and Z is —OH. In another embodiment, X₁ is H₂; A₂ is a single bond; R₁, R₂, R₃, and R₄ are each independently methyl or ethyl; and Z is ═O. In these embodiments, R₁, R₂, R₃, and R₄ are advantageously each methyl.

In certain embodiments, the haloalkyl is fluoroalkyl. Advantageously, fluoroalkyl is fluoromethyl or trifluoromethyl.

Additional embodiments of this aspect of the invention include compounds X₁ is H₂; A₂ is a triple bond; R₁ and R₂ are each C₁-C₄ deuteroalkyl; R₃ and R₄ are each haloalkyl; and Z is hydrogen. In other embodiments, X₁ is CH₂; A₂ is a triple bond; R₁ and R₂ are each C₁-C₄ deuteroalkyl; R₃ and R₄ are each haloalkyl; and Z is hydrogen.

In these embodiments, R₁ and R₂ are advantageously each deuteromethyl and R₃ and R₄ are advantageously each trifluoromethyl.

Specific compounds of the invention include: 1,25-Dihydroxy-21-(2R,3-dihydroxy-3-methyl-butyl)-20R-cholecalciferol:

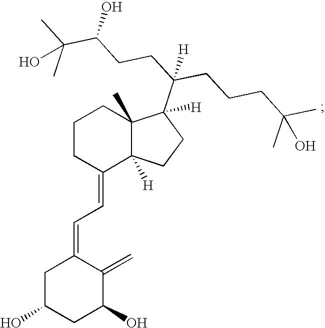

### 1,25-Dihydroxy-21-(2R,3-dihydroxy-3-methyl-butyl)-20S-cholecalciferol

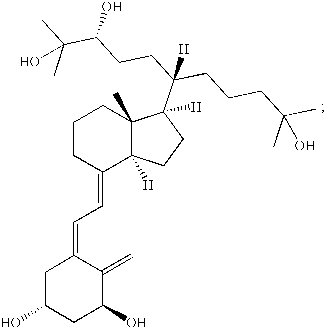

### 1,25-Dihydroxy-20S-21-(3-hydroxy-3-methyl-butyl)-24-keto-19-nor-cholecalciferol

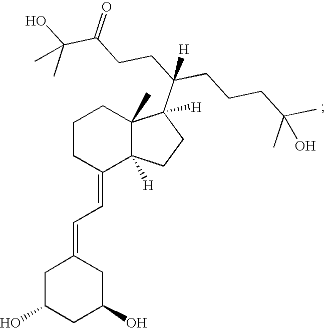

### and

### 1,25-Dihydroxy-21(3-hydroxy-3-trifluoromethyl-4-trifluoro-butynyl)-26,27-hexadeutero-20S-cholecalciferol

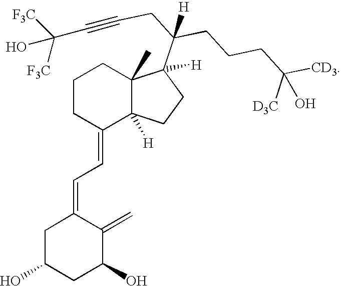

In still further specific embodiments of the invention, the vitamin D compound is a geminal compound of formula (X):

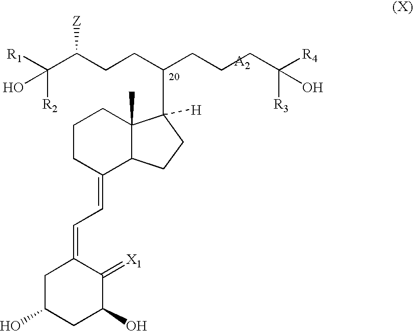

wherein:

X₁ is H₂ or CH₂;

A₂ is a single, a double or a triple bond;

R₁, R₂, R₃ and R₄ are each independently C₁-C₄ alkyl, hydroxyalkyl, or haloalkyl, e.g., fluoroalkyl, e.g., fluoromethyl and trifluoromethyl;

Z is —OH, Z may also be ═O, —NH₂ or —SH; and

the configuration at C₂₀ is R or S,

and pharmaceutically acceptable esters, salts, and prodrugs thereof.

In a further embodiment, X₁ is CH₂. In another embodiment, A₂ is a single bond. In another, R₁, R₂, R₃, and R₄ are each independently methyl or ethyl. In a further embodiment, Z is —OH. In another, X₁ is CH₂; A₂ is a single bond; R₁, R₂, R₃, and R₄ are each independently methyl or ethyl; and Z is —OH. In an even further embodiment, R₁, R₂, R₃, and R₄ are each methyl.

In a further embodiment of the invention, the vitamin D compound is a geminal compound of the formula:

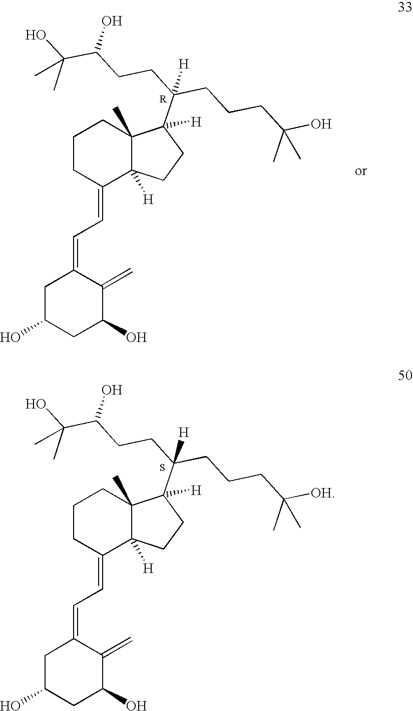

The chemical names of compounds 33 and 50 mentioned above are 1,25-dihydroxy-21-(2R,3-dihydroxy-3-methyl-butyl)-20R-cholecalciferol and 1,25-dihydroxy-21-(2R,3-dihydroxy-3-methyl-butyl)-20S-cholecalciferol respectively.

Additional embodiments of geminal compounds include the following vitamin D compounds for use in accordance with the invention:

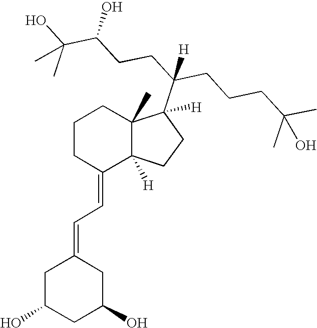

(1,25-Dihydroxy-21-(2R,3-dihydroxy-3-methyl-butyl)-20S-19-nor-cholecalciferol)

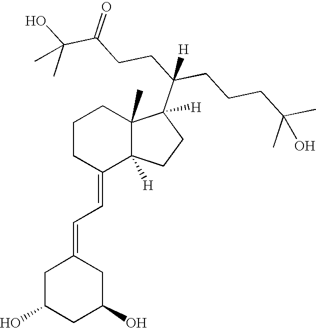

(1,25-Dihydroxy-20S-21-(3-hydroxy-3-methyl-butyl)-24-keto-19-nor-cholecalciferol)

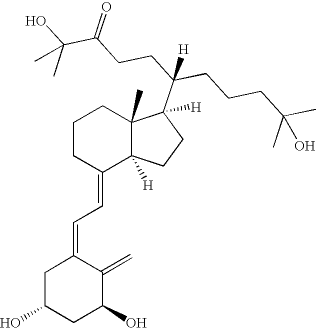

(1,25-Dihydroxy-20S-21-(3-hydroxy-3-methyl-butyl)-24-keto-cholecalciferol)

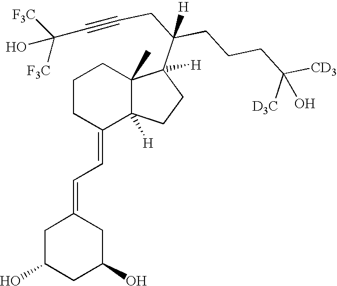

(1,25-Dihydroxy-21(3-hydroxy-3-trifluoromethyl-4-trifluoro-butynyl)-26,27-hexadeutero-19-nor-20S-cholecalciferol)

and

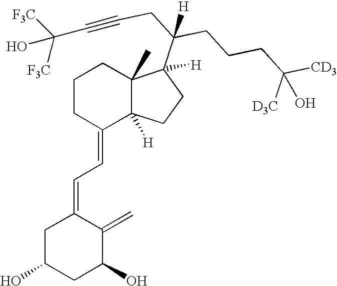

(1,25-Dihydroxy-21(3-hydroxy-3-trifluoromethyl-4-trifluoro-butynyl)-26,27-hexadeutero-20S-cholecalciferol).

In further embodiments of the invention, the vitamin D compound is a compound of formula (XI):

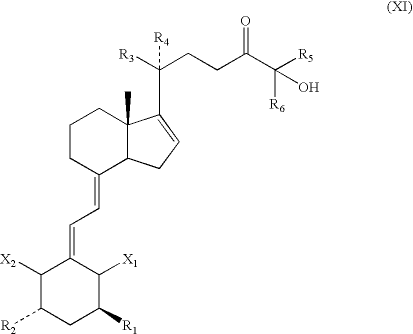

wherein:

X₁ and X₁ are each independently H₂ or ═CH₂, provided X₁ and X₁ are not both ═CH₂;

R₁ and R₂ are each independently, hydroxyl, OC(O)C₁-C₄ alkyl, OC(O)hydroxyalkyl, OC(O)fluororalkyl;

R₃ and R₄ are each independently hydrogen, C₁-C₄ alkyl, hydroxyalkyl or haloalkyl, or R₃ and R₄ taken together with C₂₀ form C₃-C₆ cylcoalkyl; and

R₅ and R₆ are each independently C₁-C₄ alkyl or haloalkyl;

and pharmaceutically acceptable esters, salts, and prodrugs thereof.

Suitably R₃ and R₄ are each independently hydrogen, C₁-C₄ alkyl, or R₃ and R₄ taken together with C₂₀ form C₃-C₆ cylcoalkyl.

In one example set of compounds R₅ and R₆ are each independently C₁-C₄ alkyl.

In another example set of compounds R₅ and R₆ are each independently haloalkyl e.g., C₁-C₄ fluoroalkyl.

When R₃ and R₄ are taken together with C20 to form C₃-C₆ cycloalkyl, an example is cyclopropyl.

In one embodiment, X₁ and X₁ are each H₂. In another embodiment, R₃ is hydrogen and R₄ is C₁-C₄ alkyl. In a preferred embodiment R₄ is methyl.

In another embodiment, R₅ and R₆ are each independently methyl, ethyl, fluoromethyl or trifluoromethyl. In a preferred embodiment, R₅ and R₆ are each methyl.

In yet another embodiment, R₁ and R₁ are each independently hydroxyl or OC(O)C₁-C₄ alkyl.

In a preferred embodiment, R₁ and R₁ are each OC(O)C₁-C₄ alkyl. In another preferred embodiment, R₁ and R₁ are each acetyloxy.

An example of such a compound is 1,3-O-diacetyl-1,25-dihydroxy-16-ene-24-keto-19-nor-cholecalciferol, having the following structure:

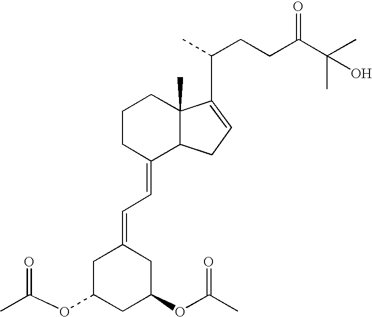

In another embodiment of the invention the vitamin D compound for use in accordance with the invention is 2-methylene-19-nor-20(S)-1-alpha,25-hydroxyvitamin D₃:

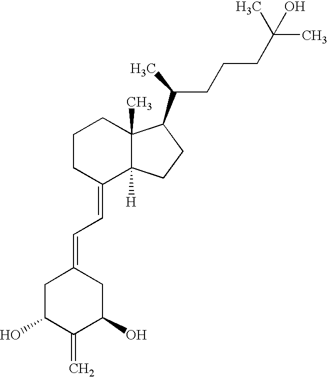

The synthesis of this and related compounds is described in WO02/05823 and U.S. Pat. No. 5,536,713 which are herein incorporated in their entirety by reference.

In another embodiment of the invention, the vitamin D compound is a compound of the formula (XII):

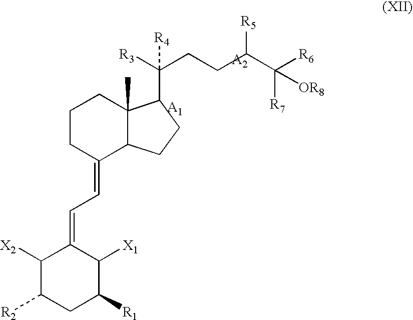

wherein:

A₁ is single or double bond;

A₂ is a single, double or triple bond;

X₁ and X₂ are each independently H or ═CH₂, provided X₁ and X₂ are not both ═CH₂;

R₁ and R₂ are each independently H, OC(O)C₁-C₄ alkyl (for example OAc), OC(O)hydroxyalkyl, OC(O)haloalkyl; such as OC(O)C₁-C₄ alkyl (for example OAc), OC(O)hydroxyalkyl;

R₃, R₄ and R₅ are each independently hydrogen, C₁-C₄ alkyl, hydroxyalkyl, or haloalkyl, or R₃ and R₄ taken together with C₂₀ form C₃-C₆ cycloalkyl; and

R₆ and R₇ are each independently C₁₋₄alkyl or haloalkyl; and

R₈ is H, —COC₁-C₄alkyl (e.g. Ac), —COhydroxyalkyl or —COhaloalkyl; and

pharmaceutically acceptable esters, salts, and prodrugs thereof.

When R₃ and R₄ are taken together with C₂₀ to form C₃-C₆ cycloalkyl an example is cyclopropyl.

Suitably R₆ and R₇ are each independently haloalkyl. R₈ may suitably represent H or Ac.

In one embodiment, A₁ is a single bond and A₂ is a single bond, E or Z double bond, or a triple bond, for example A₁ is a single bond and A₂ is a single bond. In another embodiment, A₁ is a double bond and A₂ is a single bond, E or Z double bond, or a triple bond. One of ordinary skill in the art will readily appreciate that when A₂ is a triple bond, R₅ is absent

In one embodiment, X₁ and X₂ are each H. In another embodiment, X₁ is CH₂ and X₂ is H₂. In another embodiment, R₃ is hydrogen and R₄ is C₁-C₄ alkyl. In a preferred embodiment R₄ is methyl.

In another embodiment R₃ and R₄ taken together with C₂₀ form C₃-C₆ cycloalkyl eg cyclopropyl.

In another example set of compounds R₁ and R₂ are OH or OC(O)C₁-C₄ alkyl, for example R₁ and R₂ both represent OAc.

In one set of example compounds R₆ and R₇ are each independently C₁₋₄alkyl. In another set of example compounds R₆ and R₇ are each independently haloalkyl. In another embodiment, R₆ and R₇ are each independently methyl, ethyl or fluoroalkyl, for example they are both methyl. In a preferred embodiment, R₆ and R₈ are each trifluoroalkyl, e.g., trifluoromethyl.

Suitably R₅ represents hydrogen.

Suitably R₈ represents hydrogen.

In another embodiment, R₁ and R₂ are OH or OC(O)C₁-C₄ alkyl, X₁ is ═CH₂ and X₂ is H, A₁ is single bond, A₂ is a single bond, R₃ and R₄ taken together with C₂₀ form C₃-C₆ cycloalkyl, R₅ is hydrogen, R₆ and R₇ are each independently C₁₋₄alkyl, and R₈ is H. In yet another embodiment, the invention provides for the method formulation, compound or kit, wherein R₁ and R₂ are OH or OAc, R₃ and R₄ taken together with C₂₀ form cyclopropyl, and R₆ and R₇ are each methyl.

Thus, in certain embodiments, vitamin D compounds for use in accordance with the invention are represented by formula (XII):

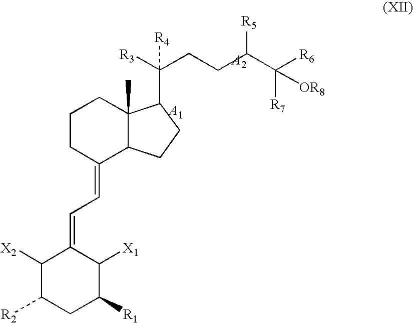

wherein:

A₁ is single or double bond;

A₂ is a single, double or triple bond;

X₁ and X₂ are each independently H or ═CH₂, provided X₁ and X₂ are not both ═CH₂;

R₁ and R₂ are each independently OC(O)C₁-C₄ alkyl, OC(O)hydroxyalkyl, or OC(O)haloalkyl;

R₃, R₄ and R₅ are each independently hydrogen, C₁-C₄ alkyl, hydroxyalkyl, or haloalkyl, with the understanding that R₅ is absent when A₂ is a triple bond, or R₃ and R₄ taken together with C₂₀ form C₃-C₆ cycloalkyl;

R₆ and R₇ are each independently alkyl or haloalkyl; and

R₈ is H, C(O)C₁-C₄ alkyl, C(O)hydroxyalkyl, or C(O)haloalkyl; and

pharmaceutically acceptable esters, salts, and prodrugs thereof.

In preferred embodiments, when A₁ is single bond, R₃ is hydrogen, and R₄ is methyl, then A2 is a double or triple bond.

An example compound of the above-described formula (XII) which is one of the preferred compounds in the context of the present invention is 1,3-di-O-acetyl-1,25-dihydroxy-16,23Z-diene-26,27-hexafluoro-19-nor-cholecalciferol:

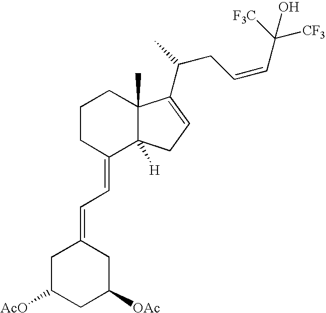

In another preferred embodiment the compound is one of formula (XIII), wherein R₁ and R₂ are each OAc; A₁ is a double bond; A₂ is a triple bond; and R₈ is either H or Ac:

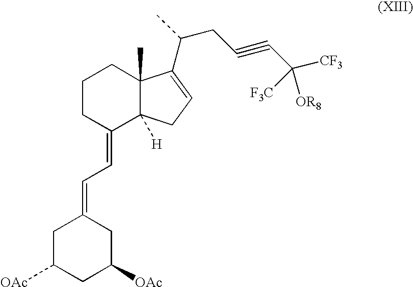

In certain embodiments of the above-represented formula (XII), vitamin D compounds for use in accordance with the invention are represented by the formula (XIV):

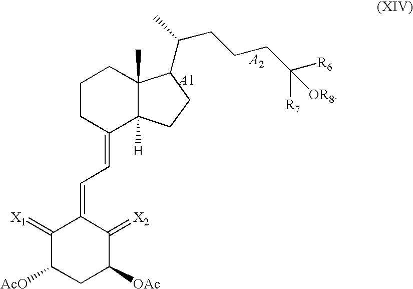

Other example compounds of the above-described formula (XIV) include:
1,3-di-O-acetyl-1,25-dihydroxy-23-yne-cholecalciferol;
1,3-di-O-acetyl-1,25-dihydroxy-16-ene-23-yne-cholecalciferol;
1,3-di-O-acetyl-1,25-dihydroxy-16,23E-diene-cholecalciferol;
1,3-di-O-acetyl-1,25-dihydroxy-16-ene-cholecalciferol;
1,3,25-Tri-O-acetyl-1,25-dihydroxy-16-ene-23-yne-26,27-hexafluoro-cholecalciferol:
1,3-di-O-acetyl-1,25-dihydroxy-16-ene-23-yne-26,27-hexafluoro-cholecalciferol;
1,3-Di-O-acetyl-1,25-dihydroxy-16,23E-diene-25R-26-trifluoro-cholecalciferol;
1,3-Di-O-acetyl-1,25-Dihydroxy-16-ene-23-yne-26,27-hexafluoro-19-nor-cholecalciferol;
1,3,25-Tri-O-acetyl-1,25-Dihydroxy-16-ene-23-yne-26,27-hexafluoro-19-nor-cholecalciferol;
1,3-di-O-acetyl-1,25-dihydroxy-16-ene-19-nor-cholecalciferol;
1,3-Di-O-acetyl-1,25-dihydroxy-16-ene-23-yne-19-nor-cholecalciferol;
1,3-Di-O-acetyl-1,25-dihydroxy-16-ene-23-yne-26,27-bishomo-19-nor-cholecalciferol;

In certain other embodiments of the above-represented formula (XII), the vitamin D compounds for use in accordance with the invention are represented by the formula (XV):

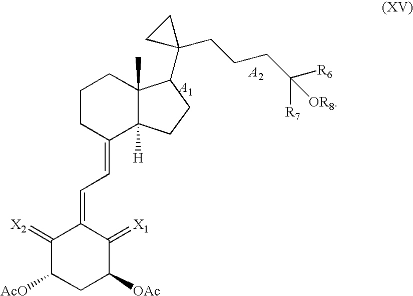

In a preferred embodiment, X₁ is ═CH₂ and X₂ is H₂. When A₁ is a single bond, and A₂ is a triple bond, it is preferred that R₈ is H or C(O)CH₃, and R₆ and R₇ are alkyl, preferably methyl. When A₁ is a single bond, and A₂ is a single bond, it is preferred that R₈ is H or C(O)CH₃, and R₆ and R₇ are alkyl, preferably methyl. When A₁ is a double bond, and A₂ is a single bond, it is preferable that R₈ is H or C(O)CH₃, and R₆ and R₇ are alkyl, preferably methyl.

In another preferred embodiment, X₁ and X₂ are each H₂. When A₁ is a single bond, and A₂ is a triple bond, it is preferred that R₈ is H or C(O)CH₃, and R₆ and R₇ are alkyl or haloalkyl. It is preferred that the alkyl group is methyl, and the haloalkyl group is trifluoroalkyl, preferably trifluoromethyl. When A₁ is a single bond, and A₂ is a double bond, it is preferred that R₈ is H or C(O)CH₃, R₆ and R₇ are haloalkyl, preferably trifluoroalkyl, preferably trifluoromethyl. When A₁ is a double bond, and A₂ is a single bond, it is preferred that R₈ is H or C(O)CH₃, R₆ and R₇ are alkyl, preferably methyl.

Other example compounds of the above-described formula (XV) include:
1,3-Di-O-acetyl-1,25-dihydroxy-20-cyclopropyl-23-yne-19-nor-cholecalciferol;
1,3,25-tri-O-acetyl-1,25-dihydroxy-20-cyclopropyl-23-yne-26,27-hexafluoro-19-nor-cholecalciferol;
1,3-di-O-acetyl-1,25-dihydroxy-20-cyclopropyl-23-yne-26,27-hexafluoro-19-nor-cholecalciferol;
1,3-di-O-acetyl-1,25-dihydroxy-20-cyclopropyl-23-yne-cholecalciferol;
1,3-di-O-acetyl-1,25-dihydroxy-20-cyclopropyl-23Z-ene-26,27-hexafluoro-19-nor-cholecalciferol;
1,3-di-O-acetyl-1,25-dihydroxy-20-cyclopropyl-cholecalciferol;
1,3-di-O-acetyl-1,25-dihydroxy-16-ene-20-cyclopropyl-19-nor-cholecalciferol; and
1,3-Di-O-acetyl-1,25-dihydroxy-16-ene-20-cyclopropyl-cholecalciferol.

A preferred compound of formula XV is 1,3-di-O-acetyl-1,25-dihydroxy-20-cyclopropyl-23E-ene-26,27-hexafluoro-19-nor-cholecalciferol:

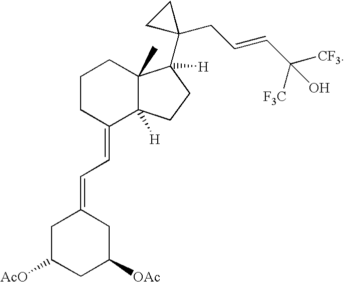

An example of a preferred compound is 1,3-Di-O-acetyl-1,25-dihydroxy-20-cyclopropyl-cholecalciferol (referred to as “Compound A”) having the formula:

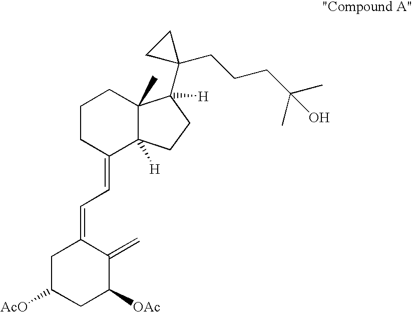

Such compounds are described in WO2005/030222, the contents of which are herein incorporated by reference in their entirety. The invention also embraces use of esters and salts of Compound A. Esters include pharmaceutically acceptable labile esters that may be hydrolysed in the body to release Compound A. Salts of Compound A include adducts and complexes that may be formed with alkali and alkaline earth metal ions and metal ion salts such as sodium, potassium and calcium ions and salts thereof such as calcium chloride, calcium malonate and the like. However, although Compound A may be administered as a pharmaceutically acceptable salt or ester thereof, preferably Compound A is employed as is i.e., it is not employed as an ester or a salt thereof.

Another compound is 1,25-dihydroxy-20,21,28-cyclopropyl-cholecalciferol having the formula:

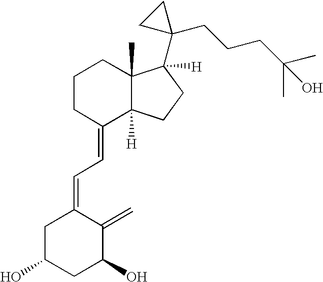

The compound is described in U.S. Pat. No. 6,492,353, the contents of which are herein incorporated by reference in their entirety.

The invention also embraces use of esters and salts of 1,25-dihydroxy-20,21,28-cyclopropyl-cholecalciferol. Esters include pharmaceutically acceptable labile esters that may be hydrolysed in the body to release 1,25-dihydroxy-20,21,28-cyclopropyl-cholecalciferol. Salts of 1,25-dihydroxy-20,21,28-cyclopropyl-cholecalciferol include adducts and complexes that may be formed with alkali and alkaline earth metal ions and metal ion salts such as sodium, potassium and calcium ions and salts thereof such as calcium chloride, calcium malonate and the like. However, although 1,25-dihydroxy-20,21,28-cyclopropyl-cholecalciferol may be administered as a pharmaceutically acceptable salt or ester thereof, preferably it is employed as is i.e., it is not employed as an ester or a salt thereof.

In a further embodiment, vitamin D compounds for use in the invention are compounds of the formula (XVI):

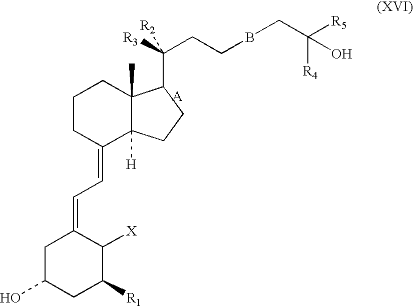

wherein:

X is H₂ or CH₂;

R₁ is hydrogen, hydroxy or fluorine;

R₂ is hydrogen or methyl;

R₃ is hydrogen or methyl provided that when R₂ or R₃ is methyl, R₃ or R₂ must be hydrogen;

R₄ is methyl, ethyl or trifluoromethyl;

R₅ is methyl, ethyl or trifluoromethyl;

A is a single or double bond;

B is a single, E-double, Z-double or triple bond.

In preferred compounds, each of R₄ and R₅ is methyl or ethyl, for example 1-alpha-fluoro-25-hydroxy-16,23E-diene-26,27-bishomo-20-epi-cholecalciferol, herein after referred to as “Compound X”, having the formula:

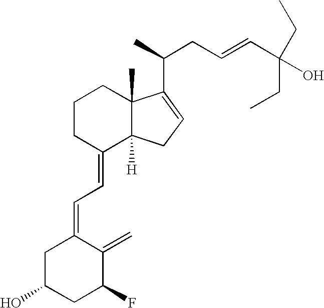

Such compounds and methods of synthesis are described in Radinov et al. J. Org. Chem. 2001, 66, 6141; Daniewski et al. U.S. Pat. No. 6,255,501; Batcho et al. U.S. Pat. No. 5,939,408, and EP808833, the contents of which are herein incorporated by reference in their entirety. An improved synthesis of 1-alpha-fluoro-25-hydroxy-16,23E-diene-26,27-bishomo-20-epi-cholecalciferol is described below in Example 1.

Other preferred vitamin D compounds for use in accordance with the invention include those having formula (XVII):

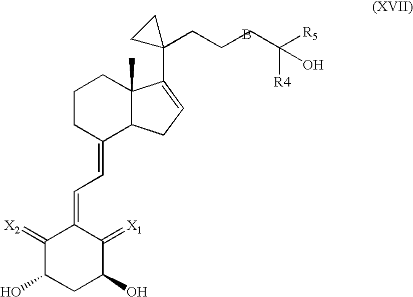

wherein:

B is single, double, or triple bond;

X₁ and X₂ are each independently H₂ or CH₂, provided X₁ and X₂ are not both CH₂; and

R₄ and R₅ are each independently alkyl or haloalkyl.

Examples of compounds of formula (XVII) include the following:

### 1,25-Dihydroxy-16-ene-23-yne-20-cyclopyl-cholecalciferol

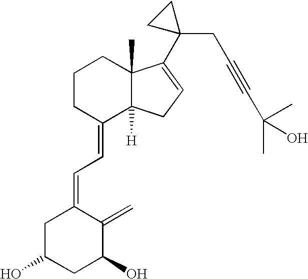

### 1,25-Dihydroxy-16-ene-23-yne-20-cyclopropyl-19-nor-cholecalciferol

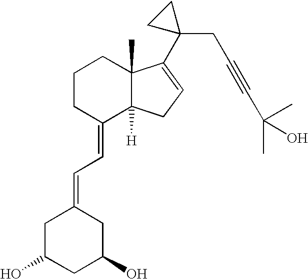

### 1,25-Dihydroxy-16-ene-20-cyclopropyl-23-yne-26,27-hexafluoro-19-nor-cholecalciferol

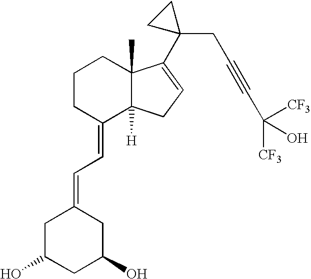

### 1,25-Dihydroxy-16-ene-20-cyclopropyl-23-yne-26,27-hexafluoro-cholecalciferol

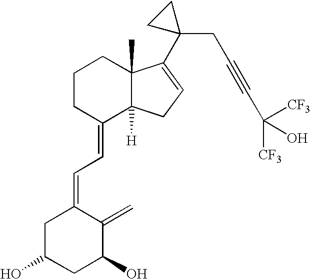

### 1,25-Dihydroxy-16,23E-diene-20-cyclopropyl-26,27-hexafluoro-19-nor-cholecalciferol

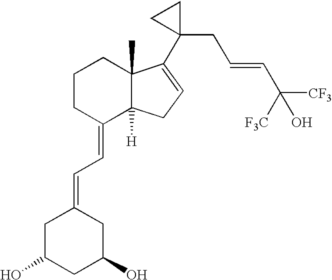

### 1,25-Dihydroxy-16,23E-diene-20-cyclopropyl-26,27-hexafluoro-cholecalciferol

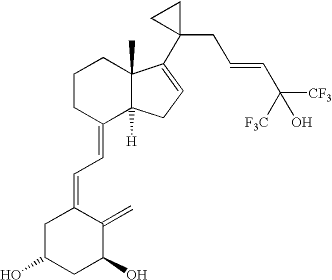

### 1,25-Dihydroxy-16,23Z-diene-20-cyclopropyl-26,27-hexafluoro-19-nor-cholecalciferol

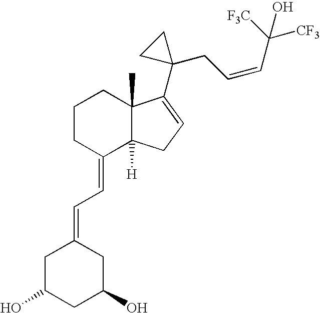

### 1,25-Dihydroxy-16,23Z-diene-20-cyclopropyl-26,27-hexafluoro-cholecalciferol

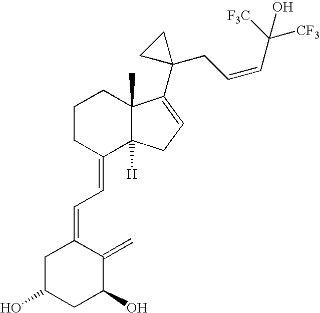

### 1,25-Dihydroxy-16-ene-20-cyclopropyl-19-nor-cholecalciferol

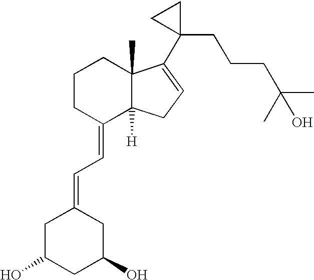

### 1,25-Dihydroxy-16-ene-20-cyclopropyl-cholecalciferol

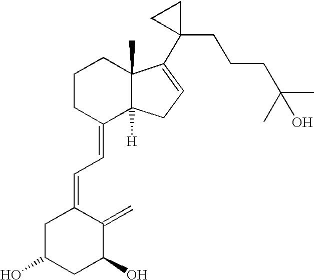

Another vitamin D compound of the invention is 1,25-dihydroxy-21(3-hydroxy-3-trifluoromethyl-4-trifluoro-butynyl)-26,27-hexadeutero-19-nor-20S-cholecalciferol.

Still other preferred vitamin D compounds for use in accordance with the invention include those having formula (XVIII):

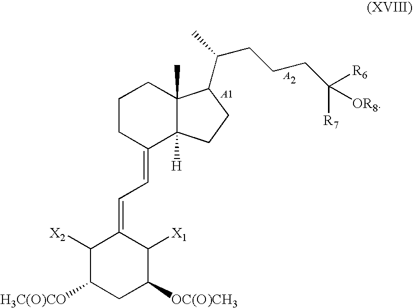

In a preferred embodiment, A₁ is a double bond, and X₁ is ═CH₂ and X₂ is H₂. When A₂ is a triple bond, it is preferred that R₈ is H or C(O)CH₃, and R₆ and R₇ are alkyl or haloalkyl. It is preferred that the alkyl group is methyl and the haloalkyl group is trifluoroalkyl, preferably trifluoromethyl. When A₂ is a double bond, it is preferred that R₈ is H or C(O)CH₃, and R₆ and R₇ are alkyl, preferably methyl. It is also preferred that R₆ and R₇ are independently alkyl and haloalkyl. When A₂ is a single bond, it is preferred that R₈ is H or C(O)CH₃, and R₆ and R₇ are alkyl, preferably methyl.

In a preferred embodiment, A₁ is a double bond, and X₁ and X₂ are each H₂. When A₂ is a triple bond, it is preferred that R₈ is H or C(O)CH₃, and R₆ and R₇ are alkyl or haloalkyl. It is preferred that the alkyl group is methyl or ethyl and the haloalkyl group is trifluoroalkyl, preferably trifluoromethyl. When A₂ is a double bond, it is preferred that R₈ is H or C(O)CH₃, and R₆ and R₇ are haloalkyl, preferably trifluoroalkyl, preferably trifluoromethyl. When A₂ is a single bond, it is preferred that R₈ is H or C(O)CH₃, and R₆ and R₇ are alkyl, preferably methyl.

In another embodiment of the invention of formula (XVIII), R₁ and R₂ are OC(O)CH₃, A₁ is a single bond, and A₂ is a single, double or triple bond, except that when R₃ is H and R₄ is methyl, A₂ is a double or triple bond. In a preferred embodiment, R₃ is H, R₄ is methyl, R₅ is absent, R₈ is H or C(O)CH₃, and R₆ and R₇ are alkyl, preferably methyl.

Preferred compounds of the present include the following: 1,3-Di-O-acetyl-1,25-dihydroxy-16,23Z-diene-26,27-hexafluoro-19-nor-cholecalciferol, 1,3-Di-O-acetyl-1,25-Dihydroxy-16-ene-23-yne-26,27-hexafluoro-19-nor-cholecalciferol, 1,3,25-Tri-O-acetyl-1,25-Dihydroxy-16-ene-23-yne-26,27-hexafluoro-19-nor-cholecalciferol, 1,3-Di-O-acetyl-1,25-dihydroxy-16-ene-23-yne-cholecalciferol, 1,3-Di-O-acetyl-1,25-dihydroxy-16,23E-diene-cholecalciferol, 1,3-Di-O-acetyl-1,25-dihydroxy-16-ene-cholecalciferol, 1,3,25-Tri-O-acetyl-1,25-dihydroxy-16-ene-23-yne-26,27-hexafluoro-cholecalciferol, 1,3-Di-O-acetyl-1,25-dihydroxy-16-ene-23-yne-26,27-hexafluoro-cholecalciferol, 1,3-Di-O-acetyl-1,25-dihydroxy-16,23E-diene-25R,26-trifluoro-cholecalciferol, 1,3-Di-O-acetyl-1,25-dihydroxy-16-ene-19-nor-cholecalciferol, 1,3-Di-O-Acetyl-1,25-dihydroxy-16-ene-23-yne-19-nor-cholecalciferol, 1,3-Di-O-acetyl-1,25-dihydroxy-16-ene-23-yne-26,27-bishomo-19-nor-cholecalciferol and 1,3-Di-O-acetyl-1,25-dihydroxy-23-yne-cholecalciferol.

These compounds can be prepared, e.g., as described in PCT Publication WO2005030222.

Yet further preferred vitamin D compounds for use in accordance with the invention include those having formula (XIX):

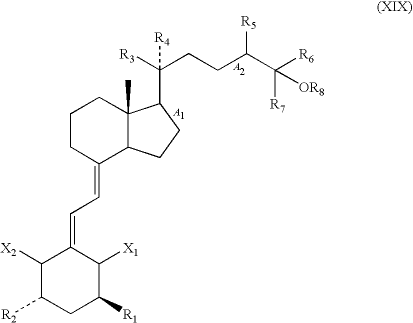

wherein:

A₁ is single or double bond;

A₂ is a single, double or triple bond,

X₁ and X₂ are each independently H₂ or CH₂, provided X₁ and X₂ are not both CH₂;

R₁ and R₂ are each independently OC(O)C₁-C₄ alkyl, OC(O)hydroxyalkyl, or OC(O)haloalkyl;

R₃, R₄ and R₅ are each independently hydrogen, C₁-C₄ alkyl, hydroxyalkyl, or haloalkyl, or R₃ and R₄ taken together with C₂₀ form C₃-C₆ cylcoalkyl;

R₆ and R₇ are each independently haloalkyl; and

R₈ is H, OC(O)C₁-C₄ alkyl, OC(O)hydroxyalkyl, or OC(O)haloalkyl; and

pharmaceutically acceptable esters, salts, and prodrugs thereof. In preferred embodiments, R₆ and R₇ are each independently trihaloalkyl, especially trifluoromethyl.

These compounds can be prepared, e.g., as described in PCT Publication WO2005030222, the contents of which are incorporated herein by reference.

Gemini 20-alkyl, e.g., methyl, vitamin D3 compounds are contemplated by the instant invention. In one aspect, the invention provides a vitamin D₃ compound having formula (XX):

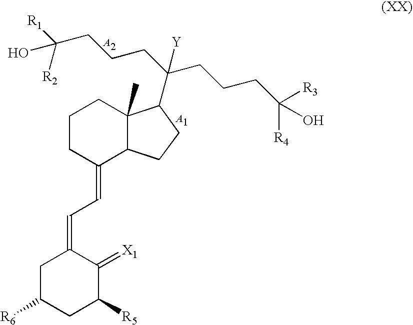

wherein:

A₁ is a single or double bond;

A₂ is a single, a double or a triple bond;

R₁, R₂, R₃ and R₄ are each independently alkyl, deuteroalkyl, hydroxyalkyl, or haloalkyl;

R₅ is halogen, hydroxyl, OC(O)alkyl, OC(O)hydroxyalkyl, or OC(O)haloalkyl;

R₆ is halogen, hydroxyl, OC(O)alkyl, OC(O)hydroxyalkyl, or OC(O)haloalkyl;

X₁ is H₂ or CH₂;

Y is alkyl;

and pharmaceutically acceptable esters, salts, and prodrugs thereof.

In one aspect, the invention provides a vitamin D₃ compound having formula (XX-a):

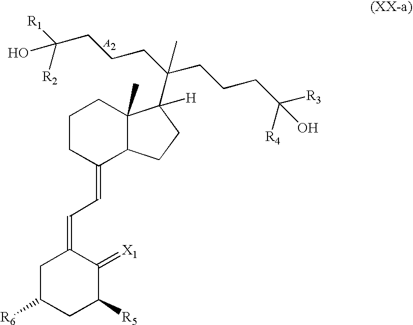

wherein:

A₂ is a single, a double or a triple bond;

R₁, R₂, R₃ and R₄ are each independently alkyl, hydroxyalkyl, or haloalkyl;

R₅ is halogen, hydroxyl, OC(O)alkyl, OC(O)hydroxyalkyl, or OC(O)haloalkyl;

R₆ is hydroxyl, OC(O)alkyl, OC(O)hydroxyalkyl, or OC(O)haloalkyl;

X₁ is H₂ or CH₂;

and pharmaceutically acceptable esters, salts, and prodrugs thereof.

In certain aspects, the invention provides a compound having formula (XX-b):

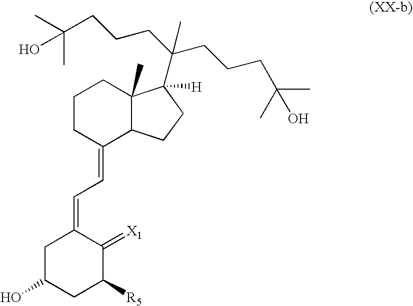

wherein:

R₅ is fluoro or hydroxyl;

X₁ is H₂ or CH₂;

and pharmaceutically acceptable esters, salts, and prodrugs thereof.

In other aspects, the invention provides a compound having formula (XX-c):

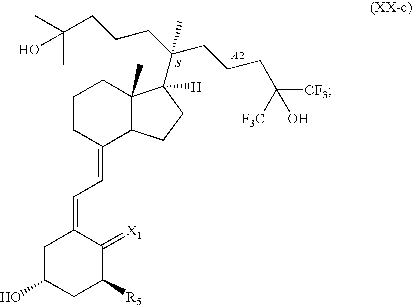

wherein:

A₂ is a single, a double or a triple bond;

R₅ is fluoro or hydroxyl;

X₁ is H₂ or CH₂;

and pharmaceutically acceptable esters, salts, and prodrugs thereof.

In another aspect, the invention provides a compound having formula (XX-d):

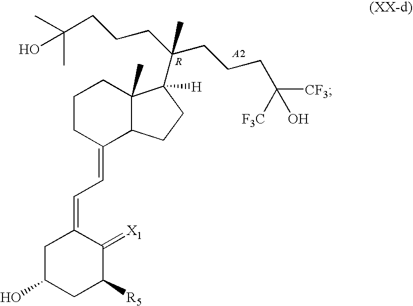

wherein:

A₂ is a single, a double or a triple bond;

R₅ is fluoro or hydroxyl;

X₁ is H₂ or CH₂;

and pharmaceutically acceptable esters, salts, and prodrugs thereof.

In yet another aspect, the invention provides a compound having formula (XX-e):

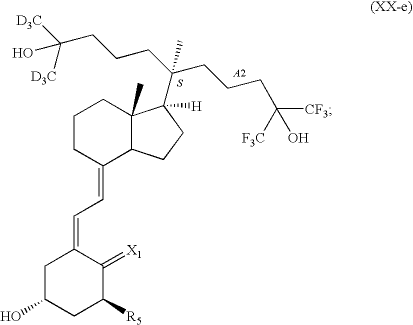

wherein:

A₂ is a single, a double or a triple bond;

R₅ is fluoro or hydroxyl;

X₁ is H₂ or CH₂;

and pharmaceutically acceptable esters, salts, and prodrugs thereof.

In still another aspect, the invention provides a compound having formula (XX-f):

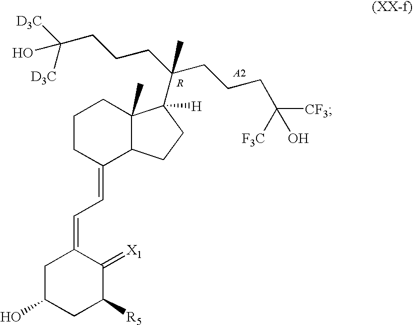

wherein:

A₂ is a single, a double or a triple bond;

R₅ is fluoro or hydroxyl;

X₁ is H₂ or CH₂;

and pharmaceutically acceptable esters, salts, and prodrugs thereof.

Preferred compounds of the invention include the following compounds, which are further exemplified in Chart 1. The syntheses of compounds of formula (XX) are included at Examples 3-41 below.

CHART 1

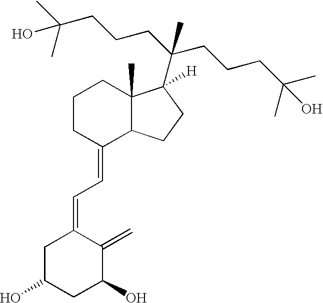

1,25-Dihydroxy-20-(4-hydroxy-4-methyl-
pentyl)-cholecalciferol

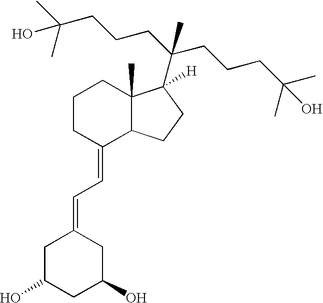

1,25-Dihydroxy-20-(4-hydroxy-4-methyl-
pentyl)-19-nor-cholecalciferol

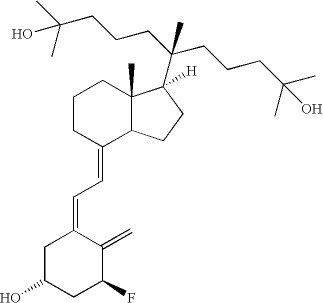

1α-Fluoro-25-hydroxy-20-(4-hydroxy-4-
methyl-pentyl)-cholecalciferol

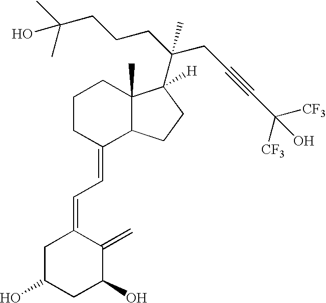

(20S)-1,25-Dihydroxy-20-(5,5,5-trifluoro-
4-hydroxy-4-trifluoromethyl-pent-2-ynyl)-
cholecalciferol

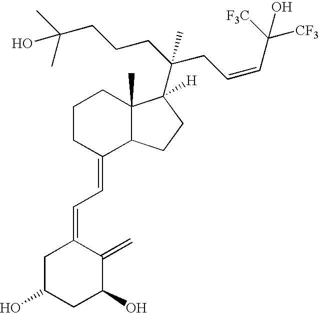

(20S)-1,25-Dihydroxy-20-((2Z)-5,5,5-
trifluoro-4-hydroxy-4-trifluoromethyl-
pent-2-enyl)cholecalciferol

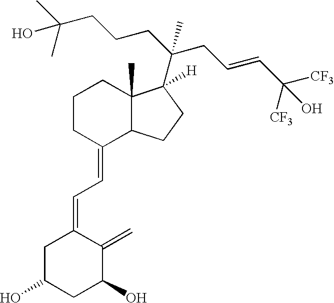

(20S)-1,25-Dihydroxy-20-[(2E)-5,5,5-
trifluoro-4-hydroxy-4-trifluoromethyl-
pent-2-enyl]-cholecalciferol

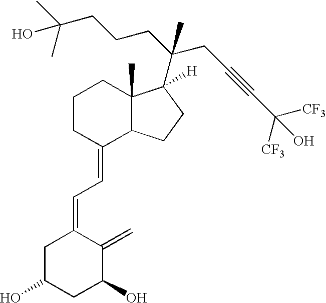

(20R)-1,25-Dihydroxy-20-(5,5,5-trifluoro-
4-hydroxy-4-trifluoromethyl-pent-2-ynyl)-
cholecalciferol

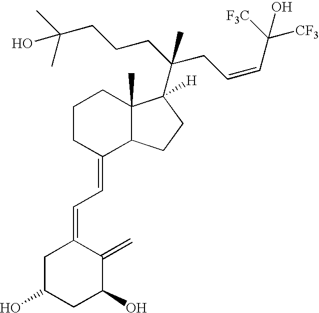

(20R)-1,25-Dihydroxy-20-[(2Z)-5,5,5-
trifluoro-4-hydroxy-4-trifluoromethyl-
pent-2-enyl]-cholecalciferol

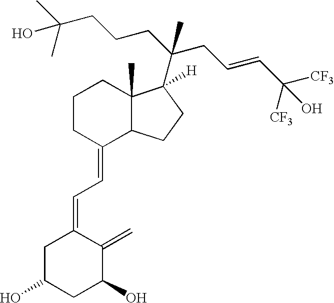

(20R)-1,25-Dihydroxy-20-[(2E)-5,5,5-
trifluoro-4-hydroxy-4-trifluoromethyl-
pent-2-enyl]-cholecalciferol

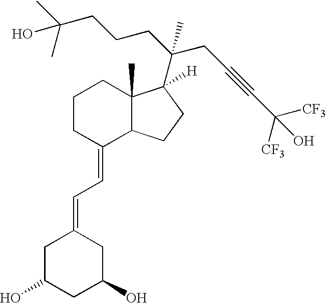

(20S)-1,25-Dihydroxy-20-(5,5,5-trifluoro-
4-hydroxy-4-trifluoromethyl-pent-2-ynyl)-
19-nor-cholecalciferol

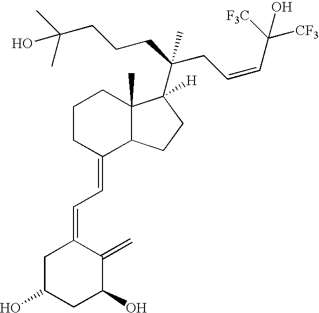

(20S)-1,25-Dihydroxy-20-[(2Z)-5,5,5-
trifluoro-4-hydroxy-4-trifluoromethyl-
pent-2-enyl]-19-nor-cholecalciferol

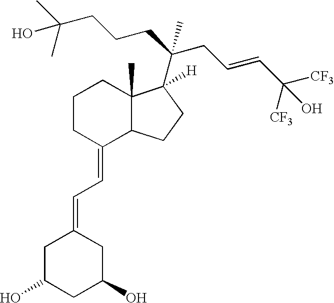

(20S)-1,25-Dihydroxy-20-[(2E)-5,5,5-
trifluoro-4-hydroxy-4-trifluoromethyl-
pent-2-enyl]-19-nor-cholecalciferol

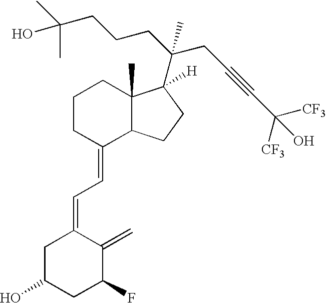

(20S)-1α-Fluoro-25-hydroxy-20-(5,5,5-
trifluoro-4-hydroxy-4-trifluoromethyl-
pent-2-ynyl)-cholecalciferol

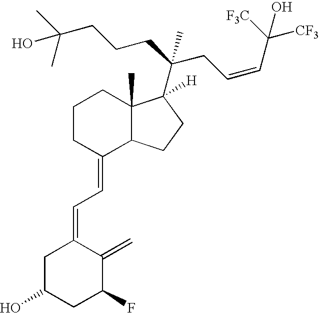

(20S)-1α-Fluoro-25-hydroxy-20-[(2Z)-
5,5,5-trifluoro-4-hydroxy-4-
trifluoromethyl-pent-2-enyl]-
cholecalciferol

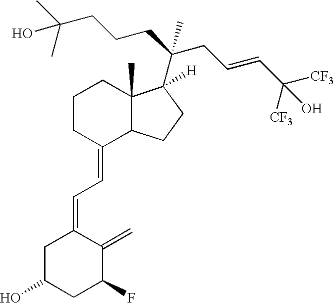

(20S)-1α-Fluoro-25-hydroxy-20-[(2E)-
5,5,5-trifluoro-4-hydroxy-4-trifluoro
methyl-pent-2-enyl]-cholecalciferol

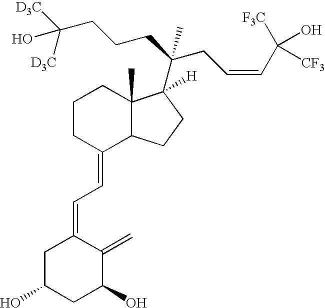

(20S)-1,25-Dihydroxy-20-((2Z)-5,5,5-
trifluoro-4-hydroxy-4-trifluoromethyl-
pent-2-enyl)-26,27-hexadeutero-
cholecalciferol

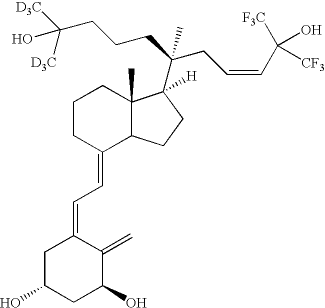

(20S)-1,25-Dihydroxy-20-((2Z)-5,5,5-
trifluoro-4-hydroxy-4-trifluoromethyl-
pent-2-enyl)-26,27-hexadeutero-19-nor-
cholecalciferol

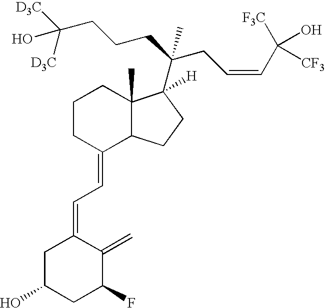

(20S)-1α-Fluoro-25-hydroxy-20-((2Z)-
5,5,5-trifluoro-4-hydroxy-4-
trifluoromethyl-pent-2-enyl)-26,27-
hexadeutero-cholecalciferol

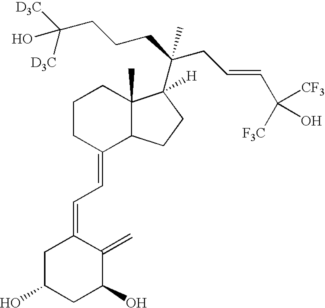

(20S)-1,25-Dihydroxy-20-((2E)-5,5,5-
trifluoro-4-hydroxy-4-trifluoromethyl-
pent-2-enyl)-26,27-hexadeutero-
cholecalciferol

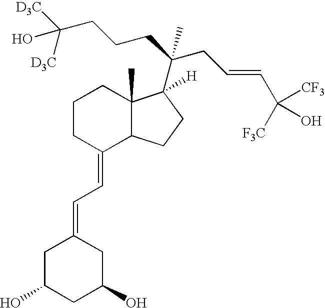

(20S)-1,25-Dihydroxy-20-((2E)-5,5,5-
trifluoro-4-hydroxy-4-trifluoromethyl-
pent-2-enyl)-26,27-hexadeutero-19-nor-
cholecalciferol

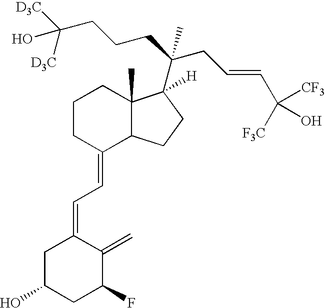

(20S)-1α-Fluoro-25-hydroxy-20-((2E)-
5,5,5-trifluoro-4-hydroxy-4-
trifluoromethyl-pent-2-enyl)-26,27-
hexadeutero-cholecalciferol

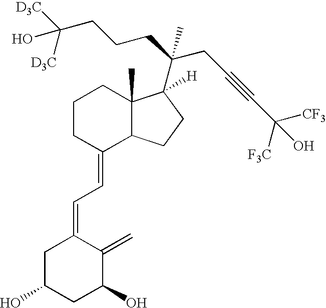

(20S)-1,25-Dihydroxy-20-(5,5,5-trifluoro-
4-hydroxy-4-trifluoromethyl-pent-2-ynyl)-
26,27-hexadeutero-cholecalciferol

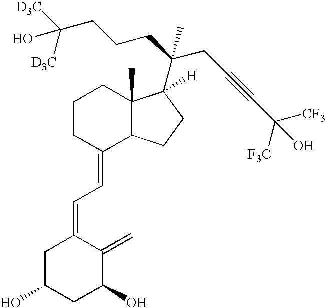

(20S)-1,25-Dihydroxy-20-(5,5,5-trifluoro-
4-hydroxy-4-trifluoromethyl-pent-2-ynyl)-
26,27-hexadeutero-19-nor-cholecalciferol

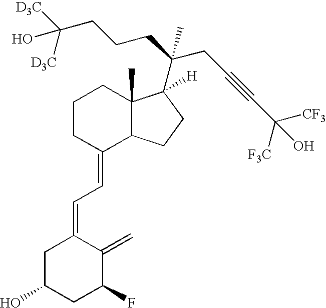

(20S)-1α-Fluoro-25-hydroxy-20-(5,5,5-
trifluoro-4-hydroxy-4-trifluoromethyl-
pent-2-ynyl)-26,27-hexadeutero-
cholecalciferol

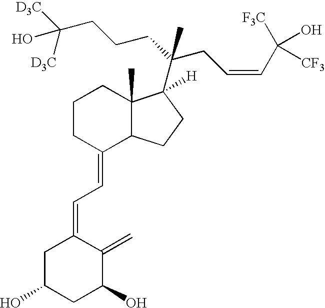

1,25-Dihydroxy-20R-20-(4-hydroxy-5,5,5-
trideutero-4-trideuteromethyl-pentyl)-23Z-
ene-26,27-hexafluorocholecalciferol

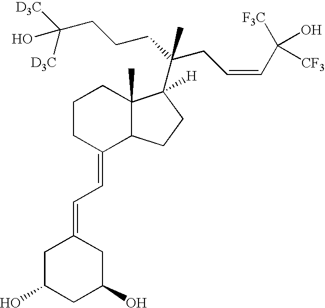

1,25-Dihydroxy-20R-20-(4-hydroxy-5,5,5-
trideutero-4-trideuteromethyl-pentyl)-23Z-
ene-26,27-hexafluoro-19-nor-
cholecalciferol

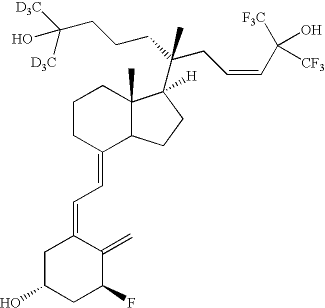

1α-Fluoro-25-hydroxy-20R-20-(4-
hydroxy-5,5,5-trideutero-4-
trideuteromethyl-pentyl)-23Z-ene-26,27-
hexafluorocholecalciferol

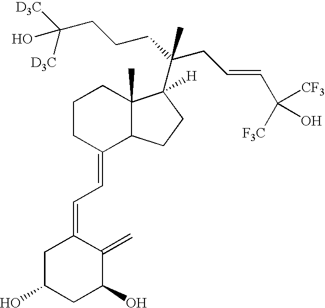

1,25-Dihydroxy-20R-20-(4-hydroxy-5,5,5-
trideutero-4-trideuteromethyl-pentyl)-23E-
ene-26,27-hexafluorocholecalciferol

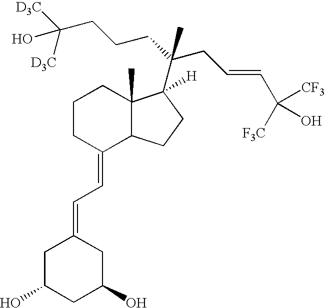

1,25-Dihydroxy-20R-20-(4-hydroxy-5,5,5-
trideutero-4-trideuteromethyl-pentyl)-23E-
ene-26,27-hexafluoro-19-nor-
cholecalciferol

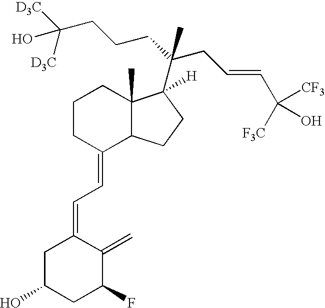

1α-Fluoro-25-hydroxy-20R-20-(4-
hydroxy-5,5,5-trideutero-4-
trideuteromethyl-pentyl)-23E-ene-26,27-
hexafluorocholecalciferol

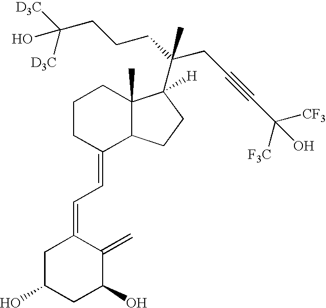

1,25-Dihydroxy-20R-20-(4-hydroxy-5,5,5-
trideutero-4-trideuteromethyl-pentyl)-23-
yne-26,27-hexafluorocholecalciferol

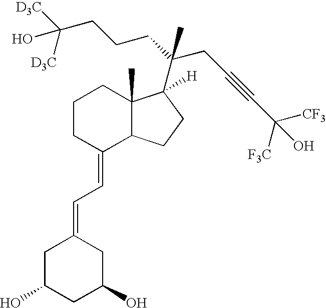

1,25-Dihydroxy-20R-20-(4-hydroxy-5,5,5-
trideutero-4-trideuteromethyl-pentyl)-23-
yne-26,27-hexafluoro-19-nor-
cholecalciferol

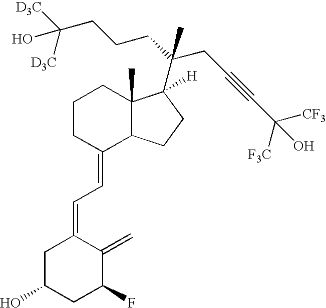

1α-Fluoro-25-hydroxy-20R-20-(4-
hydroxy-5,5,5-trideutero-4-
trideuteromethyl-pentyl)-23-yne-26,27-
hexafluorocholecalciferol

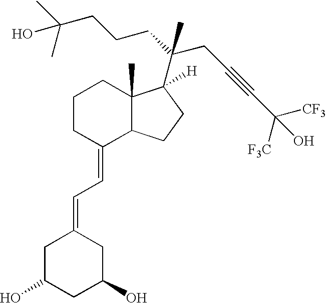

1,25-Dihydroxy-20R-20-(4-hydroxy-4-
methyl-pentyl)-23-yne-26,27-hexafluoro-
cholecalciferol

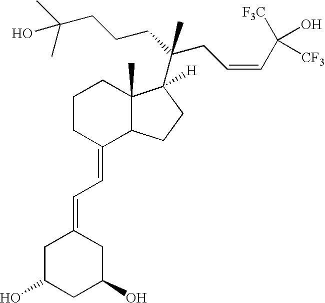

1,25-Dihydroxy-20R-20-(4-hydroxy-4-
methyl-pentyl)-23Z-ene-26,27-hexafluoro-
cholecalciferol

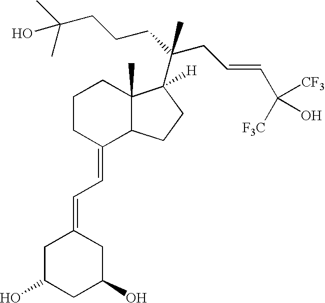

1,25-Dihydroxy-20R-20-(4-hydroxy-4-
methyl-pentyl)-23E-ene-26,27-hexafluoro-
cholecalciferol

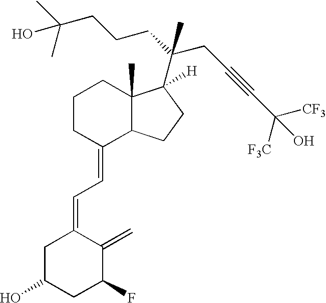

1α-Fluoro-25-hydroxy-20R-20-(4-
hydroxy-4-methyl-pentyl)-23-yne-26,27-
hexafluorocholecalciferol

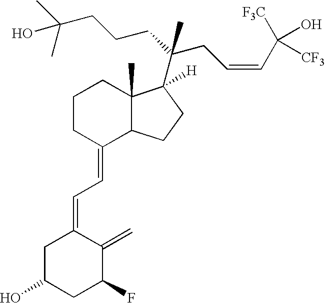

1α-Fluoro-25-hydroxy-20R-20-(4-
hydroxy-4-methyl-pentyl)-23Z-ene-26,27-
hexafluorocholecalciferol

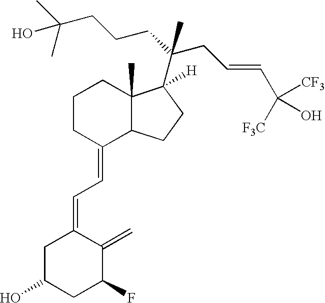

1α-Fluoro-25-hydroxy-20R-20-(4-
hydroxy-4-methyl-pentyl)-23E-ene-26,27-
hexafluorocholecalciferol

In another aspect, the invention provides a vitamin D₃ compound of formula XXII:

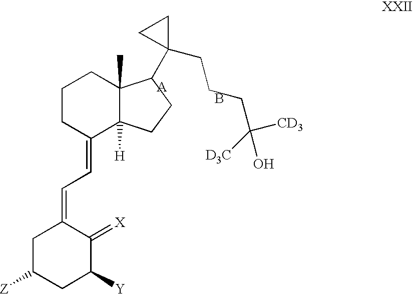

wherein: A is single or double bond; B is a single, double, or triple bond; X is H₂ or CH₂;

Y is hydroxyl, OC(O)C₁-C₄ alkyl, OC(O)hydroxyalkyl, OC(O)haloalkyl; or halogen; Z is hydroxyl, OC(O)C₁-C₄ alkyl, OC(O)hydroxyalkyl, or OC(O)haloalkyl; and pharmaceutically acceptable esters, salts, and prodrugs thereof.

Preferred compounds of the present invention are summarized in Table 1 and the syntheses of such compounds are detailed in Examples 42-50 below.

TABLE 1

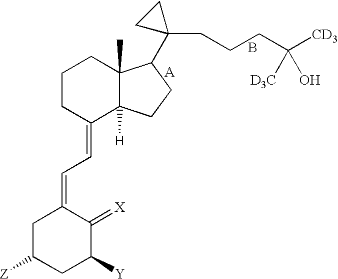

| Compound | X | Z | Y | A | B |
|---|---|---|---|---|---|
| 1,25-Dihydroxy-20-cyclopropyl-26,27- | H₂ | OH | OH | — | — |
| hexadeutero-19-nor-cholecalciferol | | | | | |
| 1,3-Di-O-acetyl-1,25-dihydroxy-20- | H₂ | OAc | OAc | — | — |
| cyclopropyl-26,27-hexadeutero-19-nor- | | | | | |
| cholecalciferol | | | | | |
| 1,25-Dihydroxy-20-cyclopropyl-26,27- | CH₂ | OH | OH | — | — |
| hexadeutero-cholecalciferol | | | | | |
| 1,3-Di-O-acetyl-1,25-dihydroxy-20- | CH₂ | OAc | OAc | — | — |
| cyclopropyl-26,27-hexadeutero- | | | | | |
| cholecalciferol | | | | | |
| 1α-Fluoro-25-hydroxy-20-cyclopropyl- | CH₂ | OH | F | — | — |
| 26,27-hexadeutero-cholecalciferol | | | | | |
| 3-O-acetyl-1α-fluoro-25-hydroxy-20- | CH₂ | OAc | F | — | — |
| cyclopropyl-cholecalciferol | | | | | |
| 1,25-Dihydroxy-16-ene-20 cyclopropyl- | H₂ | OH | OH | ═ | — |
| 26,27-hexadeutero-19-nor-cholecalciferol | | | | | |
| 1,25-Dihydroxy-16-ene-20-cyclopropyl- | CH₂ | OH | OH | ═ | — |
| 26,27-hexadeutero-cholecalciferol | | | | | |
| 1α-Fluoro-25-hydroxy-16-ene-20-cyclo | CH₂ | OH | F | ═ | — |
| propyl-26,27-hexadeutero-cholecalciferol | | | | | |

The use of compounds having the structures given above is extended to pharmaceutically acceptable esters, salts, and prodrugs thereof.

A vitamin D compound of particular interest is calcitriol. Another vitamin D compound of particular interest is Compound X. Another vitamin D compound of particular interest is Compound Y.

Other example compounds of use in the invention which are vitamin D receptor agonists include paricalcitol (ZEMPLAR™) (see U.S. Pat. No. 5,587,497), tacalcitol (BONALFA™) (see U.S. Pat. No. 4,022,891), doxercalciferol (HECTOROL™) (see Lam et al. (1974) Science 186, 1038), maxacalcitol (OXAROL™) (see U.S. Pat. No. 4,891,364), calcipotriol (DAIVONEX™) (see U.S. Pat. No. 4,866,048), and falecalcitriol (FULSTANT™).

Other compounds include ecalcidene, calcithiazol and tisocalcitate.

Other compounds include atocalcitol, lexacalcitol and seocalcitol.

Another compound of possible interest is secalciferol (“OSTEO D”).

Other non-limiting examples of vitamin D compounds that may be of use in accordance with the invention include those described in published international applications: WO2006/036813, WO2005/082375, WO2005/030223, WO2005/030222, WO2005/027923, WO2004/098522, WO2004/098507, WO2002/094247, WO98/49138, WO 01/40177, WO0010548, WO0061776, WO0064869, WO0064870, WO0066548, WO0104089, WO0116099, WO0130751, WO0140177, WO0151464, WO0156982, WO0162723, WO0174765, WO0174766, WO0179166, WO0190061, WO0192221, WO0196293, WO02066424, WO0212182, WO0214268, WO03004036, WO03027065, WO03055854, WO03088977, WO04037781, WO04067504, WO8000339, WO8500819, WO8505622, WO8602078, WO8604333, WO8700834, WO8910351, WO9009991, WO9009992, WO9010620, WO9100271, WO9100855, WO9109841, WO9112239, WO9112240, WO9115475, WO9203414, WO9309093, WO9319044, WO9401398, WO9407851, WO9407852, WO9408958, WO9410139, WO9414766, WO9502577, WO9503273, WO9512575, WO9527697, WO9616035, WO9616036, WO9622973, WO9711053, WO9720811, WO9737972, WO9746522, WO9818759, WO9824762, WO9828266, WO9841500, WO9841501, WO9849138, WO9851663, WO9851664, WO9851678, WO9903829, WO9912894, WO9915499, WO9918070, WO9943645, WO9952863, those described in U.S. Pat. Nos.: U.S. Pat. No. 3,856,780, U.S. Pat. No. 3,994,878, U.S. Pat. No. 4,021,423, U.S. Pat. No. 4,026,882, U.S. Pat. No. 4,028,349, U.S. Pat. No. 4,225,525, U.S. Pat. No. 4,613,594, U.S. Pat. No. 4,804,502, U.S. Pat. No. 4,898,855, U.S. Pat. No. 4,929,609, U.S. Pat. No. 5,039,671, U.S. Pat. No. 5,087,619, U.S. Pat. No. 5,145,846, U.S. Pat. No. 5,247,123, U.S. Pat. No. 5,342,833, U.S. Pat. No. 5,393,900, U.S. Pat. No. 5,428,029, U.S. Pat. No. 5,451,574, U.S. Pat. No. 5,612,328, U.S. Pat. No. 5,747,478, U.S. Pat. No. 5,747,479, U.S. Pat. No. 5,804,574, U.S. Pat. No. 5,811,414, U.S. Pat. No. 5,856,317, U.S. Pat. No. 5,872,113, U.S. Pat. No. 5,888,994, U.S. Pat. No. 5,939,408, U.S. Pat. No. 5,962,707, U.S. Pat. No. 5,981,780, U.S. Pat. No. 6,017,908, U.S. Pat. No. 6,030,962, U.S. Pat. No. 6,040,461, U.S. Pat. No. 6,100,294, U.S. Pat. No. 6,121,312, U.S. Pat. No. 6,329,538, U.S. Pat. No. 6,331,642, U.S. Pat. No. 6,392,071, U.S. Pat. No. 6,452,028, U.S. Pat. No. 6,479,538, U.S. Pat. No. 6,492,353, U.S. Pat. No. 6,537,981, U.S. Pat. No. 6,544,969, U.S. Pat. No. 6,559,138, U.S. Pat. No. 6,667,298, U.S. Pat. No. 6,683,219, U.S. Pat. No. 6,696,431, U.S. Pat. No. 6,774,251, and those described in published US patent applications: US2001007907, US2003083319, US2003125309, US2003130241, US2003171605, US2004167105, US2004214803 and US2005065124.

It will be noted that the structures of some of the compounds of the invention include asymmetric carbon atoms. Accordingly, it is to be understood that the isomers arising from such asymmetry (e.g., all enantiomers and diastereomers) are included within the scope of this invention, unless indicated otherwise. Such isomers can be obtained in substantially pure form by classical separation techniques and/or by stereochemically controlled synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

The invention also provides a pharmaceutical composition, comprising an effective amount of a vitamin D compound as described herein and a pharmaceutically acceptable carrier. In a further embodiment, the effective amount is effective to prevent adhesions as described previously.

In an embodiment, the vitamin D compound is administered to the subject using a pharmaceutically-acceptable formulation, e.g., a pharmaceutically-acceptable formulation that provides sustained delivery of the vitamin D compound to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject or intraperitoneal administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension, (3) topical application, for example, as a cream, ointment or spray applied to the skin; (4) intravaginally or intrarectally, for example, as a pessary, cream or foam; (5) aerosol, for example, as an aqueous aerosol, liposomal preparation or solid particles containing the compound; or (6) intraperitoneal delivery e.g. as a sterile solution or suspension (such as an aqueous solution or suspension).

The phrase “pharmaceutically acceptable” refers to those vitamin D compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The compositions may conveniently be presented in unit dosage form and may be prepared by any methods well known in the art of pharmacy.

The amount of active ingredient which can be combined with a carrier material to produce a single dosage form will vary depending upon the subject intended to receive the dose and the particular mode of administration. The amount of active ingredient which can be combined with a carrier material to produce a single dosage form will generally be that amount of the compound which produces a prophylactic effect. Generally, out of one hundred percent, this amount will range from about 0.1 percent to about ninety-nine percent of active ingredient, preferably from about 5 percent to about 70 percent, most preferably from about 10 percent to about 30 percent.

Methods of preparing these compositions include the step of bringing into association a vitamin D compound(s) with the carrier and, optionally, one or more accessory ingredients. In general, the formulations are prepared by uniformly and intimately bringing into association a vitamin D compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a vitamin D compound(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes.

The active ingredient can also be in micro-encapsulated form, if appropriate, with one or more of the above-described excipients.

Liquid dosage forms for oral administration of the vitamin D compound(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active vitamin D compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more vitamin D compound(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a vitamin D compound(s) include powders, sprays, ointments, pastes, creams, lotions, gels, solutions, patches and inhalants. The active vitamin D compound(s) may be mixed under sterile conditions with a pharmaceutically-acceptable carrier, and with any preservatives, buffers, or propellants which may be required.

The ointments, pastes, creams and gels may contain, in addition to vitamin D compound(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a vitamin D compound(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The vitamin D compound(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a vitamin D compound(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Pharmaceutical compositions of the invention suitable for parenteral or intraperitoneal administration comprise one or more vitamin D compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Compositions containing a vitamin D compound according to the invention may suitably include an agent to increase the viscosity of the composition, especially in order to facilitate contact or to increase contact time between the composition and tissues when administered intra-peritoneally.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form.

Alternatively, delayed absorption of a parenterally-administered drug form is accomplished by dissolving or suspending the drug in an oil vehicle.

Injectable depot forms are made by forming microencapsule matrices of vitamin D compound(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The invention also provides kits for prevention of adhesions. In one embodiment, the kit includes an effective amount of a vitamin D compound in unit dosage form, together with instructions for administering the vitamin D compound to a subject susceptible to adhesions.

In preferred embodiments, the kit comprises a sterile container which contains the vitamin D compound; such containers can be boxes, ampules, bottles, vials, tubes, bags, pouches, blister-packs, or other suitable container form known in the art. Such containers can be made of plastic, glass, laminated paper, metal foil, or other materials suitable for holding medicaments.

The instructions will generally include information about the use of the compound for prophylaxis of adhesions; in preferred embodiments, the instructions include at least one of the following: description of the compound; dosage schedule and administration for prophylaxis of adhesions; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

When the vitamin D compound(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the vitamin D compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. One exemplary dose range is from 0.1 to 300 ug per day.

A preferred dose of the vitamin D compound for the present invention is the maximum that a patient can tolerate and not develop hypercalcemia. Preferably, the vitamin D compound of the present invention is administered at a concentration of about 0.001 ug to about 100 ug per kilogram of body weight, about 0.001-about 10 ug/kg or about 0.001 ug-about 100 ug/kg of body weight. Ranges intermediate to the above-recited values are also intended to be part of the invention. For example, another typical dose amount may be 0.1-600 ug/kg e.g. 1-100 ug/kg.

The vitamin D compound may be administered separately, sequentially or simultaneously in separate or combined pharmaceutical formulations with a second medicament for the prevention and/or treatment of adhesions (for example a second vitamin D compound of the present invention, or an antibiotic, or an anti-inflammatory compound). Such combination therapy may increase the efficacy of the overall treatment or may permit the second medicament to be used in a lower amount than without the vitamin D compound. In the case of prevention of post-surgical adhesions, it may be desired to administer an antibiotic or an anti-inflammatory compound prior to surgery e.g. 24 hours prior, usually systemically (eg orally), such that an efficacious blood plasma (or local) level is achieved at the time of surgery.

Suitably the vitamin D compounds of the invention are not co-administered or co-formulated with crosslinked compositions or crosslinkable compositions which inter-react in an aqueous environment to form a three-dimensional matrix, such as with homogeneous dry powder compositions comprised of a first component having a core substituted with m nucleophilic groups, where m is greater than or equal to 2; and a second component having a core substituted with n electrophilic groups, where n is greater than or equal to 2 and m+n is greater than 4; wherein the nucleophilic and electrophilic groups are non-reactive in a dry environment but are rendered reactive upon exposure to an aqueous environment such that the components inter-react in the aqueous environment to form a three-dimensional matrix (see US2005/0281883).

Where adhesion formation is associated with a particular underlying disease or disorder, the vitamin D compound for use in the prevention of adhesions may be administered with a further medicament for the treatment or prevention of the underlying disease or disorder.

In the case of prevention of post-surgical adhesions a convenient administration regime involves systemically administering the vitamin D compound prior to surgery, e.g. by oral administration. Alternatively the vitamin D compound may be locally administered directly at the site of surgery (e.g. to the peritoneum via i.p. route) at the conclusion of surgery. Suitably the vitamin D compound may be locally administered directly to the site of surgery at the conclusion of surgery in a controlled release form in order to allow continued exposure locally over a period of time (e.g. a number of days).

A solution of the vitamin D compound may be used to wash the site of surgery before, during or after surgery.

Suitably the vitamin D compound is systemically administered prior to surgery, e.g. by oral administration, and additionally the vitamin D compound is locally administered directly to the site of surgery (e.g. the peritoneum) at the conclusion of surgery.

Upon conclusion of surgery (eg immediately prior to closure of the incision) a volume of composition (eg aqueous composition) containing the vitamin D compound may be instilled at the site of surgery. Volumes of 100-2000 ml eg 1000 ml may be suitable.

Synthesis of Compounds of the Invention

The syntheses of compounds of the invention have been described in the art, for example, in WO2006/036813, WO2005/082375, WO2005/030223, WO2005/030222, WO2005/027923, WO2004/098522, WO2004/098507, WO2002/094247, WO98/49138, U.S. Pat. No. 6,492,353, U.S. Pat. No. 6,030,962 and U.S. Pat. No. 5,939,408, the contents of which are incorporated herein by reference in their entirety.

### EXEMPLIFICATION OF THE INVENTION

The present invention will now be described with reference to the following non-limiting examples.

### SYNTHETIC EXAMPLES

All operations involving vitamin D₃ analogs were conducted in amber-colored glassware in a nitrogen atmosphere. Tetrahydrofuran was distilled from sodium-benzophenone ketyl just prior to its use and solutions of solutes were dried with sodium sulfate. Melting points were determined on a Thomas-Hoover capillary apparatus and are uncorrected. Optical rotations were measured at 25° C. ¹H NMR spectra were recorded at 400 MHz in CDCl₃ unless indicated otherwise. TLC was carried out on silica gel plates (Merck PF-254) with visualization under short-wavelength UV light or by spraying the plates with 10% phosphomolybdic acid in methanol followed by heating. Flash chromatography was carried out on 40-65 μm mesh silica gel. Preparative HPLC was performed on a 5×50 cm column and 15-30 μm mesh silica gel at a flow rate of 100 ml/min.

### Example 1

### Synthesis of 1-alpha-fluoro-25-hydroxy-16,23E-diene-26,27-bishomo-20-epi-cholecalciferol

### t-Butyl-dimethyl-(4-methylene-3-{2-[7a-methyl-1-(1,4,5-trimethyl-hex-2-enyl)-octahydro-inden-4-ylidene]-ethylidene}-cyclohexyloxy)-silane

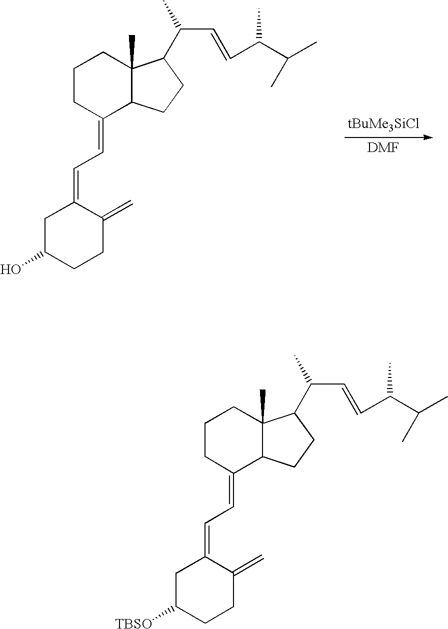

To a stirred solution of 4-Methylene-3-{2-[7a-methyl-1-(1,4,5-trimethyl-hex-2-enyl)-octahydro-inden-4-ylidene]-ethylidene}-cyclohexanol (100.00 g, 0.25 mol) in DMF (250 mL), imidazole (40.80 g, 0.6 mol) and (t-butyldimethyl)silyl chloride (45.40 g, 0.3 mol) were added successively. The reaction mixture was stirred at room temperature for 1 h, diluted with hexane (750 mL), washed with water (500 mL), 1N HCl (500 mL), brine (500 mL) and dried over Na₂SO₄. The residue (155 g) after evaporation of the solvent was filtered through a plug of silica gel (500 g, 5% AcOEt in hexane) to give the title compound (115.98 g, 0.23 mol, 92%).

¹H-NMR: δ 0.04 and 0.08 (2s, 6H), 0.59 (s, 3H), 0.90 (d, 3H, J=6.6 Hz), 0.92 (d, 3H, J=6.6 Hz), 0.98 (s, 9H), 0.99 (d, 3H, J=7.0 Hz), 1.06 (d, 3H, J=6.8 Hz), 1.10-2.95 (m, 21H), 5.11 (br s, 2H), 5.22 (m, 2H), 6.49 (br s, 2H).

### 2-[5-(tert-Butyl-dimethyl-silanyloxy)-2-methylene-cyclohexylidene]-ethanol and 1-(2-Hydroxy-1-methyl-ethyl)-7a-methyl-octahydro-inden-4-ol

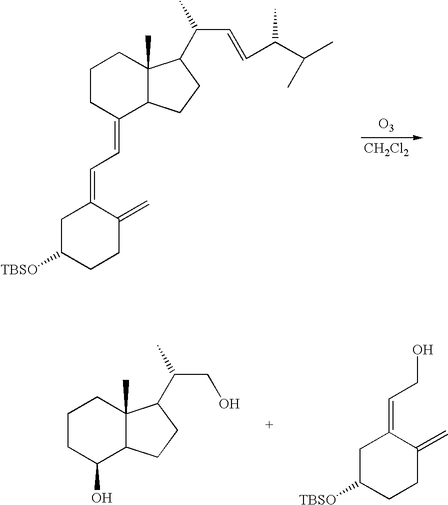

A stream of ozone was passed through a stirred solution of t-Butyl-dimethyl-(4-methylene-3-{2-[7a-methyl-1-(1,4,5-trimethyl-hex-2-enyl)-octahydro-inden-4-ylidene]-ethylidene}-cyclohexyloxy)-silane (23.4 g, 45.8 mmol), pyridine (5.0 mL) and Sudane Red 7B (15.0 mg) in dichloromethane (550 mL), at −55 to −60° C. until Sudane Red decolorized (55 min). Sodium borohydride (6.75 g, 180 mmol) was then added followed by ethanol (250 mL). The reaction was allowed to warm to room temperature and stirred at room temperature for 1 h. Acetone (15 mL) was added and, after 30 min brine (300 mL) was added. The mixture was diluted with ethyl acetate (500 mL) and washed with water (600 mL). The aqueous phase was extracted with AcOEt (300 mL). The combined organic phases were dried over Na₂SO₄. The residue (26.5 g), after evaporation of the solvent, was filtered through a plug of silica gel (500 g, 15%, 30% and 50% AcOEt in hexane) to give: Fraction A (5.9 g, mixture containing the desired A-ring (ca 83% pure by NMR) ¹H NMR: δ 5.38 (1H, t, J=6.4 Hz), 4.90 (1H, brs), 4.57 (1H, brs), 4.22 (1H, dd, J=7.3, 12.5 Hz), 4.13 (1H, dd, J=6.3, 12.5 Hz), 3.78 (1H, m), 2.40-1.30 (6H, m), 0.83 (9H, s), 0.01 (3H, s), 0.00 (3H, s); Fraction A was used for the synthesis of the A-ring precursor. Fraction B (14.6 g, mixture containing a CD-rings fragments on a different stage of oxidation). Fraction B was further ozonolyzed in order to obtain the Lythgoe diol. A stream of ozone was passed through a stirred solution of Fraction B (14.6 g) and Sudane Red 7B (3.0 mg) in ethanol (225 mL) at −55 to −60° C. for 30 min (Sudane Red decolorized). Sodium borohydride (3.75 g, 100 mmol) was added and the reaction was allowed to warm to room temperature and stirred at room temperature for 1 h. Acetone (5 mL) was added and, after 30 min brine (200 mL) was added. The mixture was diluted with dichloromethane (300 mL) and washed with water (250 mL). The aqueous phase was extracted with dichloromethane (200 mL). The combined organic phases were, evaporated to dryness (the last portion was evaporated with addition of toluene 100 mL). The residue (16.2 g) was dissolved in dichloromethane (100 mL), concentrated to a volume of ca 20 mL diluted with petroleum ether (30 mL) and set aside in the fridge for crystallization. The white powder was filtered of (4.05 g), the mother liquor was concentrated and filtered through silica gel (100 g, 5% MeOH in CH₂Cl₂) to give yellow oil (9.4 g), which was recrystallized (20 mL, dichloromethane; petroleum ether 1:2) to give white powder (1.79 g). Thus the total yield of the Lythgoe diol was (5.84 g, 27.5 mmol, 60% from D₂) ¹H NMR: δ 4.08 (1H, m), 3.64 (1H, dd, J=3.3, 10.6 Hz), 3.39 (1H, dd, J=6.6, 10.6 Hz), 2.04-1.14 (15H, m), 1.03 (3H, d, J=6.6 Hz), 0.96 (3H, s).

### 1-(2-Hydroxy-1-methyl-ethyl)-7a-methyl-octahydro-inden-4-ol and 1-(2-Hydroxy-1-methyl-ethyl)-7a-methyl-octahydro-inden-4-ol

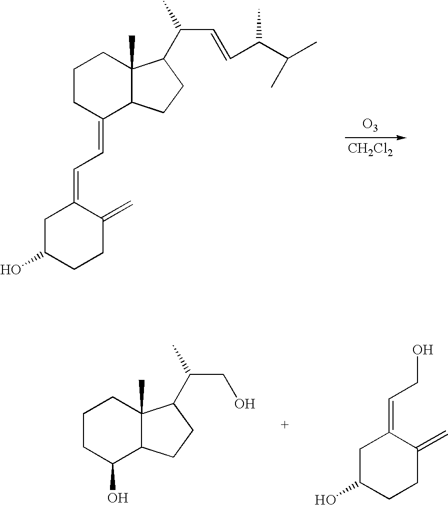

t-Butyl-dimethyl-(4-methylene-3-{2-[7a-methyl-1-(1,4,5-trimethyl-hex-2-enyl)-octahydro-inden-4-ylidene]-ethylidene}-cyclohexyloxy)-silane (98.8 g, 249 mmol) was dissolved in dichloromethane (900 mL) and ethanol (400 mL), pyridine (25.0 mL) and Sudane Red 7B (30.0 mg) were added and the mixture was cooled down to −65 to −70° C. A stream of ozone was passed through for 3 h. (until Sudane Red decolorized, reaction was also followed by TLC and decolorization of Sudane Red corresponds to consumption of Vitamin D₂). Sodium borohydride (24.0 g, 0.64 mol) was added portion-wise and the reaction was allowed to warm to room temperature and stirred at room temperature for 1 h. Acetone (75 mL) was added portion-wise (to keep temperature under 35° C.) and the reaction mixture was stored overnight in the fridge. The mixture was washed with water (600 mL). The aqueous phase was extracted with dichloromethane (6×300 mL). The combined organic phases were dried over Na₂SO₄. The residue (118 g) after evaporation of the solvent was passed through a plug of silica gel (0.5 kg, 30%, 50% AcOEt in hexane) to give: Fraction A (69.7 g, CD-rings fragments); Fraction B (4.8 g of a pure Lythgoe diol after crystallization from hexane:AcOEt 3:1); Fraction C (12.3 g of a pure compound starting material, after crystallization from AcOEt); Fraction D (11.5 g, mixture of the starting material and 4-Methylene-cyclohexane-1,3-diol).

Fraction A was further ozonolyzed in order to obtain the diol. A stream of ozone was passed through a stirred solution of Fraction A (69.7 g) in ethanol (500 mL), dichloromethane (600 mL) and Sudane Red 7B (3.0 mg) at −65 to −70° C. for 3 h. (Sudane Red decolorized). Sodium borohydride (22.5 g, 0.6 mol) was added and the reaction was allowed to warm to room temperature and stirred at room temperature for 1 h. Acetone (125 mL) was added portion-wise (to keep temperature under 35° C.) and the reaction mixture was stored overnight in the fridge. The mixture was washed with water (600 mL). The aqueous phase was extracted with dichloromethane (2×300 mL) and with AcOEt (300 mL). The combined organic phases were dried over Na₂SO₄ and evaporated to dryness. The residue (55.0 g) was purified by crystallization (AcOEt:Hexane 1:2) to give: Fraction E (15.7 g of a pure crystalline diol); Fraction F (35 g, of mixture containing Lythgoe diol). Fraction F (35 g) was passed through a plug of silica gel (0.5 kg, 30%, 50% AcOEt in hexane) to give after crystallization (AcOEt:Hexane 1:2) Fraction G (18.9 g), thus the overall yield of diol was 39.4 g 74.5% from the starting material).

¹H NMR: δ 5.38 (1H, t, J=6.4 Hz), 4.90 (1H, brs), 4.57 (1H, brs), 4.22 (1H, dd, J=7.3, 12.5 Hz), 4.13 (1H, dd, J=6.3, 12.5 Hz), 3.78 (1H, m), 2.40-1.30 (6H, m), 0.83 (9H, s), 0.01 (3H, s), 0.00 (3H, s);

Fraction D (11.5 g) was passed through a plug of silica gel (0.3 kg, 50% AcOEt in hexane) to give (after crystallization (AcOEt): Fraction H (1.1 g of a pure crystalline 1-(2-Hydroxy-1-methyl-ethyl)-7a-methyl-octahydro-inden-4-ol, 2.8%); Fraction I (10.2 g, mixture of the desired compound. Thus the overall yield of the isolated (S)-(Z)-3-(2-Hydroxy-ethylidene)-4-methylene-cyclohexanol is 13.4 g, 34.8%

¹H NMR: δ 5.51 (1H, t, J=6.6 Hz), 5.03 (1H, brs), 4.66 (1H, brs), 4.24 (2H, m), 3.94 (1H, m), 2.55 (1H, dd, J=3.9, 13.2 Hz), 2.41 (1H, m), 2.25 (1H, dd, J=7.8, 12.9 Hz), 1.94 (1H, m), 1.65 (1H, m).

### (S)-(Z)-2-[5-(tert-butyldimethyl)silanyloxy)-2-methylene-cyclohexylidene]-ethanol

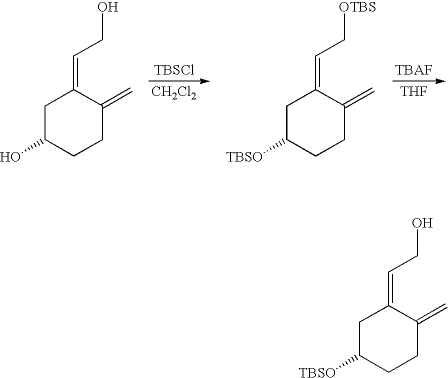

To a stirred solution (S)-(Z)-3-(2-Hydroxy-ethylidene)-4-methylene-cyclohexanol (4.04 g, 26.3 mmol) in dichloromethane (40 mL), imidazole (5.36 g, 78.7 mmol) and (tert-butyldimethyl)silyl chloride (9.50 g, 63.0 mmol) were added successively. The reaction mixture was stirred at room temperature for 100 min. after which water (25 mL) was added. After 15 min. the mixture was diluted with hexane (350 mL), washed with water (2×100 mL) and brine (50 mL) and dried over Na₂SO₄. The residue (10.7 g) after evaporation of the solvent was dissolved in tetrahydrofurane (50 mL), Bu₄NF (26.5 mL, 1M/THF) was added at +5° C. and the mixture was stirred at +5° C. for 45 min. and additional 30 min. at room temperature. The mixture was diluted with water (100 mL) and ethyl acetate (250 mL). After separation organic layer was washed with water (100 mL) and brine (50 mL). Aqueous layers were extracted with ethyl acetate (5×50 mL). The combined organic layers were dried over Na₂SO₄. The residue after evaporation of the solvent was purified by FC (150 g, 10%, 50% and 100% AcOEt in hexane) to give the titled compound. (6.43 g, 85% pure by NMR, 78% of the title compound,)

¹H NMR: δ 5.38 (1H, t, J=6.4 Hz), 4.90 (1H, brs), 4.57 (1H, brs), 4.22 (1H, dd, J=7.3, 12.5 Hz), 4.13 (1H, dd, J=6.3, 12.5 Hz), 3.78 (1H, m), 2.40-1.30 (6H, m), 0.83 (9H, s), 0.01 (3H, s), 0.00 (3H, s).

### Synthesis of the A-Ring Precursor

### (2R,3S,7S)-[7-(t-butyldimethyl)silanyloxy)-4-methylene-1-oxa-spiro[2.5]oct-2-yl]-methanol

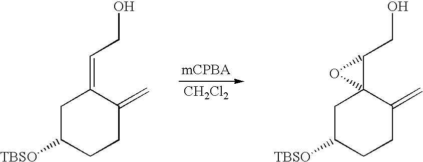

To a stirred solution of a crude (S)-(Z)-2-[5-(tert-butyldimethyl)silanyloxy)-2-methylene-cyclohexylidene]-ethanol (5.9 g, ca 18.3 mmol, Fraction A from ozonolsysis) in dichloro-methane (120 mL) at room temperature, AcONa (2.14 g, 26.1 mmol) was added followed by 72% mCPBA (4.32 g, 18.0 mmol). The reaction mixture was then stirred at 10° C. for ½ h then diluted with hexane (200 mL) washed with 10% K₂CO₃ (3×150 mL), and dried over Na₂SO₄. The residue after evaporation of solvent (6.6 g) was filtered through a plug of silica gel (150 g, 10% AcOEt in hexane) to give the crude title compound (4.87 g, ca 15.4 mmol, 84%) ¹H-NMR: δ 0.063 and 0.068 (2s, 6H), 0.88 (s, 9H), 1.38-1.49 (m, 1H), 1.54 (m, 1H, OH), 1.62 (m, 1H), 1.96 (m, 3H), 2.43 (m, 1H), 3.095 (t, 1H, J=5.6 Hz), 3.60 (m, 2H), 3.86 (m, 1H), 4.91 (m, 1H).

### Benzoic acid (2R,3S,7S)-7-(t-butyldimethyl)silanyloxy)-4-methylene-1-oxa-spiro[2.5]oct-2-yl methyl ester

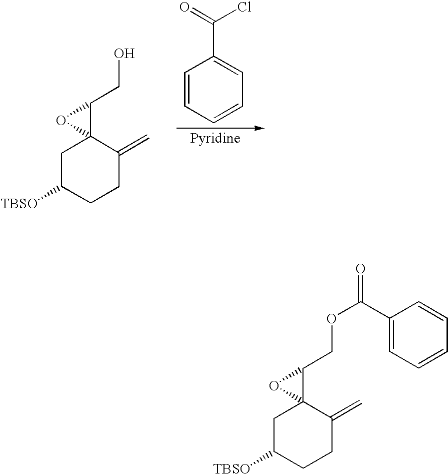

To a stirred solution of (2R,3S,7S)-[7-(t-butyldimethyl)silanyloxy)-4-methylene-1-oxa-spiro[2.5]oct-2-yl]-methanol (4.87 g, ca 15.4 mmol) in pyridine (25 mL) at room temperature, benzoyl chloride (2.14 mL, 18.4 mmol) was added and the reaction mixture was stirred for 1 h. Water (25 mL) was added and after stirring for 45 min at room temperature the mixture was diluted with hexane (80 mL), washed with saturated NaHCO₃ solution (50 mL), and dried over Na₂SO₄. The residue after evaporation of solvent (17.5 g) was purified by FC (150 g, 10% AcOEt in hexane) to give the title compound (5.44 g, 14.0 mmol, 91%) ¹H NMR: δ 8.04-7.80 (2H, m), 7.56-7.50 (1H, m), 7.44-7.37 (2H, m), 4.94 (1H, brs), 4.92 (1H, brs), 4.32 (1H, dd, J=4.8, 11.9 Hz), 4.14 (1H, dd, J=6.2, 11.9 Hz), 3.83 (1H, m), 3.21 (1H, dd, J=4.8, 6.2 Hz), 2.42 (1H, m), 2.04-1.90 (3H, m), 1.64-1.34 (2H, m), 0.83 (9H, s), 0.02 (3H, s), 0.01 (3H, s).

### Benzoic acid (2R,3S,5R,7S)-7-(t-butyldimethyl)silanyloxy)-5-hydroxy-4-methylene-1-oxa-spiro[2.5]oct-2-yl methyl ester

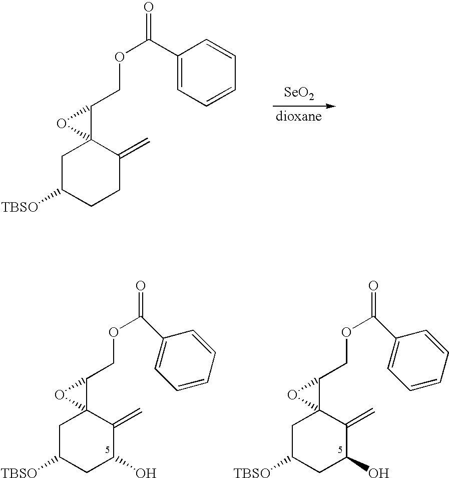

To a stirred solution of Benzoic acid (2R,3S,7S)-7-(t-butyldimethyl)silanyloxy)-4-methylene-1-oxa-spiro[2.5]oct-2-yl methyl ester (10.0 g, 25.7 mmol)) in dioxane (550 mL) at 85° C. was added selenium dioxide, (3.33 g, 30.0 mmol) followed by t-butyl hydrogen peroxide (9.0 mL, 45.0 mmol, 5-6 M in nonane) and the reaction mixture was stirred at 85° C. for 16 h, after which selenium dioxide (1.11 g, 10.0 mmol) was added followed by t-butyl hydrogen peroxide (3.0 mL, 15.0 mmol, 5-6 M in nonane) and the reaction mixture was stirred at 85° C. for additional 6 h. The solvent was removed under vacuum and the residue (15.3 g) was filtered through a plug of silica gel (300 g, 20% AcOEt in hexane) to give: starting material (970 mg, 10%) and a mixture of produce epimer a and epimer b (8.7 g). This mixture was divided into 3 portion (2.9 g each) and purified twice by FC (200 g, 5% isopropanol in hexane, same column was used for all six chromatographs) to give: Epimer b (1.83 g, as a 10:1 mixture of 10b:10a ca 16% of 5α-hydroxy compound); Epimer a (6.0 g, 14.8 mmol, 58%) as white crystals. The structure of Epimer a was confirmed by X-ray crystallography.

¹H NMR: δ 8.02-7.90 (2H, m), 7.58-7.50 (1H, m), 7.46-7.38 (2H, m), 5.25 (1H, br s), 5.11 (1H, br s), 4.26 (1H, dd, J=5.5, 12.1 Hz), 4.15 (1H, dd, J=5.9, 12.1 Hz), 4.07 (1H, m), 3.87 (1H, m), 3.19 (1H, dd, J=5.5, 5.9 Hz), 2.34-1.10 (5H, m), 0.81 (9H, s), 0.01 (3H, s), 0.00 (3H, s).

### Benzoic acid (2R,3S,5S,7R)-7-(t-butyldimethyl)silanyloxy)-5-fluoro-4-methylene-1-oxa-spiro[2.5]oct-2-ylmethyl ester

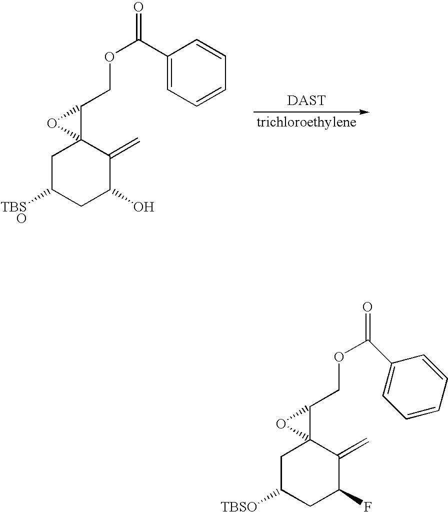

To a stirred solution of a diethylaminosulfur trifluoride (DAST) (2.0 mL, 16.0 mmol) in trichloroethylene (20 mL) a solution of Benzoic acid (2R,3S,5R,7S)-7-(t-butyldimethyl)silanyloxy)-5-hydroxy-4-methylene-1-oxa-spiro[2.5]oct-2-yl methyl ester (2.78 g, 6.87 mmol) in trichloroethylene (126 mL was added at −75° C. After stirring for 20 min at −75° C. methanol (5.5 mL) was added followed by saturated NaHCO₃ solution (6 mL) and the resulting mixture was diluted with hexane (150 mL) and washed with saturated NaHCO₃ solution (100 mL), dried over Na₂SO₄ and concentrated. The residue (4.5 g) was purified by FC (150 g, DCM:hexane:AcOEt 10:20:0.2) to give the title compound (2.09 g, 5.14 mmol, 75%) ¹H NMR: δ 8.02-7.99 (2H, m), 7.53-7.45 (1H, m), 7.40-7.33 (2H, m), 5.26 (2H, m), 5.11 (1H, dt, J=3.0, 48.0 Hz), 4.46 (1H, dd, J=3.3, 12.5 Hz), 4.21 (1H, m), 3.94 (1H, dd, J=7.7, 12.5 Hz), 3.29 (1H, dd, J=3.3, 7.7 Hz), 2.44-1.44 (4H, m), 0.80 (9H, s), 0.01 (3H, s), 0.00 (3H, s).

### Benzoic acid 2-[5-(tert-butyl-dimethyl-silanyloxy)-3-fluoro-2-methylene-cyclohexylidene]-ethyl ester

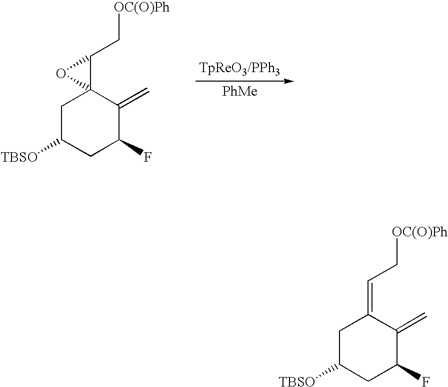

A mixture of tris(3,5-dimethylpyrazoyl)hydridoborate rhenium trioxide (265 mg, 0.50 mmol), triphenylphosphine (158 mg, 0.6 mmol), Benzoic acid (2R,3S,5S,7R)-7-(t-butyldimethyl)silanyloxy)-5-fluoro-4-methylene-1-oxa-spiro[2.5]oct-2-ylmethyl ester (203 mg, 0.5 mmol) and toluene (8 mL) was sealed in an ampule under argon and heated at 100° C. for 14 h. (TLC, 10% AcOEt in hexane, mixture of substrate and product, ca 1:1). Rhenium oxide did not completely solubilized. A solution of triphenylphosphine (158 mg, 0.6 mmol) in toluene (4 mL) was added and the heating continued for 6 h. The reaction mixture was cooled to room temperature filtered through a plug of silica gel and then the residue after evaporation of the solvent was purified by FC (20 g, 5% AcOEt in hexane) to give: titled compound (120 mg, 0.31 mmol, 61% of the desire product) and 70 mg of the starting material plus minor contaminations, ca 34%.

### (1Z,3S,5R)-2-[5-(t-butyldimethyl)silanyloxy)-3-fluoro-2-methylene-cyclohexylidene]-ethanol

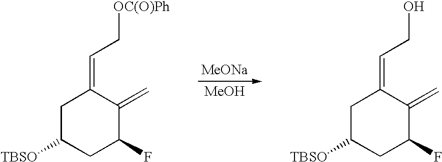

To a solution of Benzoic acid 2-[5-(tert-butyl-dimethyl-silanyloxy)-3-fluoro-2-methylene-cyclohexylidene]-ethyl ester (150 mg, 0.38 mmol) in methanol (3 mL) was added sodium methoxide (0.5 mL, 15% in methanol). After stirring for 1 h at room temperature water was added (6 mL) and the mixture was extracted with methylene chloride (3×10 mL). The combined organic layers was dried over Na₂SO₄ and evaporated to dryness. The residue (0.2 g) was purified by FC (20 g, 15% AcOEt in hexane) to give the titled compound (80 mg, 0.28 mmol, 73% of the product).

### (1R,3Z,5S)-t-butyl-[3-(2-chloro-ethylidene)-5-fluoro-4-methylene-cyclohexyloxy]-dimethylsilane

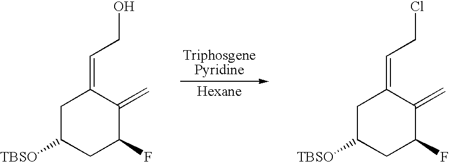

To a solution of (1Z,3S,5R)-2-[5-(t-butyldimethyl)silanyloxy)-3-fluoro-2-methylene-cyclohexylidene]-ethanol (8.07 g, 28.2 mmol) and triphosgene (4.18 g, 14.1 mmol) in hexane (150 mL) at 0° C. was added over 30 min a solution of pyridine (4.5 mL, 55.6 mmol) in hexane (20 mL) and the reaction mixture was stirred at this temperature for 30 min and at room temperature for another 30 min. The reaction mixture was washed with CuSO₄ aq (3×200 mL). The combined aqueous layers were back-extracted with hexane (2×100 mL). The organic layers were combined, dried (MgSO₄), and concentrated in vacuo to give the title compound (9.0 g, overweight). This material was used immediately in the next step without further purification. [α]²⁵_{D} +73.0° (c 0.28, CHCl₃); IR (CHCl₃) 1643, 838 cm⁻¹; ¹H-NMR δ 0.08 (s, 6H), 0.88 (s, 9H), 1.84-2.03 (m, 1H), 2.12 (br s, 1H), 2.24 (m, 1H), 2.48 (br d, J=13 Hz, 1H), 4.06-4.26 (m, 3H), 5.10 (br d, J=48 Hz), 5.16 (s, 1H), 5.35 (s, 1H), 5.63 (br t, J=6 Hz, 1H).

### (1S,3Z,5R)-1-fluoro-5-(t-butyldimethyl)silanyloxy)-2-methenyl-3-(diphenylphosphinoyl)ethylidene cyclohexane

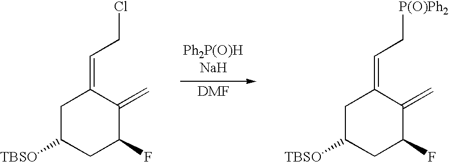

Diphenylphosphine oxide (6.70 g, 33.1 mmol) was added portionwise, over 15 min to a suspension of NaH (1.33 g, 33.1 mmol, 60% dispersion in mineral oil) in DMF (50 mL) at 10° C. The resulting solution was stirred at room temperature for 30 min and cooled to −60° C. The solution of crude (1R,3Z,5S)-t-butyl-[3-(2-chloro-ethylidene)-5-fluoro-4-methylene-cyclohexyloxy]-dimethylsilane (9.0 g) in DMF (20 mL) was then added dropwise. The reaction mixture was stirred at −60° C. for 2 h and at room temperature for 1 h, diluted with diethyl ether (600 mL) and washed with water (3×200 mL). The aqueous layers were extracted with diethyl ether (200 mL). The combined organic layers were dried (MgSO₄) and concentrated under reduced pressure to give white solid. The crude product was recrystallized from diisopropyl ether (25 mL). The resulting solid was collected by filtration, washed with cold diisopropyl ether (5 mL) and dried under high vacuum to give the title compound (7.93 g). The mother liquor was concentrated and the residue was subjected to chromatography on silca gel (50 g, 30%-50% AcOEt in hexane) to give title compound (2.22 g). Thus the total yield of the of (1S,3Z,5R)-1-fluoro-5-(t-butyldimethyl)silanyloxy)-2-methenyl-3-(diphenyl phosphinoyl)ethylidene cyclohexane was (10.1 g, 21.5 mmol, 76% overall from (1Z,3S,5R)-2-[5-(t-butyldimethyl)silanyloxy)-3-fluoro-2-methylene-cyclohexylidene]-ethanol. [α]²⁵_{D} +50.2° (c 0.84, CHCl₃); IR (CHCl₃) 835, 692 cm⁻¹; UVλₘₐₓ (ethanol) 223 (ε 22770), 258 (1950), 265 (1750), 272 nm (1280); MS, m/e 470 (M⁺), 455 (4), 450 (8), 413 (98), 338 (9), 75 (100); ¹H-NMR: δ 0.02 (s, 6H), 0.84 (s, 9H), 1.76-1.93 (m, 1H), 2.16 (m, 2H), 2.42 (br d, 1H), 3.28 (m, 2H), 4.01 (m, 1H), 5.02 (dm, J=44 Hz, 1H), 5.14 (s, 1H), 5.30 (s, 1H), 5.5 (m, 1H), 7.5 (m, 6H), 7.73 (m, 4H). Analysis Calcd for C₂₇H₃₆O₂FPSi: C, 68.91; H, 7.71; F, 4.04. Found: C, 68.69; H, 7.80; F 3.88.

### Larger Scale Synthesis of the A-Ring Precursor

### (2R,3S,7S)-17-(t-butyldimethyl)silanyloxy)-4-methylene-1-oxa-spiro[2.5]oct-2-yl-methanol

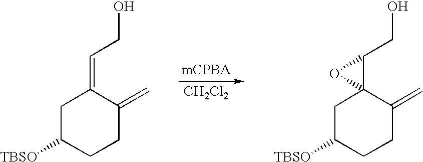

To a stirred solution of crude (S)-(Z)-2-[5-(tert-butyldimethyl)silanyloxy)-2-methylene-cyclohexylidene]-ethanol (13.5 g, ca 40 mmol) in dichloromethane (100 mL) at room temperature, was added AcONa (4.5 g, 54.8 mmol), followed by 77% mCPBA (8.96 g, 40.0 mmol) at +5° C. The reaction mixture was then stirred at +5° C. for 1.5 h, diluted with hexane (500 mL), washed with water (200 mL) and NaHCO₃ (2×200 mL) and dried over Na₂SO₄. The residue after evaporation of solvent (12.36 g) was used for the next step without further purification. ¹H-NMR: δ 0.063 and 0.068 (2s, 6H), 0.88 (s, 9H), 1.38-1.49 (m, 1H), 1.54 (m, 1H, OH), 1.62 (m, 1H), 1.96 (m, 3H), 2.43 (m, 1H), 3.095 (t, 1H, J=5.6 Hz), 3.60 (m, 2H), 3.86 (m, 1H), 4.91 (m, 1H).

### Benzoic acid (2R,3S,7S)-7-(t-butyldimethyl)silanyloxy)-4-methylene-1-oxa-spiro[2.5]oct-2-yl methyl ester

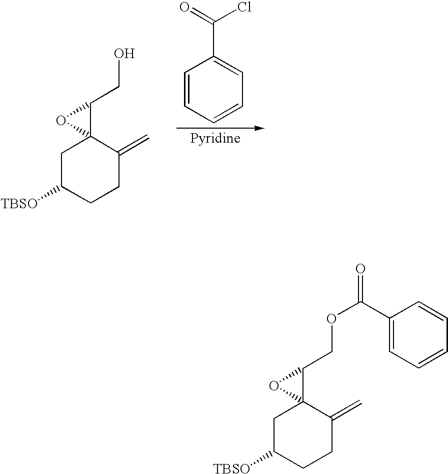

To a stirred solution of (2R,3S,7S)-[7-(tert-butyldimethyl)silanyloxy)-4-methylene-1-oxa-spiro[2.5]oct-2-yl]-methanol (12.36 g) in pyridine (50 mL) at room temperature, was added benzoyl chloride (8.5 mL, 73 mmol) and the reaction mixture was stirred for 2 h. Water (60 mL) was added and after stirring for 45 min at room temperature the mixture was diluted with hexane (250 mL), washed with NaHCO_{3aq} (2×250 mL), brine (250 mL) and dried over Na₂SO₄. The residue after evaporation of the solvent (15.28 g) was used for the next step without further purification. ¹H NMR: δ 8.04-7.80 (2H, m), 7.56-7.50 (1H, m), 7.44-7.37 (2H, m), 4.94 (1H, brs), 4.92 (1H, brs), 4.32 (1H, dd, J=4.8, 11.9 Hz), 4.14 (1H, dd, J=6.2, 11.9 Hz), 3.83 (1H, m), 3.21 (1H, dd, J=4.8, 6.2 Hz), 2.42 (1H, m), 2.04-1.90 (3H, m), 1.64-1.34 (2H, m), 0.83 (9H, s), 0.02 (3H, s), 0.01 (3H, s).

### Benzoic acid (2R,3S,5R,7S)-7-(t-butyldimethyl)silanyloxy)-5-hydroxy-4-methylene-1-oxa-spiro[2.5]oct-2-yl methyl ester

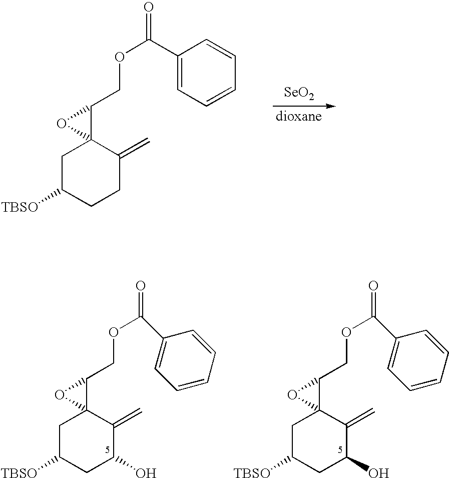

To a stirred solution of benzoic acid (2R,3S,7S)-7-(tert-butyldimethyl)silanyloxy)-4-methylene-1-oxa-spiro[2.5]oct-2-yl methyl ester (15.28 g)) in dioxane (450 mL) at 85° C. was added selenium dioxide (4.26 g, 38.4 mmol), followed by tert-butyl hydrogen peroxide (7.7 mL, 38.4 mmol, 5-6 M in nonane) and the reaction mixture was stirred at 85° C. for 13 h, after which selenium dioxide (2.39 g, 21.5 mmol) was added, followed by tert-butyl hydrogen peroxide (4.3 mL, 21.5 mmol, 5-6 M in nonane) and the reaction mixture was stirred at 85° C. for additional 24 h. The mixture was filtered off through a plug of silica gel (0.5 kg, AcOEt). The solvent was removed under vacuum and the residue was dissolved in AcOEt (250 mL) and washed with water (3×100 mL). The organic layer was dried over Na₂SO₄ and evaporated under vacuum. The residue (16 g) was purified by flash chromatography (0.5 kg, 10, 15 and 20% AcOEt in hexane) to give: Fraction A (1.1 g, of a starting material); Fraction B (0.78 g, of epimer b); Fraction C (3.01 g, 65:35 (epimer b:epimer a); Fraction D (6.22 g, 5:95 (epimer b:epimer a); Fraction D was crystallized two times (each time using the remaining oil) from hexane to give pale yellow solid Fraction E (6.0 g in total) and yellow-red oil Fraction F (0.2 g in total). Fractions C and F were purified by flash chromatography (300 g, 20% AcOEt in hexane) to give: Fraction G (0.8 g, of epimer b); Fraction H (2.4 g, 8:92 epimer b:epimer a). Fraction H was crystallized two times (each time using the remaining oil) from hexane to give pale yellow solid Fraction I (2.2 g in total) and yellow-red oil Fraction J (0.2 g in total). Fractions E and I were combined to give epimer a (8.2 g, 20.3 mmol, 50.7% total yield. [α]²²_{D} −10.6° (c 0.35, EtOH); ¹H NMR: δ 8.04 (2H, m), 7.58 (1H, m), 7.46 (2H, m), 5.32 (1H, br s), 5.18 (1H, br s), 4.33 (1H, dd, J=5.2, 11.9 Hz), 4.21 (1H, dd, J=6.0, 11.9 Hz), 4.14 (1H, ddd, J=2.6, 4.9, 10.0 Hz), 3.94 (1H, m), 3.25 (1H, dd, J=5.5, 5.9 Hz), 2.38 (1H, m), 2.05 (1H, t, J=11.5 Hz), 1.64 (1H, ddd, J=1.9, 4.3, 12.2 Hz), 1.52 dt, J=11.1, 11.7 Hz), 1.28 (1H, m), 0.87 (9H, s), 0.07 (3H, s), 0.06 (3H, s); ¹³C NMR: 166.31 (0), 145.52 (0), 133.29 (1), 129.65 (1), 129.54 (0), 128.46 (1), 107.44 (2),68.51 (1), 65.95 (1), 62.75 (2), 61.62 (1), 61.09 (0), 45.23 (2), 44.33 (2), 25.72 (3), 18.06 (0), −4.72 (3); MS HR-ES: Calcd. For C₂₂H₃₂O₅Si: M+Na 427.1911 Found: 427.1909.

### Benzoic acid (2R,3S,5S,7R)-7-(t-butyldimethyl)silanyloxy)-5-fluoro-4-methylene-1-oxa-spiro[2.5]oct-2-ylmethyl ester

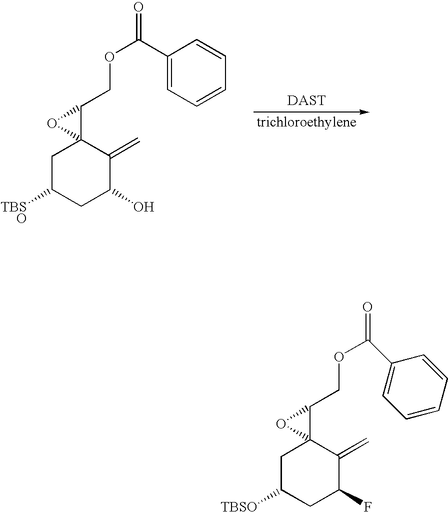

To a stirred solution of diethylaminosulfur trifluoride (16.5 mL, 126.0 mmol) in trichloroethylene (140 mL) was added a solution of benzoic acid (2R,3S,5R,7S)-7-(tert-butyldimethyl)silanyloxy)-5-hydroxy-4-methylene-1-oxa-spiro[2.5]oct-2-yl methyl ester epimer a (18.7 g, 46.2 mmol) in trichloroethylene (100 mL at −75° C. After stirring for 20 min. at −75° C. methanol (40 mL) was added, followed by NaHCO_{3aq} (50 mL) and the resulting mixture was diluted with hexane (700 mL) and washed with NaHCO_{3aq} (600 mL), dried over Na₂SO₄ and concentrated on rotary evaporator. The residue (25.6 g) was purified by flash chromatography (500 g, DCM:hexane:AcOEt 10:20:0.2) to give the titled compound (13.9 g, 34.2 mmol, 74%); [α]²⁹_{D} +38.9° (c 0.8, CHCl₃); ¹H NMR: δ 8.07 (2H, m), 7.57 (1H, m), 7.44 (2H, m), 5.33 (2H, m), 5.20 (1H, dt, J=2.9, 48 Hz), 4.55 (1H, dd, J=3.2, 12.3 Hz), 4.29 (1H, m), 4.02 (1H, dd, J=7.9, 12.3 Hz), 3.37 (1H, dd, J=3.2, 7.7 Hz), 2.45 (1H, m), 2.05 (1H, t, J=11.9 Hz), 1.73 (1H, dm), 1.62 (1H, m), 0.88 (9H, s), 0.08 (3H, s), 0.06 (3H, s); ¹³C NMR: 166.25 (0), 139.95 (0, d, J=17 Hz), 132.97 (1), 129.75 (0), 129.62 (1), 128.24 (1), 116.32 (2, d, J=9 Hz), 92.11 (1, d, J=162 Hz), 65.23 (1), 63.78 (2), 62.29 (1), 60.35 (0), 44.38 (2), 41.26 (2, d, J=23 Hz), 25.81 (3), 18.13 (0), −4.66(3); MS HR-ES: Calcd. For C₂₂H₃₁O₄SiF: M+H 407.2049 Found: 407.2046.

### (1E,3S,5R)-2-[5-(tert-Butyldimethyl)silanyloxy)-3-fluoro-2-methylene-cyclohexylidene]-ethanol

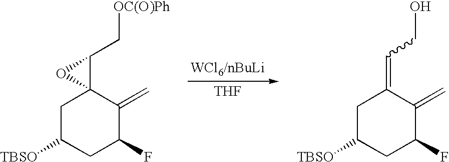

Tungsten hexachloride (36.4 g, 91 mmol) was added at −75° C. to THF (800 mL). The temperature was adjusted to −65° C. and nBuLi (73 mL, 182.5 mmol, 2.5M solution in hexane) was added maintaining temperature below −20° C. After the addition was completed the reaction mixture was allowed to come to room temperature and it was stirred for 30 min., cooled down to 0° C., when a solution of benzoic acid (2R,3S,5S,7R)-7-(tert-butyldimethyl)silanyloxy)-5-fluoro-4-methylene-1-oxa-spiro[2.5]oct-2-yl methyl ester (18.5 g, 45.5 mmol) in THF (50 mL) was added. Thus formed mixture was allowed to come to room temperature (2 h) and stirred for 16 h. Methanol (400 mL) was added followed by sodium methoxide (250 mL, 15% in methanol), the resulting mixture was stirred for 30 min then diluted with AcOEt (1 L) and washed with water (1 L) and brine (500 mL). The residue (21.6 g) after evaporation of the dried (Na₂SO₄) solvent was used for the next step without further purification.

¹H-NMR (CDCl₃); δ 0.09 (s, 6H), 0.81 (s, 9H), 1.80-2.22 (m, 3H), 2.44 (m, 1H), 4.10 (m, 1H), 4.14 (d, 2H, J=6.9 Hz), 4.98 (br s, 1H), 5.10 (d, 1H, J=50.0 Hz), 5.11 (s, 1H), 5.79 (t, 1H, J=6.8 Hz).

### (1Z,3S,5R)-2-[5-(tert-Butyldimethyl)silanyloxy)-3-fluoro-2-methylene-cyclohexylidene]-ethanol

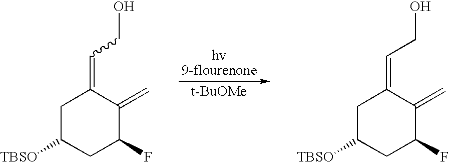

A solution of (1E,3S,5R)-2-[5-(tert-butyldimethyl)silanyloxy)-3-fluoro-2-methylene-cyclohexylidene]-ethanol (21.6 g, crude containing ca 10% of the Z isomer) and 9-fluorenone (1.8 g, 10 mmol) in tert-Butyl-methyl ether (650 mL) was irradiated with 450 W hanovia lamp with uranium core filter for 8 h. The residue after evaporation of solvent (23.95 g) was purified by flash chromatography (750 g, 5%,20%, AcOEt in hexane) to give the title compound (10.4 g, 36.3 mmol, 80% from benzoic acid (2R,3S,5S,7R)-7-(tert-butyldimethyl)silanyloxy)-5-fluoro-4-methylene-1-oxa-spiro[2.5]oct-2-yl methyl ester). [α]³⁰_{D} +40.1° (c 0.89, EtOH)

¹H-NMR: δ 5.65(1H, t, J=6.8 Hz), 5.31 (1H, dd, J=1.5, 1.7 Hz), 5.10 (1H, ddd, J=3.2, 6.0, 49.9 Hz), 4.95 (1H, d, J=1.7 Hz), 4.28 (1H, dd, J=7.3, 12.6 Hz), 4.19 (1H, ddd, J=1.7, 6.4, 12.7 Hz), 4.15 (1H, m), 2.48 (1H, dd, J=3.8, 13.0 Hz), 2.27-2.13 (2H, m), 1.88 (1H, m), 0.87 (9H, s), 0.07 (6H, s). ¹³C-NMR: 142.54(0,d, J=17 Hz), 137.12 (0, d, J=2.3 Hz), 128.54 (1), 115.30 (2, d, J=10 Hz), 92.11 (1, d, J=168 Hz), 66.82 (1, d, J=4.5 Hz), 59.45 (2), 45.15 (2), 41.44 (2, d, J=21 Hz), 25.76 (3), 18.06 (0), −4.75(3), −4.85(3).

### (1R,3Z,5S)-t-butyl-[3-(2-chloro-ethylidene)-5-fluoro-4-methylene-cyclohexyloxy]-dimethylsilane

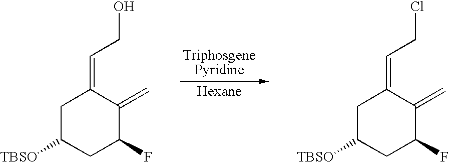

To a solution of (1Z,3S,5R)-2-[5-(tert-Butyldimethyl)silanyloxy)-3-fluoro-2-methylene-cyclohexylidene]-ethanol (8.07 g, 28.2 mmol) and triphosgene (4.18 g, 14.1 mmol) in hexane (150 mL) at 0° C. was added over 30 min a solution of pyridine (4.5 mL, 55.6 mmol) in hexane (20 mL) and the reaction mixture was stirred at this temperature for 30 min and at room temperature for another 30 min. The reaction mixture was washed with CuSO₄ aq (3×200 mL). The combined aqueous layers were back-extracted with hexane (2×100 mL). The organic layers were combined, dried (MgSO₄), and concentrated in vacuo to give the title compound (9.0 g, overweight). This material was used immediately in the next step without further purification. [α]²⁵_{D} +73.0° (c 0.28, CHCl₃); IR (CHCl₃) 1643, 838 cm⁻¹; ¹H-NMR δ 0.08 (s, 6H), 0.88 (s, 9H), 1.84-2.03 (m, 1H), 2.12 (br s, 1H), 2.24 (m, 1H), 2.48 (br d, J=13 Hz, 1H), 4.06-4.26 (m, 3H), 5.10 (br d, J=48 Hz), 5.16 (s, 1H), 5.35 (s, 1H), 5.63 (br t, J=6 Hz, 1H).

### (1S,3Z,5R)-1-fluoro-5-(t-butyldimethyl)silanyloxy)-2-methenyl-3-(diphenylphosphinoyl)ethylidene cyclohexane

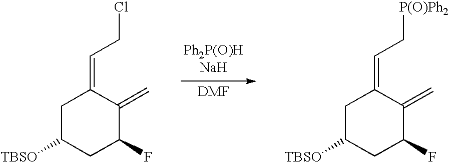

Diphenylphosphine oxide (6.70 g, 33.1 mmol) was added portionwise, over 15 min to a suspension of NaH (1.33 g, 33.1 mmol, 60% dispersion in mineral oil) in DMF (50 mL) at 10° C. The resulting solution was stirred at room temperature for 30 min and cooled to −60° C. The solution of crude (1R,3Z,5S)-t-butyl-[3-(2-chloro-ethylidene)-5-fluoro-4-methylene-cyclohexyloxy]-dimethylsilane (9.0 g) in DMF (20 mL) was then added dropwise. The reaction mixture was stirred at −60° C. for 2 h and at room temperature for 1 h, diluted with diethyl ether (600 mL) and washed with water (3×200 mL). The aqueous layers were extracted with diethyl ether (200 mL). The combined organic layers were dried (MgSO₄) and concentrated under reduced pressure to give white solid. The crude product was recrystallized from diisopropyl ether (25 mL). The resulting solid was collected by filtration, washed with cold diisopropyl ether (5 mL) and dried under high vacuum to give the title compound (7.93 g). The mother liquor was concentrated and the residue was subjected to chromatography on silca gel (50 g, 30%-50% AcOEt in hexane) to give title compound (2.22 g). Thus the total yield of the of the titled compound was (10.1 g, 21.5 mmol, 76% overall from (1Z,3S,5R)-2-[5-(tert-Butyldimethyl)silanyloxy)-3-fluoro-2-methylene-cyclohexylidene]-ethanol. [α]²⁵_{D} +50.2° (c 0.84, CHCl₃); IR (CHCl₃) 835, 692 cm¹; UVλₘₐₓ (ethanol) 223 (ε 22770), 258 (1950), 265 (1750), 272 nm (1280); MS, m/e 470 (M⁺), 455 (4), 450 (8), 413 (98), 338 (9), 75 (100); ¹H-NMR: δ 0.02 (s, 6H), 0.84 (s, 9H), 1.76-1.93 (m, 1H), 2.16 (m, 2H), 2.42 (br d, 1H), 3.28 (m, 2H), 4.01 (m, 1H), 5.02 (dm, J=44 Hz, 1H), 5.14 (s, 1H), 5.30 (s, 1H), 5.5 (m, 1H), 7.5 (m, 6H), 7.73 (m, 4H). Analysis Calcd for C₂₇H₃₆O₂FPSi: C, 68.91; H, 7.71; F, 4.04;. Found: C, 68.69; H, 7.80; F, 3.88.

### Synthesis of C,D-Ring/Side Chain Precursor

### (S)-2-((1R,3aR,4S,7aR)-4-hydroxy-7a-methyl-octahydro-inden-1-yl)-propionaldehyde

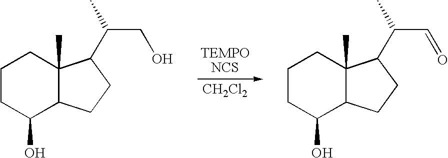

A 250-mL flask was charged with 0.99 g (4.67 mmol) of Lythgoe diol, 75 mg (0.48 mmol) of TEMPO, 146 mg (0.53 mmol) of tetrabutylammonium chloride hydrate, and dichloromethane (50 mL). To this vigorously stirred solution was added a buffer solution (50 mL) prepared by dissolving sodium hydrogen carbonate (4.2 g) and potassium carbonate (0.69 g) in a volume of 100 mL of water. The mixture was stirred vigorously and 839 mg (6.28 mmol) of N-chlorosuccinimide was added. TLC (1:2, ethyl acetate-heptane) showed the gradual conversion of educt (Rf 0.32) to the titled aldehyde (Rf 0.61). After 18 h an additional quantity of 830 mg (6.28 mmol) of N-chlorosuccinimide was added and one hour later 20 mg of TEMPO was added and the mixture was stirred for 24 h. The organic layer was separated and the aqueous layer re-extracted with dichloromethane (3×50 mL). The combined organic extracts were washed with brine, dried and concentrated in vacuo. The residue was purified by column chromatography (SiO₂, ethyl acetate/heptane=1:3) to furnish 876 mg of crude aldehyde (89%) ¹H NMR: δ 9.58 (1H, d, J=2.8 Hz), 4.12 (1H, m), 2.50-2.30 (1H, m), 2.10-1.10 (13H, m), 1.11 (3H, d, J=7.0 Hz), 0.99 (3H, s).

### (1R,3aR,4S,7aR)-7a-methyl-1-(S)-1-methyl-2-oxo-ethyl)-octahydroinden-4-yl ester

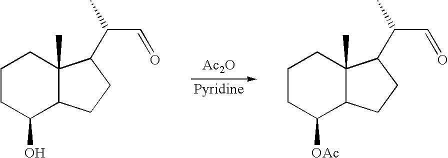

Crude (S)-2-((1R,3aR,4S,7aR)-4-hydroxy-7a-methyl-octahydro-inden-1-yl)-propionaldehyde (255 mg, 1.21 mmol) was dissolved in pyridine (1 mL), the soln. cooled in an ice bath and DMAP (5 mg) and acetic anhydride (0.5 mL) were added. The mixture was stirred at room temperature for 24 h then diluted with water (10 mL), stirred for 10 min and equilibrated with ethyl acetate (30 mL). The organic layer was washed with a mixture of water (10 mL) and 1 N sulfuric acid (14 mL), then with water (10 mL) and saturated sodium hydrogen carbonate solution (10 mL), then dried and evaporated. The resulting residue (201 mg) was chromatographed on a silica gel column using 1:4 ethyl acetate-hexane as mobile phase. The fractions containing the product were pooled and evaporated to give the title compound as a colorless syrup (169 mg, 0.67 mmol, 67%). ¹H NMR (300 MHz, CDCl₃): δ 9.56 (1H, d, J=2.0 Hz), 5.20 (1H, br s), 2.44-2.16 (1H, m), 2.03 (3H, s), 2.00-1.15 (12H, m), 1.11 (3H, d, J=7.0 Hz), 0.92 (3H, s).

### Acetic acid (3aR,4S,7aR)-1-E-ethylidene-7a-methyl-octahydroinden-4-yl ester

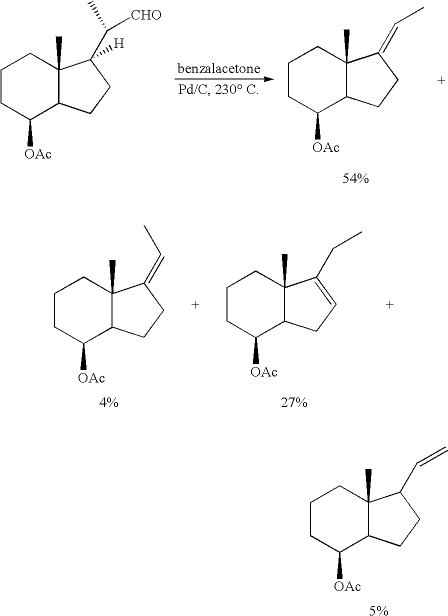

To a solution of (1R,3aR,4S,7aR)-7a-methyl-1-((S)-1-methyl-2-oxo-ethyl)-octahydroinden-4-yl ester (480 mg, 1.90 mmol) in diethylether (5 mL) was added 10% Pd on Carbon (25 mg). The suspension was stirred at room temperature for 20 min., filtered through a path of Celite and the filtrate was concentrated in vacuo. To the residue was added benzalacetone (350 mg, 2.40 mmol, distilled) and 10% Pd on Carbon (50 mg). The suspension was degassed by evacuating the flask and refilling with nitrogen (2×). Then the flask was immersed in a 230° C. heating bath for 40 min. After cooling at room temperature the suspension was diluted with ethyl acetate, filtered through a path of Celite and the filtrate was concentrated in vacuo. The residue was purified by column chromatography (SiO₂, ethyl acetate/heptane=1:9) affording 290 mg (68%) of a mixture of CD olefins. GC analysis: titled product (54%); Z isomer (4%); internal olefin (27%); terminal olefin (5%); other impurities (10%).

### (2R,3aR,4S,7aR)-1-E-ethylidene-2-hydroxy-7a-methyl-octahydroinden-4-yl ester (a) and acetic acid (2S,3aR,4S,7aR)-1-E)-ethylidene-2-hydroxy-7a-methyl-octahydroinden-4-yl ester (b)

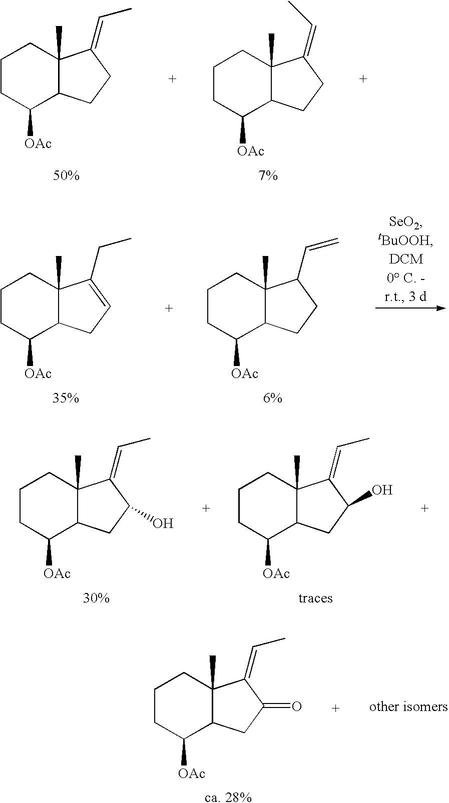

To a suspension of SeO₂ (460 mg, 4.15 mmol) in dichloromethane (30 mL) was added tert.-butylhydroperoxide (9.0 mL, 70 w/w-% solution in water, 65.7 mmol). The suspension was stirred at room temperature for 30 min., cooled at 0° C. and a solution of CD-isomers (9.13 g, 41.1 mmol, contains ca 50% of 16) in dichloromethane (35 mL) was added dropwise within 30 min. The reaction mixture was allowed to reach room temperature overnight and stirring was continued at 30° C. for 2 days. Conversion was checked by GC. The reaction was quenched by addition of water and the aqueous layer was extracted with dichloromethane (3×). The combined organic layers were washed with water (4×), washed with brine, dried (Na₂SO₄), filtered and the filtrate was concentrated in vacuo. The residue was purified by column chromatography (SiO₂, ethyl acetate/heptane=1:3) affording three main fractions: Fraction 1: Ketone (2.08 g, 42% yield); contaminated with 2 impurities; purity ˜75%; Fraction 2: mixed fraction of alcohol epimer a+unwanted isomer (1.32 g); Fraction 3: Alcohol epimer a (2.10 g, 42% yield); contaminated with ca. 12% byproduct, but pure enough for further synthesis. Fraction 2 was purified again by column chromatography affording 1.01 g (20% yield) of alcohol epimer a contaminated with ca. 20% of an unwanted isomer, but pure enough for further synthesis. *Note: During the oxidation reaction the formation of both isomers epimer a and epimer b was observed by tlc and GC. After prolonged reaction times the intensity of the lower spot on the (mixture of epimer b and other isomers) decreased and the formation of ketone was observed. It is important that not only conversion of starting material to alcohol epimer a and epimer b is complete but also that epimer epimer b is completely oxidized to ketone. Epimer epimer b can not be separated from unwanted isomers. Retention times on GC: starting material ret. Time=8.06 min; product ret. Time=9.10 min; epimer b ret. Time=9.30 or 9.34 min; ketone ret. Time=9.60 min. epimer a: ¹H NMR: δ 0.94 (s, 3H), 1.30 (m, 1H), 1.40-1.46 (m, 1H), 1.46-1.80 (m, 4H), 1.77 (dd, J=7.2, 1.2 Hz, 3H), 1.80-1.94 (m, 4H), 2.02 (s, 3H), 4.80 (br. s, 1H), 5.23 (m, 1H), 5.47 (qd, J=7.2, 1.2 Hz, 1H). GC-MS: m/e 223 (M−15), 178 (M−60), 163 (M−75). epimer b: ¹H NMR: δ 1.24 (s, 3H), 1.38-1.60 (m, 5H), 1.68-1.88 (m, 3H), 1.72 (dd, J=7.2, 1.2 Hz, 3H), 1.99 (ddd, J=11.0, 7.0, 3.7 Hz, 1H), 2.03 (s, 3H), 2.26 (m, 1H), 4.36 (m, 1H), 5.14 (m, 1H), 5.30 (qd, J=7.2, 1.2 Hz, 1H). GC-MS: m/e 223 (M−15), 178 (M−60), 163 (M−75).

### Reduction of Ketone to Alcohol Epimer b

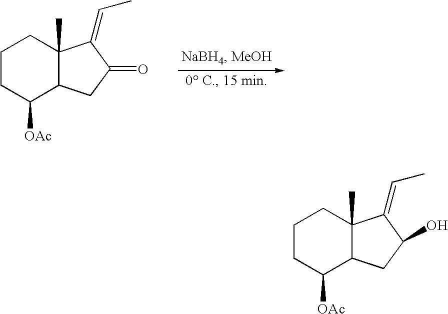

A solution of ketone (2.08 g, contaminated with 2 impurities) in methanol (8 mL) was cooled at 0° C. and sodium borohydride (0.57 g, 15.1 mmol) was added in portions. After stirring at 0° C. for 1 h, the showed complete conversion (no UV active compound visible on the). The reaction mixture was quenched by addition of sat. aqueous NH₄Cl solution (30 mL). Water was added and the aqueous layer was extracted with ethyl acetate (3×). The combined organic layers were washed with brine, dried (Na₂SO₄), filtered and the filtrate was concentrated in vacuo. The residue was purified by column chromatography (SiO₂, ethyl acetate/heptane=1:3) affording alcohol epimer b (1.20 g, 24% over two steps) as a colorless oil.

### Acetic acid (3aR,4S,7aR)-7a-methyl-1-(1-(R)-methyl-3-oxo-propyl)-3a,4,5,6,7,7a-hexahydro-3H-inden-4-yl ester

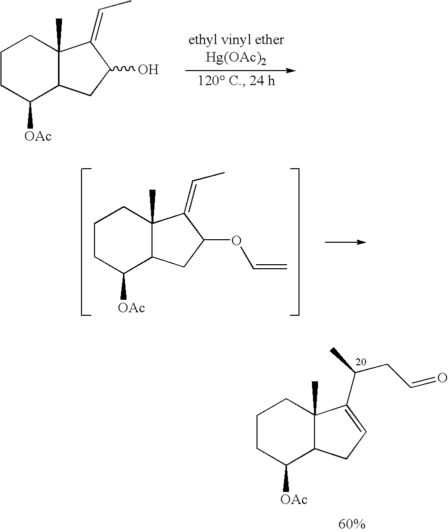

Both (2R,3aR,4S,7aR)-1-E-ethylidene-2-hydroxy-7a-methyl-octahydroinden-4-yl ester (a) and acetic acid (2S,3aR,4S,7aR)-1-E)-ethylidene-2-hydroxy-7a-methyl-octahydroinden-4-yl ester (b) (4.3 g, 18.1 mmol, purity 90%) were converted to compound Acetic acid (3aR,4S,7aR)-7a-methyl-1-(1-(R)-methyl-3-oxo-propyl)-3a,4,5,6,7,7a-hexahydro-3H-inden-4-yl ester in three batches. To a solution of epimer a (2.1 g, 8.82 mmol) in ethyl vinyl ether (20 mL) was addedHg(OAc)₂ (2.23 g, 7.00 mmol). The suspension was poured into a pyrex pressure tube, flushed with N₂ and closed tightly. The mixture was stirred at 120° C. for 24 h, cooled at room temperature and filtered. The filtrate was concentrated in vacuo and the residue was combined with the crude product of the two other batches and purified twice* by column chromatography (SiO₂, ethyl acetate/heptane=1:4) affording the titled compound (2.58 g, 60%) as a slightly yellow oil. The product solidified upon storage in the freezer. A second purification by column chromatography was advantageous due to the byproducts present in the starting material.

To a solution of epimers a and b (173 mg, 0.73 mmol) in toluene (2 mL) was added a catalytic amount of [Ir(COD)Cl]₂ (5 mg), Na₂CO₃ (46 mg, 0.44 mmol) and vinyl acetate (0.13 mL, 1.45 mmol). After heating the suspension at 100° C. for 2 h, the indicates ca. 20% conversion to intermediate. (J. Am. Chem. Soc., 2002, 134, 1590-1591.) More vinyl acetate (0.15 mL) was added and stirring at 100° C. was continued for 18 h. According the a mixture of intermediate and the titled compound was formed but conversion of the starting material was still not complete. More vinyl acetate (2 mL) was added and stirring at 100° C. was continued for 24 h. Tlc shows complete conversion of the starting material to a mixture of intermediate and the titled compound. The suspension was concentrated in vacuo and the residue was purified by column chromatography (SiO₂, ethyl acetate/heptane=1:9) affording 60 mg of intermediate (31%) and 45 mg of the titled compound (23%). ¹H NMR: δ 1.02 (s, 3H), 1.14 (d, J=7.1 Hz, 3H), 1.36 (M, 1H), 1.47-1.62 (m, 2H), 1.72-1.90 (m, 4H), 2.03 (s, 3H), 2.02-2.14 (m, 2H), 2.33 (ddd, J=16.2, 7.3, 2.6 Hz, 1H), 2.53 (ddd, J=16.2, 5.8, 1.8 Hz, 1H), 2.72 (m, 1H), 5.19 (m, 1H), 5.40 (m, 1H), 9.68 (s, 1H).

### 5(R)-((3aR,4S,7aR)-4-acetoxy-7a-methyl-3a,4,5,6,7,7a-hexahydro-3H-inden-1-yl)-hex-2-E-enoic acid ethyl ester

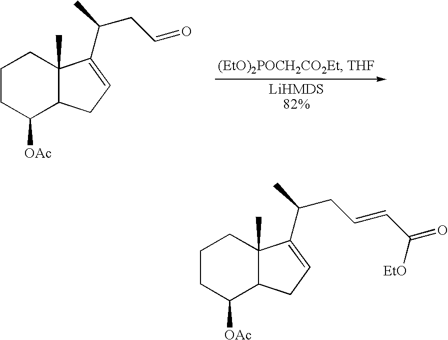

Acetic acid (3aR,4S,7aR)-7a-methyl-1-(1-(R)-methyl-3-oxo-propyl)-3a,4,5,6,7,7a-hexahydro-3H-inden-4-yl ester (2.24 g, 8.47 mmol) and triethyl phosphonoacetate (5.74 g, 25.6 mmol, 3 eq.) were dissolved under N₂ atmosphere in THF (40 mL, freshly distilled over Na/benzophenone). The mixture was cooled at −100° C. and a solution of LiHMDS in hexanes (16.8 mL, 1 M solution, 2 eq.) was added dropwise within 20 min. After stirring at −100° C. [symbol] −78° C. for 70 min. the reaction was quenched by dropwise addition of water (10 mL) and subsequently addition of sat. NH₄Cl solution (10 mL). Water was added and it was extracted with tert. butyl methyl ether (3×). The combined organic layers were washed with water (2×), brine (1×), dried (Na₂SO₄), filtered and the filtrate was concentrated in vacuo. The residue was purified by column chromatography (SiO₂, ethyl acetate/heptane=1:10) affording ester the titled compound (2.15 g, 76%) as a colorless oil; purity according NMR: >95% (no Z-isomer detected). ¹H NMR: δ 0.99 (s, 3H), 1.06 (d, J=7.2 Hz, 3H), 1.27 (t, J=7.1 Hz, 3H), 1.36 (td, J=13.3, 4.0 Hz, 1H), 1.46-1.62 (m, 2H), 1.72-1.90 (m, 4H), 1.96-2.17 (m, 3H), 2.03 (s, 3H), 2.22-2.39 (m, 2H), 4.17 (q, J=7.2 Hz, 2H), 5.20 (br. s, 1H), 5.37 (br. s, 1H), 5.78 (dm, J=15.4 Hz, 1H), 6.88 (dt, J=15.4, 7.3 Hz, 1H). HPLC: purity >99% (218 nm). HPLC-MS: m/e 357 (M+23), 275 (M−59).

### (3aR,4S,7aR)-1-((S,E)-5-ethyl-5-hydroxy-1-methyl-hept-3-enyl)-7a-methyl-3a,4,5,6,7,7a-hexahydro-3H-inden-4-ol

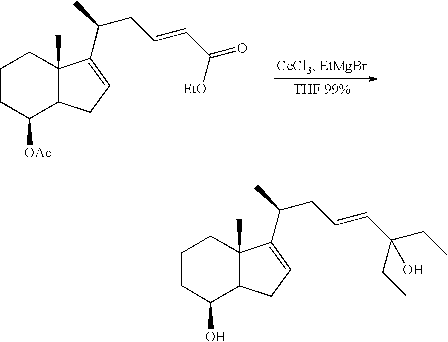

CeCl₃×7H₂O (29.1 g) was dried in vacuo (10⁻³ mbar) in a three-necked flask at 160° C. for 6 h affording anhydrous CeCl₃ (18.7 g, 76.0 mmol, 12 eq.). After cooling at room temperature the flask was purged with nitrogen. THF (200 mL, freshly distilled over Na/benzophenone) was added and the mixture was stirred at room temperature for 18 h. Subsequently the suspension was cooled at 0° C. and a solution of EtMgBr in THF (75 mL, 1 M solution) was added dropwise within 20 min. After stirring the light brown suspension at 0° C. for 2 h a solution of 5(R)-((3aR,4S,7aR)-4-acetoxy-7a-methyl-3a,4,5,6,7,7a-hexahydro-3H-inden-1-yl)-hex-2-E-enoic acid ethyl ester (2.15 g, 6.42 mmol) in THF (30 mL, freshly distilled over Na/benzophenone) was added dropwise within 10 min. After stirring at 0° C. for 30 min. the showed complete conversion and the reaction was quenched by addition of water (60 mL). More water was added and the mixture was extracted with 50% ethyl acetate in heptane (3×). The combined organic layers were washed with sat. NaHCO₃ solution (2×), brine (1×), dried (Na₂SO₄), filtered and the filtrate was concentrated in vacuo affording a slightly yellow oil. The crude product (2.4 g) was combined with a 2^{nd} batch (600 mg crude (3aR,4S,7aR)-1-((S,E)-5-ethyl-5-hydroxy-1-methyl-hept-3-enyl)-7a-methyl-3a,4,5,6,7,7a-hexahydro-3H-inden-4-ol obtained from 550 mg of starting material). Purification by column chromatography (SiO₂, ethyl acetate/heptane=1:3) afforded (3aR,4S,7aR)-1-((S,E)-5-ethyl-5-hydroxy-1-methyl-hept-3-enyl)-7a-methyl-3a,4,5,6,7,7a-hexahydro-3H-inden-4-ol (2.45 g, 99%) as a colorless oil. ¹H NMR: δ 0.84 (t, J=7.3 Hz, 6H), 1.04 (d, J=7.2 Hz, 3H), 1.05 (s, 3H), 1.23-1.60 (m, 9H), 1.67-2.02 (m, 6H), 2.12-2.32 (m, 3H), 4.17 (m, 1H), 5.33 (m, 1H), 5.35 (dm, J=15.4 Hz, 1H), 5.51 (ddd, J=15.4, 7.4, 6.5 Hz, 1H). HPLC: purity=98% (212 nm). HPLC-MS: m/e 330 (M+24), 289 (M−17), 271 (M−35).

### (3aR,4S,7aR)-1-((S,E)-5-ethyl-5-hydroxy-1-methyl-hept-3-enyl)-7a-methyl-3a,4,5,6,7,7a-hexahydro-3H-inden-4-one

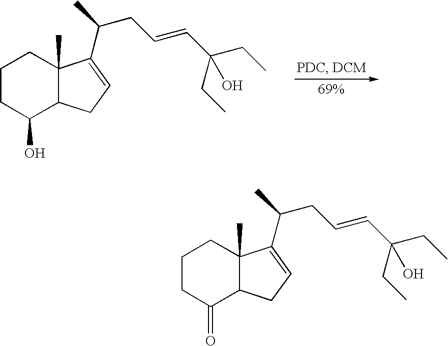

A solution of (3aR,4S,7aR)-1-((S,E)-5-ethyl-5-hydroxy-1-methyl-hept-3-enyl)-7a-methyl-3a,4,5,6,7,7a-hexahydro-3H-inden-4-ol (465 mg, 1.52 mmol) in dichloromethane (30 mL) was cooled in an ice-bath and treated portion-wise with pyridinium dichromate (1.28 g, 3.40 mmol, 2.2 eq.). The reaction mixture was stirred at 0° C. for 6 h and at room temperature for 18 h. The reaction mixture was filtered through a path of Celite. The filtercake was washed with dichloromethane and the combined filtrates were concentrated in vacuo. The residue was purified by column chromatography (SiO₂, 25% ethyl acetate in heptane) affording the titled compound (320 mg, 69%) as a colorless oil. ¹H NMR: δ 0.82 (s, 3H), 0.85 (br. t, J=7.2 Hz, 6H), 1.05 (d, J=6.9 Hz, 3H), 1.34 (br. s, 1H), 1.52 (br. q, J=6.9 Hz, 4H), 1.65 (td, J=12.1, 5.6 Hz, 1H), 1.84-1.93 (m, 1H), 1.93-2.16 (m, 4H), 2.16-2.33 (m, 4H), 2.42 (ddt, J=15.4, 10.4, 1.6 Hz, 1H), 2.82 (dd, J=10.4, 6.0 Hz, 1H), 5.30 (m, 1H), 5.38 (dm, J=15.6 Hz, 1H), 5.54 (ddd, J=15.6, 7.1, 6.0 Hz, 1H).

### Larger Scale Synthesis of C,D-Ring/Side Chain Precursor

### Acetic acid (1R,3aR,4S,7aR)-1-((S)-1-hydroxypropan-2-yl)-7a-methyl-octahydro-1H-inden-4-yl ester

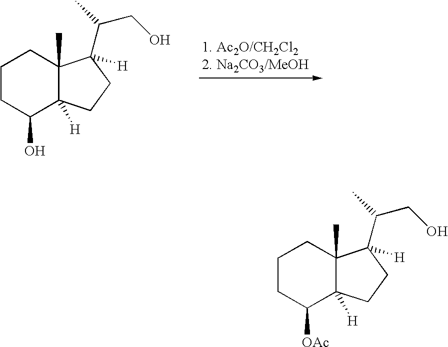

A 1 l round bottom flask equipped with stirring bar and Claisen adapter with rubber septum was charged with Lythgoee diol starting material (38.41 g, 180.9 mmol), dichloromethane (400 mL), pyridine (130 mL) and DMAP (5.00 g, 40.9 mmol). Acetic anhydride (150 mL) was added slowly and the mixture was stirred at room temperature for 14.5 h. Methanol (70 mL) was added dropwise (exothermic reaction) to the reaction mixture and the solution was stirred for 30 min. Water (1 L) was added and the aqueous layer was extracted with dichloromethane (2×250 mL). The extracts were washed with 1N HCl (200 mL) and solution of NaHCO₃ (200 mL), dried (Na₂SO₄) and evaporated to dryness with toluene (150 mL). The residue was dissolved in methanol (300 mL) and sodium carbonate (40.0 g) was added. The suspension was stirred for 24 h. Additional portion of sodium carbonate (10.0 g) was added and the reaction mixture was stirred for 18 h. Methanol was removed on a rotary evaporator. Water (500 mL) was added and the mixture was extracted with ethyl acetate (3×250 mL), dried (Na₂SO₄) and concentrated in vacuo. The residue was purified by FC (0.4 kg of silica gel, 20%, 30% hexane-ethyl acetate) to give the title compound Acetic acid (1R,3aR,4S,7aR)-1-((S)-1-hydroxypropan-2-yl)-7a-methyl-octahydro-1H-inden-4-yl ester (45 g, 98%). ¹H NMR (DMSO-D6) 5.03 (1H, br s), 4.26 (1H, dd, J=5.9, 5.1 Hz), 3.42-3.36 (1H, m), 3.10-3.02 (1H, m), 1.99 (3H, s), 1.96-1.91 (1H, m), 1.77-1.58 (3H, m), 1.50-1.08 (9H, m), 0.93 (3H, d, J=6.6 Hz), 0.85 (3H, s).

### Acetic acid (1R,3aR,4S,7aR)-7a-methyl-1-((S)-oxopropan-2-yl)-octahydro-1H-inden-4-yl ester

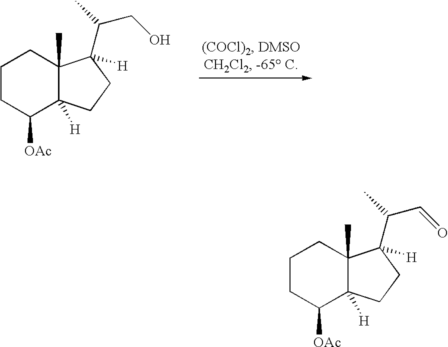

To a cooled solution (−65° C.). of oxalyl chloride (17 mL, 195 mmol) in dichloromethane (150 mL) was added within 35 min. a solution of DMSO (27 mL, 380 mmol) in dichloromethane (200 mL), keeping the temperature below −65° C. After complete addition stirring at −65° C. was continued for 15 min. Subsequently a solution of acetic acid (1R,3aR,4S,7aR)-1-((S)-1-hydroxypropan-2-yl)-7a-methyl-octahydro-1H-inden-4-yl ester (41 g, 161 mmol) in dichloromethane (300 mL) was added dropwise within 80 min., keeping the temperature below −65° C. During addition a solid precipitated. After complete addition stirring at −65° C. was continued for 1 h. Subsequently a solution of triethylamine (110 mL) in dichloromethane (200 mL) was added dropwise within 30 min. After complete addition stirring at −65° C. was continued for 45 min. The cooling bath was removed and the reaction mixture was allowed to warm to 5° C. within 1 h. Dichloromethane (ca. 600 mL) was removed by distillation under reduced pressure and to the residue was added water (600 mL) and tert-Butyl-methyl ether (500 mL). The organic layer was separated and the aqueous layer was extracted with tert-Butyl-methyl ether (2×200 mL). The combined organic layers were dried (Na₂SO₄), filtered and concentrated in vacuo. The residue was purified by column chromatography (800 g of silica gel, 15% ethyl acetate in heptane) affording 38 g (94%) of the title compound as a slightly yellow oil. ¹H NMR (CDCl₃): δ 9.56 (1H, d, J=2.0 Hz), 5.20 (1H, br s), 2.44-2.16 (1H, m), 2.03 (3H, s), 2.00-1.15 (12H, m), 1.11 (3H, d, J=7.0 Hz), 0.92 (3H, s).

### Acetic acid (3aR,4S,7aR)-1-E-ethylidene-7a-methyl-octahydroinden-4-yl ester

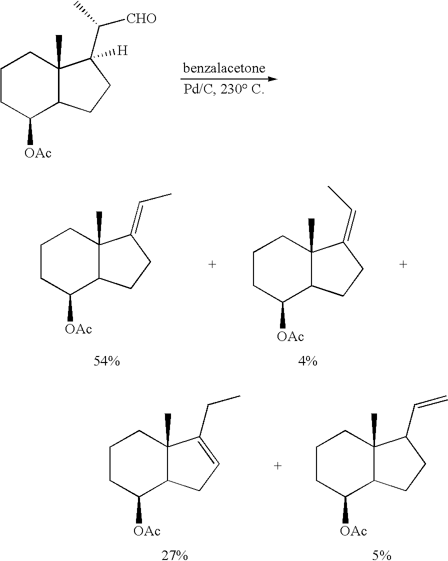

Benzalacetone was purified by bulb to bulb distillation (130° C., 10⁻² mbar) before use. To a solution of acetic acid (1R,3aR,4S,7aR)-7a-methyl-1-((S)-oxopropan-2-yl)-octahydro-1H-inden-4-yl ester (38.3 g, 0.15 mol) in diethyl ether (240 mL) was added 10% palladium on charcoal (1.8 g). The suspension was stirred at room temperature for 45 min., filtered through a path of Celite and the filtrate was concentrated in vacuo. To the residue was added benzalacetone (28.3 g, 0.19 mol) and 10% palladium on charcoal (1.8 g). The suspension was degassed by evacuating the flask and refilling with nitrogen. Then the flask was partially immersed in a 230° C. oil bath for 40 min. After cooling at room temperature the suspension was diluted with ethyl acetate, filtered through a path of Celite and the filtrate was concentrated in vacuo. The residue was purified by column chromatography (1800 g of SiO₂, 5-10% ethyl acetate in heptane) affording 21.6 g (65%) of a mixture of ^{17E}, Δ^{17Z}, Δ¹⁶ and Δ²⁰ indene olefins, which are present in 51%, 4%, 25%, and 1%, respectively (GC analysis). The mixture of isomers was used in the next step without further purification.

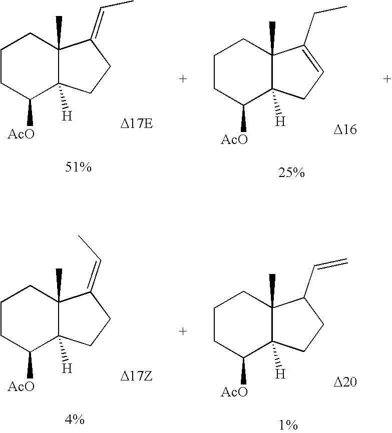

¹H NMR (CDCl₃, signals of the desired Δ^{17E} isomer): 5.21 (m, 1H), 4.98-5.07 (m, 1H), 2.15-2.35 (m, 2H), 2.05 (s, 3H), 1.53 (d, 3H, J=7 Hz), δ 0.96 (s, 3H).

In a different experiment the desired product was isolated from the mixture of olefins (Δ^{17E}:Δ^{17Z}:Δ₁₆:Δ²⁰=65:4:27:4) by silver nitrate impregnated silica gel medium pressure chromatography in a 55% yield (U.S. Pat. No. 5,939,408).

### (2R,3aR,4S,7aR)-1-E-ethylidene-2-hydroxy-7a-methyl-octahydroinden-4-yl ester (17a) and acetic acid (2S,3aR,4S,7aR)-1-E)-ethylidene-2-hydroxy-7a-methyl-octahydroinden-4-yl ester

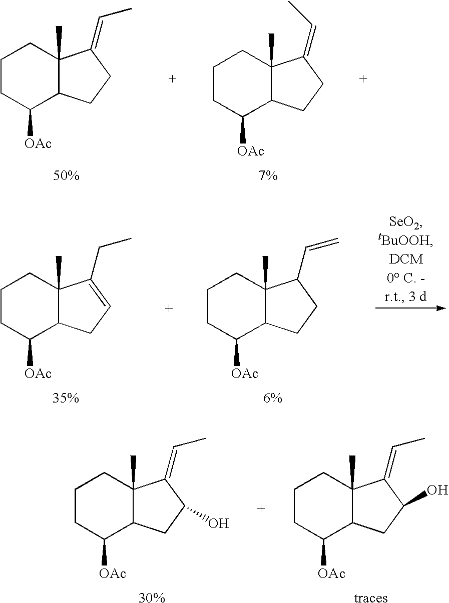

To a suspension of SeO₂ (1.4 g; 12.6 mmol) in dichloromethane (55 mL) was added t.-butyl-hydroperoxide (17 mL, 70 w/w-% solution in water, 124 mmol). The suspension was stirred at room temperature for 30 min, cooled at 0° C. and a solution of acetic acid (3aR,4S,7aS,E)-1-ethylidene-7a-methyl-octahydro-1H-inden-4-yl ester (18.8 g, 84.5 mmol, as part of a mixture of Δ^{17E}, Δ^{17Z}, Δ¹⁶ and Δ^{\tilde{2}}ndene olefins; contains 51% of desired isomer Acetic acid (3aR,4S,7aR)-1-E-ethylidene-7a-methyl-octahydroinden-4-yl ester) in dichloromethane (70 mL) was added dropwise. The reaction mixture was stirred at 0° C. for 1 h, at room temperature for 18 h and subsequently at 30° C. for 3 days. To the reaction mixture was added water (350 mL) and ethyl acetate (400 mL). The layers were separated and the aqueous layer was extracted with ethyl acetate (1×400 mL, 1×350 mL, 1×150 mL). Water (600 ml) was added to the combined organic fractions and the layers were mixed thoroughly for 60 min by magnetic stirring. The organic layer was separated, dried (Na₂SO₄) and concentrated in vacuo. The residue was purified by column chromatography (1 kg SiO₂; eluting with 4 L 20% AcOEt in heptane, 4 L 25% AcOEt in heptane, 4 L 33% AcOEt in heptane) affording: Fraction A (4.2 g, mixture containing ca. 75% of a ketone fragment); Fraction B (7.2 g of alcohol Acetic acid (3aR,4S,7aR)-1-E-ethylidene-7a-methyl-octahydroinden-4-yl ester, purity ca. 90%). Fraction A was dissolved in methanol (100 mL) and cooled at 0° C. Sodium borohydride (1.1 g, 29 mmol) was added in portions. After stirring at 0° C. for 40 min., the showed complete conversion. The reaction mixture was quenched by addition of sat. aqueous NH₄Cl solution (30 mL) and was extracted with ethyl acetate (3×). The combined organic layers were washed with brine, dried (Na₂SO₄), filtered and the filtrate was concentrated in vacuo. The residue (4.5 g) was purified by column chromatography (SiO₂, ethyl acetate/heptane=1:3) to give: Fraction C (3.2 g, of alcohol acetic acid (2S,3aR,4S,7aR)-1-E)-ethylidene-2-hydroxy-7a-methyl-octahydroinden-4-yl ester (b)). Fraction B and C were combined affording 10.4 g of a mixture of alcohol (2R,3aR,4S,7aR)-1-E-ethylidene-2-hydroxy-7a-methyl-octahydroinden-4-yl ester (a) and acetic acid (2S,3aR,4S,7aR)-1-E)-ethylidene-2-hydroxy-7a-methyl-octahydroinden-4-yl ester (b) (93% yield based on the amount of 51% of starting material in the mixture of CD olefins) as a colorless oil.

Alcohol a: ¹H NMR (CDCl₃): δ 5.47 (qd, J=7.2, 1.2 Hz, 1H), 4.80 (br. s, 1H), 5.23 (m, 1H), 1.80-1.94 (m, 4H), 2.02 (s, 3H), 1.77 (dd, J=7.2, 1.2 Hz, 3H), 1.46-1.80 (m, 4H), 1.40-1.46 (m, 1H), 1.30 (m, 1H), 0.94 (s, 3H); GC-MS: m/e 223 (M−15), 178 (M−60), 163 (M−75); MS: m/e 223 (M−15), 178 (M−60), 163 (M−75).

Alcohol b: ¹H NMR (CDCl₃): δ 5.30 (qd, J=7.2, 1.2 Hz, 1H), 5.14 (m, 1H), 4.36 (m, 1H), 2.26 (m, 1H), 2.03 (s, 3H), 1.99 (ddd, J=11.0, 7.0, 3.7 Hz, 1H), 1.72 (dd, J=7.2, 1.2 Hz, 3H), 1.68-1.88 (m, 3H), 1.38-1.60 (m, 5H), 1.24 (s, 3H); GC-MS: m/e 223 (M−15), 178 (M−60), 163 (M−75); MS: m/e 223 (M−15), 178 (M−60), 163 (M−75).

### Acetic acid (3aR,4S,7aR)-7a-methyl-1-(1-(R)-methyl-3-oxo-propyl)-3a,4,5,6,7,7a-hexahydro-3H-inden-4-yl ester

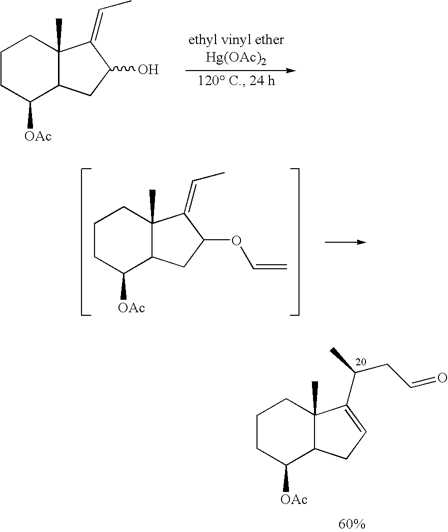

A mixture of acetic acid (2R,3aR,4S,7aR,Z)-1-ethylidene-2-hydroxy-7a-methyl-octahydro-1H-inden-4-yl ester and acetic acid (2S,3aR,4S,7aS,Z)-1-ethylidene-2-hydroxy-7a-methyl-octahydro-1H-inden-4-yl ester (12.5 g, 47 mmol) was dissolved in ethyl vinyl ether (150 mL). Hg(OAc)₂ (14.1 g, 44 mmol) was added and the suspension was poured into a pyrex pressure tube, flushed with N₂ and closed tightly. The mixture was stirred at 130° C. for 18 h, cooled at room temperature and concentrated in vacuo. The residue was purified by column chromatography (SiO₂, 7.5-30% ethyl acetate in heptane) to give: Fraction A (8.1 g (65%) of the titled compound); Fraction B (1.8 g, mixture containing ca 50% of the titled compound). Fraction B was purified by column chromatography (SiO₂, 7.5-30% ethyl acetate in heptane) to give: Fraction C (0.6 g of the titled compound). Fraction A and C were combined affording 8.7 g (70%) of the titled compound as a colorless oil. ¹H NMR (CDCl₃): δ 9.68 (s, 1H), 5.40 (m, 1H), 5.19 (m, 1H), 2.72 (m, 1H), 2.53 (ddd, J=16.2, 5.8, 1.8 Hz, 1H), 2.33 (ddd, J=16.2, 7.3, 2.6 Hz, 1H), 2.03 (s, 3H), 2.02-2.14 (m, 2H), 1.72-1.90 (m, 4H), 1.47-1.62 (m, 2H), 1.36 (M, 1H), 1.14 (d, J=7.1 Hz, 3H), 1.02 (s, 3H).

### 5(R)-((3aR,4S,7aR)-4-acetoxy-7a-methyl-3a,4,5,6,7,7a-hexahydro-3H-inden-1-yl)-hex-2-E-enoic acid ethyl ester

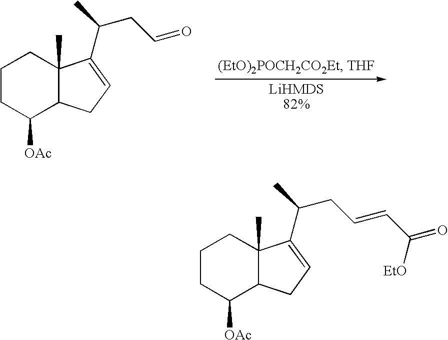

Acetic acid (3aR,4S,7aS)-7a-methyl-1-(S)-4-oxobutan-2-yl)-3a,4,5,6,7,7a-hexahydro-3H-inden-4-yl ester (16.2 g; 61 mmol) and triethyl phosphonoacetate (36 ml; 183 mmol, 3 eq.) were dissolved under N₂ atmosphere in THF (200 mL, freshly distilled over Na/benzophenone). The mixture was cooled to −90° C. and a solution of LiHMDS in hexanes (122 mL, 1 M solution, 2 eq.) was added dropwise within 45 min. keeping the temperature below −90° C. After complete addition the reaction mixture was allowed to warm to −78° C. and stirring was continued at this temperature for 70 min. The reaction was quenched by dropwise addition of a mixture of water (64 ml) and sat. NH₄Cl solution (32 mL). To the reaction mixture was added tert-butyl methyl ether (400 ml) and water (400 mL), the organic layer was separated and concentrated in vacuo affording fraction A. The aqueous layer was extracted with tert-butyl methyl ether (1×400 ml, 1×200 ml). The organic layers were combined with fraction A, washed with water (2×200 ml), washed with brine (1×150 ml), dried (Na₂SO₄), filtered and the filtrate was concentrated in vacuo. The residue was purified by column chromatography (SiO₂, ethyl acetate/heptane=1:10) affording the title compound (18 g, 88%) as a E/Z-mixture (E:Z=10:1). ¹H NMR (CDCl₃): δ 6.88 (dt, J=15.4, 7.3 Hz, 1H), 5.78 (dm, J=15.4 Hz, 1H), 5.37 (br. s, 1H), 5.20 (br. s, 1H), 4.17 (q, J=7.2 Hz, 2H), 2.03 (s, 3H), 2.22-2.39 (m, 2H), 1.96-2.17 (m, 3H), 1.72-1.90 (m, 4H), 1.46-1.62 (m, 2H), 1.36 (td, J=13.3, 4.0 Hz, 1H), 1.27 (t, J=7.1 Hz, 3H), 1.06 (d, J=7.2 Hz, 3H), 0.99 (s, 3H); MS: m/e 357 (M+23), 275 (M−59).

### (3aR,4S,7aR)-1-((S,E)-5-ethyl-5-hydroxy-1-methyl-hept-3-enyl)-7a-methyl-3a,4,5,6,7,7a-hexahydro-3H-inden-4-ol

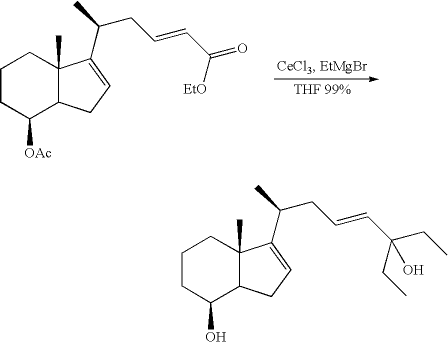

A 1 L round bottom flask was charged with cerium(III) chloride heptahydrate (234 g, 0.63 mol) and water (ca. 70 g) was removed in vacuo (10⁻² mbar) via bulb to bulb distillation by heating slowly at 70° C. (30 min), 95° C. (3 h), 120° C. (1 h) and 160° C. (3 h), respectively. After cooling overnight and under vacuo at room temperature the off-white cerium(III)chloride monohydrate (162 g) was transferred into a 3 L three-necked flask equipped with a magnetic stirring bar. The last equivalent of water was removed by stirring and heating in vacuo (10⁻² mbar) at 90° C. (1 h), 120° C. (1 h), 160° C. (1 h) and 210° C. (4 h), respectively. Condensate water on top of the flask was removed by heating with a hot gun. When no more formation of condensate was observed, removal of water was complete. The flask was cooled at room temperature and flushed with nitrogen. THF (1.3 L) was added and the mixture was stirred at room temperature for 18 h. The milky suspension was cooled at 0° C. and a solution of EtMgBr in THF (610 mL, 1 M solution) was added dropwise within 1 h. After stirring at 0° C. for 2 h a solution of (S,E)-5-((3aR,4S,7aS)-4-acetoxy-7a-methyl-3a,4,5,6,7,7a-hexahydro-3H-inden-1-yl)-hexenoic acid ethyl ester (16.2 g, 48.4 mmol, contaminated with ca. 10% of the corresponding Z-isomer) in THF (75 mL) was added dropwise within 1 h. After stirring at 0° C. for 1 h the showed complete conversion and the reaction was quenched by slow addition of water (150 mL, exothermic reaction), upon which a sticky solid precipitated. The solution (Fraction A) was decanted and the residual solid was mixed thoroughly with water (1 L) to give an aqueous suspension (Fraction B). Fraction A and B were combined and extracted four times with a mixture of ethyl acetate (500 mL) and heptane (500 mL). The combined organic layers were washed with sat. NaHCO₃ solution (2×), brine (1×), dried (Na₂SO₄), filtered and the filtrate was concentrated in vacuo. The residue (17 g) was purified by column chromatography (1 kg SiO₂, 20% ethyl acetate in heptane) affording the title compound (13.4 g, 98%) as a slightly yellow oil. Purity according HPLC: 93.1% (λ=212 nm). The product was purified again by column chromatography (1 kg SiO₂, 20% ethyl acetate in heptane) to give: Fractions A 11.91 g, (86% yield) of the titled compound as a colorless oil; purity according HPLC: >96.5% (λ=212 nm); Fraction B 1.40 g, (10% yield) of the titled compound as a colorless oil; purity according HPLC: 86.9% (λ=212 nm); ¹H NMR (CDCl₃): δ 5.51 (ddd, J=15.4, 7.4, 6.5 Hz, 1H), 5.35 (dm, J=15.4 Hz, 1H), 5.33 (m, 1H), 4.17 (m, 1H), 2.12-2.32 (m, 3H), 1.67-2.02 (m, 6H), 1.23-1.60 (m, 9H), 1.05 (s, 3H), 1.04 (d, J=7.2 Hz, 3H), 0.84 (t, J=7.3 Hz, 6H); MS: m/e 329 (M+23), 289 (M−17), 271 (M−35).

### (3aR,4S,7aR)-1-((S,E)-5-ethyl-5-hydroxy-1-methyl-hept-3-enyl)-7a-methyl-3a,4,5,6,7,7a-hexahydro-3H-inden-4-one

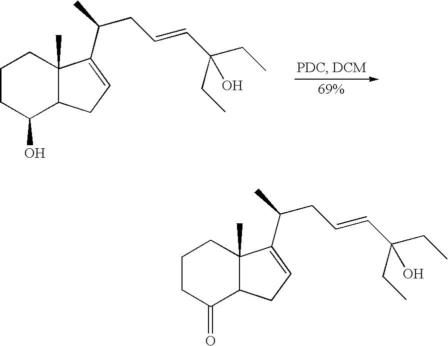

A solution of (3aR,4S,7aS)-1-((S,E)-6-ethyl-6-hydroxyoct-4-en-2-yl)-7a-methyl-3a,4,5,6,7,7a-hexahydro-3H-inden-4-ol (4.70 g, 15.3 mmol, purity according HPLC: 96.5% (λ=212 nm) in dichloromethane (200 mL) was cooled in an ice-bath and treated portionwise with pyridinium dichromate (13.1 g, 34.9 mmol, 2.2 eq.). The reaction mixture was allowed to warm at room temperature overnight, filtered through a path of Celite and the filtercake was washed with dichloromethane. The combined filtrates were washed with a 2 M KHCO₃ solution, washed with brine, dried (Na₂SO₄) and concentrated in vacuo. the residue was purified by column chromatography (SiO₂, 25% ethyl acetate in heptane) affording the title compound (4.0 g, 85%) as a colorless oil. ¹H NMR (CDCl₃): δ 5.54 (ddd, J=15.6, 7.1, 6.0 Hz, 1H), 5.38 (dm, J=15.6 Hz, 1H), 5.30 (m, 1H), 2.82 (dd, J=10.4, 6.0 Hz, 1H), 2.42 (ddt, J=15.4, 10.4, 1.6 Hz, 1H), 2.16-2.33 (m, 4H), 1.93-2.16 (m, 4H), 1.84-1.93 (m, 1H), 1.65 (td, J=12.1, 5.6 Hz, 1H), 1.52 (br. q, J=6.9 Hz, 4H), 1.34 (br. s, 1H), 1.05 (d, J=6.9 Hz, 3H), 0.85 (br. t, J=7.2 Hz, 6H), 0.82 (s, 3 H).

### Coupling and Synthesis

### 1-(5-Ethyl-1-methyl-5-trimethylsilanyloxy-hept-3-enyl)-7a-methyl-3,3a,5,6,7,7a-hexahydro-inden-4-one

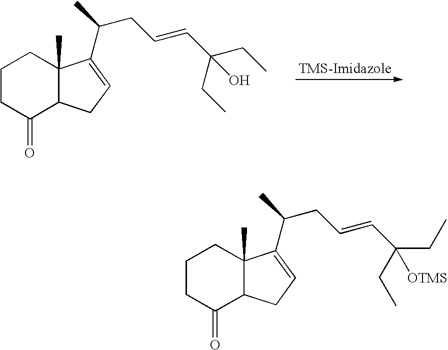

To a solution of (3aR,4S,7aR)-1-((S,E)-5-ethyl-5-hydroxy-1-methyl-hept-3-enyl)-7a-methyl-3a,4,5,6,7,7a-hexahydro-3H-inden-4-one (320 mg, 1.05 mmol) in dichloromethane (20 mL) was added 1-(trimethylsilyl)imidazole (0.2 mL, 1.34 mmol). The reaction mixture was stirred at room temperature for 4 d. Reaction control (tlc) showed complete conversion. The mixture was concentrated in vacuo and the residue was purified by column chromatography (SiO₂, 10% ethyl acetate in heptane) affording the titled compound (377 mg, 95%) as a colorless oil.

### 1α-Fluoro-25-hydroxy-16-23E-diene-26,27-bishomo-20-epi-cholecalciferol

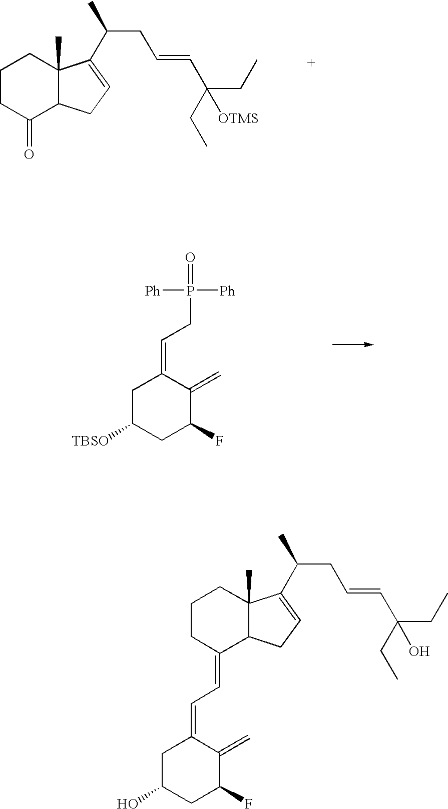

To a stirred solution of 240 mg (0.51 mmole) of (1S,3Z,5R)-1-fluoro-5-(t-butyldimethyl)silanyloxy)-2-methenyl-3-(diphenylphosphinoyl)ethylidene cyclohexane in 5 ml of anhydrous tetrahydrofuran at −78° C. was added 0.319 ml (0.51 mmole) of 1.6M n-butyllithium in hexane, dropwise under argon. After stirring for 5 min, to thus obtained red solution was added a solution of 103 mg (0.273 mmole) of 1-(5-Ethyl-1-methyl-5-trimethylsilanyloxy-hept-3-enyl)-7a-methyl-3,3a,5,6,7,7a-hexahydro-inden-4-one in 4 ml of anhydrous tetrahydrofuran, dropwise over a 10 min period. The reaction mixture was stirred at −78° C. for 2 hrs, then placed in freezer (−20° C.) for one hour, quenched by addition of 10 ml of a 1:1 mixture of 2N Rochelle salt and 2N potassium bicarbonate and warmed up to room temperature. After dilution with additional 25 ml of the same salts mixture, it was extracted with 3×90 ml of ethyl acetate. The combined organic layers were washed three times with water and brine, dried over sodium sulfate and evaporated to dryness. The residue was purified by FLASH chromatography on a 30 mm×7″ silica gel column with hexane-ethyl acetate (1:4), to give 145 mg of disilylated title compound. To a solution of 145 mg of disilyl intermediate in 3 ml anhydrous tetrahydrofuran was added 1.7 ml (1.7 mmole) of 1M tetrabutyl-ammonium fluoride in tetrahydrofuran under argon. The reaction mixture was stirred at room temperature for 18 hrs, and then quenched by addition of 10 ml water and stirring for 15 min. It was diluted with 20 ml of water and brine and extracted with 3×80 ml ethyl acetate. The organic layers were washed four times with water and brine, dried over sodium sulfate, and evaporated to dryness. The crude product was purified by FLASH chromatography on a 30 mm×5″ silica gel column with hexane-ethyl acetate (3:2), and by HPLC on a YMC 50 mm×50 cm silica gel column with hexane-ethyl acetate (1:1). It gave 90 mg (74%) of the title compound, crystallization from methyl acetate-hexane.

### Larger Scale Coupling and Synthesis

### 1-(5-Ethyl-1-methyl-5-trimethylsilanyloxy-hept-3-enyl)-7a-methyl-3,3a,5,6,7,7a-hexahydro-inden-4-one

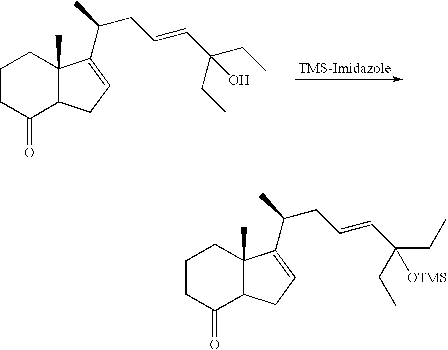

To a solution of (3aR,7aS)-1-((S,E)-6-ethyl-6-hydroxyoct-4-en-2-yl)-7a-methyl-3,3a,5,6,7,7a-hexahydro-3H-inden-4-one (4.0 g, 13.1 mmol) in dichloromethane (200 mL) was added 1-(trimethylsilyl)imidazole (2.2 mL, 14.9 mmol). The reaction mixture was stirred at room temperature for 18 h. According the conversion was not complete and additional 1-(trimethylsilyl)imidazole (4.3 mL, 29.1 mmol) was added and stirring was continued for 5 h. The mixture was concentrated in vacuo at 30° C. and the residue was purified by column chromatography (200 g SiO₂, 10% ethyl acetate in heptane) affording the title compound (4.6 g, 93%) as a colorless oil. Purity according HPLC: 100% (λ=265 nm); ¹H NMR (CDCl₃): δ 5.28-5.52 (m, 3H), 2.83 (dd, J=10.4, 6.1 Hz, 1H), 2.43 (ddm, J=15.4, 10.4 Hz, 1H), 2.18-2.32 (m, 4H), 1.94-2.18 (m, 4H), 1.85-1.93 (m, 1H), 1.76 (td, J=12.4, 5.6 Hz, 1H), 1.53 (br. q, J=7.3 Hz, 4H), 1.16 (d, J=6.9 Hz, 3H), 0.83 (s, 3H), 0.81 (br. t, J=7.1 Hz, 6H), 0.47 (s, 9H); MS: m/e 376 (M), 361 (M−15), 347 (M−29).

### 1α-Fluoro-25-hydroxy-16-23E-diene-26,27-bishomo-20-epi-cholecalciferol

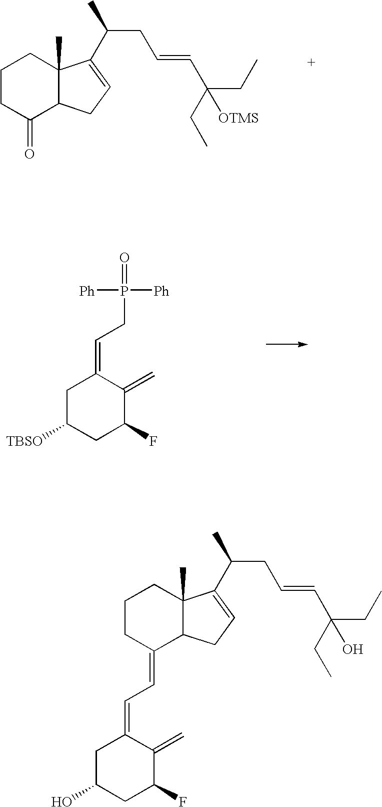

A 25 ml flask was charged with (1S,3Z,5R)-1-Fluoro-5-(tert-Butyldimethyl)silanyloxy)-2-methenyl-3-(diphenylphosphinoyl)ethylidene cyclohexane (748 mg, 1.59 mmol, 1.2 eq) and (3aR,7aS)-1-(S,E)-6-ethyl-6-(trimethylsilyloxy)oct-4-en-2-yl)-7a-methyl-3,3a,5,6,7,7a-hexahydro-3H-inden-4-one (499 mg, 1.32 mmol). The mixture was co-evaporated with toluene (3×5 mL), dissolved in THF (10 mL, freshly distilled over Na/benzophenone) and cooled to −55° C. LiHMDS (1.65 mL, 1 M solution in THF, 1.2 eq.) was added dropwise within 5 min. The deep red solution was allowed to warm to −25° C. within 1.5 h. TBAF (9 mL, 1 M solution in THF) was added (color turns to orange) and the mixture was allowed to warm to room temperature overnight. The reaction was quenched by pouring slowly into an ice-cold 1 M aqueous solution of KHCO₃. Thus formed mixture was extracted with ethyl acetate (3×25 mL). The combined organic layers were washed with water, brine (3×), dried (Na₂SO₄) and concentrated in vacuo at 30° C. The residue was purified by column chromatography (25% ethyl acetate in heptane), affording: Fraction A: 35 mg (7%) of epimerized CD-block epi-(3aR,7aS)-1-((S,E)-6-ethyl-6-(trimethylsilyloxy)oct-4-en-2-yl)-7a-methyl-3,3a,5,6,7,7a-hexahydro-3H-inden-4-one. Fraction B: traces of Vitamin D-related byproducts. Fraction C: 27 mg (5%) of the titled compound as a white solid; purity according HPLC: 96.8% (λ=265 nm). Fraction D: 450 mg (75%) of the titled compound as a white solid; purity according HPLC: 93.7% (λ=265 nm). Fraction E: 30 mg (5%) of the titled compound as a white solid; purity according HPLC: 92.9% (λ=265 nm). Fraction D was dissolved in methyl formate (3-4 mL). Heptane (15 mL) was added and the flask was flushed with nitrogen gas until the solution became cloudy. The product started to crystallize and for complete crystallization the flask was stored at 4° C. for 1 h. The solvent was decanted and the remaining solid was washed with cold heptane (3×5 mL). After flushing with nitrogen gas the solid was dried in vacuo affording: Fraction F: 331 mg (56% yield) of the titled compound as a white solid; purity according HPLC: 100% (λ=265 nm); ¹H NMR (CD₃CN): δ 6.42 (br d, 1H), 6.10 (br d, 1H), 5.51 (ddd, 1H), 5.39 (br d, 1H), 5.36 (br s, 1H), 5.35 (br d, 1H), 5.13 (ddd, 1H), 5.07 (br s, 1H), 3.97-4.05 (m, 1H), 2.92 (d, 1H), 2.85 (dd, 1H), 2.57 (dd, 1H), 2.38 (dd, 1H), 2.14-2.29 (m, 5H), 1.96-2.04 (m, 2H), 1.84-1.89 (m, 1H), 1.73-1.82 (m, 3H), 1.64-1.72 (m, 1H), 1.53 (ddd, 1H), 1.45 (br. q, 4H), 1.04 (d, 3H), 0.81 (t, 6H), 0.69 (s, 3H); ¹³C NMR (CD₃CN): 160.12, 143.37 (d, J=17 Hz), 142.83, 137.33, 133.21 (d, J=2 Hz), 126.96, 124.84, 120.83, 117.33 (d, J=32 Hz), 115.40 (d, J=10 Hz), 93.74, 91.51, 74.83, 65.72 (d, J=5 Hz), 58.19, 50.31, 45.14, 40.94 (d, J=21 Hz), 39.78, 35.21, 33.34, 33.33, 32.46, 29.33, 28.63, 23.56, 20.33, 16.74, 1.41. ¹⁹F NMR (CD₃CN): δ-177.55; MS: m/e 482 (M+39), 465 (M+23), 425 (M−17). UV λmax: 244 nm (ε 13747), 270 nm (ε 13756) (CH₃OH). [α]^{D}₂₅+101 (c 1.92, CH₃OH).

### Alternate Coupling and Synthesis

### 1α-Fluoro-25-hydroxy-16-23E-diene-26,27-bishomo-20-epi-cholecalciferol

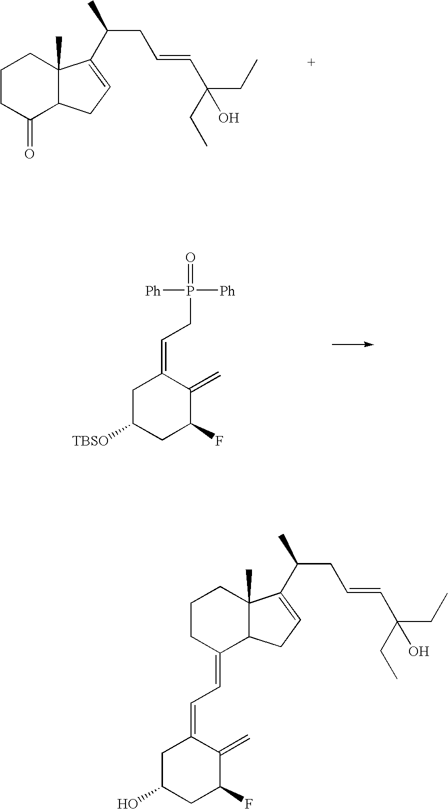

A solution of 1S,3Z,5R)-1-Fluoro-5-(tert-Butyldimethyl)silanyloxy)-2-methenyl-3-(diphenylphosphinoyl)ethylidene cyclohexane (278 mg, 0.59 mmol, 3.6 eq.) in THF (10 mL, distilled over Na-benzophenone) was cooled at −75° C. and n-BuLi (0.23 mL, 2.5 M solution in hexanes, 0.57 mmol) was added dropwise. The red solution was stirred for 20 min. during which the temperature was allowed to rise to −50° C. A solution of (3aR,7aS)-1-((S,E)-6-ethyl-6-hydroxyoct-4-en-2-yl)-7a-methyl-3,3a,5,6,7,7a-hexahydro-3H-inden-4-one (50 mg, 0.164 mmol) in THF (2 mL, distilled over Na-benzophenone) was added dropwise at −50° C. within 5 min. Stirring was continued for 2 h during which the temperature was allowed to rise to −10° C. Tlc showed ca. 20% conversion. To the yellow solution was added dropwise TBAF (1.8 mL, 1 M solution in THF, containing ca. 5% water) upon which the solution turned red-brown. The reaction mixture was allowed to reach room temperature overnight. The reaction mixture was quenched by addition of an ice-cold aqueous 1 M KHCO₃ solution (3 g in 30 mL of water) and the mixture was extracted with ethyl acetate (2×40 mL). The combined organic layers were washed with water and brine, dried (Na₂SO₄), filtered and the filtrate was concentrated in vacuo at 30° C. The residue was purified by column chromatography (SiO₂, 25% ethyl acetate in heptane) affording the titled compound (13 mg, 18%) as a white foam.

### Example 2

### Synthesis of 21-(3-Hydroxy-3-methylbutyl)-1,25-dihydroxy-19-nor-cholecalciferol

### [1R-[1α(2E,4E,7E),3aβ,4α,7aα]]-5-[4-[[(1,1-dimethylethyl)dimethylsilyl]oxy]Octahydro-7a-methyl-1H-inden-1-yl]-2,4,7-nonatrienedioic acid diethyl ester

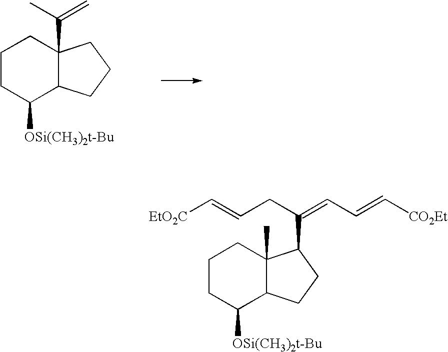

To a stirred solution of 3.08 g (10.0 mmol) of [1R-(1α,3aβ,4α,7aα)]-(1,1-dimethylethyl)dimethyl[[octahydro-7a-methyl-1-(1-methylethenyl)-1H-inden-4-yl]oxy]silane and 3.92 g (40.0 mmol) of ethyl propiolate in 20 mL of dichloromethane was added 40 mL (40.0 mmol) of a 1.0 M solution of ethylaluminum dichloride in hexanes. The mixture was stirred under argon at room temperature for 24 hrs, treated with 981 mg (10 mmol) of ethyl propiolate and 7.5 mL (7.5 mmol) of a 1.0 M solution of ethylaluminum dichloride in hexanes and stirred for an additional 18 hrs. The resultant orange-red solution was added portion-wise to a mixture of 200 mL ethyl acetate and 100 mL of 50% brine, and, after the fizzing had subsided, the organic phase was collected and the aqueous phase was re-extracted with 3×100 mL of ethyl acetate. The combined organic extracts were washed with 2×100 mL of 50% brine, dried (Na₂SO₄), and evaporated to give 5.76 g of a reddish gum, which was subjected to flash chromatography on 120 g of silica gel (40-65 μm mesh, 3.5 cm diameter column) with 10% ethyl acetate in hexanes as eluent, collecting 20-mL fractions. Fractions 21-32 were combined and evaporated to give 2.18 g of crude product. Further purification was achieved by HPLC (15-30 μm mesh silica gel, 50 cm×50 mm column, flow rate of 70 mL/min) with 7.5% ethyl acetate in hexanes as eluent to give 1.62 g (32%) of the titled compound, R_{T} 25 minutes, as a pale yellow gum: [α]²⁵_{D} +83.50° (EtOH, c=0.98); UV (MeOH) 284 (ε=28,173), 207 (ε=16,884) nm; IR (CHCl₃) 1708, 1651, 1628 cm⁻¹; ¹H NMR (CDCl₃) δ 0.006 (6H, s), 0.80, 3H, s), 0.88 (9H, s), 1.16 (1H, t, J=7.6 Hz), 1.28 (6H, overlapping t, J=7 Hz), 1.67-1.78, (6H, m), 2.16 (1H, t, J=9 Hz 3.00, (1H, dd, J=6, 16, Hz), 3.35 (1H, dd, J=16.4 Hz), 4.02 (1H, s), 4.16 (4H, overlapping q, J=7 Hz), 5.75 (1H, d, J=16 Hz), 5.84 (1H, d, J=15 Hz), 6.17 (1H, d, J=11 Hz), 6.88 (1H, dt, J=16.6 Hz), 7.50 (1H, dd, J=11, 15, Hz); MS (EI) m/z 504 (M⁺¹, 23). Anal. Calcd for C₂₉H₄₈ O₅Si: C, 69.00; H, 9.58; Si, 5.56. Found: C, 68.94; H, 9.69; Si, 5.67.

### [1R-(1α,3aβ,4α,7aα)]-5-[4-[[(1,1-dimethylethyl)dimethylsilyl]oxy]Octahydro-7a-methyl-1H-inden-1-yl]nonanedioic acid diethyl ester

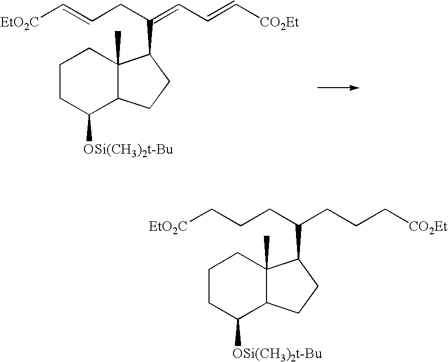

A stirred solution of 1.009 g (2.0 mmol) of [1R-[1α(2E,4E,7E),3aβ,4α,7aα]]-5-[4-[[(1,1-dimethyl ethyl)dimethylsilyl]oxy]octahydro-7a-methyl-1H-inden-1-yl]-2,4,7-nonatriene dioic acid diethyl ester in 50 mL of ethyl acetate was hydrogenated over 200 mg of 10% palladium on charcoal at room temperature and atmospheric pressure until hydrogen absorption ceased (140 mL of hydrogen was absorbed during 2.5 hrs). The mixture was filtered over a pad of Celite, which was washed with 4×50 mL of ethyl acetate, and the combined filtrate and washings were evaporated to give 1.07 g of a colorless oil. This was purified by flash chromatography on 60 g of silica gel (40-65 μm mesh, 3.5 cm diameter column) with 12% ethyl acetate in hexanes as eluent, collecting 20-mL fractions. Fractions 7-12 were combined and evaporated to give 964 mg (94%) of the titled compound as a colorless oil: [α]²⁵_{D} +32.1° (CHCl₃, c=1.04); IR (CHCl₃) 1726 cm⁻¹; ¹H NMR (CDCl₃) δ 0.00 (3H, s), 0.01 (3H, s), 0.87 (9H, s), 0.88 (3H, s), 1.27 (6H, t, J=7 Hz), 1.28-1.90 (21H, m), 2.25 (4H, br t), 3.98 (1H, s), 4.11 (4H, q, J=7 Hz); MS (FAB) m/z 511 (M⁺1, 100). Anal. Calcd for C₂₉ H₅₄ O₅ Si: C, 68.11; H, 10.66; Si, 5.50. Found: C, 68.21; H, 10.85; Si, 5.43.

### [1R-(1α,3aβ,4α, 7aα)]-6-[4-[[(1,1-dimethylethyl)dimethylsilyl]oxy]Octahydro-7a-methyl-1H-inden-1-yl]-2,10-dimethyl-2,10-undecane diol

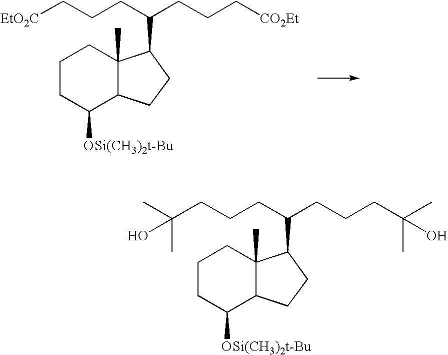

To a stirred solution of 868 mg (1.7 mmol) of [1R-(1α,3aβ,4α, 7aα)]-5-[4-[[(1,1-dimethylethyl)dimethylsilyl]oxy]octahydro-7a-methyl-1H-inden-1-yl]nonanedioic acid diethyl ester in 12 mL of anhydrous THF was added dropwise, with cooling (ice bath), 5.0 mL (15 mmol) of a 3.0 M solution of methylmagnesium bromide in ether. The mixture was stirred at room temperature for 45 minutes, cooled to 5° C., and quenched by the dropwise addition of 3.0 mL of saturated NH₄Cl. After the fizzing had subsided, 15 mL of ethyl acetate and 15 mL of saturated NH₄Cl were added, stirring was continued for 20 minutes, and the mixture was poured into 100 mL of ethyl acetate and 50 mL of saturated NH₄Cl. The organic phase was collected and the aqueous phase was re-extracted with 3×60 mL of ethyl acetate. The combined organic extracts were washed with 2×100 mL of 50% brine, dried (Na₂SO₄), and evaporated to give 814 mg of a colorless gum, which was purified by flash chromatography on 100 g of silica gel (40-65 μm mesh, 3.5 cm diameter column) with 50% ethyl acetate in hexanes as eluent taking 20-mL fractions. Fractions 19-20 were combined and evaporated to give, after high vacuum drying (17 hrs), 763 mg (93%) of the titled compound as a colorless foam: [α]²⁵_{D} +35.8° (EtOH, c=1.02); IR (CHCl₃) 3608 cm⁻¹; ¹H NMR (CDCl₃) δ 0.00 (6H, s), 0.88 (9H, s), 0.90 (3H, s), 1.20 (12H, s), 1.23-1.90. (27H, m), 3.99 (1H, s); MS (EI) m/z 482 (3, M⁺). Anal. Calcd for C₂₉H₅₈ O₃Si: C, 72.14; H, 12.11; Si, 5.82. Found: C, 72.18; H, 11.99; Si, 5.69.

### [1S-(1α,3aβ,4α, 7aα)]Octahydro-1-[5-hydroxy-1-(4-hydroxy-4-methylpentyl)-5-methylhexyl]-7a-methyl-4H-inden-4-ol

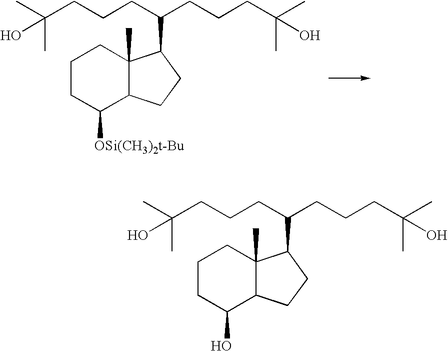

To a stirred solution of 700 mg (1.45 mmol) of [1R-(1α,3aβ,4α, 7aα)]-6-[4-[[(1,1-dimethylethyl)dimethylsilyl]oxy]octahydro-7a-methyl-1H-inden-1-yl]-2,10-dimethyl-2,10-undecanediol in 5 mL of THF and 15 mL of CH₃CN contained in a Teflon bottle was added 3.0 mL of an approximately 30% aqueous solution of fluorosilicic acid (prepared according to A. S. Pilcher and P. DeShong, J. Org. Chem., 1993, 58, 5130) and the mixture was stirred under argon at room temperature for 1.0 h. Four 2.0-mL portions of the fluorosilicic acid solution were then added at hourly intervals, for a total of 11 mL of reagent and a reaction time of 5 hrs. The reaction mixture was poured cautiously into a mixture of 125 mL of ethyl acetate and 75 mL of saturated aqueous KHCO₃ solution. After the fizzing had subsided, the organic phase was collected and the aqueous phase was re-extracted with 3×75 mL of ethyl acetate. The organic extracts were washed with 125 mL of 50% brine, dried (Na₂SO₄), and evaporated to give 534 mg of a gum, which was purified by flash chromatography on 70 g of silica gel (40-65 μm mesh, 3.5 cm diameter column) with 70% ethyl acetate as eluent, taking 20-mL fractions. Fractions 17-30 were combined, filtered and evaporated, and the residue was kept under high vacuum for 4 hrs to give 458 mg (85%) of the titled compound as a colorless foam: [α]²⁵_{D} +26.2° (CHCl₃, c=0.76); IR (CHCl₃) 3608 cm⁻¹; ¹H NMR (CDCl₃) δ 0.93 (3H, s), 1.21 (12H, s), 1.24-1.60 (24H, m), 1.79-1.95 (4H, m), 4.07 (1H, s); MS (FAB) m/z 369 (M'+H).

### [1S-(1α,3aβ,4α, 7aα)]Octahydro-1-[5-hydroxy-1-(4-hydroxy-4-methylpentyl)-5-methylhexyl]-7a-methyl-4H-inden-4-one

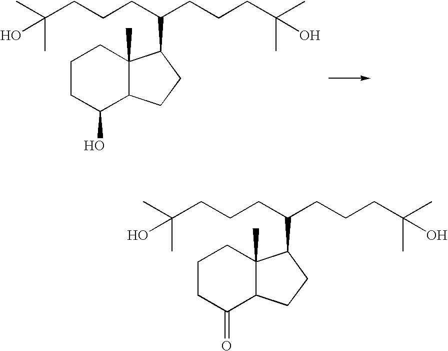

To a stirred solution of 400 mg (1.08 mmol) of [1S-(1α,3aβ,4α, 7aα)]octahydro-1-[5-hydroxy-1-(4-hydroxy-4-methylpentyl)-5-methylhexyl]-7a-methyl-4H-inden-4-ol in 8.0 mL of dichloromethane was added 1.30 g (3.45 mmol) of pyridinium dichromate and the mixture was stirred at room temperature for 4.75 hrs. It was diluted with 20 mL of diisopropyl ether, stirred for a further 15 minutes and filtered over a pad of Celite. The Celite was washed with 4×40 mL of diisopropyl ether and the combined filtrate and washings were evaporated to give 405 mg of a pale yellow gum, which was purified by flash chromatography on 70 g of silica gel (40-65 μm mesh, 3.5 cm diameter column) with 75% ethyl acetate in hexanes as eluent taking 20-mL fractions. Fractions 17-30 were combined and evaporated to give a colorless gum, which was kept under high vacuum for 4.5 hrs to give 372 mg (94%) of the titled compound as a colorless gum: [α]²⁵_{D} 0.45° (EtOH, c=0.92); IR (CHCl₃) 3608,1706 cm⁻¹; ¹H NMR (CDCl₃) δ 0.63 (3H, s), 1.22 (12H, s), 1.30-2.10 (22H, m), 2.20-2.28 (2H, m), 2.45 (1H, dd, J=7.6.11 Hz); MS m/z 348 (M⁺−18).

### [1S-(1α,3aβ,4α, 7aα)]Octahydro-7a-methyl-1-[4-methyl-1-[4-methyl-4-[(trimethylsilyl)oxy]pentyl]-5-[(trimethylsilyl)oxy]hexyl]-4H-inden-4-one

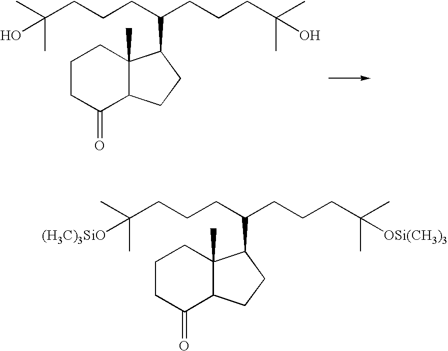

To a stirred solution of 366.6 mg (1.0 mmol) of [1S-(1α,3aβ,4α, 7aα)]octahydro-1-[5-hydroxy-1-(4-hydroxy-4-methylpentyl)-5-methylhexyl]-7a-methyl-4H-inden-4-one in 10.0 mL of dichloromethane was added 1.25 mL (8.5 mmol) of 1-(trimethylsilyl) imidazole and the mixture was stirred under argon at room temperature for 4.25 hrs. It was diluted with 7.0 mL of water, stirred for a further 15 minutes, and poured into a mixture of 75 mL of ethyl acetate and 50 mL of 50% brine. The organic phase was collected and the aqueous phase was re-extracted with 3×50 mL of ethyl acetate. The combined organic extracts were washed with 3×75 mL of 50% brine, dried (Na₂SO₄), and evaporated to give a colorless oil, which was purified by flash chromatography on 65 g of silica gel (40-65 μm mesh, 3.5 cm diameter column) with 20% ethyl acetate in hexanes as eluent, taking 20-mL fractions. Fractions 5-7 were combined, concentrated to ca. 5 mL, filtered through a 0.45 μm filter (Millex-HV) and evaporated to give a colorless oil, which was kept under high vacuum for 18 hrs to give 469 mg (91%) of the titled compound: [α]²⁵_{D}-3.21° (CHCl₃, c=0.87); IR (CHCl₃); 1706 cm⁻¹; ¹H NMR (CDCl₃) δ 0.01 (18H, s), 0.63 (3H, s), 1.20 (6H, s), 1.21 (6H, s),1.26-1.49 (14H, m), 1.50-2.10 (8H, m), 2.21-2.31 (2H, m), 2.46 (1H, dd, J=12.11 Hz); MS (EI) m/z 495 (M⁺-15). Anal. Calcd for C₂₉H₅₈O₃Si₂: C, 68.17; H, 11.44; Si, 10.99. Found: C, 68.19; H, 11.41; Si, 11.07.

### (1α, 3β,5Z,7E)-21-(3-hydroxy-3-methylbutyl)-9,10-Secocholesta-5,7,10,(19)-triene-1,3,25-triol

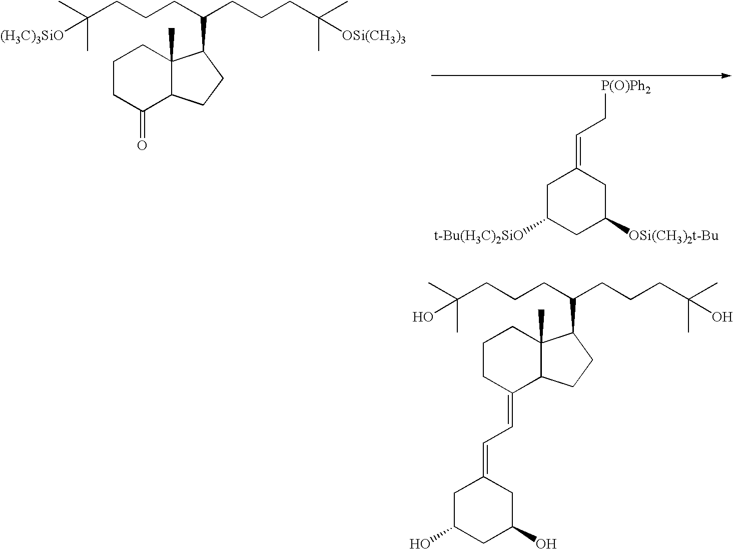

To a stirred, cold (−78° C.) solution of 571 mg (1.0 mmol) of the reagent [3R-(3α,5β,Z)]-[3,5-bis[[(1,1-dimethylethyl)dimethylsilyl]oxy]cyclohexylidene]ethyl]diphenylphosphine oxide in 6.0 mL of anhydrous THF was added 0.65 mL (1.04 mmol) of a 1.6 M solution of n-butyllithium in hexanes. The resultant deep red solution was stirred at −78° C. for 10 minutes, treated with 204.4 mg (0.40 mmol) of [1R-((1α,3aβ,4α, 7aα)]octahydro-7a-methyl-1-[5-methyl-1-[4-methyl-4-[(trimethylsilyl) oxy]pentyl]-5-[(trimethylsilyl)oxy]hexyl]-4H-inden-4-one in 2.5 mL of anhydrous THF, and stirred at −78° C. for 3 hrs. The mixture was allowed to warm to room temperature, stirred for 15 minutes and quenched with 15 mL of a 1:1 mixture of 1N Rochelle salt solution and 1N KHCO₃ solution. After 10 minutes, the mixture was poured into a mixture of 70 mL of ethyl acetate and 40 mL of 1:1 mixture of 1N Rochelle salt solution and 1N KHCO₃ solution. The organic phase was separated and the aqueous phase was re-extracted with 3×70 mL of ethyl acetate. The combined organic extracts were washed with 100 mL of 10% brine, dried (Na₂SO₄), and evaporated to give 760 mg of a colorless gum, which was purified by flash chromatography on 60 grams of silica gel (40-65 μm mesh; 3.5 cm diameter column) with 5% ethyl acetate in hexanes as eluent, taking 15 mL fractions. Fractions 5-10 were combined and evaporated to give 304 mg of a colorless gum. The latter was dissolved in 4.0 mL of THF, treated with 5.0 mL of a 1.0 M solution of tetra-n-butylammonium fluoride in THF, and the solution was stirred under argon at room temperature for 42 hours. It was diluted with 15 mL of water, stirred for 15 minutes, and poured into a mixture of 75 mL of ethyl acetate and 50 mL of 10% brine. The organic phase was separated and the aqueous phase was re-extracted with 3×70 mL of ethyl acetate. The combined organic extracts were washed with 5×100 mL of water, dried (Na₂SO₄) and evaporated to give 186 mg of a semi-solid, which was purified by flash chromatography on 50 g of silica gel (40-65 μm mesh; 3.5 cm diameter column) with 7.5% 2-propanol in ethyl acetate as eluent, taking 15-mL fractions. Fractions 11-29 were combined and evaporated. The residue was dissolved in 20 mL of anhydrous methyl formate and the solution was filtered through a 0.4 μm filter. Evaporation of the filtrate gave 154 mg of the title compound as a colorless solid: [α]²⁵_{D} +50.93° (MeOH, c=0.32); ¹H NMR (CDCl₃) δ 0.54 (3H, s), 1.21 (12H, s), 1.2-2.0 (27H, m), 2.20 (2H, m) 2.48 (1H, d, J=12 Hz), 2.25 (2H, m), 2.82 (1H, s), 4.06 (1H, br s) 4.10 (1H, br s), 5.85 (1H, d, J=12 Hz), 6.30 (1H, d, J=12 Hz); MS (FAB) m/z 490.4 (M⁺, 30).

### Example 3

### Synthesis of 1,25-Dihydroxy-20-(4-hydroxy-4-methyl-pentyl)cholecalciferol

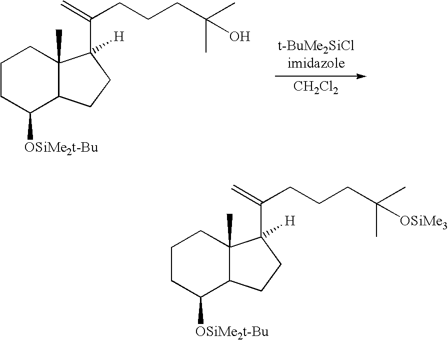

### (1R,3aR,4S,7aR)-4-(tert-Butyl-dimethyl-silanyloxy)-7a-methyl-1-(5-methyl-1-methylene-5-trimethylsilanyloxy-hexyl)-octahydro-indene

A 50 ml round bottom flask equipped with stir bar and Claisen adapter with rubber septum was charged with 1.78 g (4.510 mmol) of 6-[(1R,3aR,4S,7aR)-4-(tert-butyl-dimethyl-silanyloxy)-7a-methyl-octahydro-inden-1-yl]-2-methyl-hept-6-en-2-ol and 15 ml of dichloromethane. A 1.98 ml (13.53 mmol) of 1-(trimethylsilyl)imidazole was added dropwise. The mixture was stirred at room temperature for 2 h. A 15 ml of water was added and the mixture was stirred for 10 min. The resulting mixture was dissolved by the addition of 100 ml of water. The aqueous layer was extracted three times with 50 ml of dichloromethane. The combined organic layers were washed with 30 ml of brine dried over Na₂SO₄ and evaporated. The oil residue was chromatographed on column (75 cm³) using hexane:ethyl acetate (10:1) as mobile phase. Fractions containing product were pooled and evaporated to give 2.037 g (96%) of product as colorless oil.

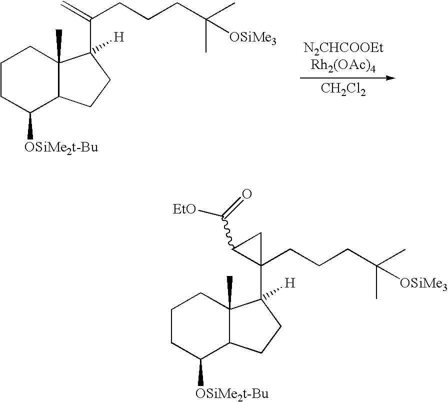

### 2-[(1S,3aR,4S,7aR)-4-(tert-Butyl-dimethyl-silanyloxy)-7a-methyl-octahydro-inden-1-yl]-2-(4-methyl-4-trimethylsilanyloxy-pentyl)-cyclopropanecarboxylic acid ethyl ester

A 100 ml round bottom flask equipped with stir bar and Claisen adapter with rubber septum was charged with 1.275 g (2.731 mmol) of (1R,3aR,4S,7aR)-4-(tert-butyl-dimethyl-silanyloxy)-7a-methyl-1-(5-methyl-1-methylene-5-trimethylsilanyloxy-hexyl)-octahydro-indene, 25 mg of Rh₂(OAc)₄ and 10 ml of dichloromethane. A solution of 935 mg (8.202 mmol) of ethyl diazoacetate in 20 ml of dichloromethane was added dropwise (5 ml/h) at room temperature. The mixture was stirred for 30 min. The reaction mixture was concentrated in vacuo and the remaining residue was chromatographed on column (100 cm³) using dichloromethane as mobile phase to give 1.236 g (82%) of products as mixture of isomers.

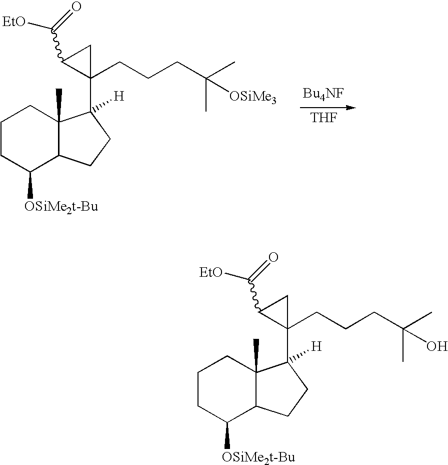

### 2-[(1S,3aR,4S,7aR)-4-(tert-Butyl-dimethyl-silanyloxy)-7a-methyl-octahydro-inden-1-yl]-2-(4-hydroxy-4-methyl-pentyl)-cyclopropanecarboxylic acid ethyl ester

A 25 ml round bottom flask equipped with stir bar and Claisen adapter with rubber septum was charged with 1.236 g (2.235 mmol) of 2-[(1S,3aR,4S,7aR)-4-(tert-butyl-dimethyl-silanyloxy)-7a-methyl-octahydro-nden-1-yl]-2-(4-methyl-4-trimethylsilanyloxy-pentyl)-cyclopropanecarboxylic acid ethyl ester, 4 ml of 1M tetrabutylammonium fluoride in tetrahydrofurane and 4 ml of tetrahydrofurane. The reaction mixture was stirred at room temperature for 2 h. The mixture was dissolved by the addition of 100 ml of ethyl acetate and extracted five times with 50 ml of water:brine (2:1) and 50 ml of brine, dried over Na₂SO₄ and evaporated to give 1.081 g of product as colorless oil (product was used to the next reaction without purification).

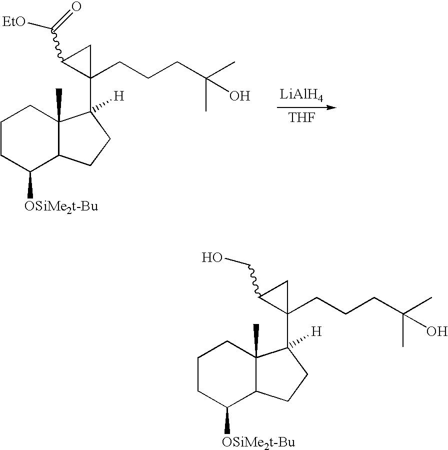

### 5-{1-[(1S,3aR,4S,7aR)-4-(tert-Butyl-dimethyl-silanyloxy)-7a-methyl-octahydro-inden-1-yl]-2-hydroxymethyl-cyclopropyl}-2-methyl-pentan-2-ol

A 50 ml round bottom flask equipped with stir bar and Claisen adapter with rubber septum was charged with crude (ca. 2.2 mmol) of 2-[(1S,3aR,4S,7aR)-4-(tert-butyl-dimethyl-silanyloxy)-7a-methyl-octahydro-inden-1-yl]-2-(4-hydroxy-4-methyl-pentyl)-cyclopropanecarboxylic acid ethyl ester and 6 ml of tetrahydrofurane. A 6 ml of 1M lithium aluminium hydride in tetrahydrofurane was added dropwise and the reaction mixture was stirred at room temperature for 1.5 h. Then the flask was placed into an ice bath and 5 ml of water was added dropwise. The mixture was dissolved by the addition of 50 ml of saturated solution of ammonium chloride, 50 ml of water and 25 ml of 1M H₂SO₄, extracted three times with 50 ml of ethyl acetate, dried over Na₂SO₄ and evaporated. The residue was purified over silica gel (350 cm³) using hexane:ethyl acetate (2:1, 1:1) to give 876 mg (90%) of products as a mixture of isomers.

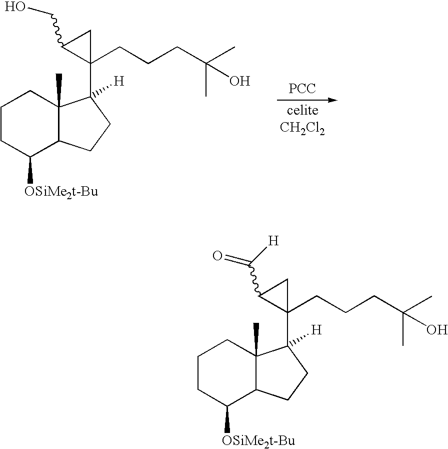

### 2-[(1S,3aR,4S,7aR)-4-(tert-Butyl-dimethyl-silanyloxy)-7a-methyl-octahydro-inden-1-yl]-2-(4-hydroxy-4-methyl-pentyl)-cyclopropanecarbaldehyde

A 25 ml round bottom flask equipped with stir bar and Claisen adapter with rubber septum was charged with 575 mg (2.667 mmol) of pyridinium chlorochromate, 650 mg of celite and 12 ml of dichloromethane. The 562 mg (1.128 mmol) of 5-{1-[(1S,3aR,4S,7aR)-4-(tert-butyl-dimethyl-silanyloxy)-7a-methyl-octahydro-inden-1-yl]-2-hydroxymethyl-cyclopropyl}-2-methyl-pentan-2-ol in 4 ml of dichloromethane was added dropwise and mixture was stirred in room temperature for 2 h. The reaction mixture was filtrated through column with silica gel (50 cm³) and celite (3 cm) using dichloromethane, dichloromethane:ethyl acetate (4:1, 3:1). The fractions containing product were pooled and evaporated to give 550 mg of product as yellow oil (product was used to the next reaction without purification).

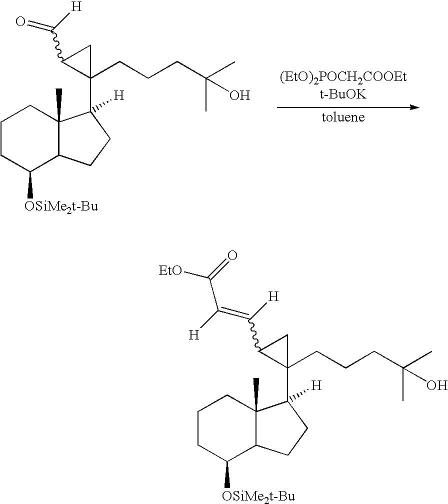

### 3-[2-[(1S,3aR,4S,7aR)-4-(tert-Butyl-dimethyl-silanyloxy)-7a-methyl-octahydro-inden-1-yl]-2-(4-hydroxy-4-methyl-pentyl)-cyclopropyl]-acrylic acid ethyl ester

A 50 ml round bottom flask equipped with stir bar and Claisen adapter with rubber septum was charged with 15 ml of toluene and 4.5 ml of 1M potassium tert-butoxide in tetrahydrofurane was added. A 1.005 g (4.482 mmol) of triethyl phosphonoacetate in 0.5 ml of toluene was added dropwise at ca. 5° C. The mixture was stirred at room temperature for 1 h. Then the mixture was cooled to −15° C. and crude (ca. 1.281 mmol) of 2-[(1S,3aR,4S,7aR)-4-(tert-butyl-dimethyl-silanyloxy)-7a-methyl-octahydro-inden-1-yl]-2-(4-hydroxy-4-methyl-pentyl)-cyclopropanecarbaldehyde in 4 ml of toluene was added and stirring was continued at −10° C. for 4 h. The reaction mixture was quenched with 50 ml of saturated solution of ammonium chloride and diluted with 50 ml of ethyl acetate and the inorganic layer was extracted twice with 50 ml of ethyl acetate, washed with 25 ml of brine, dried and evaporated. The residue was purified over silica gel (150 cm³) using hexane:ethyl acetate (5:1, 3:1) as a mobile phase to give 518 mg (80% for two steps) of products as a mixture of isomers.

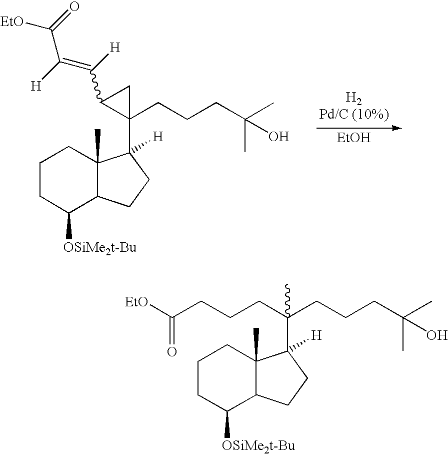

### 5-[(1R,3aR,4S,7aR)-4-(tert-Butyl-dimethyl-silanyloxy)-7a-methyl-octahydro-inden-1-yl]-9-hydroxy-5,9-dimethyl-decanoic acid ethyl ester

A 550 mg (1.085 mmol) of 3-[2-[(1S,3aR,4S,7aR)-4-(tert-butyl-dimethyl-silanyloxy)-7a-methyl-octahydro-inden-1-yl]-2-(4-hydroxy-4-methyl-pentyl)-cyclopropyl]-acrylic acid ethyl ester was hydrogenated over 200 mg of 10% Pd/C in 4 ml of ethanol at ambident temperature and atmospheric pressure of hydrogen. The reaction was monitoring by TLC (hexane:ethyl acetate—3:1). After 16 h the catalyst was filtered off and solvent evaporated. The residue was purified over silica gel (100 cm³) using hexane:ethyl acetate (10:1, 8:1, 3:1) as a mobile phase to give 549 mg (99%) of product as a colorless oil (mixture of isomers).

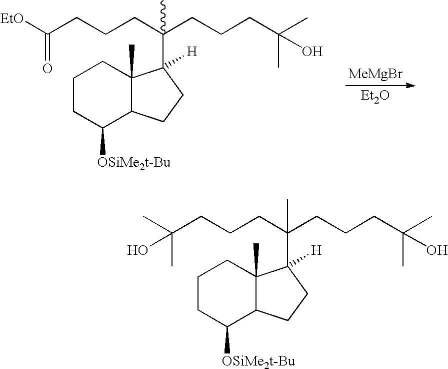

### 6-[(1R,3aR,4S,7aR)-4-(tert-Butyl-dimethyl-silanyloxy)-7a-methyl-octahydro-inden-1-yl]-2,6,10-trimethyl-undecane-2,10-diol

A 50 ml round bottom flask equipped with stir bar, Claisen adapter with rubber septum was charged with 1.099 mg (2.151 mmol) of 5-[(1R,3aR,4S,7aR)-4-(tert-butyl-dimethyl-silanyloxy)-7a-methyl-octahydro-inden-1-yl]-9-hydroxy-5,9-dimethyl-decanoic acid ethyl ester and 15 ml of diethyl ether. The solution was cooled in ace-water bath and 4.10 ml (12.792 mmol) of 3.12M solution of methylmagnesium bromide in diethyl ether was added dropwise. After completion of the addition the mixture was stirred at room temperature for 3.5 h then cooled again in an ice bath. A 10 ml of saturated solution of ammonium chloride was added dropwise. The resulting precipitate was dissolved by the addition of 50 ml of water. The aqueous layer was re-extracted three times with 50 ml of ethyl acetate. The combined ether layers were dried over Na₂SO₄ and evaporated. The oil residue was chromatographed on column (200 cm³) using hexane:ethyl acetate (3:1, 2:1, 1:1) as mobile phase. The chromatography (200 cm³) was repeated for mixture fractions to give 1.017 g (95%) of product as colorless oil.

[α]_{D}³¹=+36° c=0.36, CHCl₃

¹H NMR (CDCl₃): 3.98 (1H, br s), 2.00-1.95 (1H, m), 1.84-1.73 (1H, m), 1.66-1.63 (1H, m), 1.60-1.47 (4H, m), 1.43-1.30 (11H, m), 1.29-1.14 (8H, m), 1.20 (12H, s), 1.04 (3H, s), 0.90 (3H, s), 0.88 (9H, s), 0.00 (3H, s), −0.01(3H, s)

¹³C NMR (CDCl₃): 71.07, 71.05, 69.67, 57.05, 53.05, 45.03, 44.98, 43.82, 41.63, 39.87, 39.37, 39.31, 34.44, 29.45, 29.39, 29.36, 29.33, 25.89, 23.09, 22.87, 21.99, 18.47, 18.11, 17.97, 17.86, 16.78, −4.69, −5.04

| | | | | |
|---|---|---|---|---|
| MS HRES | Calculated for: | C₃₀H₆₀O₃Si | [M + Na]⁺ | 519.4204 |
| | Observed: | | [M + Na]⁺ | 519.4203 |

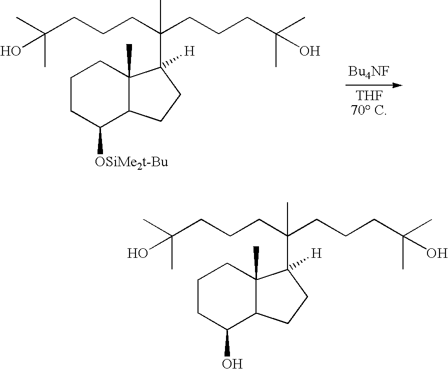

### 6-[(1R,3aR,4S,7aR)-4-Hydroxy-7a-methyl-octahydro-inden-1-yl]-2,6,10-trimethyl-undecane-2,10-diol

A 25 ml round bottom flask equipped with stir bar and Claisen adapter with rubber septum was charged with 884 mg (1.779 mmol) of 6-[(1R,3aR,4S,7aR)-4-(tert-butyl-dimethyl-silanyloxy)-7a-methyl-octahydro-inden-1-yl]-2,6,10-trimethyl-undecane-2,10-diol and 10 ml of 1M tetrabutylammonium fluoride in tetrahydrofurane. The reaction mixture was stirred at 70° C. for 48 h. (The new portion 5 ml of 1M tetrabutylammonium fluoride in tetrahydrofurane was added after 24 h). The mixture was dissolved by the addition of 150 ml of ethyl acetate and extracted six times with 50 ml of water:brine (1:1) and 50 ml of brine, dried over Na₂SO₄22 and evaporated. The oil residue was chromatographed on column (175 cm³) using hexane:ethyl:acetate (2:1, 1:1) as mobile phase to give 590 mg (87%) of product as colorless oil.

[α]_{D}³²=+11.4° c=0.35, CHCl₃

¹H NMR (CDCl₃): 4.07 (1H, br s), 2.02 (1H, br d, J=12.6 Hz), 1.84-1.76 (2H, m), 1.64-1.16 (24H, m), 1.21 (12H, s), 1.06 (3H, s), 0.91 (3H, s)

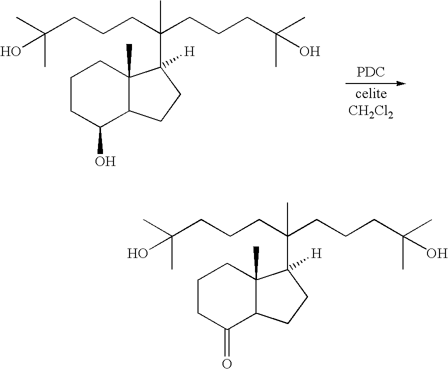

### (1R,3aR,4S,7aR)-1-[5-Hydroxy-1-(4-hydroxy-4-methyl-pentyl)-1,5-dimethyl-hexyl]-7a-methyl-octahydro-inden-4-one

A 25 ml round bottom flask equipped with stir bar and Claisen adapter with rubber septum was charged with 1.745 g (4.638 mmol) of pyridinium dichromate, 2.00 g of celite and 15 ml of dichloromethane. A 590 mg (1.542 mmol) of 6-[(1R,3aR,4S,7aR)-4-hydroxy-7a-methyl-octahydro-inden-1-yl]-2,6,10-trimethyl-undecane-2,10-diol in 4 ml of dichloromethane was added dropwise and mixture was stirred in room temperature for 5 h. The reaction mixture was filtrated through column with silica gel (50 cm³) and celite (3 cm) using dichloromethane, dichloromethane:ethyl acetate (2:1, 1:1) as a mobile phase. The fractions containing product were pooled and evaporated to give 577 mg (98%) of ketone.

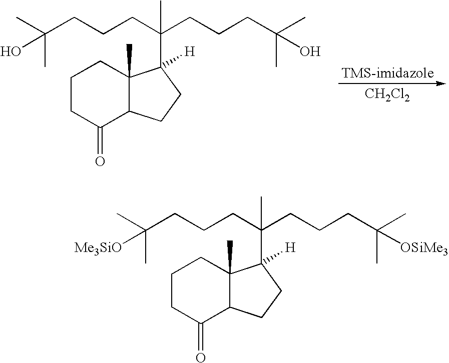

### (1R,3aR,4S,7aR)-1-[1,5-Dimethyl-1-(4-methyl-4-trimethylsilanyloxy-pentyl)-5-trimethylsilanyloxy-hexyl]-7a-methyl-octahydro-inden-4-one

A 25 ml round bottom flask equipped with stir bar and Claisen adapter with rubber septum was charged with 577 mg (1.516 mmol) of (1R,3aR,4S,7aR)-1-[5-hydroxy-1-(4-hydroxy-4-methyl-pentyl)-1,5-dimethyl-hexyl]-7a-methyl-octahydro-inden-4-one and 10 ml of dichloromethane. A 1.80 ml (12.269 mmol) of 1-(trimethylsilyl) imidazole was added dropwise. The mixture was stirred at room temperature for 2 h 30 min. The resulting mixture was dissolved by the addition of 100 ml of water. The aqueous layer was extracted four times with 50 ml of ethyl acetate. The combined organic layers were washed with 50 ml of brine, dried over Na₂SO₄ and evaporated.

The residue was purified over silica gel (50 cm³) using hexane:ethyl acetate (10:1) as a mobil phase to give a 739 mg (93%) of product as colorless oil.

¹H NMR (CDCl₃): 2.42 (1H, dd, J=9.9, 7.3 Hz), 2.30-2.13 (3H, m), 2.04-1.50 (9H, m), 1.42-1.14 (11H, m), 1.21 (6H, s), 1.20 (6H, s), 0.90 (3H, s), 0.73 (3H, s), 0.11 (9H, s), 0.10 (9H, s)

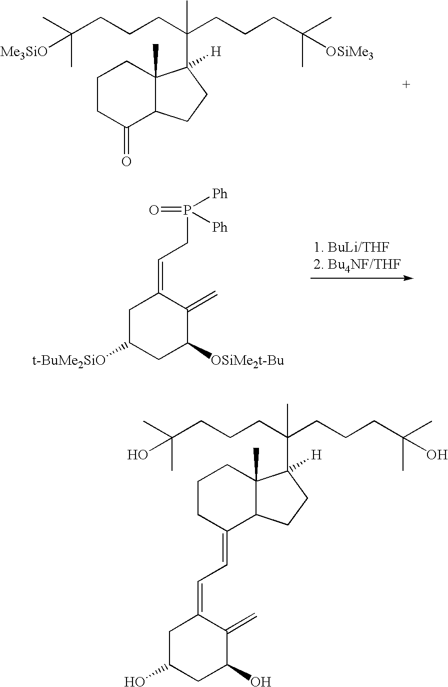

### 1,25-Dihydroxy-20-(4-hydroxy-4-methyl-pentyl)cholecalciferol

A 25 ml round bottom flask equipped with stir bar and Claisen adapter with rubber septum was charged with 700 mg (1.201 mmol) of (1S,5R)-1,5-bis-((tert-butyldimethyl)silanyloxy)-3-[2-(diphenylfosphinoyl)-eth-(Z)-ylidene]-2-methylene-cyclohexane and 5 ml of tetrahydrofurane. The reaction mixture was cooled to −70° C. and 0.75 ml (1.200 mmol) of 1.6M n-butyllithium was added dropwise. The resulting deep red solution was stirred at −78° C. for 25 min and 300 mg (0.571 mmol) of (1R,3aR,4S,7aR)-1-[1,5-dimethyl-1-(4-methyl-4-trimethylsilanyloxy-pentyl)-5-trimethylsilanyloxy-hexyl]-7a-methyl-octahydro-inden-4-one was added dropwise in 1 ml of tetrahydrofurane. The reaction mixture was stirred for 5 h and then the bath was removed and the mixture was poured into 50 ml of ethyl acetate and 100 ml of brine. The water fraction was extracted four times with 50 ml of ethyl acetate, dried over Na₂SO₄ and evaporated. The oil residue was chromatographed on column (50 cm³, protected from light) using hexane:ethyl acetate (20:1) as mobile phase. Fractions containing product were pooled and evaporated to give colorless oil (ca. 430 mg) which was treated with 5 ml of 1M tetrabutylammonium fluoride in tetrahydrofurane. The reaction mixture was stirred at room temperature for 24 h. The mixture was dissolved by the addition of 150 ml ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give colorless oil. Oil was crystallized from methyl acetate to give 183 mg (62%) of product.

[α]_{D}²⁹=+12.3° c=0.40, EtOH

UV λmax (EtOH): 213 nm (ε 14606), 264 nm (ε 17481)

¹H NMR (CDCl₃): 6.18(1H, d, J=11.1 Hz), 5.97 (1H, d, J=11.3 Hz), 5.23 (1H, d, J=1.3 Hz), 4.86 (1H, d, J=4.7 Hz), 4.75 (1H, d, J=1.7 Hz), 4.54 (1H, d, J=3.8 Hz), 4.20-4.16 (1H, m), 4.05 (1H, s), 4.04 (1H, s), 4.01-3.96 (1H, m), 2.77 (1H, br d, J=11.7 Hz), 2.35 (1H, br d, J=11.5 Hz), 2.17 (1H, dd, J=13.5, 5.2 Hz), 2.01-1.94 (2H, m), 1.83-1.78 (1H, m), 1.68-1.52 (6H, m), 1.48-1.05 (16H, m), 1.06 (12H, s), 0.86 (3H, s), 0.60 (3H, s)

¹³C NMR (CDCl₃): 149.41, 139.87, 135.74, 122.37, 117.81, 109.72, 68.72, 68.69, 68.34, 65.07, 56.64, 56.05, 46.17, 44.85, 44.79, 43.11, 40.53, 40.12, 39.56, 38.89, 29.48, 29.45, 29.18, 28.34, 23.15, 22.98, 21.89, 21.59, 18.07, 17.56, 14.70

| | | | | |
|---|---|---|---|---|
| MS HRES | Calculated for: | C₃₃H₅₆O₄ | [M + Na]⁺ | 539.4071 |
| | Observed: | | [M + Na]⁺ | 539.4066 |

### Example 4

### Synthesis of 1,25-Dihydroxy-20-(4-hydroxy-4-methyl-pentyl)-19-nor-cholecalciferol

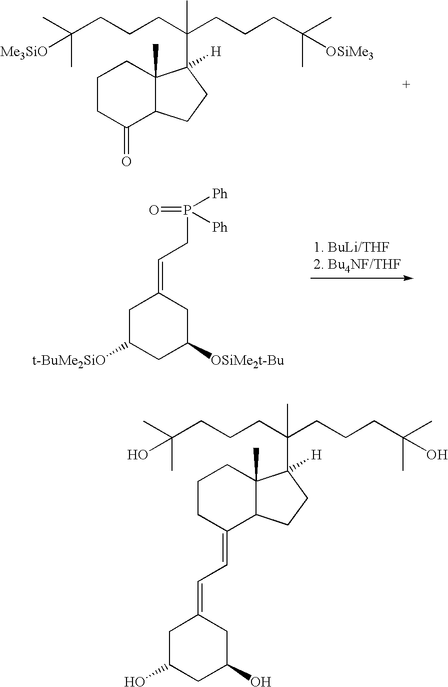

### 1,25-Dihydroxy-20-(4-hydroxy-4-methyl-pentyl)-19-nor-cholecalciferol

A 25 ml round bottom flask equipped with stir bar and Claisen adapter with rubber septum was charged with 1.023 g (1.792 mmol) of (1R,3R)-1,3-bis-((tert-butyldimethyl)silanyloxy)-5-[2-(diphenylfosphinoyl)ethylidene]-cyclohexane and 5 ml of tetrahydrofurane. The reaction mixture was cooled to −70° C. and 1.12 ml (1.792 mmol) of 1.6M n-butyllithium BuLi was added dropwise. The resulting deep red solution was stirred at −78° C. for 25 min and 350 mg (0.667 mmol) of (1R,3aR,4S,7aR)-1-[1,5-dimethyl-1-(4-methyl-4-trimethylsilanyloxy-pentyl)-5-trimethylsilanyloxy-hexyl]-7a-methyl-octahydro-inden-4-one in 1 ml of tetrahydrofurane. The reaction mixture was stirred for 5 h and then the dry ice was removed from bath and the solution was allowed to warm up to −40° C. in 1 h. The mixture was poured into 50 ml of ethyl acetate and 100 ml of brine. The water fraction was extracted four times with 50 ml of ethyl acetate, dried over Na₂SO₄ and evaporated. The oil residue was chromatographed on column (50 cm³, protected from light) using hexane:ethyl acetate (30:1 and 10:1) as mobile phase. Fractions containing product were pooled and evaporated to give colorless oil (ca. 500 mg) which was treated with 6 ml of 1M tetrabutylammonium fluoride in tetrahydrofurane. The reaction mixture was stirred at room temperature for 20 h. The new portion 3 ml of 1M tetrabutylammonium fluoride in tetrahydrofurane was added and the mixture was stirred for 22 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (2 times) to give 285 mg (85%) of product as a white solid.

[α]₂²³=+38.2° c=0.38, CHCl₃

UV λmax (EtOH): 243 nm (ε 33019), 251 nm (ε 38843), 261 nm (ε 26515)

¹H NMR (CDCl₃): 6.29(1H, d, J=11.1 Hz), 5.83 (1H, d, J=11.1 Hz), 4.12-4.09 (1H, m), 4.06-4.00 (1H, m), 2.80-2.71 (2H, m), 2.47 (1H, dd, J=13.3, 3.1 Hz), 2.23-2.17 (2H, m), 2.05-1.91 (3H, m), 1.78 (1H, ddd, J=13.1, 8.3, 3.1 Hz), 1.67-1.16 (24H, m), 1.21 (12H, s), 0.89 (3H, s), 0.63 (3H, s)

¹³C NMR (CDCl₃): 142.76, 131.16, 123.67, 115.63, 71.04, 67.38, 67.15, 57.18, 56.69, 46.73, 44.97, 44.92, 44.66, 42.20, 41.15, 39.70, 39.54, 39.37, 37.22, 29.44, 29.39, 29.36, 28.90, 23.48, 23.14, 22.41, 21.97, 18.44, 17.95, 15.12

| | | | | |
|---|---|---|---|---|
| MS HRES | Calculated for: | C₃₂H₅₆O₄ | [M + Na]⁺ | 527.4071 |
| | Observed: | | [M + Na]⁺ | 527.4073 |

### Example 5

### Synthesis of 1α-Fluoro-25-hydroxy-20-(4-hydroxy-4-methyl-pentyl)-cholecalciferol

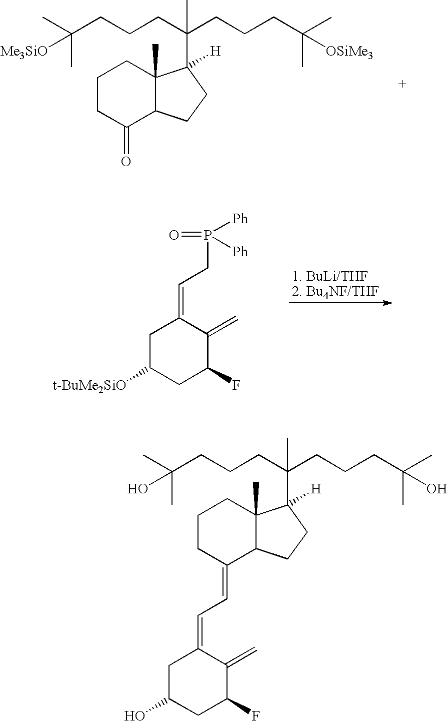

### 1α-Fluoro-25-hydroxy-20-(4-hydroxy-4-methyl-pentyl)-cholecalciferol

A 25 ml round bottom flask equipped with stir bar and Claisen adapter with rubber septum was charged with 680 mg (1.445 mmol) of (1S,5R)-1-((tent-butyldimethyl)silanyloxy)-3-[2-(diphenylfosphinoyl)-eth-(Z)-ylidene]-5-fluoro-2-methylene-cyclohexane and 5 ml of tetrahydrofurane. The reaction mixture was cooled to −70° C. and 0.9 ml (1.44 mmol) of 1.6M n-butyllithium was added dropwise. The resulting deep red solution was stirred at −78° C. for 25 min and 300 mg (0.571 mmol) of (1R,3aR,4S,7aR)-1-[1,5-dimethyl-1-(4-methyl-4-trimethylsilanyloxy-pentyl)-5-trimethylsilanyl oxy-hexyl]-7a-methyl-octahydro-inden-4-one was added dropwise in 1 ml of tetrahydrofurane. The reaction mixture was stirred for 4 h and then the dry ice was removed from bath and the solution was allowed to warm up to −40° C. in 1 h. The mixture was poured into 50 ml of ethyl acetate and 100 ml of brine. The water fraction was extracted three times with 50 ml of ethyl acetate, dried over Na₂SO₄ and evaporated.

The oil residue was chromatographed on column (50 cm³, protected from light) using hexane:ethyl acetate (30:1 and 10:1) as mobile phase. Fractions containing product were pooled and evaporated to give colorless oil (ca. 399 mg) which was treated with 5 ml of 1M tetrabutylammonium fluoride in tetrahydrofurane. The reaction mixture was stirred at room temperature for 20 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate:hexane (2:1 and 3:1) as mobile phase. Fractions containing product were pooled and evaporated to give product as colorless oil. The product was dissolved in methyl acetate and evaporated (2 times) to give 243 mg (82%) of product as white foam.

[α]_{D}²⁸=+9.3° c=0.40, CHCl₃

UV λmax (EtOH): 208 nm (ε 16024), 242 nm (ε 14965), 270 nm (ε 15024)

¹H NMR (CDCl₃): 6.39(1H, d, J=11.1 Hz), 6.01 (1H, d, J=11.3 Hz), 5.38 (1H, s), 5.13 (1H, ddd, J=49.9, 6.8, 3.7 Hz), 5.09 (1H, s), 4.25-4.18 (1H, m), 2.82-2.77 (1H, m), 2.61 (1H, dd, J=13.3, 3.7 Hz), 2.30 (1H, dd, J=13.3, 7.6 Hz), 2.22-2.13 (1H, m), 2.07-1.94 (3H, m), 1.76-1.15 (24H, m), 1.21 (12H, s), 0.89 (3H, s), 0.63 (3H, s)

¹³C NMR (CDCl₃): 143.30, 143.06(d, J=16.7 Hz), 131.40, 125.47, 117.37, 114.71(d, J=9.9 Hz), 91.53 (d, J=172.6 Hz), 71.05, 71.05, 66.53, 66.47, 57.17, 56.74, 46.89, 44.96, 44.90, 41.17, 40.87, 40.67, 39.67, 39.51, 39.36, 29.41, 29.35, 29.07, 23.56, 23.11, 22.37, 21.90, 18.43, 17.94, 15.05

### Example 6

### Synthesis of (20S)-1,25-Dihydroxy-20-(5,5,5-trifluoro-4-hydroxy-4-trifluoromethyl-pent-2-ynyl)cholecalciferol

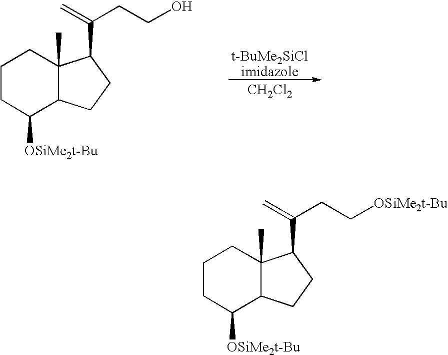

### (1R,3aR,4S,7aR)-4-(tert-Butyl-dimethyl-silanyloxy)-1-[3-(tert-butyl-dimethyl-silanyloxy)-1-methylene-propyl]-7a-methyl-octahydro-indene

A 250 ml round bottom flask equipped with stir bar, Claisen adapter with rubber septum and nitrogen sweep was charged with 17.53 g (51.77 mmol) of 3-[(1R,3aR,4S,7aR)-4-(tert-butyl-dimethyl-silanyloxy)-7a-methyl-octahydro-inden-1-yl]-but-3-en-1-ol and 75 ml of dichloromethane. A 7.05 g (103.54 mmol) imidazole was added followed by 9.36 g (62.124 mmol) of t-butyldimethylsilyl chloride. The mixture was stirred for 2.5 h.

The mixture was then diluted with 100 ml of water and extracted four times with 50 ml of dichloromethane. The combined organic layers were dried over Na₂SO₄ and evaporated. The oil residue was chromatographed on column (400 cm³) using hexane, hexane:ethyl acetate (50:1, 25:1) as mobile phase and collecting ca. 40 ml fractions to give 22.32 g (95%) of product as a colorless oil.

¹H NMR (CDCl₃): 4.87 (1H, s), 4.80 (1H, s), 4.02 (1H, br s), 3.67 (2H, t, J=7.3 Hz), 2.34-2.14 (2H, m), 2.06-2.00 (1H, m), 1.85-1.27 (9H, m), 1.20-1.08 (2H, m), 0.89 (18H, s), 0.79 (3H, s), 0.05 (6H, s), 0.02 (3H, s), 0.01 (3H, s).

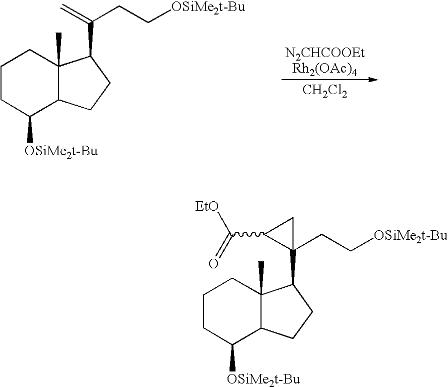

### 2-[2-(tert-Butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropanecarboxylic acid ethyl ester

A 250 ml round bottom flask equipped with stir bar and Claisen adapter with rubber septum was charged with 10.00 g (22.08 mmol) of (1R,3aR,4S,7aR)-4-(tert-butyl-dimethyl-silanyloxy)-1-[3-(tert-butyl-dimethyl-silanyloxy)-1-methylene-propyl]-7a-methyl-octahydro-indene, 200 mg of Rh₂(OAc)₄ and 40 ml of dichloromethane. A solution of 5.304 g (46.486 mmol) of ethyl diazoacetate in 30 ml of dichloromethane was added dropwise (12 ml/h) at room temperature. The reaction mixture was concentrated in vacuo and the remaining residue was filtrated on column (200 cm³) using hexane:ethyl acetate (1:1) as mobile phase. The solvent was evaporated and the oil residue was chromatographed on column (250 cm³) using hexane:ethyl acetate (25:1, 10:1 and 5:1) as mobile phase to give 8.44 g (71%) of products as a mixture of isomers.

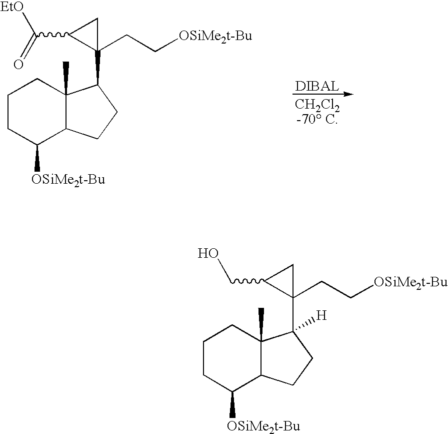

### {2-[2-(tert-Butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropyl}-methanol

A 50 ml round bottom flask equipped with stir bar and Claisen adapter with rubber septum was charged with 4.140 g (7.682 mmol) of 2-[2-(tert-butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropanecarboxylic acid ethyl ester and 20 ml of dichloromethane. The reaction mixture was cooled to −70° C. and 10.0 ml (15.0 mmol) of 1.5M DIBAL-H in toluene was added dropwise during 45 min. The reaction was stirred at −70° C. for 1 h and then 5 ml of saturated solution of ammonium chloride was added dropwise.

The mixture was dissolved by the addition of 100 ml of water and 50 ml of 1N HCl, extracted three times with 50 ml of ethyl acetate, dried over Na₂SO₄ and evaporated.

The oil residue was chromatographed on column (200 cm³) using hexane:ethyl acetate (10:1, 3:1) as mobile phase. The fractions containing product were pooled and evaporated to give 3.610 g, (94%) of products (mixture of isomers) as colorless oil.

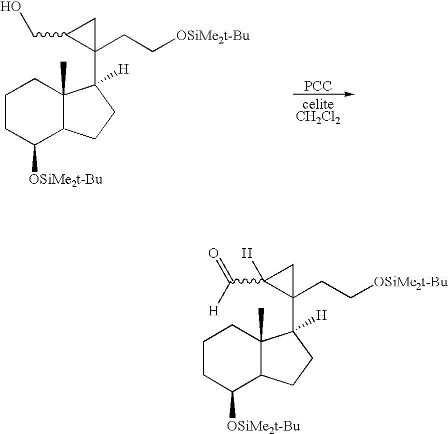

### 2-[2-(tert-Butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropanecarbaldehyde

A 250 ml round bottom flask equipped with stir bar and Claisen adapter with rubber septum was charged with 6.074 g (28.178 mmol) of pyridinium chlorochromate, 7.00 g of celite and 100 ml of dichloromethane. A 6.970 g (14.027 mmol) of {2-[2-(tert-butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropyl}-methanol in 10 ml of dichloromethane was added dropwise and mixture was stirred in room temperature for 1 h. The reaction mixture was filtrated through column with silica gel (200 cm³) and celite (2 cm) and using dichloromethane as a mobile phase. The fractions containing product were pooled and evaporated to give oil (ca. 5.71 g). Product was used to the next reaction without purification.

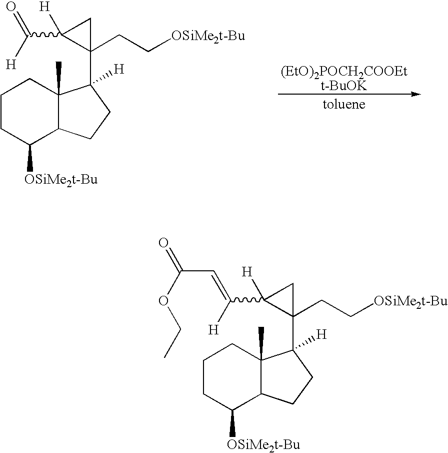

### 3-{2-[2-(tert-Butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropyl}-acrylic acid ethyl ester

A 250 ml round bottom flask equipped with stir bar and Claisen adapter with rubber septum was charged with 80 ml of toluene and 35.0 ml (35.0 mmol) of 1M potassium tert-butoxide in tetrahydrofurane was added. A 7.850 g (35.015 mmol) of triethyl phosphonoacetate in 5 ml of toluene was added dropwise at ca. 5° C. The mixture was stirred at room temperature for 1 h. Then the mixture was cooled to −15° C. and crude (ca. 11.54 mmol) 2-[2-(tert-butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropanecarbaldehyde in 5 ml of toluene was added and stirring was continued at −10° C. for 3 h. The reaction mixture was quenched with 10 ml of aqueous saturated solution of ammonium chloride, diluted with 100 ml of saturated solution of ammonium chloride and extracted four times with 50 ml of toluene and then 50 ml of ethyl acetate. The organic layer was washed with 50 ml of brine, dried and evaporated. The residue was purified over silica gel (200 cm³) using hexane:ethyl acetate (20:1) as a mobile phase to give 5.750 g (88%) of products (mixture of isomers).

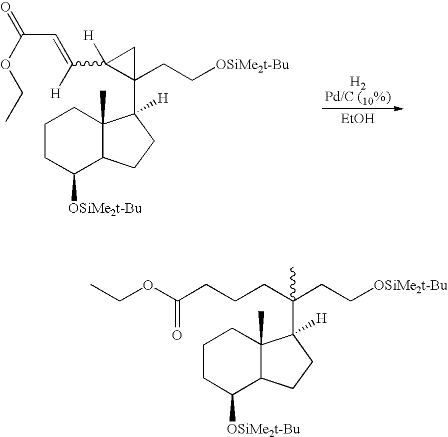

### 7-(tert-Butyl-dimethyl-silanyloxy)-5-[(1R,3aR,4S,7aR)-4-(tert-butyl-dimethyl-silanyloxy)-7a-methyl-octahydro-inden-1-yl]-5-methyl-heptanoic acid ethyl ester

A 5.750 g (10.177 mmol) of 3-{2-[2-(tert-butyl-dimethyl-silanyloxy)-ethyl]-2-[(1S,3aR, 4S,7aR)-4-(tert-butyl-dimethyl-silanyloxy)-7a-methyl-octahydro-inden-1-yl]-cyclopropyl}-acrylic acid ethyl ester was hydrogenated over 1.60 g of 10% Pd/C in 40 ml of ethanol at room temperature and atmospheric pressure of hydrogen. The reaction was monitoring by TLC (hexane:ethyl acetate—50:1). After 18 h the catalyst was filtered off and solvent evaporated. The residue was purified over silica gel (300 cm³) using hexane:ethyl acetate (100:1, 50:1, 20:1) as a mobile phase to give 5.150 g (89%) of products (mixture of isomers).

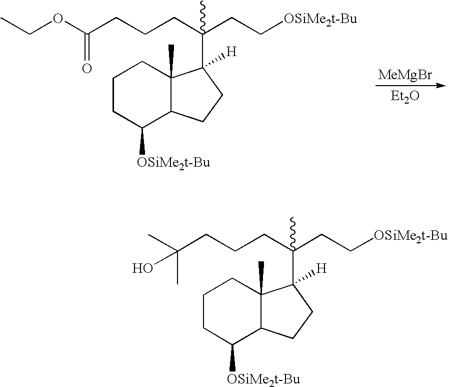

### 8-(tert-Butyl-dimethyl-silanyloxy)-6-[(1R,3aR,4S,7aR)-4-(tert-butyl-dimethyl-silanyloxy)-7a-methyl-octahydro-inden-1-yl]-2,6-dimethyl-octan-2-ol

A 250 ml round bottom flask equipped with stir bar, Claisen adapter with rubber septum was charged with 5.110 g (8.980 mmol) of 7-(tert-butyl-dimethyl-silanyloxy)-5-[(1R,3aR,4S, 7aR)-4-(tert-butyl-dimethyl-silanyloxy)-7a-methyl-octahydro-inden-1-yl]-5-methyl-heptanoic acid ethyl ester ester and 80 ml of diethyl ether. The solution was cooled in ace-water bath and 17.4 ml (54.3 mmol) of 3.12M solution of methyl magnesium bromide in diethyl ether was added dropwise. After completion of the addition the mixture was stirred at room temperature for 2.5 h then cooled again in an ice bath. A 10 ml of saturated solution of ammonium chloride was added dropwise. The resulting precipitate was dissolved by the addition of 50 ml of saturated solution of ammonium chloride. The aqueous layer was extracted three times with 100 ml of ethyl acetate. The combined organic layers were dried (Na₂SO₄) and evaporated. The product was used to the next reaction without farther purification.

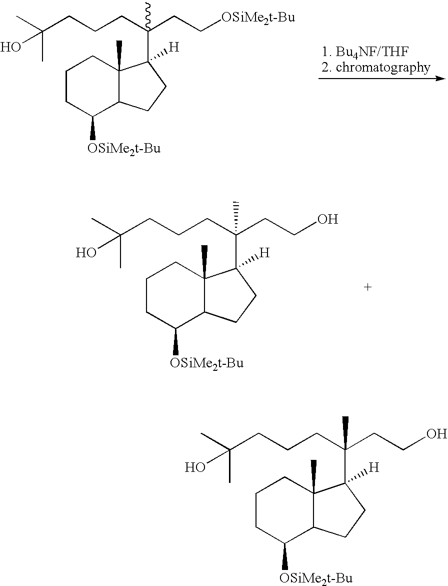

### 3-[(1R,3aR,4S,7aR)-4-(tert-Butyl-dimethyl-silanyloxy)-7a-methyl-octahydro-inden-1-yl]-3,7-dimethyl-octane-1,7-diol

A 50 ml round bottom flask equipped with stir bar and Claisen adapter with rubber septum was charged with crude (ca. 8.98 mmol) 8-(tert-butyl-dimethyl-silanyloxy)-6-[4-(tert-butyl-dimethyl-silanyloxy)-7a-methyl-octahydro-inden-1-yl]-2,6-dimethyl-octan-2-ol, 10 ml of tetrahydrofurane and 15.0 ml (15.0 mmol) of 1M tetrabutylammonium fluoride in tetrahydrofurane. The reaction mixture was stirred at room temperature for 2.5 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed four times on columns (400 cm³) using hexane:ethyl acetate (1:1) as a mobile phase to give: 1^{st}—1.456 g (low polar epimer); 2^{nd}—0.852 g, (mixture of epimers)′ 3^{rd}—1.132 g (more polar epimer)′ All products 3.440 g (88% two steps).

### Low polar epimer: (3S)-3-[(1R,3aR,4S,7aR)-4-(tert-Butyl-dimethyl-silanyloxy)-7a-methyl-octahydro-inden-1-yl]-3,7-dimethyl-octane-1,7-diol

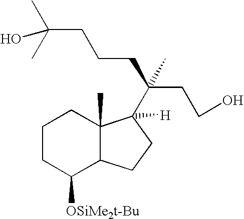

[α]_{D}³¹=+26.1° c=0.44, CHCl₃

¹H NMR (CDCl₃): 3.90 (1H, br s), 3.67 (2H, br t, J=8.1 Hz), 2.06-1.99 (1H, m), 1.87-1.50 (4H, m), 1.73 (2H, t, J=7.9 Hz), 1.40-1.06 (14H, m), 1.22 (6H, s), 1.06 (3H, s), 0.95 (3H, s), 1.95-0.82 (1H, m), 0.88 (9H, s), 0.00 (3H, s), −0.01(3H, s)

¹³C NMR (CDCl₃): 71.03, 69.58, 59.79, 57.32, 52.99, 44.78, 43.81, 41.64, 41.58, 40.26, 38.68, 34.37, 29.48, 29.36, 25.86, 23.49, 22.78, 21.72, 18.18, 18.09, 17.78, 16.78, −4.70, −5.07

| | | | | |
|---|---|---|---|---|
| MS HRES | Calculated for: | C₂₆H₅₂O₃Si | [M + Na]⁺ | 463.3578 |
| | Observed: | | [M + Na]⁺ | 463.3580 |

### More polar epimer: (3R)-3-[(1R,3aR,4S,7aR)-4-(tert-Butyl-dimethyl-silanyloxy)-7a-methyl-octahydro-inden-1-yl]-3,7-dimethyl-octane-1,7-diol

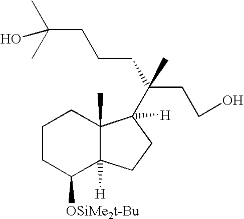

[α]_{D}³¹=+22.7° c=0.44, CHCl₃

¹H NMR (CDCl₃): 3.99-3.97 (1H, m), 3.65-3.61 (2H, m), 1.97 (1H, br d, J=12.3 Hz), 1.84-1.72 (1H, m), 1.66-1.50 (6H, m), 1.45-1.15 (14H, m), 1.21 (6H, s), 1.05 (3H, s), 0.95 (3H, s), 0.87 (9H, s), −0.01(3H, s), −0.02(3H, s)

¹³C NMR (CDCl₃): 71.05, 69.57, 59.47, 57.46, 53.02, 44.87, 43.90, 41.83, 41.61, 39.99, 38.93, 34.37, 29.43, 29.42, 25.87, 23.42, 22.84, 22.12, 18.57, 18.09, 17.81, 16.79, −4.69, −5.06

| | | | | |
|---|---|---|---|---|
| MS HRES | Calculated for: | C₂₆H₅₂O₃Si | [M + Na]⁺ | 463.3578 |
| | Observed: | | [M + Na]⁺ | 463.3575 |

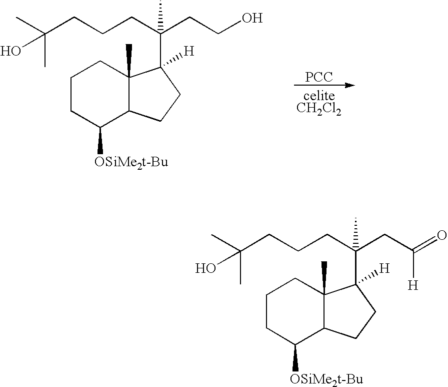

### (3S)-3-[(1R,3aR,4S,7aR)-4-(tert-Butyl-dimethyl-silanyloxy)-7a-methyl-octahydro-inden-1-yl]-7-hydroxy-3,7-dimethyl-octanal

A 25 ml round bottom flask equipped with stir bar and Claisen adapter with rubber septum was charged with 1.572 g (7.292 mmol) of pyridinium chlorochromate, 1.60 g of celite and 25 ml of dichloromethane. A 1.607 g (3.646 mmol) of (3S)-3-[(1R,3aR,4S,7aR)-4-(tert-butyl-dimethyl-silanyloxy)-7a-methyl-octahydro-inden-1-yl]-3,7-dimethyl-octane-1,7-diol in 6 ml of dichloromethane was added dropwise and mixture was stirred at room temperature for 1 h 45 min and additional portion 300 mg (1.392 mmol) of pyridinium chlorochromate was added. The reaction was stirred for next 1 h 15 min. The reaction mixture was filtrated through column with silica gel (50 cm³) and celite (1 cm) using dichloromethane, dichloromethane:ethyl acetate (4:1). The fractions containing product were pooled and evaporated to give 1.58 g of product as yellow oil. The product was used to the next reaction without further purification.

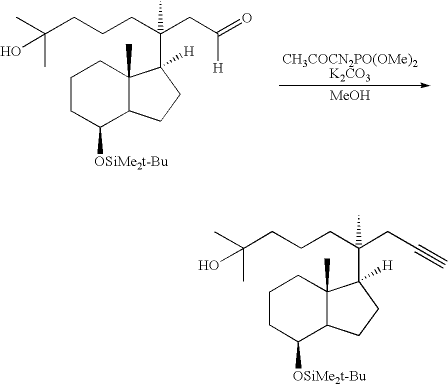

### (6S)-6-[(1R,3aR,4S,7aR)-4-(tert-Butyl-dimethyl-silanyloxy)-7a-methyl-octahydro-inden-1-yl]-2,6-dimethyl-non-8-yn-2-ol

A 50 ml round bottom flask equipped with stir bar and Claisen adapter with rubber septum was charged with 1.58 g (3.601 mmol) of (3S)-3-[(1R,3aR,4S,7aR)-4-(tert-butyl-dimethyl-silanyloxy)-7a-methyl-octahydro-inden-1-yl]-7-hydroxy-3,7-dimethyl-octanal and 30 ml of methanol. A 1.416 g (7.37 mmol) of 1-diazo-2-oxo-propyl)-phosphonic acid dimethyl ester in 3 ml of methanol was added and the resulting mixture was cooled in an ice bath. A 1.416 g (10.245 mmol) of potassium carbonate was added and the reaction mixture was stirred in the ice bath for 30 min and then at room temperature for 3 h. A 100 ml of water was added and the mixture was extracted three times with 80 ml of ethyl acetate, dried over Na₂SO₄ and evaporated. The oil residue was chromatographed on column (250 cm³) using hexane:ethyl acetate (7:1) as mobile phase. Fractions containing product were pooled and evaporated to give 1.310 g (83%, 2 steps) of product as colorless oil.

[α]_{D}³⁰=+15.7° c=0.61, CHCl₃

¹H NMR (CDCl₃): 3.98 (1H, br s), 2.28 (2H, d, J=2.1 Hz), 1.95-1.91 (2H, m), 1.78 (1H, dt, J=13.4, 3.8 Hz), 1.68-1.62 (1H, m), 1.58-1.48 (6H, m), 1.44-1.17 (15H, m), 1.22 (6H, s), 1.04 (3H, s), 1.00 (3H, s), 0.93-0.83 (1H, m), 0.88 (9H, s), −0.00(3H, s), −0.01(3H, s)

¹³C NMR (CDCl₃): δ3.09, 71.03, 69.84, 69.64, 56.68, 52.95, 44.80, 43.71, 41.31, 40.21, 39.28, 34.33, 29.44, 29.29, 28.80, 25.85, 22.74, 22.69, 22.18, 18.14, 18.05, 17.73, 16.68, −4.77, −5.13

| | | | | |
|---|---|---|---|---|
| MS HRES | Calculated for: | C₂₇H₅₀O₂Si | [M + Na]⁺ | 457.3472 |
| | Observed: | | [M + Na]⁺ | 457.3473 |

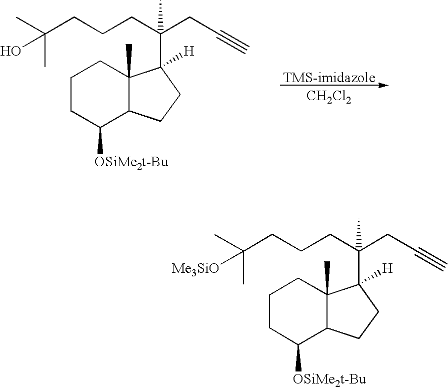

### (1R,3aR,4S,7aR)-4-(tert-Butyl-dimethyl-silanyloxy)-1-[(1S)-1,5-dimethyl-1-prop-2-ynyl-5-trimethylsilanyloxy-hexyl]-7a-methyl-octahydro-indene

A 50 ml round bottom flask equipped with stir bar and Claisen adapter with rubber septum was charged with 1.300 g (2.990 mmol) of (6S)-6-[(1R,3aR,4S,7aR)-4-(tert-butyl-dimethyl-silanyloxy)-7a-methyl-octahydro-inden-1-yl]-2,6-dimethyl-non-8-yn-2-ol and 25 ml of dichloromethane. A 2.00 ml (13.63 mmol) of 1-(trimethylsilyl) imidasole was added dropwise. The mixture was stirred at room temperature for 1 h.

A 100 ml of water was added and the mixture was extracted three times with 80 ml of hexane, dried over Na₂SO₄ and evaporated. The oil residue was chromatographed on column (75 cm³) using hexane:ethyl acetate (25:1) as mobile phase. Fractions containing product were pooled and evaporated to give 1.409 g (93%) of product as colorless oil.

¹H NMR (CDCl₃): 3.98 (1H, br s), 2.27 (2H, d, J=2.9 Hz), 1.97-1.91 (2H, m), 1.82-1.75 (1H, m), 1.69-1.62 (1H, m), 1.59-1.50 (2H, m), 1.42-1.20 (12H, m), 1.20 (6H, s), 1.05 (3H, s), 1.00 (3H, s), 0.93-0.85 (1H, m), 0.88 (9H, s), 0.10 (9H, s), 0.00 (3H, s), −0.01(3H, s)

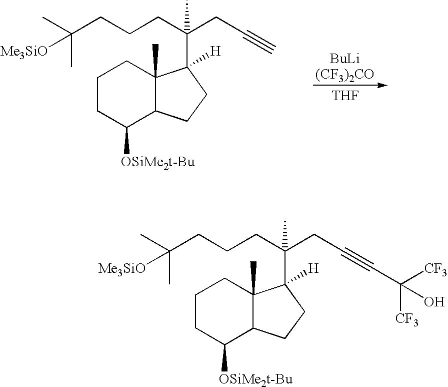

### (6S)-6-[(1R,3aR,4S,7aR)-4-(tert-Butyl-dimethyl-silanyloxy)-7a-methyl-octahydro-inden-1-yl]-1,1,1-trifluoro-6,10-dimethyl-2-trifluoromethyl-10-trimethylsilanyloxy-undec-3-yn-2-ol

A two neck 50 ml round bottom flask equipped with stir bar, Claisen adapter with rubber septum and funnel (with cooling bath) was charged with 1.390 g (2.742 mmol) of (1R,3aR,4S, 7aR)-4-(tert-butyl-dimethyl-silanyloxy)-1-[(1S)-1,5-dimethyl-1-prop-2-ynyl-5-trimethylsilanyloxy-hexyl]-7a-methyl-octahydro-indene and 30 ml of tetrahydrofurane. The funnel was connected to container with hexafluoroacetone and cooled (acetone, dry ice). The reaction mixture was cooled to −70° C. and 5.00 ml (8.00 mmol) of 1.6M n-butyllithium in tetrahydrofurane was added dropwise. After 30 min hexafluoroacetone was added (the contener's valve was opened three times). The reaction was stirred at −70° C. for 2 h then 5.0 ml of saturated solution of ammonium chloride was added. The mixture was dissolved by the addition of 100 ml of saturated solution of ammonium chloride and extracted three times with 80 ml of ethyl acetate, dried over Na₂SO₄ and evaporated. The oil residue was chromatographed twice to remove a large amount of polymer compounds. The first column (100 cm³) using hexane:ethyl:acetate (10:1) as mobile phase. The second column (100 cm³) using hexane:ethyl acetate (25:1, 15:1) as mobile phase. Fractions containing product were pooled and evaporated to give 1.959 g of colorless oil. Product was used to the next reaction without farther purification.

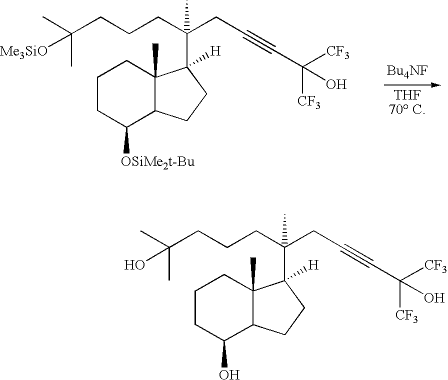

### (6S)-1,1,1-Trifluoro-6-[(1R,3aR,4S,7aR)-4-hydroxy-7a-methyl-octahydro-inden-1-yl]-6,10-dimethyl-2-trifluoromethyl-undec-3-yne-2,10-diol

A 25 ml round bottom flask equipped with stir bar and Claisen adapter with rubber septum was charged with crude (ca. 2.74 mmol) (6S)-6-[(1R,3aR,4S,7aR)-4-(tert-butyl-dimethyl-silanyloxy)-7a-methyl-octahydro-inden-1-yl]-1,1,1-trifluoro-6,10-dimethyl-2-trifluoromethyl-10-trimethylsilanyloxy-undec-3-yn-2-ol and 12.0 ml (12.0 mmol) of 1M tetrabutylammonium fluoride in tetrahydrofurane and reaction was stirred at 70° C. After 18 h new portion 5.0 ml of 1M tetrabutylammonium fluoride in tetrahydrofurane was added. The reaction mixture was stirred at 70° C. for next 80 h.

The mixture was dissolved by the addition of 150 ml of ethyl acetate and extracted six times with 50 ml of water:brine (1:1) and 50 ml of brine and dried over Na₂SO₄ and evaporated. The oil residue was chromatographed on column (200 cm³) using hexane:ethyl acetate (3:1, 2:1) as mobile phase. The fractions containing product were pooled and evaporated. The residue was crystallized from hexane-ethyl acetate to give 917 mg (69%, two steps) of product as a white crystal.

m.p. 146-147° C.

[α]_{D}³⁰=−3.5° c=0.43, CHCl₃

¹H NMR (CDCl₃): 4.08 (1H, br s), 2.45 (1H, AB, J=17 Hz), 2.36 (1H, AB, J=17 Hz), 1.98-1.92 (1H, m), 1.85-1.74 (2H, m), 1.67-1.18 (18H, m), 1.25 (6H, s), 1.07 (3H, s), 1.02 (3H, s)

| | | | | |
|---|---|---|---|---|
| MS HRES | Calculated for: | C₂₄H₃₆F₆O₃ | [M + Na]⁺ | 509.2461 |
| | Observed: | | [M + Na]⁺ | 509.2459 |

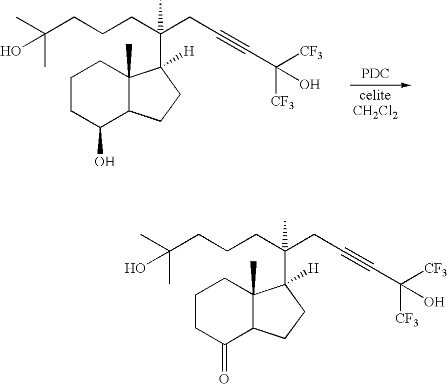

### (1R,3aR,4S,7aR)-7a-Methyl-1-[(1S)-6,6,6-trifluoro-5-hydroxy-1-(4-hydroxy-4-methyl-pentyl)-1-methyl-5-trifluoromethyl-hex-3-ynyl]-octahydro-inden-4-one

A 25 ml round bottom flask equipped with stir bar and Claisen adapter with rubber septum was charged with 300 mg (0.617 mmol) of (6S)-1,1,1-trifluoro-6-[(1R,3aR,4S,7aR)-4-hydroxy-7a-methyl-octahydro-inden-1-yl]-6,10-dimethyl-2-trifluoromethyl-undec-3-yne-2,10-diol and 10 ml of dichloromethane. A 696 mg (1.851 mmol) of pyridinium dichromate and 710 mg of celite were added and mixture was stirred in room temperature for 3 h. The reaction mixture was filtrated through column with silica gel (50 cm³) and celite (2 cm) and using dichloromethane: ethyl acetate (4:1) as a mobile phase. The fractions containing product were pooled and evaporated to give yellow oil. The product was used to the next reaction without farther purification.

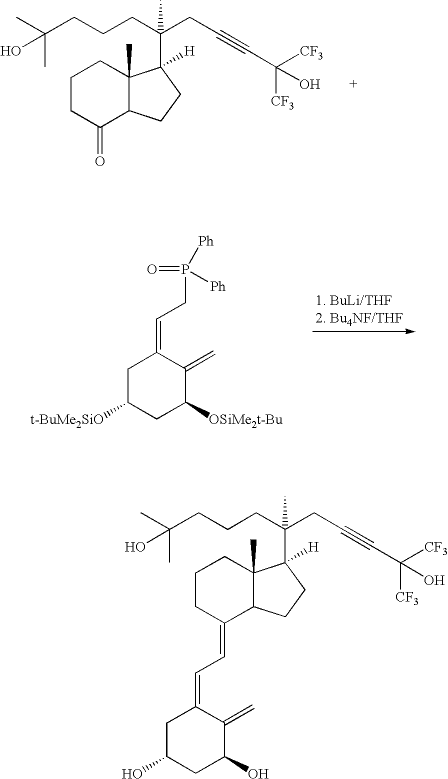

### (20S)-1,25-Dihydroxy-20-(5,5,5-trifluoro-4-hydroxy-4-trifluoromethyl-pent-2-ynyl)cholecalciferol

A 25 ml round bottom flask equipped with stir bar and Claisen adapter with rubber septum was charged with 1.798 g (3.084 mmol) of (1S,5R)-1,5-bis-((tert-butyldimethyl)silanyloxy)-3-[2-(diphenylfosphinoyl)-eth-(Z)-ylidene]-2-methylene-cyclohexane and 12 ml of tetrahydrofurane. The reaction mixture was cooled to −78° C. and 1.9 ml (3.04 mmol) of 1.6M n-butyllithium in tetrahydrofurane was added dropwise. The resulting deep red solution was stirred at −78° C. for 20 min and crude (ca 0.617 mmol) (1R,3aR,4S,7aR)-7a-methyl-1-[(1S)-6,6,6-trifluoro-5-hydroxy-1-(4-hydroxy-4-methyl-pentyl)-1-methyl-5-trifluoromethyl-hex-3-ynyl]-octahydro-inden-4-one was added dropwise in 1.5 ml of tetrahydrofurane. The reaction mixture was stirred for 5 h and then the bath was removed and the mixture was poured into 50 ml of ethyl acetate and 100 ml of brine. The water fraction was extracted three times with 50 ml of ethyl acetate, dried over Na₂SO₄ and evaporated. The oil residue was chromatographed on column (75 cm³, protected from light) using hexane:ethyl acetate (5:1) as mobile phase. Fractions containing product were pooled and evaporated to give colorless oil (293 mg) which was treated with 5 ml of 1M tetrabutylammonium fluoride in tetrahydrofurane. The reaction mixture was stirred at room temperature for 40 h.

The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give 190 mg (50% three steps) of product as white foam.

[α]_{D}³⁰=−4.6° c=0.35, CHCl₃

UV λmax (EtOH): 205.50 nm (ε 16586), 266.00 nm (ε 14319)

¹H NMR (CDCl₃): 6.36(1H, d, J=11.3 Hz), 6.23 (1H, br s), 6.00 (1H, d, J=11.1 Hz), 5.32 (1H, s), 4.98 (1H, s), 4.43 (1H, dd, J=7.7, 4.3 Hz), 4.25-4.20 (1H, m), 2.82-2.79 (1H, m), 2.59 (1H, dd, J=13.1, 3.1 Hz), 2.44 (1H, AB, J=17.2 Hz), 2.37 (1H, AB, J=17.2 Hz), 2.30 (1H, dd, J=13.2, 6.2 Hz,), 2.06-1.87 (4H, m), 1.72-1.36 (11H, m), 1.26-1.21 (1H, m), 1.24 (6H, s), 0.99 (3H, s), 0.64 (3H, s)

¹³C NMR (CDCl₃): 147.48, 142.29, 133.16, 124.72, 121.32(q, J=287.1 Hz), 117.59, 11.68, 90.08, 72.62, 71.39, 70.73, 66.89, 57.28, 56.52, 46.65, 45.18, 43.20, 42.81, 41.04, 40.89, 40.03, 29.79, 29.35, 28.95, 23.45, 22.86, 22.60, 21.84, 17.77, 14.93

| | | | | |
|---|---|---|---|---|
| MS HRES | Calculated for: | C₃₃H₄₆F₆O₄ | [M + Na]⁺ | 643.3192 |
| | Observed: | | [M + Na]⁺ | 643.3192 |

### Example 7

### Synthesis of (20S)-1,25-Dihydroxy-20-(5,5,5-trifluoro-4-hydroxy-4-trifluoromethyl-pent-2-ynyl)-19-nor-cholecalciferol

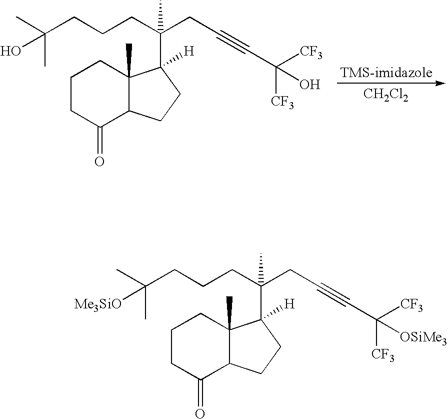

### (1R,3aR,4S,7aR)-7a-Methyl-1-[(1S)-6,6,6-trifluoro-1-methyl-1-(4-methyl-4-trimethylsilanyloxy-pentyl)-5-trifluoromethyl-5-trimethylsilanyloxy-hex-3-ynyl]-octahydro-inden-4-one

A 25 ml round bottom flask equipped with stir bar and Claisen adapter with rubber septum was charged with 585 mg (1.207 mmol) of (1R,3aR,4S,7aR)-7a-methyl-1-[(1S)-6,6,6-trifluoro-5-hydroxy-1-(4-hydroxy-4-methyl-pentyl)-1-methyl-5-trifluoromethyl-hex-3-ynyl]-octahydro-inden-4-one and 10 ml of dichloromethane. A 1.5 ml (10.2 mmol) of 1-(trimethylsilyl)imidazole was added dropwise. The mixture was stirred at room temperature for 3 h. A 150 ml of ethyl acetate was added and the mixture was washed three times with 50 ml of water, dried over Na₂SO₄ and evaporated.

The oil residue was chromatographed on column (50 cm³) using hexane:ethyl acetate (10:1) as mobile phase. Fractions containing product were pooled and evaporated to give 660 mg (87%) of product as colorless oil.

¹H NMR (CDCl₃): 2.44-2.39 (3H, m), 2.32-2.16 (2H, m), 2.10-1.99 (2H, m), 1.95-1.84 (2H, m), 1.77-1.56 (4H, m), 1.38-1.19 (7H, m), 1.20 (6H, s), 1.03 (3H, s), 0.74 (3H, s), 0.28 (9H, s), 0.10 (9H, s)

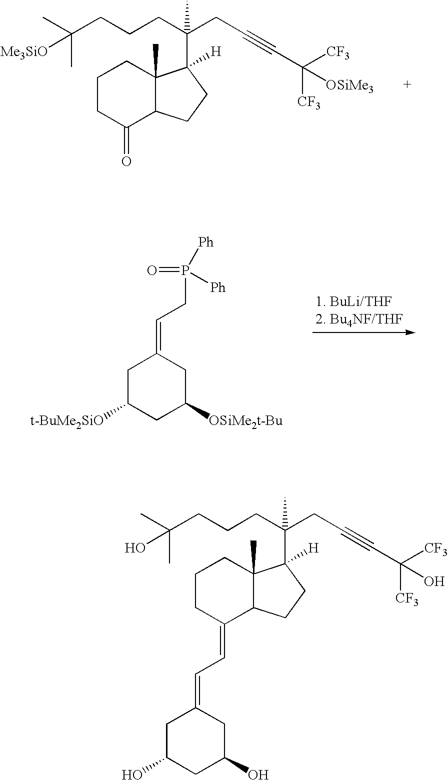

### (20S)-1,25-Dihydroxy-20-(5,5,5-trifluoro-4-hydroxy-4-trifluoromethyl-pent-2-ynyl)-19-nor-cholecalciferol

A 25 ml round bottom flask equipped with stir bar and Claisen adapter with rubber septum was charged with 618 mg (1.083 mmol) of (1R,3R)-1,3-bis-((tent-butyldimethyl)silanyloxy)-5-[2-(diphenylfosphinoyl)ethylidene]-cyclohexane and 10 ml of tetrahydrofurane. The reaction mixture was cooled to −70° C. and 0.67 ml (1.07 mmol) of 1.6M n-butyllithium BuLi was added dropwise. The resulting deep red solution was stirred at −70° C. for 20 min and 335 mg (0.532 mmol) of (1R,3aR,4S,7aR)-7a-methyl-1-[(1S)-6,6,6-trifluoro-1-methyl-1-(4-methyl-4-trimethylsilanyloxy-pentyl)-5-trifluoromethyl-5-trimethylsilanyloxy-hex-3-ynyl]-octahydro-inden-4-one in 1.5 ml of tetrahydrofurane. The reaction mixture was stirred for 5 h and then the dry ice was removed from bath and the solution was allowed to warm up to −40° C. in 1 h. The mixture was poured into 50 ml of ethyl acetate and 100 ml of brine. The water fraction was extracted four times with 50 ml of ethyl acetate, dried over Na₂SO₄ and evaporated.

The oil residue was chromatographed on column (50 cm³, protected from light) using hexane:ethyl acetate (10:1) as mobile phase. Fractions containing product were pooled and evaporated to give colorless oil (ca. 440 mg) which was treated with 10 ml of 1M tetrabutylammonium fluoride in tetrahydrofurane. The reaction mixture was stirred at room temperature for 29 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (2 times) to give 308 mg (95%) of product as white foam.

[α]_{D}²⁶=+38.8 c=0.42, EtOH

UV λmax (EtOH): 243 nm (ε 29530), 252 nm (ε 33645), 261 nm (ε 23156) ¹H NMR (CDCl₃): 6.28(1H, d, J=11.3 Hz), 5.83 (1H, d, J=11.1 Hz), 4.12-4.09 (1H, m), 4.05-4.01 (1H, m), 2.80-2.72 (2H, m), 2.46 (1H, dd, J=13.4, 3.0 Hz), 2.42 (1H, AB, J=16.8 Hz), 2.36 (1H, AB, J=16.8 Hz), 2.22-2.16 (2H, m), 2.04-1.86 (6H, m), 1.80-1.38 (17H, m), 1.23 (6H, s), 0.99 (3H, s), 0.63 (3H, s)

¹³C NMR (CDCl₃): 142.13, 131.41, 123.55, 121.36(q, J=286.9 Hz, 115.88, 72.40, 71.40, 67.40, 67.15, 27.19, 56.47, 46.50, 44.44, 43.40, 41.94, 40.91, 40.83, 39.97, 37.09, 29.65, 29.29, 29.26, 28.79, 23.35, 22.79, 22.60, 21.81, 17.79, 15.00

| | | | | |
|---|---|---|---|---|
| MS HRES | Calculated for: | C₃₂H₄₆F₆O₄ | [M + Na]⁺ | 631.3192 |
| | Observed: | | [M + Na]⁺ | 631.3191 |

### Example 8

### Synthesis of (20S)-1α-Fluoro-25-hydroxy-20-(5,5,5-trifluoro-4-hydroxy-4-trifluoromethyl-pent-2-ynyl)-cholecalciferol

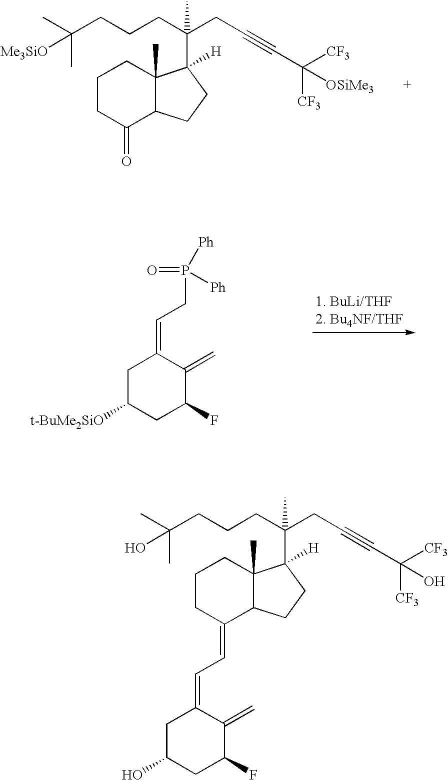

### (20S)-1α-Fluoro-25-hydroxy-20-(5,5,5-trifluoro-4-hydroxy-4-trifluoromethyl-pent-2-ynyl)-cholecalciferol

A 25 ml round bottom flask equipped with stir bar and Claisen adapter with rubber septum was charged with 495 mg (1.052 mmol) of (1S,5R)-1-((tent-butyldimethyl)silanyloxy)-3-[2-(diphenylfosphinoyl)-eth-(Z)-ylidene]-5-fluoro-2-methylene-cyclohexane and 10 ml of tetrahydrofurane. The reaction mixture was cooled to −70° C. and 0.65 ml (1.04 mmol) of 1.6M n-butyllithium was added dropwise. The resulting deep red solution was stirred at −70° C. for 20 min and 300 mg (0.477 mmol) of (1R,3aR,4S,7aR)-7a-methyl-1-[(1S)-6,6,6-trifluoro-1-methyl-1-(4-methyl-4-trimethylsilanyloxy-pentyl)-5-trifluoromethyl-5-trimethylsilanyloxy-hex-3-ynyl]-octahydro-inden-4-one was added dropwise in 1.5 ml of tetrahydrofurane. The reaction mixture was stirred for 4 h and then the dry ice was removed from bath and the solution was allowed to warm up to −40° C. in 1 h. The mixture was poured into 50 ml of ethyl acetate and 100 ml of brine. The water fraction was extracted three times with 50 ml of ethyl acetate, dried over Na₂SO₄ and evaporated. The oil residue was chromatographed on column (50 cm³, protected from light) using hexane:ethyl acetate (10:1) as mobile phase. Fractions containing product were pooled and evaporated to give colorless oil (ca. 429 mg) which was treated with 10 ml of 1M tetrabutylammonium fluoride in tetrahydrofurane. The reaction mixture was stirred at room temperature for 18 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate:hexane (1:1) as mobile phase. Fractions containing product were pooled and evaporated to give product as colorless oil. The product was dissolved in methyl acetate and evaporated (2 times) to give 274 mg 92%) of product as white foam.

[α]_{D}³⁰=+27.0 c=0.50, EtOH

UV λmax (EtOH): 212 nm (ε 34256), 243 nm (ε 15866), 271 nm (ε 16512)

¹H NMR (CDCl₃): 6.38(1H, d, J=11.3 Hz), 6.01 (1H, d, J=11.3 Hz), 5.38 (1H, s), 5.13 (1H, ddd, J=49.9, 6.6, 3.6 Hz), 5.09 (1H, s), 4.23-4.19 (1H, m), 2.80 (1H, dd, J=12.0, 3.5 Hz), 2.61 (1H, dd, J=13.3, 3.7 Hz), 2.43 (1H, AB, J=16.9 Hz), 2.36 (1H, AB, J=16.9 Hz), 2.30 (1H, dd, J=13.4, 7.9 Hz), 2.24-2.15 (1H, m), 2.04-1.92 (3H, m), 1.73-1.35 (17H, m), 1.26-1.21 (1H, m), 1.24 (6H, s), 0.99 (3H, s), 0.64 (3H, s)

¹³C NMR (CDCl₃): 142.97(d, J=16.8 Hz), 142.69, 131.68(d, J=2.2 Hz), 125.37, 121.34(q, J=286.9 Hz), 117.63, 114.99(d, J=10.0 Hz), 91.61 (d, J=172.4 Hz), 90.07, 72.62, 71.38, 66.56(d, J=6.0 Hz), 57.26, 56.53, 46.68, 44.91, 43.31, 40.97, 40.89, 40.68(d, J=20.6 Hz), 40.01, 29.67, 29.28, 28.98, 23.43, 22.81, 22.60, 21.78, 17.79, 14.96

| | | | | |
|---|---|---|---|---|
| MS HRES | Calculated for: | C₃₃H₄₅F₇O₃ | [M + Na]⁺ | 645.3149 |
| | Observed: | | [M + Na]⁺ | 645.3148 |

### Example 9

### Synthesis of (20S)-1,25-Dihydroxy-20-(5,5,5-trifluoro-4-hydroxy-4-trifluoromethyl-pent-(2Z)-enyl)cholecalciferol

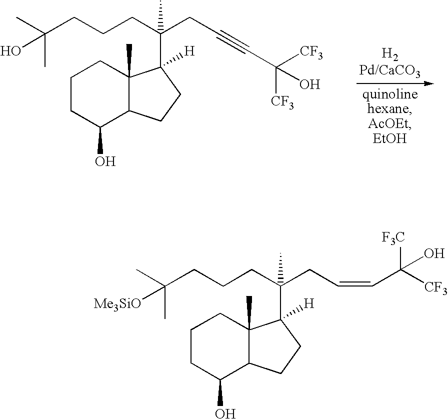

### (3Z,6S)-1,1,1-Trifluoro-6-[(1R,3aR,4S,7aR)-4-hydroxy-7a-methyl-octahydro-inden-1-yl]-6,10-dimethyl-2-trifluoromethyl-undec-3-ene-2,10-diol

A 25 ml round bottom flask was charged with 250 mg (0.514 mmol) of (6S)-1,1,1-trifluoro-6-[(1R,3aR,4S,7aR)-4-hydroxy-7a-methyl-octahydro-inden-1-yl]-6,10-dimethyl-2-trifluoromethyl-undec-3-yne-2,10-diol, 70 mg of 5% Pd/CaCO₃, 6.0 ml of hexane, 2.4 ml of ethyl acetate and 0.23 ml of solution of quinoline in ethanol (prepared from 3.1 ml of ethanol and 168 μl of quinoline). The substrate was hydrogenated at ambient temperature and atmospheric pressure of hydrogen. The reaction was monitoring by TLC (hexane:ethyl acetate—2:1). After 7 h the catalyst was filtered off and solvent evaporated. The residue was purified over silica gel (125 cm³) using hexane:ethyl acetate (2:1) as a mobile phase. Fractions containing product were pooled and evaporated to give 243 mg (97%) of product as colorless oil.

¹H NMR (CDCl₃): 6.14-6.05 (1H, m), 5.49 (1H, d, J=12.5 Hz), 4.08 (1H, br s), 2.83 (1H, dd, J=15.9, 9.7 Hz), 2.48-2.38 (1H, m), 1.85-1.75 (2H, m), 1.65-1.20 (17H, m), 1.22 (3H, s), 1.20 (3H, s), 1.08 (3H, s), 1.03-0.96 (1H, m), 1.00 (3H, s)

¹³C NMR (CDCl₃): 140.22, 117.44, 71.79, 69.66, 56.74, 52.58, 44.11, 43.45, 41.19, 40.24, 39.64, 36.88, 33.44, 30.09, 28.88, 22.55, 22.21, 21.70, 17.63, 17.58, 16.54

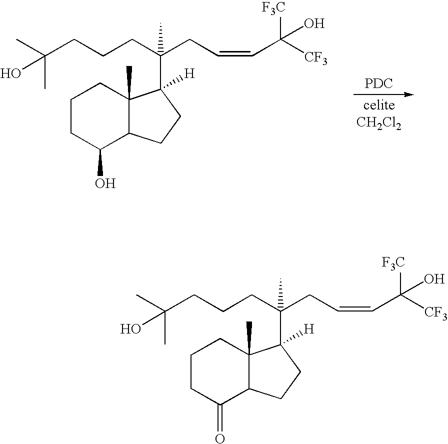

### (1R,3aR,4S,7aR)-7a-Methyl-1-[(1S,3Z)-6,6,6-trifluoro-5-hydroxy-1-(4-hydroxy-4-methyl-pentyl)-1-methyl-5-trifluoromethyl-hex-3-enyl]-octahydro-inden-4-one

A 25 ml round bottom flask equipped with stir bar and Claisen adapter with rubber septum was charged with 290 mg (0.594 mmol) of (3Z,6S)-1,1,1-trifluoro-6-[(1R,3aR,4S, 7aR)-4-hydroxy-7a-methyl-octahydro-inden-1-yl]-6,10-dimethyl-2-trifluoromethyl-undec-3-ene-2,10-diol and 10 ml of dichloromethane. A 700 mg (1.861 mmol) pyridinium dichromate and 750 mg of celite was added and mixture was stirred in room temperature for 3 h. The reaction mixture was filtrated through column with silica gel (75 cm³) and celite (2 cm) and using dichloromethane: ethyl acetate (4:1) as a mobile phase. The fractions containing product were pooled and evaporated to give yellow oil. The product was used to the next reaction without farther purification.

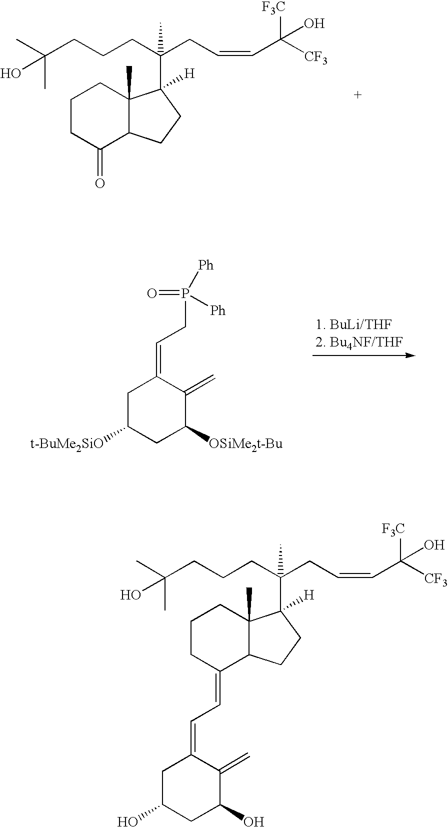

### (20S)-1,25-Dihydroxy-20-(5,5,5-trifluoro-4-hydroxy-4-trifluoromethyl-pent-(2Z)-enyl)cholecalciferol

A 25 ml round bottom flask equipped with stir bar and Claisen adapter with rubber septum was charged with 1.800 g (3.088 mmol) of (1S,5R)-1,5-bis-((tert-butyldimethyl)silanyloxy)-3-[2-(diphenylfosphinoyl)-eth-(Z)-ylidene]-2-methylene-cyclohexane and 10.0 ml of tetrahydrofurane. The reaction mixture was cooled to −78° C. and 1.9 ml (3.04 mmol) of 1.6M n-butyllithium in tetrahydrofurane was added dropwise. The resulting deep red solution was stirred at −78° C. for 20 min and 278 mg (0.571 mmol) of (1R,3aR,4S,7aR)-7a-methyl-1-[(1S,3Z)-6,6,6-trifluoro-5-hydroxy-1-(4-hydroxy-4-methyl-pentyl)-1-methyl-5-trifluoromethyl-hex-3-enyl]-octahydro-inden-4-one was added dropwise in 1.5 ml of tetrahydrofurane. The reaction mixture was stirred for 5 h (last 0.5 h at −20° C.) and then the bath was removed and the mixture was poured into 50 ml of ethyl acetate and 100 ml of brine. The water fraction was extracted three times with 50 ml of ethyl acetate, dried over Na₂SO₄ and evaporated. The oil residue was chromatographed on column (75 cm³, protected from light) using hexane:ethyl acetate (4:1) as mobile phase. Fractions containing product were pooled and evaporated to give colorless oil (309 mg) which was treated with 5 ml of 1M tetrabutylammonium fluoride in tetrahydrofurane. The reaction mixture was stirred at room temperature for 22 h.

The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give 192 mg (54%, two steps) of product as white foam.

UV λmax (EtOH): 204.08 nm (ε 27522), 266.03 nm (ε 20144)

¹H NMR (CDCl₃): 6.37(1H, d, J=11.1 Hz), 6.10 (1H, ddd, J=12.5, 9.0, 6.0 Hz), 6.00 (1H, d, J=11.3 Hz), 5.47 (1H, d, J=12.2 Hz), 5.32 (1H, s), 5.07 (1H, br, s), 4.99 (1H, s), 4.43 (1H, dd, J=7.8, 4.2 Hz), 4.25-4.20 (1H, m), 2.85-2.79 (2H, m), 2.59 (1H, dd, J=13.4, 3.0 Hz), 2.46 (1H, dd, J=16.4, 4.9 Hz), 2.31 (1H, dd, J=13.4, 6.4 Hz), 2.04-1.97 (3H, m), 1.90 (1H, ddd, J=12.0, 8.2, 3.2 Hz), 1.76-1.20 (17H, m), 1.21 (3H, s), 1.20 (3H, s), 1.06-1.00 (1H, m), 0.96(3H, s), 0.64 (3H, s)

¹³C NMR (CDCl₃): 147.51, 142.74, 140.17, 132.92, 124.88, 122.95(q, J=286.9 Hz), 122.80 (q, J=285.5 Hz), 117.52, 117.39, 111.65, 71.94, 70.73, 66.88, 56.86, 56.65, 46.79, 45.20, 43.95, 42.83, 41.06, 40.09, 39.75, 37.22, 30.35, 29.05, 28.82, 23.58, 22.50, 22.19, 21.93, 17.53, 15.04

| | | | | |
|---|---|---|---|---|
| MS HRES | Calculated for: | C₃₃H₄₈F₆O₄ | [M + Na]⁺ | 645.3349 |
| | Observed: | | [M + Na]⁺ | 645.3350 |

### Example 10

### Synthesis of (20S)-1,25-Dihydroxy-20-[(2Z)-5,5,5-trifluoro-4-hydroxy-4-trifluoromethyl-pent-2-enyl]-19-nor-cholecalciferol

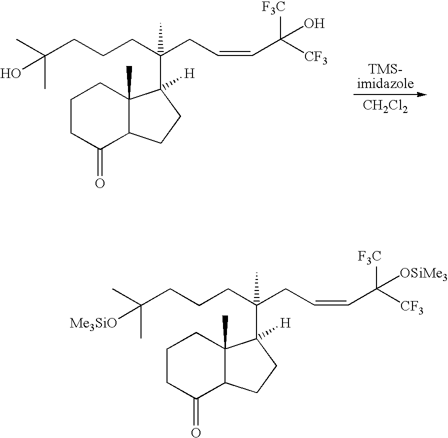

### (1R,3aR,4S,7aR)-7a-Methyl-1-[(1S,3Z)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one

A 25 ml round bottom flask equipped with stir bar and Claisen adapter with rubber septum was charged with 590 mg (1.213 mmol) of (1R,3aR,4S,7aR)-7a-methyl-1-[(1S,3Z)-6,6,6-trifluoro-5-hydroxy-1-(4-hydroxy-4-methyl-pentyl)-1-methyl-5-trifluoromethyl-hex-3-enyl]-octahydro-inden-4-one and 15 ml of dichloromethane. A 1.4 ml (9.5 mmol) of 1-(trimethylsilyl)imidazole was added dropwise. The mixture was stirred at room temperature for 4 h. A 150 ml of ethyl acetate was added and the mixture was washed three times with 50 ml of water, dried over Na₂SO₄ and evaporated.

The oil residue was chromatographed on column (50 cm³) using hexane:ethyl acetate (10:1) as mobile phase. Fractions containing product were pooled and evaporated to give 726 mg (95%) of product as colorless oil.

¹H NMR (CDCl₃): 6.07-5.99 (1H, m), 5.41 (1H, d, J=11.4 Hz), 2.52 (2H, dd, J=6.2, 2.6 Hz), 2.44-2.38 (1H, m), 2.31-1.54 (11H, m), 1.36-1.14 (6H, m), 1.19 (6H, s), 0.97 (3H, s), 0.74 (3H, s), 0.25 (9H, s), 0.09 (9H, s)

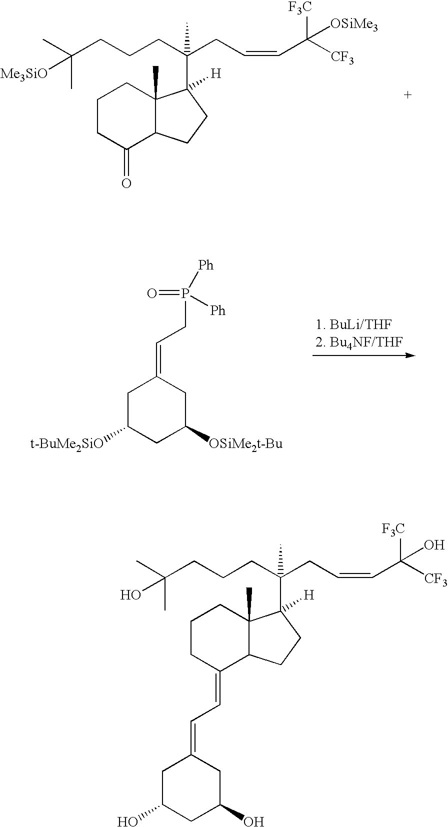

### (20S)-1,25-Dihydroxy-20-[(2Z)-5,5,5-trifluoro-4-hydroxy-4-trifluoromethyl-pent-2-enyl]-19-nor-cholecalciferol

A 25 ml round bottom flask equipped with stir bar and Claisen adapter with rubber septum was charged with 841 mg (1.473 mmol) of (1R,3R)-1,3-bis-((tent-butyldimethyl)silanyloxy)-5-[2-(diphenylfosphinoyl)ethylidene]-cyclohexane and 10 ml of tetrahydrofurane. The reaction mixture was cooled to −70° C. and 0.88 ml (1.41 mmol) of 1.6M n-butyllithium BuLi was added dropwise. The resulting deep red solution was stirred at −70° C. for 20 min and 369 mg (0.585 mmol) of (1R,3aR,4S,7aR)-7a-methyl-1-[(1S,3Z)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one in 1.5 ml of tetrahydrofurane. The reaction mixture was stirred for 5 h and then the dry ice was removed from bath and the solution was allowed to warm up to −40° C. in 1 h. The mixture was poured into 50 ml of ethyl acetate and 100 ml of brine. The water fraction was extracted three times with 50 ml of ethyl acetate, dried over Na₂SO₄ and evaporated.

The oil residue was chromatographed on column (50 cm³, protected from light) using hexane:ethyl acetate (10:1) as mobile phase. Fractions containing product were pooled and evaporated to give colorless oil (ca. 560 mg) which was treated with 8 ml of 1M tetrabutylammonium fluoride in tetrahydrofurane. The reaction mixture was stirred at room temperature for 8 h. The new portion 7 ml of 1M tetrabutylammonium fluoride in tetrahydrofurane was added and the mixture was stirred for 40 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (2 times) to give 327 mg (92%) of product as white foam.

[α]_{D}²⁸=+32° c=0.43, EtOH

UV λmax (EtOH): 243.67 nm (ε 36197), 252.00 nm (ε 41649), 261.83 nm (ε 28455)

¹H NMR (CDCl₃): 6.31(1H, d, J=11.2 Hz), 6.11 (1H, ddd, J=12.4, 9.3, 5.7 Hz), 5.84 (1H, d, J=10.7 Hz), 5.48 (1H, d, J=11.7 Hz), 4.12 (1H, br s), 4.05 (1H, br s), 2.86-2.72 (3H, m), 2.50-2.46 (2H, m), 2.24-2.18 (2H, m), 2.08-1.94 (3H, m), 1.88-1.22 (18H, m), 1.22 (6H, s), 1.06-0.91(2H, m), 0.97 (3H, s), 0.65 (3H, s)

| | | | | |
|---|---|---|---|---|
| MS HRES | Calculated for: | C₃₂H₄₈F₆O₄ | [M + Na]⁺ | 633.3349 |
| | Observed: | | [M + Na]⁺ | 633.3348 |

### Example 11

### Synthesis of (20S)-1α-Fluoro-25-hydroxy-20-[(2Z)-5,5,5-trifluoro-4-hydroxy-4-trifluoromethyl-pent-2-enyl]-cholecalciferol

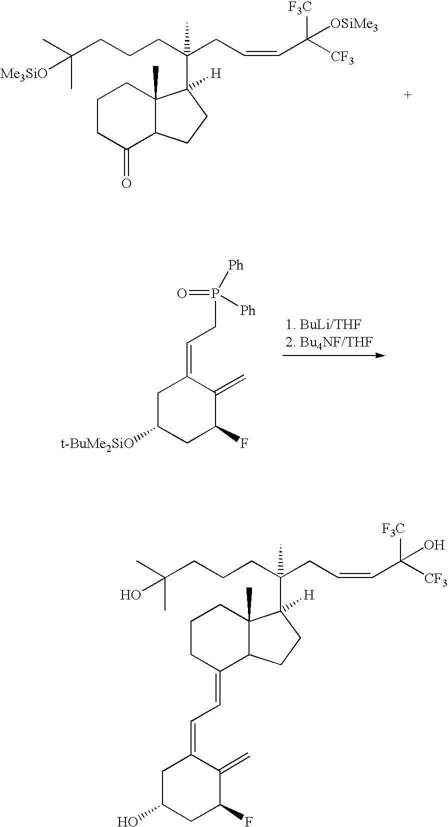

### (20S)-1α-Fluoro-25-hydroxy-20-[(2Z)-5,5,5-trifluoro-4-hydroxy-4-trifluoromethyl-pent-2-enyl]-cholecalciferol

A 25 ml round bottom flask equipped with stir bar and Claisen adapter with rubber septum was charged with 712 mg (1.513 mmol) of (1S,5R)-1-((tent-butyldimethyl)silanyloxy)-3-[2-(diphenylfosphinoyl)-eth-(Z)-ylidene]-5-fluoro-2-methylene-cyclohexane and 10 ml of tetrahydrofurane. The reaction mixture was cooled to −70° C. and 0.90 ml (1.44 mmol) of 1.6M n-butyllithium was added dropwise. The resulting deep red solution was stirred at −70° C. for 20 min and 320 mg (0.507 mmol) of (1R,3aR,4S,7aR)-7a-methyl-1-[(1S,3Z)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one was added dropwise in 1.5 ml of tetrahydrofurane. The reaction mixture was stirred for 4 h and then the dry ice was removed from bath and the solution was allowed to warm up to −40° C. in 1 h. The mixture was poured into 50 ml of ethyl acetate and 100 ml of brine. The water fraction was extracted three times with 50 ml of ethyl acetate, dried over Na₂SO₄ and evaporated. The oil residue was chromatographed on column (50 cm³, protected from light) using hexane:ethyl acetate (10:1) as mobile phase. Fractions containing product were pooled and evaporated to give colorless oil which was treated with 10 ml of 1M tetrabutylammonium fluoride in tetrahydrofurane. The reaction mixture was stirred at room temperature for 6 h 30 min.

The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate:hexane (1:1 and 2:1) as mobile phase. Fractions containing product were pooled and evaporated to give product as colorless oil. The product was dissolved in methyl acetate and evaporated (2 times) to give 300 mg 95%) of product as white foam.

[α]_{D}²⁸=+20.2° c=0.55, EtOH

UV λmax (EtOH): 207.67 nm (ε 20792), 242.33 nm (ε 17972), 270.00 nm (ε 18053)

¹H NMR (CDCl₃): 6.40(1H, d, J=11.1 Hz), 6.11 (1H, ddd, J=12.4, 9.5, 6.0 Hz), 6.02 (1H, d, J=11.1 Hz), 5.49 (1H, d, J=12.1 Hz), 5.39 (1H, s), 5.14 (1H, ddd, J=49.5, 7.2, 4.2 Hz), 5.10 (1H, s), 4.23 (1H, br s), 2.87-2.80 (2H, m), 2.62 (1H, br d, J=12.1 Hz), 2.48-2.43 (1H, m), 2.31 (1H, dd, J=12.9, 7.5 Hz), 2.22-2.14 (1H, m), 2.06-1.97 (3H, m), 1.70-1.12 (16H, m), 1.22 (3H, s), 1.21 (3H, m), 1.05-0.91 (2H, m), 0.97 (3H, s), 0.65 (3H, s)

| | | | | |
|---|---|---|---|---|
| MS HRES | Calculated for: | C₃₃H₄₇F₇O₃ | [M + Na]⁺ | 647.3305 |
| | Observed: | | [M + Na]⁺ | 647.3304 |

### Example 12

### Synthesis of (20S)-1,25-Dihydroxy-20-[(2E)-5,5,5-trifluoro-4-hydroxy-4-trifluoromethyl-pent-2-enyl]-cholecalciferol

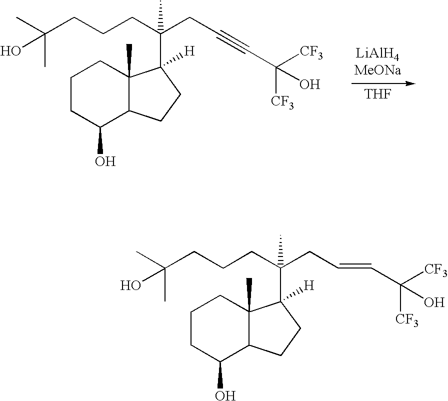

### (3E,6S)-1,1,1-Trifluoro-6-[(1R,3aR,4S,7aR)-4-hydroxy-7a-methyl-octahydro-inden-1-yl]-6,10-dimethyl-2-trifluoromethyl-undec-3-ene-2,10-diol

A 25 ml round bottom flask equipped with stir bar and condenser with nitrogen sweep was charged with 4.0 ml (4.0 mmol) of 1M lithium aluminum hydride in tetrahydrofurane. The mixture was cooled to 0° C. and 216 mg (4.00 mmol) of sodium methoxide was added slowly followed by 300 mg (0.617 mmol) of (6S)-1,1,1-trifluoro-6-([(1R,3aR,4S,7aR)-4-hydroxy-7a-methyl-octahydro-inden-1-yl]-6,10-dimethyl-2-trifluoromethyl-undec-3-yne-2,10-diol in 4.0 ml of tetrahydrofurane. The reaction mixture was stirred at 80° C. for 5 h and then was cooled to 0° C. A 1.0 ml of water, 1.0 ml of 2N NaOH and 20.0 ml of diethyl ether were added. The mixture was stirred at room temp for 30 min, 2.2 g of MgSO₄ was added and mixture was stirred for next 15 min. The suspension was filtrated and solvent evaporated. The oil residue was chromatographed on columns (100 cm³ and 30 cm³) using dichloromethane:ethyl acetate (4:1) as mobile phase. Fractions containing product were pooled and evaporated to give 279 mg (93%) of product as colorless oil.

¹H NMR (CDCl₃): 6.32(1H, dt, J=15.7, 7.8 Hz), 5.59 (1H, 15.7 Hz), 4.09 (1H, br s), 2.29 (2H, d, J=7.6 Hz), 2.01 (1H, br d, J=3.3 Hz), 1.86-1-75(2H, m), 1.63-1.04 (18H, m), 1.21 (6H, s), 1.09 (3H, s), 0.98 (3H, s)

¹³C NMR (CDCl₃): 137.07, 119.81, 71.52, 69.54, 69.57, 57.20, 52.53, 44.16, 43.50, 42.29, 41.43, 40.10, 40.04, 33.39, 29.33, 29.29, 23.01, 22.17, 21.69, 17.86, 17.51, 16.58

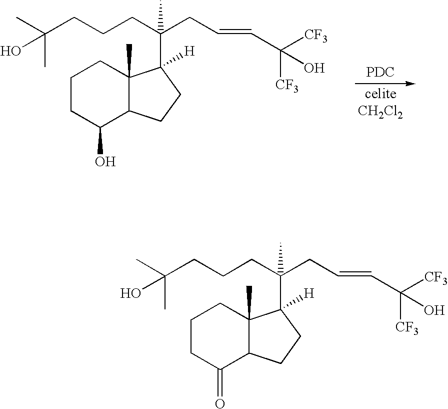

### (1R,3aR,4S,7aR)-7a-Methyl-1-[(1S,3E)-6,6,6-trifluoro-5-hydroxy-1-(4-hydroxy-4-methyl-pentyl)-1-methyl-5-trifluoromethyl-hex-3-enyl]-octahydro-inden-4-one

A 25 ml round bottom flask equipped with stir bar and Claisen adapter with rubber septum was charged with 274 mg (0.561 mmol) of (6S,3E)-1,1,1-trifluoro-6-[(1R,3aR,4S, 7aR)-4-hydroxy-7a-methyl-octahydro-inden-1-yl]-6,10-dimethyl-2-trifluoromethyl-undec-3-ene-2,10-diol and 10 ml of dichloromethane. A 704 mg (1.871 mmol) of pyridinium dichromate and 740 mg of celite was added and mixture was stirred in room temperature for 2 h. The reaction mixture was filtrated through column with silica gel (100 cm³) using dichloromethane:ethyl:acetate (4:1) as a mobile phase. The fractions containing product were pooled and evaporated to give 253 mg of yellow oil. The product was used to the next reaction without farther purification.

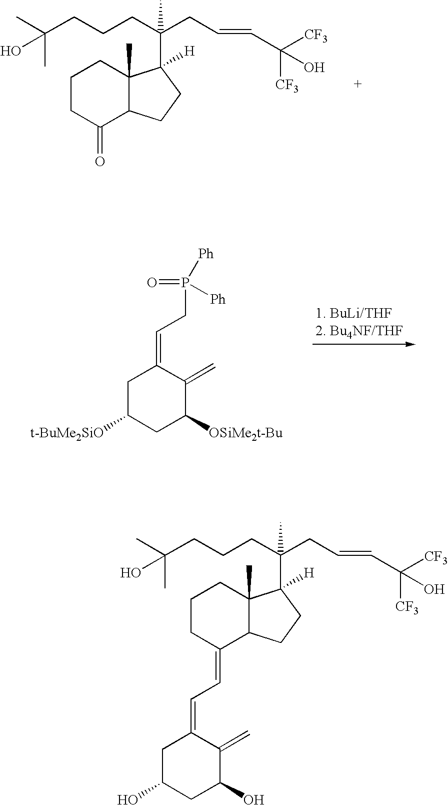

### (20S)-1,25-Dihydroxy-20-[(2E)-5,5,5-trifluoro-4-hydroxy-4-trifluoromethyl-pent-2-enyl]-cholecalciferol

A 25 ml round bottom flask equipped with stir bar and Claisen adapter with rubber septum was charged with 1.765 g (3.028 mmol) of (1S,5R)-1,5-bis-((tert-butyldimethyl)silanyloxy)-3-[2-(diphenylfosphinoyl)-eth-(Z)-ylidene]-2-methylene-cyclohexane and 10.0 ml of tetrahydrofurane. The reaction mixture was cooled to −78° C. and 1.8 ml (2.88 mmol) of 1.6M n-butyllithium in tetrahydrofurane was added dropwise. The resulting deep red solution was stirred at −78° C. for 20 min and 253 mg (0.520 mmol) of (1R,3aR,4S,7aR)-7a-methyl-1-[(1S,3E)-6,6,6-trifluoro-5-hydroxy-1-(4-hydroxy-4-methyl-pentyl)-1-methyl-5-trifluoromethyl-hex-3-enyl]-octahydro-inden-4-one was added dropwise in 1.5 ml of tetrahydrofurane. The reaction mixture was stirred for 5 h (last 0.5 h at −20° C.) and then the bath was removed and the mixture was poured into 50 ml of ethyl acetate and 100 ml of brine. The water fraction was extracted three times with 50 ml of ethyl acetate, dried over Na₂SO₄ and evaporated. The oil residue was chromatographed on column (60 cm³, protected from light) using hexane:ethyl acetate (4:1) as mobile phase. Fractions containing product were pooled and evaporated to give colorless oil (304 mg) which was treated with 5 ml of 1M tetrabutylammonium fluoride in tetrahydrofurane. The reaction mixture was stirred at room temperature for 21 h.

The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give 176 mg (54%, two steps) of product as white foam.

[α]_{D}²⁹=−4.5° c=0.33, CHCl₃

UV λmax (EtOH): 204.50 nm (ε 17846), 266.17 nm (ε 16508)

¹H NMR (CDCl₃): 6.36(1H, d, J=11.3 Hz), 6.32 (1H, dt, J=15.1, 7.5 Hz), 6.00 (1H, d, J=11.1 Hz), 5.59 (1H, d, J=15.8 Hz, 5.33(1H, s), 4.99 (1H, s), 4.53 (1H, br s), 4.43 (1H, dd, J=7.7, 4.3 Hz), 4.25-4.00 (1H, m), 2.81 (1H, dd, J=12.1, 3.8 Hz), 2.59 (1H, dd, J=13.3, 2.9 Hz), 2.34-2.29 (3H, m), 2.05-1.96 (3H, m), 1.93-1.87 (1H, m), 1.71-1.21 (17H, m), 1.21 (6H, s), 1.12-1.05 (1H, m), 0.95 (3H, s), 0.66 (3H, s)

¹³C NMR (CDCl₃): 147.48, 142.53, 136.92, 133.05, 124.83, 122.39(q, J=284.7 Hz), 119.76, 117.58, 117.49, 111, 71, 71.61, 70.73, 66.90, 57.39, 56.62, 46.79, 45.18, 43.99, 42.83, 42.48, 41.29, 40.13, 40.04, 29.62, 29.28, 28.98, 23.50, 23.06, 22.24, 21.90, 17.74, 15.11

| | | | | |
|---|---|---|---|---|
| MS HRES | Calculated for: | C₃₃H₄₈F₆O₄ | [M + Na]⁺ | 645.3349 |
| | Observed: | | [M + Na]⁺ | 645.3346 |

### Example 13

### Synthesis of (20S)-1,25-Dihydroxy-20-[(2E)-5,5,5-trifluoro-4-hydroxy-4-trifluoromethyl-pent-2-enyl]-19-nor-cholecalciferol

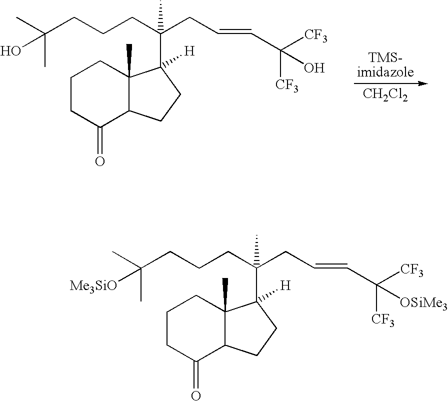

### (1R,3aR,4S,7aR)-7a-Methyl-1-[(1S,3E)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one

A 25 ml round bottom flask equipped with stir bar and Claisen adapter with rubber septum was charged with 577 mg (1.186 mmol) of (1R,3aR,4S,7aR)-7a-methyl-1-[(1S,3E)-6,6,6-trifluoro-5-hydroxy-1-(4-hydroxy-4-methyl-pentyl)-1-methyl-5-trifluoromethyl-hex-3-enyl]-octahydro-inden-4-one and 20 ml of dichloromethane. A 1.5 ml (10.2 mmol) of 1-(trimethylsilyl)imidazole was added dropwise. The mixture was stirred at room temperature for 5 h 30 min. A 150 ml of ethyl acetate was added and the mixture was washed three times with 50 ml of water, dried over Na₂SO₄ and evaporated. The oil residue was chromatographed on column (75 cm³) using hexane:ethyl acetate (10:1) as mobile phase. Fractions containing product were pooled and evaporated to give 710 mg (95%) of product as colorless oil.

¹H NMR (CDCl₃): 6.21(1H, dt, J=15.1, 7.2 Hz), 5.56 (1H, d, J=15.4 Hz), 1.22-1.19 (1H, m), 2.32-1.06 (2H, m), 2.27 (2H, d, J=7.0 Hz), 2.06-1.52 (9H, m), 1.34-1.08 (6H, m), 1.20 (3H, s), 1.19 (3H, s), 0.96 (3H, s), 0.73 (3H, s), 0.22 (9H, s), 0.10 (9H, s)

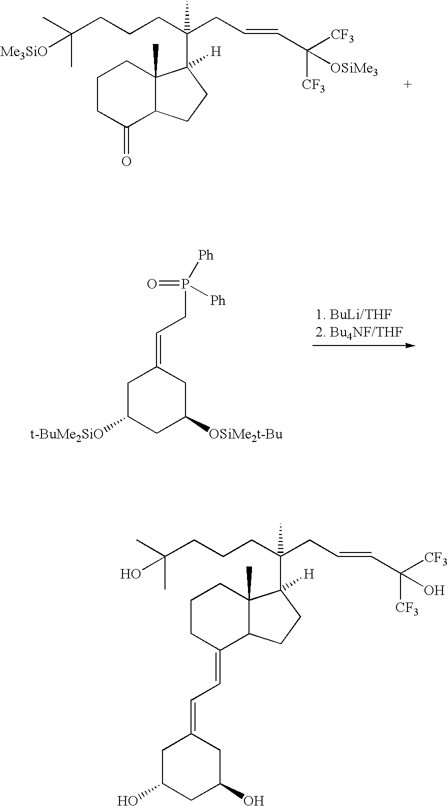

### (20S)-1,25-Dihydroxy-20-[(2E)-5,5,5-trifluoro-4-hydroxy-4-trifluoromethyl-pent-2-enyl]-19-nor-cholecalciferol

A 25 ml round bottom flask equipped with stir bar and Claisen adapter with rubber septum was charged with 836 mg (1,464 mmol) of (1R,3R)-1,3-bis-((tent-butyldimethyl)silanyloxy)-5-[2-(diphenylfosphinoyl)ethylidene]-cyclohexane and 10 ml of tetrahydrofurane. The reaction mixture was cooled to −70° C. and 0.89 ml (1.42 mmol) of 1.6M n-butyllithium BuLi was added dropwise. The resulting deep red solution was stirred at −70° C. for 20 min and 360 mg (0.571 mmol) of (1R,3aR,4S,7aR)-7a-methyl-1-[(1S,3E)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one in 1.5 ml of tetrahydrofurane. The reaction mixture was stirred for 5 h and then the dry ice was removed from bath and the solution was allowed to warm up to −40° C. in 1 h. The mixture was poured into 50 ml of ethyl acetate and 100 ml of brine. The water fraction was extracted three times with 50 ml of ethyl acetate, dried over Na₂SO₄ and evaporated.

The oil residue was chromatographed on column (50 cm³, protected from light) using hexane:ethyl acetate (10:1) as mobile phase. Fractions containing product were pooled and evaporated to give colorless oil (ca. 440 mg) which was treated with 10 ml of 1M tetrabutylammonium fluoride in tetrahydrofurane. The reaction mixture was stirred at room temperature for 26 h. The new portion 2.5 ml of 1M tetrabutylammonium fluoride in tetrahydrofurane was added and the mixture was stirred for next 6 h.

The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (2 times) to give 303 mg (87%) of product as white foam.

[α]_{D}²⁶=+41.8 c=0.44, EtOH

UV λmax (EtOH): 244 nm (ε 27480), 252 nm (ε 32212), 262 nm (ε 21694)

¹H NMR (CDCl₃): 6.33(1H, dt, J=15.6, 7.8 Hz), 6.29 (1H, d, J=9.0 Hz), 5.83 (1H, d, J=11.1 Hz), 5.58 (1H, d, J=15.6 Hz), 4.12-4.09 (1H, m), 4.05-4.02 (1H, m), 2.79-2.71 (2H, m), 2.46 (1H, dd, J=13.2, 3.0 Hz), 2.29 (2H, d, J=7.5 Hz), 2.20 (2H, dd, J=13.3, 7.1 Hz), 2.04-1.75 (7H, m), 1.68-1.46 (9H, m), 1.41-1.21 (6H, m), 1.21 (6H, s), 1.12-1.05 (1H, m), 0.95 (3H, s), 0.65 (3H, s)

¹³C NMR (CDCl₃): 142.40, 136.79, 131.25, 123.64, 122.4(q, J=286.96 Hz), 119.83, 115.76, 71.59, 67.42, 67.18, 57.33, 56.56, 46.64, 44.52, 44.04, 42.40, 42.02, 41.24, 40.10, 40.01, 37.13, 29.54, 29.26, 28.83, 23.39, 23.07, 22.25, 21.87, 17.79, 15.17

| | | | | |
|---|---|---|---|---|
| MS HRES | Calculated for: | C₃₂H₄₈F₆O₄ | [M + Na]⁺ | 633.3349 |
| | Observed: | | [M + Na]⁺ | 633.3349 |

### Example 14

### Synthesis of (205)-1α-Fluoro-25-hydroxy-20-[(2E)-5,5,5-trifluoro-4-hydroxy-4-trifluoromethyl-pent-2-enyl]-cholecalciferol

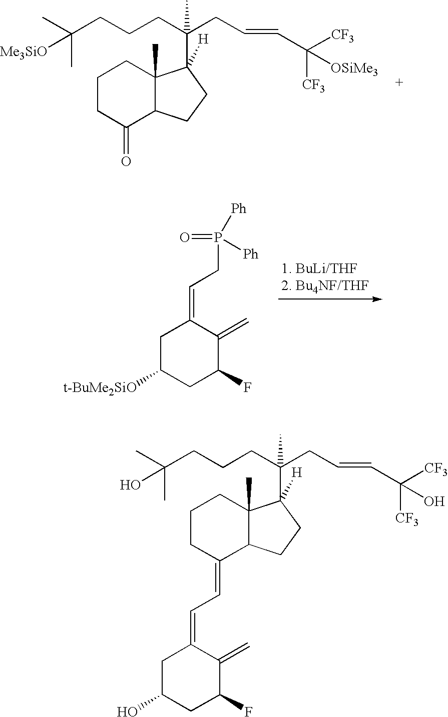

### (20S)-1α-Fluoro-25-hydroxy-20-[(2E)-5,5,5-trifluoro-4-hydroxy-4-trifluoromethyl-pent-2-enyl]-cholecalciferol

A 25 ml round bottom flask equipped with stir bar and Claisen adapter with rubber septum was charged with 521 mg (1.107 mmol) of (1S,5R)-1-((tent-butyldimethyl)silanyloxy)-3-[2-(diphenylfosphinoyl)-eth-(Z)-ylidene]-5-fluoro-2-methylene-cyclohexane and 10 ml of tetrahydrofurane. The reaction mixture was cooled to −70° C. and 0.69 ml (1.10 mmol) of 1.6M n-butyllithium was added dropwise. The resulting deep red solution was stirred at −70° C. for 20 min and 324 mg (0.514 mmol) of (1R,3aR,4S,7aR)-7a-methyl-1-[(1S,3E)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one was added dropwise in 1.5 ml of tetrahydrofurane. The reaction mixture was stirred for 4 h and then the dry ice was removed from bath and the solution was allowed to warm up to −40° C. in 1 h. The mixture was poured into 50 ml of ethyl acetate and 100 ml of brine. The water fraction was extracted three times with 50 ml of ethyl acetate, dried over Na₂SO₄ and evaporated. The oil residue was chromatographed on column (50 cm³, protected from light) using hexane:ethyl acetate (10:1) as mobile phase. Fractions containing product were pooled and evaporated to give colorless oil which was treated with 8 ml of 1M tetrabutylammonium fluoride in tetrahydrofurane. The reaction mixture was stirred at room temperature for 9 h.

The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate:hexane (1:1) as mobile phase. Fractions containing product were pooled and evaporated to give product as colorless oil. The product was dissolved in methyl acetate and evaporated (2 times) to give 305 mg 95%) of product as white foam.

[α]_{D}²⁶=+29.3 c=0.43, EtOH

UV λmax (EtOH): 210 nm (ε 13484), 243 nm (ε 13340), 271 nm (ε 13609)

¹H NMR (CDCl₃): 6.39(1H, d, J=11.3 Hz), 6.32 (1H, dt, J=15.6, 7.6 Hz), 6.01 (1H, d, J=11.3 Hz), 5.58 (1H, d, J=15.8 Hz), 2.39 (1H, s), 5.13 (1H, ddd, J=49.9, 6.3, 3.8 Hz), 5.09 (1H, s), 4.21 (1H, br s), 2.81 (1H, dd, J=11.8, 3.5 Hz), 2.61 (1H, dd, J=13.2, 3.2 Hz), 2.32-2.28 (3H, m), 2.23-2.15 (1H, m), 2.04-1.93 (3H, m), 1.70-1.48 (9H, m), 1.41-1.21 (8H, m), 1.21 (6H, s), 1.12-1.05 (1H, m), 0.95 (3H, s), 0.65 (3H, s)

¹³C NMR (CDCl₃): 142.95(d, J=16.0 Hz), 136.84, 131.54, 125.42, 122.42(q, J=286.9 Hz), 119.78, 117.53, 114.96(d, J=10.0 Hz), 71.74, 66.56(d, J=6.0 Hz), 57.35, 56.61, 46.82, 44.91, 44.04, 42.40, 41.29, 40.69(d, J=20.6 Hz), 40.10, 39.98, 29.47, 29.20, 29.01, 23.47, 23.07, 22.22, 21.82, 17.79, 15.13

| | | | | |
|---|---|---|---|---|
| MS HRES | Calculated for: | C₃₃H₄₇F₇O₃ | [M + Na]⁺ | 647.3305 |
| | Observed: | | [M + Na]⁺ | 647.3302 |

### Example 15

### Synthesis of (20R)-1,25-Dihydroxy-20-(5,5,5-trifluoro-4-hydroxy-4-trifluoromethyl-pent-2-ynyl)-cholecalciferol

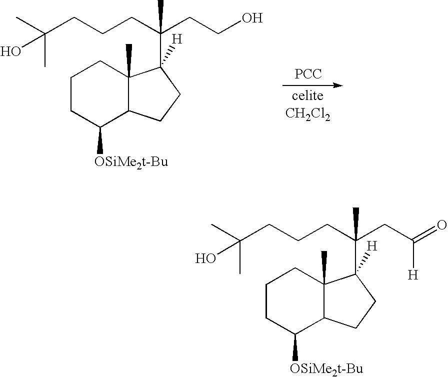

### (3R)-3-[(1R,3aR,4S,7aR)-4-(tert-Butyl-dimethyl-silanyloxy)-7a-methyl-octahydro-inden-1-yl]-7-hydroxy-3,7-dimethyl-octanal

A 50 ml round bottom flask equipped with stir bar and Claisen adapter with rubber septum was charged with 1.558 g (7.228 mmol) of pyridinium chlorochromate, 1.60 g of celite and 20 ml of dichloromethane. A 1.440 g (3.267 mmol) of (3R)-3-[(1R,3aR,4S,7aR)-4-(tert-butyl-dimethyl-silanyloxy)-7a-methyl-octahydro-inden-1-yl]-3,7-dimethyl-octane-1,7-diol in 10 ml of dichloromethane was added dropwise and mixture was stirred in room temperature for 2 h 50 min. The reaction mixture was filtrated through column with silica gel (75 cm³) and celite (2 cm) and using dichloromethane, dichloromethane:ethyl acetate (4:1) as a mobile phase. The fractions containing product were pooled and evaporated to give 1.298 g of yellow oil. The product was used to the next reaction without farther purification.

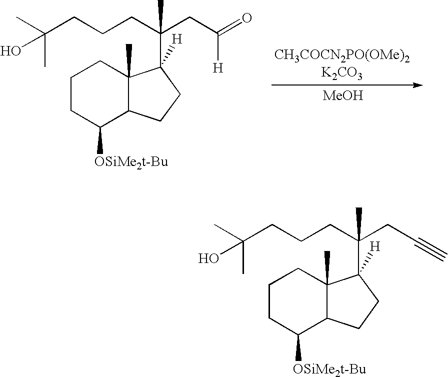

### (6R)-6-[(1R,3aR,4S,7aR)-4-(tert-Butyl-dimethyl-silanyloxy)-7a-methyl-octahydro-inden-1-yl]-2,6-dimethyl-non-8-yn-2-ol

A 50 ml round bottom flask equipped with stir bar and Claisen adapter with rubber septum was charged with 1.298 g (2.958 mmol) of (3R)-3-[(1R,3aR,4S,7aR)-4-(tert-butyl-dimethyl-silanyloxy)-7a-methyl-octahydro-inden-1-yl]-7-hydroxy-3,7-dimethyl-octanal and 30 ml of methanol. A 1.137 g (5.916 mmol) of 1-diazo-2-oxo-propyl)-phosphonic acid dimethyl ester in 3 ml of methanol was added and the resulting mixture was cooled in an ice bath to 0° C. A 1.140 g (8.248 mmol) of potassium carbonate was added and the reaction mixture was stirred in the ice bath for 30 min and then at room temperature for 2 h 50 min. A 100 ml of water was added and the mixture was extracted three times with 80 ml of ethyl acetate, dried over Na₂SO₄ and evaporated.

The oil residue was chromatographed on column (200 cm³) using hexane:ethyl acetate (7:1) as mobile phase. Fractions containing product were pooled and evaporated to give 1.151 g (81%) of product as colorless oil.

[α]_{D}²⁹=+18.3° c=0.54, CHCl₃

¹H NMR (CDCl₃): 3.99 (1H, br s), 2.16-2.07 (2H, m), 2.00-1.97 (1H, m), 1.92 (1H, t, J=2.6 Hz), 1.84-1.74 (1H, m), 1.67-1.64 (1H, m), 1.58-1.22 (16H, m), 1.22 (6H, s), 1.04 (3H, s), 0.99 (3H, s), 0.88 (9H, s), 0.00 (3H, s), −0.01(3H, s)

| | | | | |
|---|---|---|---|---|
| MS HRES | Calculated for: | C₂₇H₅₀O₂Si | [M + Na]⁺ | 457.3472 |
| | Observed: | | [M + Na]⁺ | 457.3473 |

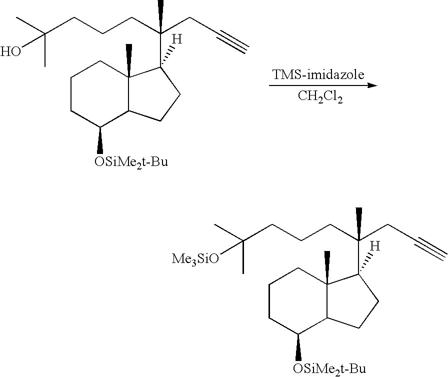

### (1R,3aR,4S,7aR)-4-(tert-Butyl-dimethyl-silanyloxy)-1-[(1R)-1,5-dimethyl-1-prop-2-ynyl-5-trimethylsilanyloxy-hexyl]-7a-methyl-octahydro-indene

A 50 ml round bottom flask equipped with stir bar and Claisen adapter with rubber septum was charged with 1.151 g (2.647 mmol) of (6R)-6-[(1R,3aR,4S,7aR)-4-(tert-butyl-dimethyl-silanyloxy)-7a-methyl-octahydro-inden-1-yl]-2,6-dimethyl-non-8-yn-2-ol and 20 ml of dichloromethane. A 2.0 ml (13.63 mmol) of 1-(trimethylsilyl)imidazole was added dropwise. The mixture was stirred at room temperature for 1 h. A 100 ml of water was added and the mixture was extracted three times with 50 ml of ethyl acetate, dried over Na₂SO₄ and evaporated. The oil residue was chromatographed on column (75 cm³) using hexane:ethyl acetate (25:1) as mobile phase. Fractions containing product were pooled and evaporated to give 1.260 g (94%) of product as colorless oil.

[α]_{D}²⁹=+18.5° c=0.46, CHCl₃

¹H NMR (CDCl₃): 3.98 (1H, br s), 2.12-2.08 (2H, m), 20.5-1.95 (2H, m), 1.92-1.90 (1H, m), 1.83-1.21 (16H, m), 1.21 (6H, s), 1.04 (3H, s), 0.98 (3H, s), 0.88 (9H, s), 0.11 (9H, s), 0.00 (3H, s), −0.01(3H, s)

¹³C NMR (CDCl₃): δ3.00, 74.07, 69.70, 69.50, 56.63, 53.03, 45.66, 43.74, 41.35, 39.59, 39.45, 34.38, 29.99, 29.60, 25.85, 22.81, 22.43, 22.06, 18.56, 18.05, 17.76, 16.49, 2.65, −4.77, −5.13

| | | | | |
|---|---|---|---|---|
| MS HRES | Calculated for: | C₃₀H₅₈O₂Si₂ | [M + Na]⁺ | 529.3867 |
| | Observed: | | [M + Na]⁺ | 529.3868 |

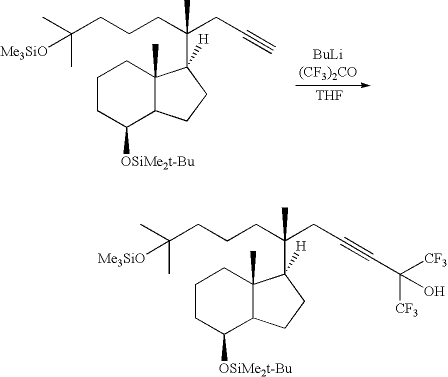

### (6R)-6-[(1R,3aR,4S,7aR)-4-(tert-Butyl-dimethyl-silanyloxy)-7a-methyl-octahydro-inden-1-yl]-1,1,1-trifluoro-6,10-dimethyl-2-trifluoromethyl-10-trimethylsilanyloxy-undec-3-yn-2-ol

A two neck 50 ml round bottom flask equipped with stir bar, Claisen adapter with rubber septum and funnel (with cooling bath) was charged with 1.252 g (2.470 mmol) of (1R,3aR,4S, 7aR)-4-(tert-butyl-dimethyl-silanyloxy)-1-[(1R)-1,5-dimethyl-1-prop-2-ynyl-5-trimethylsilanyloxy-hexyl]-7a-methyl-octahydro-indene and 25 ml of tetrahydrofurane. The funnel was connected to container with hexafluoroacetone and cooled (acetone, dry ice). The reaction mixture was cooled to −70° C. and 2.4 ml (3.84 mmol) of 1.6M n-butyllithium in tetrahydrofurane was added dropwise. After 30 min hexafluoroacetone was added (the container's valve was opened three times). The reaction was stirred at −70° C. for 2 h then 5.0 ml of saturated solution of ammonium chloride was added.

The mixture was dissolved by the addition of 100 ml of saturated solution of ammonium chloride and extracted three times with 80 ml of ethyl acetate, dried over Na₂SO₄ and evaporated. The residue was chromatographed twice on columns (75 cm³) using hexane:ethyl:acetate (10:1) as mobile phase to give 1.711 g of mixture of product and polymer (from hexafluoroacetone).

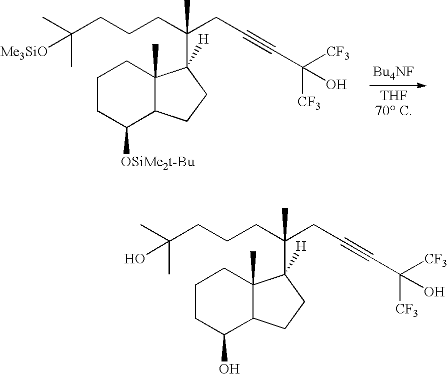

### (6R)-1,1,1-Trifluoro-6-[(1R,3aR,4S,7aR)-4-hydroxy-7a-methyl-octahydro-inden-1-yl]-6,10-dimethyl-2-trifluoromethyl-undec-3-yne-2,10-diol

A 25 ml round bottom flask equipped with stir bar and Claisen adapter with rubber septum was charged with crude (ca 2.470 mmol) (6R)-6-[(1R,3aR,4S,7aR)-4-(tert-butyl-dimethyl-silanyloxy)-7a-methyl-octahydro-inden-1-yl]-1,1,1-trifluoro-6,10-dimethyl-2-trifluoromethyl-10-trimethylsilanyloxy-undec-3-yn-2-ol and 15.0 ml (15.0 mmol) of 1M tetrabutylammonium fluoride in tetrahydrofurane. The reaction mixture was stirred at 70° C. for 96 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on columns, 200 cm³ and 75 cm³ using hexane:ethyl acetate (2:1). The fractions containing product were pooled and evaporated to give 979 mg (81%) of product as colorless oil.

[α]_{D}³⁰=+1.04° c=0.48, CHCl₃

¹H NMR (CDCl₃): 4.08 (1H, br s), 2.24 (1H, AB, J=17.2 Hz), 2.17 (1H, AB, J=17.2 Hz), 2.05-2.02 (1H, m), 1.85-1.76 (2H, m), 1.66-1.20 (18H, m), 1.26 (3H, s), 1.25 (3H, s), 1.07 (3H, s), 1.01 (3H, s)

| | | | | |
|---|---|---|---|---|
| MS HRES | Calculated for: | C₂₄H₃₆F₆O₃ | [M + Na]⁺ | 509.2461 |
| | Observed: | | [M + Na]⁺ | 509.2463 |

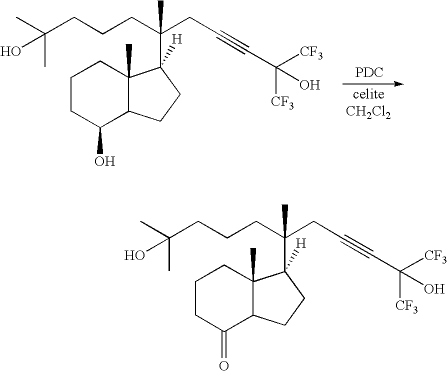

### (1R,3aR,4S,7aR)-7a-Methyl-1-[(1R)-6,6,6-trifluoro-5-hydroxy-1-(4-hydroxy-4-methyl-pentyl)-1-methyl-5-trifluoromethyl-hex-3-ynyl]-octahydro-inden-4-one

A 25 ml round bottom flask equipped with stir bar and Claisen adapter with rubber septum was charged with 291 mg (0.598 mmol) of (6R)-1,1,1-trifluoro-6-[(1R,3aR,4S,7aR)-4-hydroxy-7a-methyl-octahydro-inden-1-yl]-6,10-dimethyl-2-trifluoromethyl-undec-3-yne-2,10-diol and 10 ml of dichloromethane. A 700 mg (1.861 mmol) of pyridinium dichromate and 720 mg of celite was added and mixture was stirred in room temperature for 3 h. The reaction mixture was filtrated through column with silica gel (75 cm³) using dichloromethane, dichloromethane:ethyl acetate (4:1, 3:1). The fractions containing product were pooled and evaporated to give 271 mg (94%) of product as yellow oil.

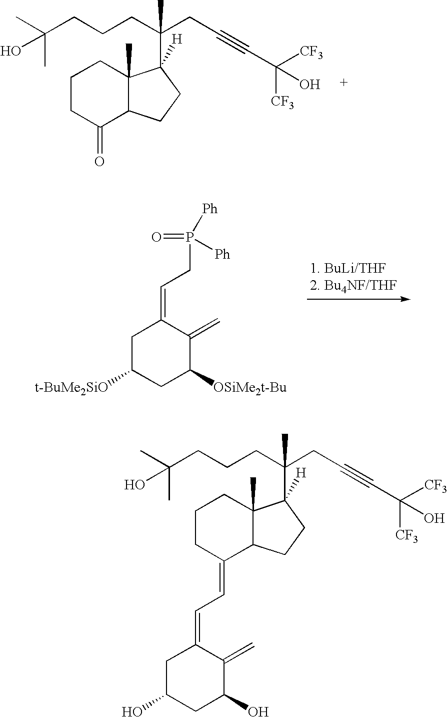

### (20R)-1,25-Dihydroxy-20-(5,5,5-trifluoro-4-hydroxy-4-trifluoromethyl-pent-2-ynyl)-cholecalciferol

A 25 ml round bottom flask equipped with stir bar and Claisen adapter with rubber septum was charged with 2.118 g (3.634 mmol) of (1S,5R)-1,5-bis-((tert-butyldimethyl)silanyloxy)-3-[2-(diphenylfosphinoyl)-eth-(Z)-ylidene]-2-methylene-cyclohexane and 10 ml of tetrahydrofurane. The reaction mixture was cooled to −78° C. and 2.2 ml (3.52 mmol) of 1.6M n-butyllithium in tetrahydrofurane was added dropwise. The resulting deep red solution was stirred at −78° C. for 20 min and 271 mg, (0.559 mmol) of (1R,3aR,4S,7aR)-7a-methyl-1-[(1R,3E)-6,6,6-trifluoro-5-hydroxy-1-(4-hydroxy-4-methyl-pentyl)-1-methyl-5-trifluoromethyl-hex-3-ynyl]-octahydro-inden-4-one was added dropwise in 1.5 ml of tetrahydrofurane. The reaction mixture was stirred at −78° C. for 5 h and then the bath was removed and the mixture was poured into 100 ml of saturated solution of ammonium chloride and extracted three times with 50 ml of ethyl acetate, dried over Na₂SO₄ and evaporated. The oil residue was chromatographed on column (50 cm³, protected from light) using hexane:ethyl acetate (4:1) as mobile phase. The fractions contains impurities was chromatographed on column (50 cm³, protected from light) using hexane:ethyl acetate (5:1) as mobile phase. Fractions containing product were pooled and evaporated to give colorless oil (250 mg) which was treated with 5 ml of 1M tetrabutylammonium fluoride in tetrahydrofurane. The reaction mixture was stirred at room temperature for 18 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give 194 mg (56%) of product as white foam.

[α]_{D}³⁰=+7.9° c=0.38, EtOH

UV λmax (EtOH): 212.33 nm (ε 14113), 265.00 nm (ε 15960)

¹H NMR (D6-DMSO): 8.93 (1H, s), 6.18 (1H, d, J=11.3 Hz), 5.96 (1H, d, J=11.3 Hz), 5.22 (1H, s), 4.86 (1H, d, J=4.83 Hz), 4.75 (1H, s), 4.54 (1H, d, J=3.63 Hz), 4.20-4.15 (1H, m), 4.06 (1H, s), 3.98 (1H, br s), 2.77 (1H, d, J=13.7 Hz), 2.40-2.33 (1H, m), 2.27-2.14 (3H, m), 2.00-1.90(2H, m), 1.82-1.78 (2H, m), 1.64-1.54 (5H, m), 1.47-1.18 (10H, m), 1.05 (3H, s), 1.05 (3H, s), 0.95 (3H, s), 0.59 (3H, s)

¹³C NMR (D6-DMSO): 149.38, 139.51, 135.94, 122.32, 121.47(q, J=287.5 Hz), 117.99, 109.77, 89.53, 70.58, 68.72, 68.35, 65.06, 56.02, 55.91, 46.06, 44.85, 44.65, 43.11, 29.30, 29.03, 28.78, 28.32, 23.05, 22.40, 21.90, 21.52, 18.27, 14.29

| | | | | |
|---|---|---|---|---|
| MS HRES | Calculated for: | C₃₃H₄₆F₆O₄ | [M + Na]⁺ | 643.3192 |
| | Observed: | | [M + Na]⁺ | 643.3190 |

### Example 16

### Synthesis of (20R)-1,25-Dihydroxy-20-(5,5,5-trifluoro-4-hydroxy-4-trifluoromethyl-pent-2-ynyl)-19-nor-cholecalciferol

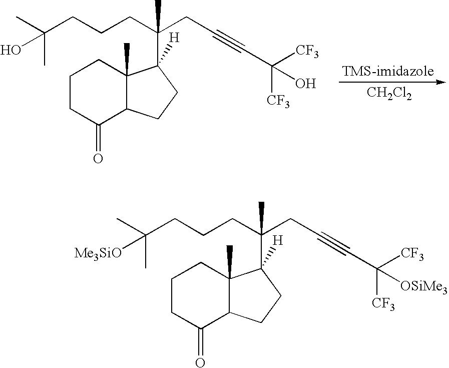

### (1R,3aR,4S,7aR)-7a-Methyl-1-[(1R)-6,6,6-trifluoro-1-methyl-1-(4-methyl-4-trimethylsilanyloxy-pentyl)-5-trifluoromethyl-5-trimethylsilanyloxy-hex-3-ynyl]-octahydro-inden-4-one

A 25 ml round bottom flask equipped with stir bar and Claisen adapter with rubber septum was charged with 399 mg (0.823 mmol) of (1R,3aR,4S,7aR)-7a-methyl-1-[(1R)-6,6,6-trifluoro-5-hydroxy-1-(4-hydroxy-4-methyl-pentyl)-1-methyl-5-trifluoromethyl-hex-3-ynyl]-octahydro-inden-4-one and 8.0 ml of dichloromethane. A 0.9 ml (6.2 mmol) of 1-(trimethylsilyl)imidazole was added dropwise. The mixture was stirred at room temperature for 4 h. A 150 ml of hexane was added and the mixture was washed three times with 50 ml of water, dried over Na₂SO₄ and evaporated. The oil residue was chromatographed on column (50 cm³) using hexane:ethyl acetate (5:1) as mobile phase. Fractions containing product were pooled and evaporated to give 492 mg (95%) of product as oil.

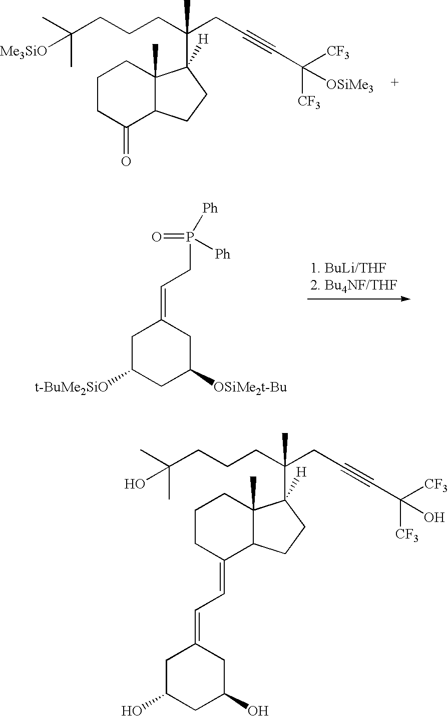

### (20R)-1,25-Dihydroxy-20-(5,5,5-trifluoro-4-hydroxy-4-trifluoromethyl-pent-2-ynyl)-19-nor-cholecalciferol

A 25 ml round bottom flask equipped with stir bar and Claisen adapter with rubber septum was charged with 490 mg (0.858 mmol) of (1R,3R)-1,3-bis-((tent-butyl dimethyl)silanyloxy)-5-[2-(diphenylfosphinoyl)ethylidene]-cyclohexane and 8 ml of tetrahydrofurane. The reaction mixture was cooled to −70° C. and 0.53 ml (0.848 mmol) of 1.6M n-butyllithium BuLi was added dropwise. The resulting deep red solution was stirred at −70° C. for 30 min and 249 mg (0.396 mmol) of (1R,3aR,4S,7aR)-7a-methyl-1-[(1R)-6,6,6-trifluoro-1-methyl-1-(4-methyl-4-trimethylsilanyloxy-pentyl)-5-trifluoromethyl-5-trimethylsilanyloxy-hex-3-ynyl]-octahydro-inden-4-one in 1.5 ml of tetrahydrofurane. The reaction mixture was stirred for 4.5 h and then the dry ice was removed from bath and the solution was allowed to warm up to −55° C. in 1 h. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate (10:1) as mobile phase. Fractions containing product were pooled and evaporated to give colorless oil (ca. 349 mg) which was treated with 10 ml of 1M tetrabutylammonium fluoride in tetrahydrofurane. The reaction mixture was stirred at room temperature for 63 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using hexane:tetrahydrofurane (1:1) as mobile phase. Fractions containing product were pooled and evaporated to give product 207 mg (86%) as white solid.

[α]_{D}²⁸=+44.7 c=0.51, EtOH

UV λmax (EtOH): 242 nm (ε 30834)

¹H NMR (DMSO-D6): 8.96 (1H, s), 6.08 (1H, d, J=10.9 Hz), 5.78 (1H, d, J=11.3 Hz), 4.48 (1H, d, J=4.3 Hz), 4.38 (1H, d, J=4.1 Hz), 4.07 (1H, s), 3.91-3.85 (1H, m), 3.84-3.77 (1H, m), 2.74 (1H, d, J=13.6 Hz), 2.43 (1H, dd, J=13.4, 3.4 Hz), 2.28-2.20 (3H, m), 2.07-1.93 (4H, m), 1.84-1.79 (1H, m), 1.69-1.21 (16H, m), 1.06 (3H, s), 1.06 (3H, s), 0.97 (3H, s), 0.60 (3H, s)

¹³C NMR (D6-DMSO): 139.09, 134.88, 121.60(q, J=286.0 Hz), 120.90, 116.56, 89.61, 70.64, 70.45(sep, J=33.3 Hz), 68.77, 65.57, 65.30, 56.00, 55.92, 45.93, 44.66, 44.59, 42.22, 36.95, 29.27, 29.02, 28.78, 28.14, 22.87, 22.38, 21.93, 21.40, 18.24, 14.35

| | | | | |
|---|---|---|---|---|
| MS HRES | Calculated for: | C₃₂H₄₆F₆O₄ | [M + Na]⁺ | 631.3192 |
| | Observed: | | [M + Na]⁺ | 631.3195 |

### Example 17

### Synthesis of (20R)-1α-Fluoro-25-hydroxy-20-(5,5,5-trifluoro-4-hydroxy-4-trifluoromethyl-pent-2-ynyl)-cholecaliferol

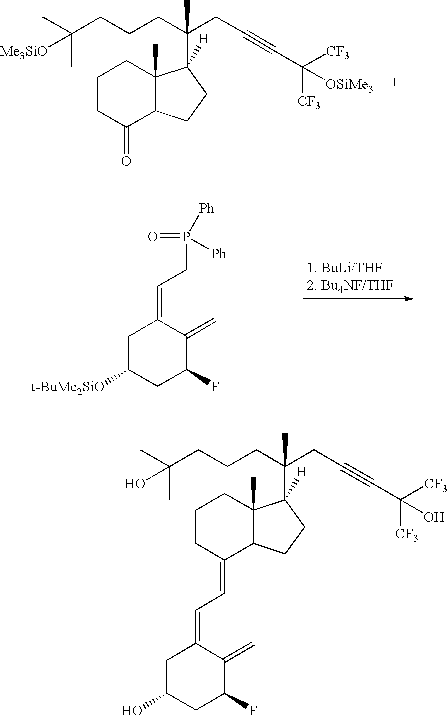

### (20R)-1α-Fluoro-25-hydroxy-20-(5,5,5-trifluoro-4-hydroxy-4-trifluoromethyl-pent-2-ynyl)-cholecalciferol

A 25 ml round bottom flask equipped with stir bar and Claisen adapter with rubber septum was charged with 460 mg (0.977 mmol) of (1S,5R)-1-((tent-butyldimethyl) silanyloxy)-3-[2-(diphenylfosphinoyl)-eth-(Z)-ylidene]-5-fluoro-2-methylene-cyclohexane and 8 ml of tetrahydrofurane. The reaction mixture was cooled to −70° C. and 0.61 ml (0.976 mmol) of 1.6M n-butyllithium was added dropwise. The resulting deep red solution was stirred at −70° C. for 20 min and 240 mg (0.382 mmol) of (1R,3aR,4S,7aR)-7a-methyl-1-[(1R)-6,6,6-trifluoro-1-methyl-1-(4-methyl-4-trimethylsilanyloxy-pentyl)-5-trifluoromethyl-5-trimethylsilanyloxy-hex-3-ynyl]-octahydro-inden-4-one was added dropwise in 1.5 ml of tetrahydrofurane. The reaction mixture was stirred for 4.5 h and then the dry ice was removed from bath and the solution was allowed to warm up to −40° C. in 1.5 h. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate (10:1) as mobile phase. Fractions containing product were pooled and evaporated to give colorless oil (ca. 239 mg) which was treated with 8 ml of 1M tetrabutylammonium fluoride in tetrahydrofurane. The reaction mixture was stirred at room temperature for 17 h.

The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate:hexane (1:2 and 1:1) as mobile phase. Fractions containing product were pooled and evaporated to give product as colorless oil. The product was dissolved in methyl acetate and evaporated (2 times) to give 196 mg (82%) of product as white foam.

[α]_{D}³⁰=+24.4 c=0.45, EtOH

UV λmax (EtOH): 241 nm (ε 17260), 273 nm (ε 16624)

¹H NMR (DMSO-D6): 8.95 (1H, s), 6.37 (1H, d, J=11.5 Hz), 5.93 (1H, d, J=11.1 Hz), 5.39 (1H, s), 5.14 (1H, br d, J=47.1 Hz), 4.99 (1H, d, J=1.9 Hz), 4.86 (1H, d, J=4.3 Hz), 4.07 (1H, s), 3.94-3.87 (1H, m), 2.83-2.80 (1H, m), 2.28-2.05 (4H, m), 2.00-1.93 (2H, m), 1.83-1.21 (17H, m), 1.06 (3H, s), 1.06 (3H, s), 0.96 (3H, s), 0.59 (3H, s)

¹³C NMR (D6-DMSO): 143.27(d, J=16.7 Hz), 141.62, 133.20, 124.14, 121.59(q, J=286.0 Hz), 117.49, 115.34(d, J=9.8 Hz), 92.05 (d, J=166.9 Hz), 89.60, 70.64, 70.44(sep, J=32.6 Hz), 68.77, 64.55(d, J=4.5 Hz), 55.99, 55.92, 46.15, 44.83, 44.65, 40.68(d, J=20.5 Hz), 40.05, 39.79, 39.41, 29.27, 29.02, 28.76, 28.30, 22.95, 22.33, 21.87, 21.39, 18.24, 14.28

| | | | | |
|---|---|---|---|---|
| MS HRES | Calculated for: | C₃₃H₄₅F₇O₃ | [M + Na]⁺ | 645.3149 |
| | Observed: | | [M + Na]⁺ | 645.3155 |

### Example 18

### Synthesis of (20)-1,25-Dihydroxy-20-[(2Z)-5,5,5-trifluoro-4-hydroxy-4-trifluoromethyl-pent-2-enyl]-cholecalciferol (8)

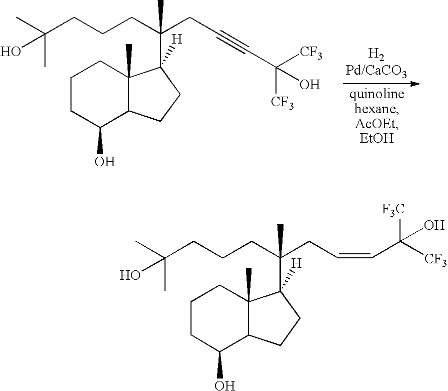

### (3Z,6R)-1,1,1-Trifluoro-6-[(1R,3aR,4S,7aR)-4-hydroxy-7a-methyl-octahydro-inden-1-yl]-6,10-dimethyl-2-trifluoromethyl-undec-3-ene-2,10-diol

A 25 ml round bottom flask was charged with 340 mg (0.699 mmol) of (6R)-1,1,1-trifluoro-6-[(1R,3aR,4S,7aR)-4-hydroxy-7a-methyl-octahydro-inden-1-yl]-6,10-dimethyl-2-trifluoromethyl-undec-3-yne-2,10-diol, 100 mg of 5% Pd/CaCO₃, 8.0 ml of hexane, 3.3 ml of ethyl acetate and 0.32 ml of solution of quinoline in ethanol (prepared from 3.1 ml of ethanol and 168 μl of quinoline). The substrate was hydrogenated at ambient temperature and atmospheric pressure of hydrogen. The reaction was monitoring by TLC (hexane:ethyl acetate—2:1). After 7 h the catalyst was filtered off and solvent evaporated. The residue was purified over silica gel (50 cm³) using hexane:ethyl acetate (2:1). Fractions containing product were pooled and evaporated to give 320 mg (94%) of product as colorless oil.

¹H NMR (CDCl₃): 6.12-6.03 (1H, m), 5.46 (1H, d, J=13.2 Hz), 4.08 (1H, br s), 2.46-2.40 (2H, m), 2.06-1.95 (1H, m), 1.86-1.76 (2H, m), 1.66-1.20 (18H, m), 1.21 (6H, s), 1.09 (3H, s), 0.99 (3H, s)

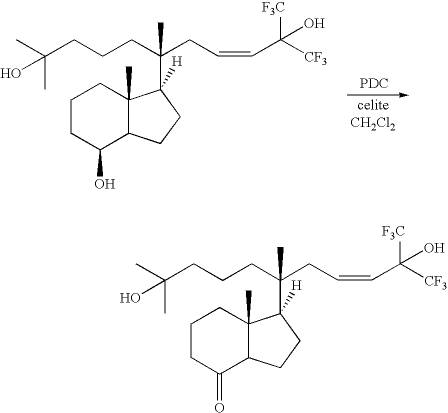

### (1R,3aR,4S,7aR)-7a-Methyl-1-[(1R,3Z)-6,6,6-trifluoro-5-hydroxy-1-(4-hydroxy-4-methyl-pentyl)-1-methyl-5-trifluoromethyl-hex-3-enyl]-octahydro-inden-4-one

A 25 ml round bottom flask equipped with stir bar and Claisen adapter with rubber septum was charged with 315 mg (0.645 mmol) of (1R,3Z)-1,1,1-trifluoro-6-[(1R,3aR,4S, 7aR)-4-hydroxy-7a-methyl-octahydro-inden-1-yl]-6,10-dimethyl-2-trifluoromethyl-undec-3-ene-2,10-diol and 12.0 ml of dichloromethane. A 780 mg (1.861 mmol) of pyridinium dichromate was added and mixture was stirred in room temperature for 3 h.

The reaction mixture was filtrated through column with silica gel (100 cm³) using dichloromethane, dichloromethane:ethyl acetate (4:1, 3:1). The fractions containing product were pooled and evaporated to give 305 mg (97%) of product as yellow oil.

[α]_{D}³⁰=−25.9° c=0.37, CHCl₃

¹H NMR (CDCl₃): 6.07(1H, dt, J=12.4, 7.3 Hz), 5.49 (1H, d, J=11.9 Hz), 4.33 (1H, br s), 2.52 (1H, dd, J=16.2, 7.7 Hz), 2.45-2.38 (2H, m), 2.31-2.10 (3H, m), 2.06-1.98 (1H, m), 1.96-1.81 (1H, m), 1.79-1.35 (12H, m), 1.23 (6H, s), 0.99 (3H, s), 0.75 (3H, s)

| | | | | |
|---|---|---|---|---|
| MS HRES | Calculated for: | C₂₄H₃₆F₆O₃ | [M + Na]⁺ | 509.2461 |
| | Observed: | | [M + Na]⁺ | 509.2463 |

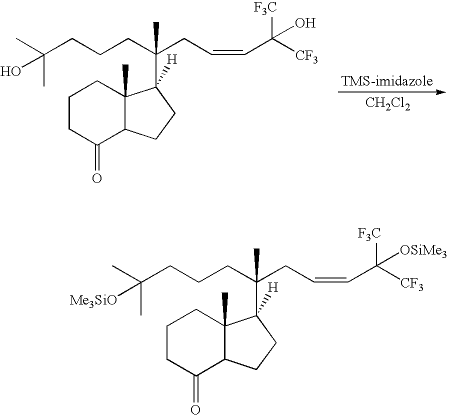

### (1R,3aR,4S,7aR)-7a-Methyl-1-[(1R,3Z)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one

A 25 ml round bottom flask equipped with stir bar and Claisen adapter with rubber septum was charged with 295 mg (0.606 mmol) of (1R,3aR,4S,7aR)-7a-methyl-1-[(1R,3Z)-6,6,6-trifluoro-5-hydroxy-1-(4-hydroxy-4-methyl-pentyl)-1-methyl-5-trifluoromethyl-hex-3-enyl]-octahydro-inden-4-one and 8.0 ml of dichloromethane. A 0.7 ml (4.8 mmol) of 1-(trimethylsilyl)imidazole was added dropwise. The mixture was stirred at room temperature for 3 h. A 100 ml of water was added and the mixture was extracted three times with 50 ml of ethyl acetate, dried over Na₂SO₄ and evaporated.

The oil residue was chromatographed on column (50 cm³) using hexane:ethyl acetate (10:1) as mobile phase. Fractions containing product were pooled and evaporated to give 362 mg (95%) of product as colorless oil.

¹H NMR (CDCl₃): 6.02-5.94 (1H, m), 5.42 (1H, d, J=11.0 Hz), 2.50-2.40 (2H, m), 2.35-2.14 (4H, m), 2.06-1.55 (7H, m), 1.43-1.14 (7H, m), 1.21 (6H, s), 0.96 (3H, s), 0.74 (3H, s), 0.24 (9H, s), 0.10 (9H, s)

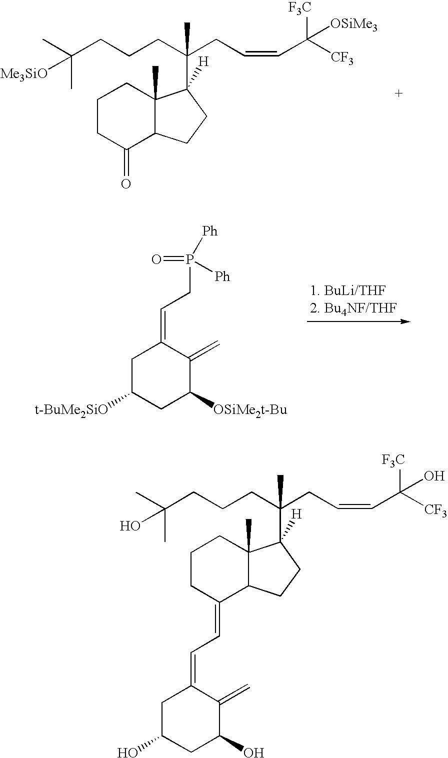

### (20)-1,25-Dihydroxy-20-[(2Z)-5,5,5-trifluoro-4-hydroxy-4-trifluoromethyl-pent-2-enyl]-cholecalciferol

A 25 ml round bottom flask equipped with stir bar and Claisen adapter with rubber septum was charged with 757 mg (1.299 mmol) of (1S,5R)-1,5-bis-((tent-butyl dimethyl)silanyloxy)-3-[2-(diphenylfosphinoyl)-eth-(Z)-ylidene]-2-methylene-cyclohexane and 10 ml of tetrahydrofurane. The reaction mixture was cooled to −78° C. and 0.8 ml (1.28 mmol) of 1.6M n-butyllithium in tetrahydrofurane was added dropwise. The resulting deep red solution was stirred at −78° C. for 20 min and 360 mg (0.571 mmol) of (1R,3aR,4S,7aR)-7a-methyl-1-[(1R,3Z)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one was added dropwise in 1.5 ml of tetrahydrofurane. The reaction mixture was stirred for 4 h 30 min (last 0.5 h at −30° C.) and then the bath was removed and the mixture was poured into 50 ml of ethyl acetate and 100 ml of brine. The water fraction was extracted three times with 50 ml of ethyl acetate, dried over Na₂SO₄ and evaporated. The oil residue was chromatographed on column (50 cm³, protected from light) using hexane:ethyl acetate (15:1) as mobile phase. Fractions containing product and some mono deprotected compound were pooled and evaporated to give colorless oil (430 mg) which was treated with 10 ml of 1M tetrabutylammonium fluoride in tetrahydrofurane. The reaction mixture was stirred at room temperature for 6 h 40 min. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give 278 mg (78%, two steps) of product as white foam.

[α]_{D}³¹=+6.50° c=0.51, EtOH

UV λmax (EtOH): 212.67 nm (ε 15573), 265.17 nm (ε 17296)

¹H NMR (D6-DMSO): 7.97 (1H, s), 6.18 (1H, d, J=11.3 Hz), 6.09 (1H, dt, J=12.1, 6.3 Hz), 5.96 (1H, d, J=11.3 Hz), 5.42 (1H, d, J=12.1 Hz), 5.22 (1H, s), 4.86 (1H, d, J=4.8 Hz), 4.75 (1H, s), 4.54 (1H, d, J=3.6 Hz), 4.20-4.36 (1H, m), 4.04 (1H, s), 4.00-3.96 (1H, m), 2.77 (1H, br d, J=11.1 Hz), 2.49-2.39 (2H, m), 2.3591H, d, J=11.9 Hz), 2.16 (1H, dd, J=13.4, 5.3 Hz), 2.00-1.86 (2H, m), 1.83-1.77 (1H, m), 1.70-1.15 (16H, m), 1.04 (3H, s), 1.04 (3H, s), 0.90 (3H, s), 0.60 (3H, s)

¹³C NMR (D6-DMSO): 149.40, 139.75, 139.21, 135.81, 122.94(q, J=287.7 Hz), 122.36, 117.87, 117.15, 109.75, 68.72, 68.34, 65.08, 56.56, 55.98, 46.15, 44.85, 44.69, 43.11, 40.35, 38.85, 36.04, 29.43, 29.12, 28.34, 23.13, 22.79, 21.83, 21.50, 17.96, 14.55

| | | | | |
|---|---|---|---|---|
| MS HRES | Calculated for: | C₃₃H₄₈F₆O₄ | [M + Na]⁺ | 645.3349 |
| | Observed: | | [M + Na]⁺ | 645.3337 |

### Example 19

### Synthesis of (20)-1,25-Dihydroxy-20-[(2Z)-5,5,5-trifluoro-4-hydroxy-4-trifluoromethyl-pent-2-enyl]-19-nor-cholecalciferol

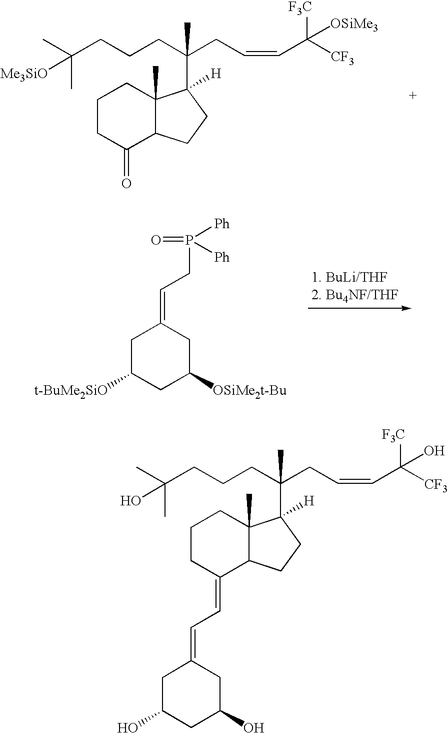

### (20)-1,25-Dihydroxy-20-[(2Z)-5,5,5-trifluoro-4-hydroxy-4-trifluoromethyl-pent-2-enyl]-19-nor-cholecalciferol

A 25 ml round bottom flask equipped with stir bar and Claisen adapter with rubber septum was charged with 804 mg (1.408 mmol) of (1R,3R)-1,3-bis-((tent-butyl dimethyl)silanyloxy)-5-[2-(diphenylfosphinoyl)ethylidene]-cyclohexane and 8 ml of tetrahydrofurane. The reaction mixture was cooled to −70° C. and 0.88 ml (1.41 mmol) of 1.6M n-butyllithium BuLi was added dropwise. The resulting deep red solution was stirred at −70° C. for 25 min and 441 mg (0.699 mmol) of (1R,3aR,4S,7aR)-7a-methyl-1-[(1R,3Z)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one in 1.5 ml of tetrahydrofurane. The reaction mixture was stirred for 6 h at −70° C. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate (25:1) as mobile phase. Fractions containing product were pooled and evaporated to give oil (ca. 615 mg) which was treated with 15 ml of 1M tetrabutylammonium fluoride in tetrahydrofurane. The reaction mixture was stirred at room temperature for 18 h. The new portion 5 ml of 1M tetrabutylammonium fluoride in tetrahydrofurane was added and the mixture was stirred for next 48 h. The mixture was dissolved by the addition of 150 ml of ethyl acetate and extracted six times with 50 ml of water:brine (1:1) and 50 ml of brine, dried over Na₂SO₄ and evaporated.

The oil residue was chromatographed on column (50 cm³, protected from light) using ethyl acetate:hexane (1:2, 1:1 and 3:1) and ethyl acetate as mobile phase. Fractions containing product were pooled and evaporated to give product as colorless oil. Oil was dissolved in methyl acetate and evaporated (2 times) to give 395 mg (92%) of product as white foam.

[α]_{D}²⁶=+42.6° c=0.50, EtOH

UV λmax (EtOH): 244 nm (ε 35888), 252 nm (ε 41722), 262 nm (ε 28261)

¹H NMR (DMSO-D6): 7.99 (1H, s), 6.14-6.08 (1H, m), 6.08 (1H, d, J=12.4 Hz), 5.78 (1H, d, J=11.3 Hz), 5.44 (1H, d, J=12.4 Hz), 4.48 (1H, d, J=4.1 Hz), 4.38 (1H, d, J=4.1 Hz), 4.05 (1H, s), 3.89-3.84 (1H, m), 3.83-3.77 (1H, m), 2.73 (1H, d, J=13.2 Hz), 2.49-2.41 (2H, m), 2.26 (1H, d, J=10.4 Hz), 2.07-1.96 (4H, m), 1.72-1.20 (18H, m), 1.05 (3H, s), 1.05 (3H, s), 0.91 (3H, s), 0.61 (3H, s)

¹³C NMR (D6-DMSO): 139.41, 139.34, 134.75, 123.07(q, J=288.2 Hz), 120.95, 117.26, 116.46, 76.83(sep, J=28.1 Hz), 68.77, 65.59, 65.31, 56.56, 55.98, 46.01, 44.71, 44.61, 42.22, 40.35, 39.01, 38.78, 36.96, 36.07, 29.44, 29.11, 22.97, 22.78, 21.88, 21.38, 17.94, 14.64

| | | | | |
|---|---|---|---|---|
| MS HRES | Calculated for: | C₃₂H₄₈F₆O₄ | [M + Na]⁺ | 633.3349 |
| | Observed: | | [M + Na]⁺ | 633.3357 |

### Example 20

### Synthesis of (20)-1α-Fluoro-25-hydroxy-20-[(2Z)-5,5,5-trifluoro-4-hydroxy-4-trifluoromethyl-pent-2-enyl]-cholecalciferol

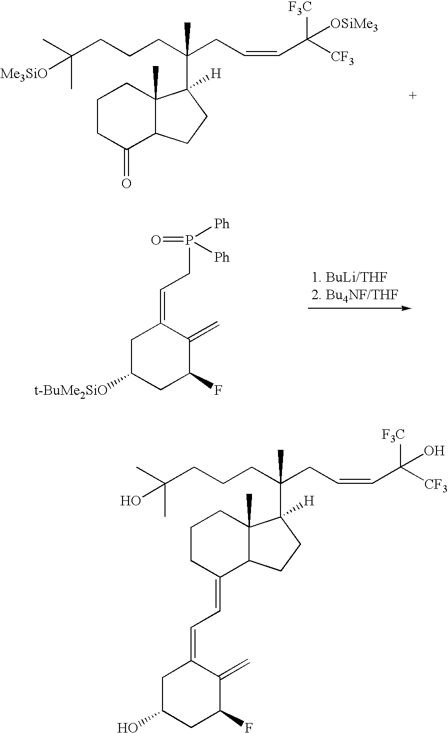

### (20)-1α-Fluoro-25-hydroxy-20-[(2Z)-5,5,5-trifluoro-4-hydroxy-4-trifluoromethyl-pent-2-enyl]-cholecalciferol

A 25 ml round bottom flask equipped with stir bar and Claisen adapter with rubber septum was charged with 673 mg (1.430 mmol) of (1S,5R)-1-((tent-butyl dimethyl)silanyloxy)-3-[2-(diphenylfosphinoyl)-eth-(Z)-ylidene]-5-fluoro-2-methylene-cyclohexane and 8 ml of tetrahydrofurane. The reaction mixture was cooled to −70° C. and 0.89 ml (1.42 mmol) of 1.6M n-butyllithium was added dropwise. The resulting deep red solution was stirred at −70° C. for 20 min and 320 mg (0.507 mmol) of (1R,3aR,4S,7aR)-7a-methyl-1-[(1R,3Z)-6,6,6-trifluoro-1-methyl-1-(4-methyl-4-trimethyl silanyloxy-pentyl)-5-trifluoromethyl-5-trimethylsilanyloxy-hex-3-enyl]-octahydro-inden-4-one was added dropwise in 1.5 ml of tetrahydrofurane. The reaction mixture was stirred for 4 h and then the dry ice was removed from bath and the solution was allowed to warm up to −40° C. in 2 h. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate (25:1) as mobile phase. Fractions containing product were pooled and evaporated to give oil (568 mg) which was treated with 10 ml of 1M tetrabutylammonium fluoride in tetrahydrofurane. The reaction mixture was stirred at room temperature for 17 h.

The mixture was dissolved by the addition of 150 ml of ethyl acetate and extracted six times with 50 ml of water:brine (1:1) and 50 ml of brine, dried over Na₂SO₄ and evaporated. The oil residue was chromatographed on two columns: 50 cm³ (protected from light) using ethyl acetate:hexane (1:1) as mobile phase and 50 cm³ (protected from light) using hexane:ethyl:acetate (2:1 and 1:1) Fractions containing product were pooled and evaporated to give product as colorless oil. The product was dissolved in methyl acetate and evaporated (2 times) to give 365 mg 81%) of product as white foam.

[α]_{D}²⁶=+22.2° c=0.49, EtOH

UV λmax (EtOH): 210 nm (ε 15393), 243 nm (ε 15181), 270 nm (ε 15115)

¹H NMR (DMSO-D6): 7.99 (1H, s), 6.36 (1H, d, J=11.3 Hz), 6.10 (1H, dt, J=12.2, 6.3 Hz), 5.93 (1H, d, J=11.3 Hz), 5.43 (1H, d, J=12.2 Hz), 5.39 (1H, s), 5.14 (1H, br d, J=47.5 Hz), 4.99(1H, d, J=1.7 Hz), 4.85 (1H, d, J=4.3 Hz), 4.05 (1H, s), 3.94-3.88 (1H, m), 2.81 (1H, d, J=13.2 Hz), 2.47-2.41 (2H, m), 2.16-2.05 (2H, m), 2.01-1.96 (2H, m), 1.83-1.18 (17H, m), 1.05 (3H, s), 1.05 (3H, s), 0.90 (3H, s), 0.60 (3H, s)

¹³C NMR (DMSO-D6): 143.30(d, J=16.7 Hz), 141.89, 139.35, 133.08, 124.18, 123.05(q, J=288.2 Hz), 117.37, 117.24, 115.26(d, J=9.1 Hz), 92.02 (d, J=167.6 Hz), 76.84 (sep, J=28.1 Hz), 68.76, 64.53, 56.55, 55.95, 46.25, 44.82, 44.70, 40.68(d, J=20.5 Hz), 40.29, 38.95, 38.77, 36.06, 29.41, 29.12, 28.32, 23.03, 22.71, 21.81, 21.37, 17.93, 14.55

| | | | | |
|---|---|---|---|---|
| MS HRES | Calculated for: | C₃₃H₄₇F₇O₃ | [M + Na]⁺ | 647.3305 |
| | Observed: | | [M + Na]⁺ | 647.3297 |

### Example 21

### Synthesis of (20R)-1,25-Dihydroxy-20-[(2E)-5,5,5-trifluoro-4-hydroxy-4-trifluoromethyl-pent-2-enyl]-cholecalciferol

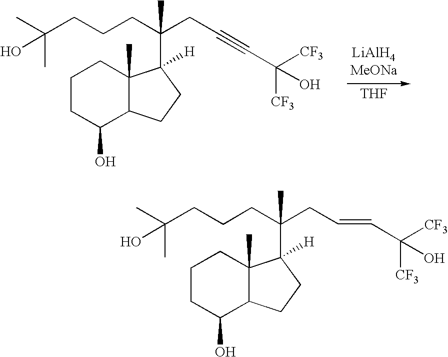

### (3E,6R)-1,1,1-Trifluoro-6-[(1R,3aR,4S,7aR)-4-hydroxy-7a-methyl-octahydro-inden-1-yl]-6,10-dimethyl-2-trifluoromethyl-undec-3-ene-2,10-diol

A 25 ml round bottom flask equipped with stir bar and condenser with nitrogen sweep was charged with 4.5 ml (4.5 mmol) of 1M lithium aluminum hydride in tetrahydrofurane and the mixture was cooled to 0° C. A 243 mg (4.50 mmol) of sodium methoxide was added slowly followed by substrate 337 mg (0.693 mmol) of (3E,6R)-1,1,1-trifluoro-6-[(1R,3aR,4S,7aR)-4-hydroxy-7a-methyl-octahydro-inden-1-yl]-6,10-dimethyl-2-trifluoromethyl-undec-3-yne-2,10-diol in 5 ml of tetrahydrofurane. The reaction mixture was stirred at 80° C. for 6 h 30 min and then was cooled to 0° C. A 1 ml of water, 1 ml of 2N NaOH and 20 ml of diethyl ether were added. The mixture was stirred at room temp for 30 min and 2.2 g of MgSO₄ was added and mixture was stirred for next 15 min. The suspension was filtrated and solvent evaporated. The oil residue was chromatographed on column (100 cm³) using dichloromethane:ethyl acetate (4:1) as mobile phase. Fractions containing product were pooled and evaporated to give 330 mg (97%) of product as colorless oil.

¹H NMR (CDCl₃): 6.28(1H, dt, J=15.7, 7.3 Hz), 5.59 (1H, d, J=15.4 Hz), 6.12 (1H, br s), 2.12 (2H, d, J=7.7 Hz), 2.06-1.98 (1H, m), 1.85-1.74 (2H, m), 1.68-1.16 (18H, m), 1.22 (6H, s), 1.08 (3H, s), 0.98 (3H, s)

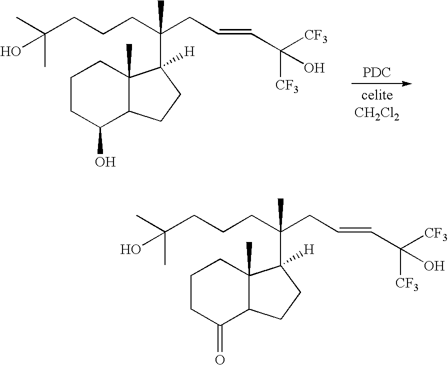

### (1R,3aR,4S,7aR)-7a-Methyl-1-[(1R,3E)-6,6,6-trifluoro-5-hydroxy-1-(4-hydroxy-4-methyl-pentyl)-1-methyl-5-trifluoromethyl-hex-3-enyl]-octahydro-inden-4-one

A 25 ml round bottom flask equipped with stir bar and Claisen adapter with rubber septum was charged with 330 mg (0.675 mmol) of (3E,6Z)-1,1,1-trifluoro-6-[(1R,3aR,4S, 7aR)-4-hydroxy-7a-methyl-octahydro-inden-1-yl]-6,10-dimethyl-2-trifluoromethyl-undec-3-ene-2,10-diol and 10 ml of dichloromethane. A 920 mg (2.445 mmol) of pyridinium dichromate was added and mixture was stirred in room temperature for 7 h.

The reaction mixture was filtrated through column with silica gel (60 cm³) using dichloromethane:ethyl acetate (4:1) as mobile phase. The fractions containing product were pooled and evaporated to give 302 mg (92%) of product as colorless oil.

[α]_{D}³⁰=−17.7° c=0.46, CHCl₃

¹H NMR (CDCl₃): 6.30(1H, dt, J=15.6, 7.7 Hz), 5.60 (1H, d, J=15.6 Hz), 2.40 (1H, dd, J=11.1, 7.3 Hz), 2.30-2.14 (6H, m), 2.06-1.98 (1H, m), 1.96-1.81 (1H, m), 1.78-1.30 (13H, m), 1.24 (3H, s), 1.23 (3H, s), 0.98 (3H, s), 0.74 (3H, s)

¹³C NMR (CDCl₃): 212.12, 136.27, 120.28, 71.45, 62.27, 57.44, 50.69, 44.28, 42.02, 40.76, 40.17, 39.69, 39.65, 29.34, 29.23, 23.98, 22.66, 22.24, 18.67, 18.19, 15.47

| | | | | |
|---|---|---|---|---|
| MS HRES | Calculated for: | C₂₄H₃₆F₆O₃ | [M + Na]⁺ | 509.2461 |
| | Observed: | | [M + Na]⁺ | 509.2463 |

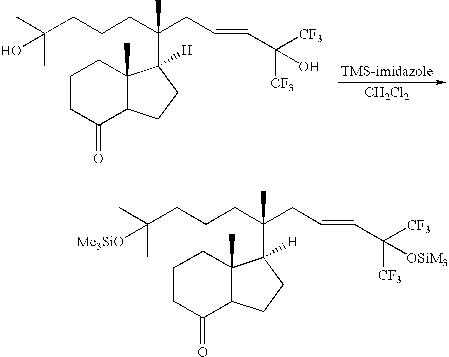

### (1R,3aR,4S,7aR)-7a-Methyl-1-[(1R,3E)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one

A 25 ml round bottom flask equipped with stir bar and Claisen adapter with rubber septum was charged with 292 mg (0.600 mmol) of (1R,3aR,4S,7aR)-7a-methyl-1-[(1R,3E)-6,6,6-trifluoro-5-hydroxy-1-(4-hydroxy-4-methyl-pentyl)-1-methyl-5-trifluoromethyl-hex-3-enyl]-octahydro-inden-4-one and 8 ml of dichloromethane. A 0.7 ml (4.8 mmol) of 1-(trimethylsilyl)imidazole was added dropwise. The mixture was stirred at room temperature for 2 h. A 100 ml of water was added and the mixture was extracted three times with 50 ml of ethyl acetate, dried over Na₂SO₄ and evaporated.

The oil residue was chromatographed on column (60 cm³) using hexane:ethyl acetate (10:1, 4:1) as mobile phase. Fractions containing product were pooled and evaporated to give 360 mg (95%) of product as colorless oil.

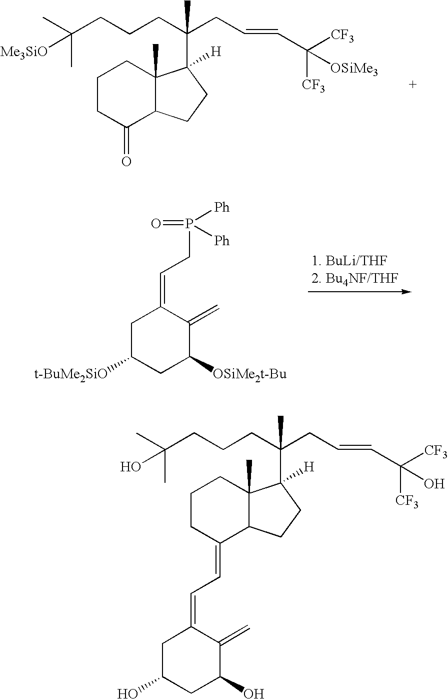

### (20R)-1,25-Dihydroxy-20-[(2E)-5,5,5-trifluoro-4-hydroxy-4-trifluoromethyl-pent-2-enyl]-cholecalciferol

A 25 ml round bottom flask equipped with stir bar and Claisen adapter with rubber septum was charged with 760 mg (1.304 mmol) of (1S,5R)-1,5-bis-((tert-butyl dimethyl)silanyloxy)-3-[2-(diphenylfosphinoyl)-eth-(Z)-ylidene]-2-methylene-cyclohexane and 10 ml of tetrahydrofurane. The reaction mixture was cooled to −78° C. and 0.8 ml (1.28 mmol) of 1.6M n-butyllithium in tetrahydrofurane was added dropwise. The resulting deep red solution was stirred at −78° C. for 20 min and 358 mg (0.567 mmol) of (1R,3aR,4S,7aR)-7a-methyl-1-[(1R,3E)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one was added dropwise in 1.5 ml of tetrahydrofurane. The reaction mixture was stirred for 4 h (last 0.5 h at −20° C.) and then the bath was removed and the mixture was poured into 50 ml of ethyl acetate and 100 ml of brine. The water fraction was extracted three times with 50 ml of ethyl acetate, dried over Na₂SO₄ and evaporated. The oil residue was chromatographed on column (50 cm³, protected from light) using hexane:ethyl acetate (10:1) as mobile phase. Fractions containing product and some mono deprotected compound were pooled and evaporated to give colorless oil (440 mg) which was treated with 10 ml of 1M tetrabutylammonium fluoride in tetrahydrofurane. The reaction mixture was stirred at room temperature for 21 h.

The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give 30 5 mg (86%, two steps) of product as colorless solid.

[α]_{D}³¹=+13.4° c=0.44, EtOH

UV λmax (EtOH): 212.76 nm (ε 15453), 265.03 (ε 17341)

¹H NMR (D6-DMSO): 8.04 (1H, s), 6.28 (1H, dt, J=15.5, 7.6 Hz), 6.18 (1H, d, J=11.1 Hz), 5.97 (1H, d, J=11.1 Hz), 5.61 (1H, d, J=15.5 Hz), 5.22 (1H, s), 4.75 (1H, s), 4.19-4.16 (1H, m), 3.98 (1H, br s), 2.77 (1H, d, 13.9 Hz), 2.35 (1H, d, J=11.7 Hz), 2.16 (1H, dd, J=13.6, 5.3 Hz), 2.07 (2H, d, J=7.3 Hz), 1.99-1.90 (2H, m), 1.81-1.78 (1H, m), 1.64-1.55 (6H, m), 1.48-1.17 (12H, m), 1.05 (6H, s), 0.90 (3H, s), 0.84 (1H, s), 0.61 (3H, s)

¹³C NMR (D6-DMSO): 149.34, 139.65, 136.40, 135.82, 122.60(q, J=287.7 Hz), 122.32, 119.80, 117.90, 109.76, 68.68, 68.36, 65.04, 56.35, 56.00, 46.18, 44.85, 44.64, 43.09, 41.05, 40.42, 29.34, 29.12, 28.31, 23.08, 22.47, 21.79, 21.58, 17.91, 14.57

| | | | | |
|---|---|---|---|---|
| MS HRES | Calculated for: | C₃₃H₄₈F₆O₄ | [M + Na]⁺ | 645.3349 |
| | Observed: | | [M + Na]⁺ | 645.3355 |

### Example 22

### Synthesis of (20R)-1,25-Dihydroxy-20-[(2E)-5,5,5-trifluoro-4-hydroxy-4-trifluoromethyl-pent-2-enyl]-19-nor-cholecaliferol

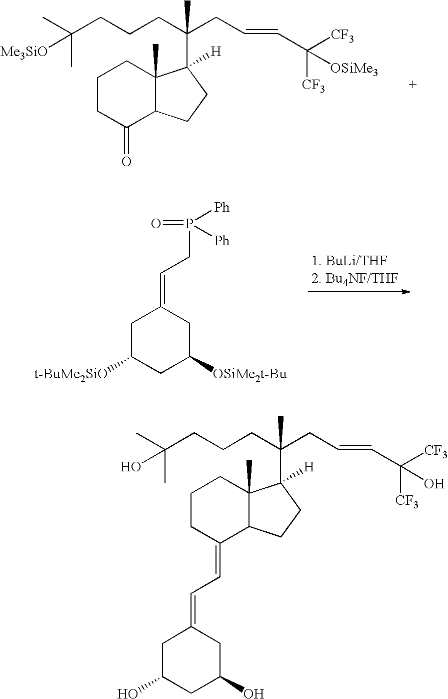

### (20R)-1,25-Dihydroxy-20-[(2E)-5,5,5-trifluoro-4-hydroxy-4-trifluoromethyl-pent-2-enyl]-19-nor-cholecalciferol

A 25 ml round bottom flask equipped with stir bar and Claisen adapter with rubber septum was charged with 493 mg (0.864 mmol) of (1R,3R)-1,3-bis-((tent-butyl dimethyl)silanyloxy)-5-[2-(diphenylfosphinoyl)ethylidene]-cyclohexane and 8 ml of tetrahydrofurane. The reaction mixture was cooled to −70° C. and 0.54 ml (0.86 mmol) of 1.6M n-butyllithium BuLi was added dropwise. The resulting deep red solution was stirred at −70° C. for 25 min and 240 mg (0.380 mmol) of (1R,3aR,4S,7aR)-7a-methyl-1-[(1R,3E)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one in 1.5 ml of tetrahydrofurane. The reaction mixture was stirred for 7 h and then the dry ice was removed from bath and the solution was allowed to warm up to −40° C. in 1 h. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (60 cm³, protected from light) using hexane:ethyl acetate (10:1) as mobile phase. Fractions containing product were pooled and evaporated to give colorless oil (ca. 380 mg) which was treated with 10 ml of 1M tetrabutylammonium fluoride in tetrahydrofurane. The reaction mixture was stirred at room temperature for 50 h. The mixture was dissolved by the addition of 150 ml of ethyl acetate and extracted six times with 50 ml of water, dried over Na₂SO₄ and evaporated. The oil residue was chromatographed on column (60 cm³, protected from light) using hexane:tetrahydrofurane (1:1, 1:2 and 1:2+10% methanol) as mobile phase. Fractions containing product were pooled and evaporated to give product 181 mg (78%) as colorless solid.

[α]_{D}³⁰=+52.8 c=0.50, EtOH

UV λmax (EtOH): 241 nm (ε 26823)

¹H NMR (DMSO-D6): 8.05 (1H, s), 6.29 (1H, dt, J=15.3, 7.7 Hz), 6.07 (1H, d, J=11.1 Hz), 5.78 (1H, d, J=11.1 Hz), 5.63 (1H, d, J=15.3 Hz), 4.48 (1H, s), 4.38 (1H, s), 4.06 (1H, s), 3.87 (1H, s), 3.80 (1H, s), 2.74 (1H, d, J=14.5 Hz), 2.43 (1H, dd, J=13.0, 3.4 Hz), 2.28-2.25 (1H, m), 2.10-1.91 (6H, m), 1.62-1.27 (17H, m), 1.06 (3H, s), 1.06 (3H, s), 0.91 (3H, s), 0.61 (3H, s)

¹³C NMR (D6-DMSO): 139.25, 136.60, 134.79, 122.73(q, J=286.8 Hz), 120.93, 119.96, 116.50, 75.55(sep, J=28.8 Hz), 68.74, 65.57, 65.29, 56.38, 56.00, 46.05, 44.67, 44.60, 42.22, 41.07, 40.43, 36.95, 29.35, 29.12, 28.14, 22.92, 22.47, 21.83, 21.47, 17.90, 14.66

| | | | | |
|---|---|---|---|---|
| MS HRES | Calculated for: | C₃₂H₄₈F₆O₄ | [M + Na]⁺ | 633.3349 |
| | Observed: | | [M + Na]⁺ | 633.3350 |

### Example 23

### Synthesis of (20R)-1α-Fluoro-25-hydroxy-20-[(2E)-5,5,5-trifluoro-4-hydroxy-4-trifluoromethyl-pent-2-enyl]-cholecalciferol

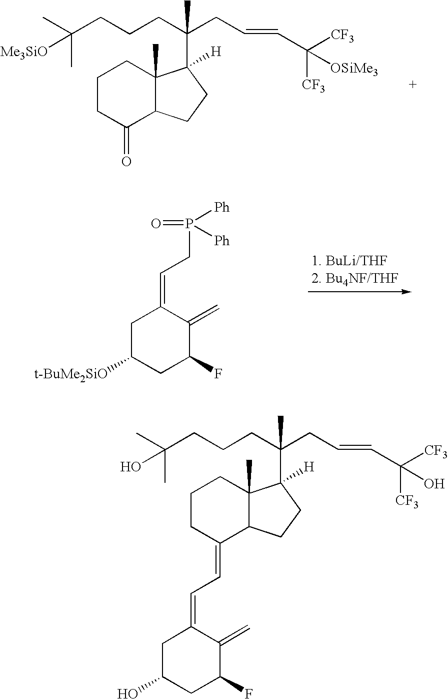

### (20R)-1α-Fluoro-25-hydroxy-20-[(2E)-5,5,5-trifluoro-4-hydroxy-4-trifluoromethyl-pent-2-enyl]-cholecalciferol

A 25 ml round bottom flask equipped with stir bar and Claisen adapter with rubber septum was charged with 439 mg (0.933 mmol) of (1S,5R)-1-((tent-butyl dimethyl)silanyloxy)-3-[2-(diphenylfosphinoyl)-eth-(Z)-ylidene]-5-fluoro-2-methylene-cyclohexane and 8 ml of tetrahydrofurane. The reaction mixture was cooled to −70° C. and 0.58 ml (0.93 mmol) of 1.6M n-butyllithium was added dropwise. The resulting deep red solution was stirred at −70° C. for 25 min and 238 mg (0.377 mmol) of (1R,3aR,4S,7aR)-7a-methyl-1-[(1R,3E)-6,6,6-trifluoro-1-methyl-1-(4-methyl-4-trimethyl silanyloxy-pentyl)-5-trifluoromethyl-5-trimethylsilanyloxy-hex-3-enyl]-octahydro-inden-4-one was added dropwise in 1.5 ml of tetrahydrofurane. The reaction mixture was stirred for 6 h and then the dry ice was removed from bath and the solution was allowed to warm up to −40° C. in 1 h. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate (10:1) as mobile phase. Fractions containing product were pooled and evaporated to give colorless oil which was treated with 8 ml of 1M tetrabutylammonium fluoride in tetrahydrofurane. The reaction mixture was stirred at room temperature for 15 h.

The mixture was dissolved by the addition of 150 ml of ethyl acetate and extracted six times with 50 ml of water, dried over Na₂SO₄ and evaporated. The oil residue was chromatographed on column (50 cm³, protected from light) using ethyl acetate:hexane (1:2 and 1:1) as mobile phase. Fractions containing product were pooled and evaporated to give product as colorless oil. The product was dissolved in methyl acetate and evaporated (2 times) to give 195 mg (83%) of product as white foam.

[α]_{D}²⁶=+29.3 c=0.43, EtOH

UV λmax (EtOH): 243 nm (ε 11639), 273 nm (ε 10871)

¹H NMR (DMSO-D6): 8.05 (1H, s), 6.37 (1H, d, J=11.3 Hz), 6.28 (1H, dt, J=15.3, 7.6 Hz), 5.93 (1H, d, J=11.3 Hz), 5.62 (1H, d, J=15.6 Hz), 5.39 (1H, s), 5.14 (1H, br d, J=47.7 Hz), 4.99 (1H, d, J=1.5 Hz), 4.87 (1H, br s), 4.06 (1H, br s), 3.93-3.88 (1H, m), 2.81 (1H, d, J=11.9 Hz), 2.16-2.06 (4H, m), 1.99-1.91 (2H, m), 1.82-1.26 (17H, m), 1.06 (3H, s), 1.06 (3H, s), 0.90 (3H, s), 0.60 (3H, s)

¹³C NMR (D6-DMSO): 143.26(d, J=17.5 Hz), 141.80, 136.57, 133.12, 124.17, 122.73(q, J=285.2 Hz), 119.96, 117.42, 115.37(d, J=9.9 Hz), 92.06 (d, J=166.9 Hz), 75.54 (sep, J=28.8 Hz), 68.74, 64.55(d, J=4.5 Hz), 56.38, 55.99, 46.28, 44.84, 44.67, 41.07, 40.69(d, J=20.5 Hz), 40.39, 29.34, 29.14, 28.31, 22.99, 22.42, 21.76, 21.47, 17.90, 14.58

| | | | | |
|---|---|---|---|---|
| MS HRES | Calculated for: | C₃₃H₄₇F₇O₃ | [M + Na]⁺ | 647.3305 |
| | Observed: | | [M + Na]⁺ | 647.3313 |

### Example 24

### Synthesis of 1,25-Dihydroxy-20S-20-(4-hydroxy-5,5,5-trideutero-4-trideuteromethyl-pentyl)-23-yne-26,27-hexafluorocholecalciferol

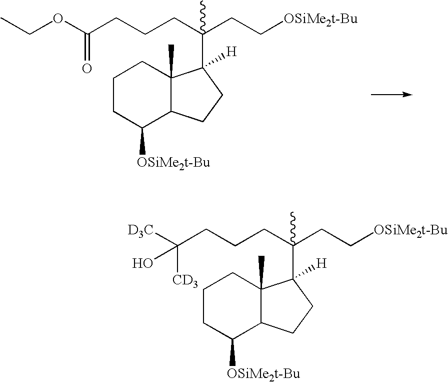

### 8-(tert-Butyl-dimethyl-silanyloxy)-6-[(1R,3aR,4S,7aR)-4-(tert-butyl-dimethyl-silanyloxy)-7a-methyl-octahydro-inden-1-yl]-1,1,1-trideutero-6-methyl-2-trideuteromethyl-octan-2-ol

A 250 ml round bottom flask equipped with stir bar, Claisen adapter with rubber septum was charged with 7-(tert-butyl-dimethyl-silanyloxy)-5-[(1R,3aR,4S,7aR)-4-(tert-butyl-dimethyl-silanyloxy)-7a-methyl-octahydro-inden-1-yl]-5-methyl-heptanoic acid ethyl ester (18.770 g, 32.987 mmol) and ether (150 ml). The solution was cooled in ace-water bath and a 1.0M solution of methyl-d₃-magnesium iodide in diethyl ether (100.0 ml, 100.0 mmol) was added dropwise. After completion of the addition the mixture was stirred at room temperature for 3 h then cooled again in an ice bath. A saturated solution of ammonium chloride (10 ml) was added dropwise. The resulting precipitate was dissolved by the addition of saturated solution of ammonium chloride (100 ml). The aqueous layer was extracted with diethyl ether (3×100 ml). The combined organic layers were dried (Na₂SO₄) and evaporated. The oil residue was used to next reaction.

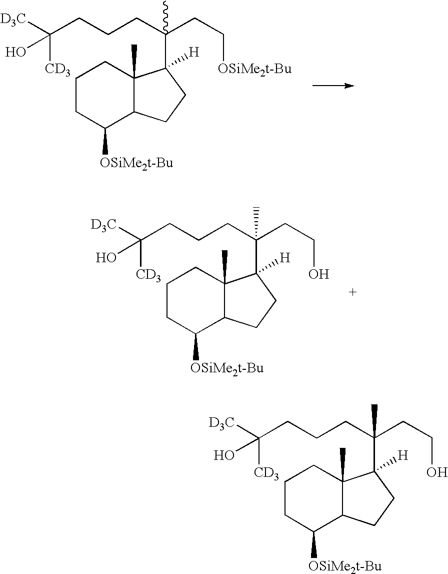

### (3S)-3-[(1R,3aR,4S,7aR)-4-(tert-Butyl-dimethyl-silanyloxy)-7a-methyl-octahydro-inden-1-yl]-8,8,8-trideutero-3-methyl-7-trideuteromethyl-octane-1,7-diol

### and

### (3R)-3-[(1R,3aR,4S,7aR)-4-(tert-Butyl-dimethyl-silanyloxy)-7a-methyl-octahydro-inden-1-yl]-8,8,8-trideutero-3-methyl-7-trideuteromethyl-octane-1,7-diol

A 250 ml round bottom flask equipped with stir bar and Claisen adapter with rubber septum was charged with 8-(tert-butyl-dimethyl-silanyloxy)-6-[(1R,3aR,4S,7aR)-4-(tert-butyl-dimethyl-silanyloxy)-7a-methyl-octahydro-inden-1-yl]-1,1,1-trideutero-6-methyl-2-trideuteromethyl-octan-2-ol (ca. 32.9 mmol), tetrahydrofuran (60 ml) and tetrabutylammonium fluoride (45.0 ml, 1M/tetrahydrofuran). The reaction mixture was stirred at room temperature for 2.5 h. The mixture was dissolved by the addition of ethyl acetate (150 ml) and washed six times with water:brine (1:1, 100 ml) and brine (50 ml), dried (Na₂SO₄) and evaporated. The oil residue was chromatographed 10 times on columns (VersaPak Cartridge, 80×150 mm and 40×150 mm, hexane/ethyl acetate—1:1) to give products (12.72 g, 87%):

### (3S)-3-[(1R,3aR,4S,7aR)-4-(tert-Butyl-dimethyl-silanyloxy)-7a-methyl-octahydro-inden-1-yl]-8,8,8-trideutero-3-methyl-7-trideuteromethyl-octane-1,7-diol (6.69 g, low polar epimer)

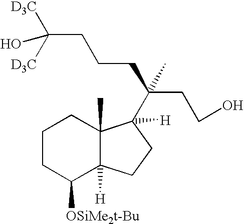

[α]_{D}³¹=+16.0 (c=0.60, EtOH)

¹H NMR (CDCl₃): 3.99 (1H, br s), 3.69-3.63 (2H, m), 2.02 (1H, br d, J=12.2 Hz), 1.82-1.48 (7H, m), 1.40-1.09 (14H, m), 1.06 (3H, s), 0.95 (3H, s), 0.88 (9H, s), 0.00 (3H, s), −0.01(3H, s)

| | | | | |
|---|---|---|---|---|
| MS HRES | Calculated for: | C₂₆H₄₆D₆O₃Si | [M + Na]⁺ | 469.3954 |
| | Observed: | | [M + Na]⁺ | 469.3956 |

### (3R)-3-[(1R,3aR,4S,7aR)-4-(tert-Butyl-dimethyl-silanyloxy)-7a-methyl-octahydro-inden-1-yl]-8,8,8-trideutero-3-methyl-7-trideuteromethyl-octane-1,7-diol (6.03 g, more polar epimer)

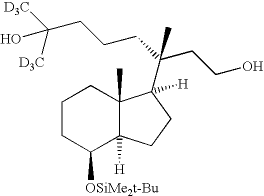

[α]_{D}³¹=+20.0 (c=0.54, EtOH)

¹H NMR (CDCl₃): 3.99-3.97 (1H, m), 3.66-3.62 (2H, m), 1.98 (1H, br d, J=12.8 Hz), 1.84-1.73 (1H, m), 1.67-1.51 (6H, m), 1.42-1.16 (14H, m), 1.05 (3H, s), 0.95 (3H, s), 0.88 (9H, s), 0.00 (3H, s), −0.01(3H, s)

| | | | | |
|---|---|---|---|---|
| MS HRES | Calculated for: | C₂₆H₄₆D₆O₃Si | [M + Na]⁺ | 469.3954 |
| | Observed: | | [M + Na]⁺ | 469.3957 |

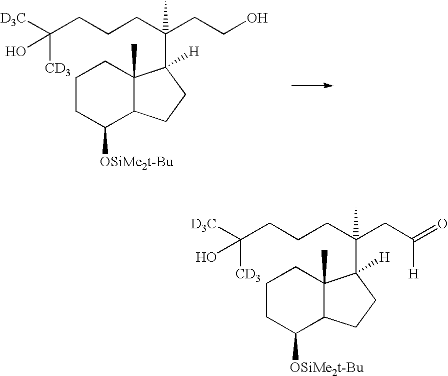

### (3S)-3-[(1R,3aR,4S,7aR)-4-(tert-Butyl-dimethyl-silanyloxy)-7a-methyl-octahydro-inden-1-yl]-8,8,8-trideutero-7-hydroxy-3-methyl-7-trideuteromethyl-octanal

A 250 ml round bottom flask equipped with stir bar and Claisen adapter with rubber septum was charged with pyridinium chlorochromate (2.90 g, 13.45 mmol), celite (4.0 g) and dichloromethane (60 ml). The (3S)-3-[(1R,3aR,4S,7aR)-4-(tert-butyl-dimethyl-silanyloxy)-7a-methyl-octahydro-inden-1-yl]-8,8,8-trideutero-3-methyl-7-trideuteromethyl-octane-1,7-diol (4.00 g, 8.95 mmol) in dichloromethane (5 ml) was added dropwise and mixture was stirred in room temperature for 2 h 40 min.

The reaction mixture was filtrated through column with silica gel (200 cm³) and celite (2 cm) using dichloromethane, dichloromethane:ethyl acetate 4:1. The fractions containing product were pooled and evaporated to give oil (3.61 g, 91%). Product was used to the next reaction without purification.

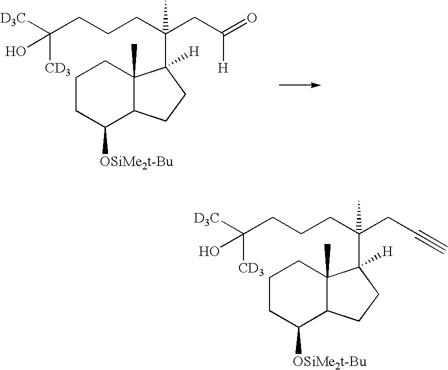

### (6S)-6-[(1R,3aR,4S,7aR)-4-(tert-Butyl-dimethyl-silanyloxy)-7a-methyl-octahydro-inden-1-yl]-1,1,1-trideutero-6-methyl-2-trideuteromethyl-non-8-yn-2-ol

A 100 ml round bottom flask equipped with stir bar and Claisen adapter with rubber septum was charged with (3S)-3-[(1R,3aR,4S,7aR)-4-(tert-butyl-dimethyl-silanyloxy)-7a-methyl-octahydro-inden-1-yl]-8,8,8-trideutero-7-hydroxy-3-methyl-7-trideuteromethyl-octanal (3.61 g, 8.116 mmol) and methanol (65 ml). 1-diazo-2-oxo-propyl)-phosphonic acid dimethyl ester (3.00 g, 15.62 mmol) in methanol (3 ml) was added and the resulting mixture was cooled in an ice bath. Potassium carbonate (3.00 g, 21.74 mmol) was added and the reaction mixture was stirred in the ice bath for 30 min and then at room temperature for 4 h. Water (100 ml) was added and the mixture was extracted with ethyl acetate (4×80 ml), dried (Na₂SO₄) and evaporated.

The oil residue was chromatographed on column (300 cm³) using hexane:ethyl acetate—9:1 and 8:1 as mobile phase. Fractions containing product were pooled and evaporated to give product as colorless oil (3.131 g, 87.5%).

[α]_{D}²⁶=+17.6 (c=0.83, EtOH)

¹H NMR (CDCl₃): 3.98 (1H, br d, J=2.13 Hz), 2.28 (1H, AB, J=17.3 Hz), 2.26 (1H, AB, J=17.3 Hz), 1.96-1.91 (2H, m), 1.84-1.73 (1H, m), 1.67-1.48 (5H, m), 1.43-1.24 (12H, m), 1.04 (3H, s), 1.00 (3H, s), 0.88 (9H, s), 0.00 (3H, s), −0.01(3H, s)

¹³C NMR (CDCl₃): δ3.06, 76.41(sep, J=29.6 Hz), 69.84, 69.55, 56.54, 52.87, 44.66, 43.68, 41.27, 40.16, 39.28, 34.32, 28.76, 25.87, 22.76, 22.69, 22.17, 18.10, 17.76, 16.78, −4.69, −5.05

| | | | | |
|---|---|---|---|---|
| MS HRES | Calculated for: | C₂₇H₄₄D₆O₂Si | [M + Na]⁺ | 463.3849 |
| | Observed: | | [M + Na]⁺ | 463.3848 |

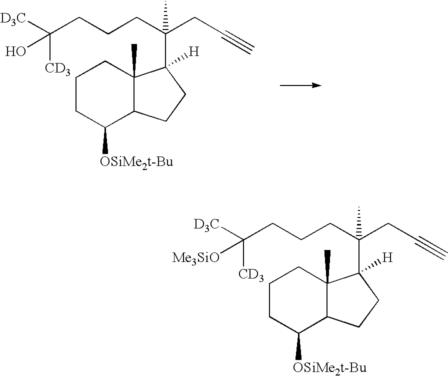

### (1R,3aR,4S,7aR)-4-(tert-Butyl-dimethyl-silanyloxy)-7a-methyl-1-[(1S)-6,6,6-trideutero-1-methyl-1-(prop-2-ynyl)-5-trideuteromethyl-5-trimethylsilanyloxy-hexyl]-octahydro-indene

A 100 ml round bottom flask equipped with stir bar and Claisen adapter with rubber septum was charged with (6S)-6-[(1R,3aR,4S,7aR)-4-(tert-butyl-dimethyl-silanyloxy)-7a-methyl-octahydro-inden-1-yl]-1,1,1-trideutero-6-methyl-2-trideuteromethyl-non-8-yn-2-ol (3.100 g, 7.033 mmol) and dichloromethane (30 ml). 1-(trimethylsilyl)imidazole (3.0 ml, 20.45 mmol) was added dropwise. The mixture was stirred at room temperature for 1 h 45 min. Water (100 ml) was added and the mixture was extracted with ethyl acetate (3×100 ml), dried (Na₂SO₄) and evaporated. The oil residue was chromatographed on column (125 cm³) using hexane:ethyl acetate—10:1 as mobile phase. Fractions containing product were pooled and evaporated to give product as colorless oil (3.36 g, 93%).

[α]_{D}²⁶=+15.4 (c=0.52, CHCl₃)

¹H NMR (CDCl₃): 3.99 (1H, br s), 2.27 (2H, br s), 2.00-1.93 (2H, m), 1.84-1.73 (1H, m), 1.65 (1H, d, J=14.3 Hz), 1.59-1.49 (3H, m), 1.42-1.20 (12H, m), 1.05 (3H, s), 1.00 (3H, s), 0.88 (9H, s), 0.10 (9H, s), 0.00 (3H, s), −0.01(3H, s)

¹³C NMR (CDCl₃): 83.18, 76.66(sep, J=28.8 Hz), 69.74, 69.58, 56.62, 52.91, 45.38, 43.67, 41.27, 40.07, 39.28, 34.34, 28.77, 25.88, 22.76, 22.16, 18.13, 18.11, 17.77, 16.76, 2.74, −4.69, −5.05

| | | | | |
|---|---|---|---|---|
| MS HRES | Calculated for: | C₃₀H₅₂D₆O₂Si₂ | [M + Na]⁺ | 535.4244 |
| | Observed: | | [M + Na]⁺ | 535.4246 |

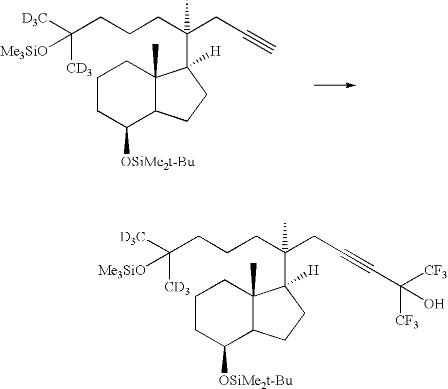

### (6S)-6-[(1R,3aR,4S,7aR)-4-(tert-Butyl-dimethyl-silanyloxy)-7a-methyl-octahydro-inden-1-yl]-6-methyl-11,11,11-trideutero-10-trideuteromethyl-1,1,1-trifluoro-2-trifluoromethyl-10-trimethylsilanyloxy-undec-3-yn-2-ol

A two neck 100 ml round bottom flask equipped with stir bar, Claisen adapter with rubber septum and funnel (with cooling bath) was charged with (1R,3aR,4S,7aR)-4-(tert-butyl-dimethyl-silanyloxy)-7a-methyl-1-[(1S)-6,6,6-trideutero-1-methyl-1-(prop-2-ynyl)-5-trideuteromethyl-5-trimethylsilanyloxy-hexyl]-octahydro-indene (3.330 g, 6.491 mmol) and tetrahydrofuran (40 ml). The funnel was connected to container with hexafluoroacetone and cooled (acetone, dry ice). The reaction mixture was cooled to −70° C. and n-butyllithium (6.10 ml, 9.76 mmol) was added dropwise. After 30 min hexafluoroacetone was added (the container's valve was opened three times). The reaction was steered at −70° C. for 2 h then saturated solution of ammonium chloride (5 ml) was added. The mixture was dissolved by the addition of saturated solution of ammonium chloride (100 ml) and extracted with ethyl acetate (3×60 ml), dried (Na₂SO₄) and evaporated. The residue was chromatographed twice on columns (300 cm³, hexane:ethyl acetate—25:1 and 20:1) to give the mixture of product and polimer (from hexafluoroacetone) (4.33 g). Product was used to the next reaction without purification.

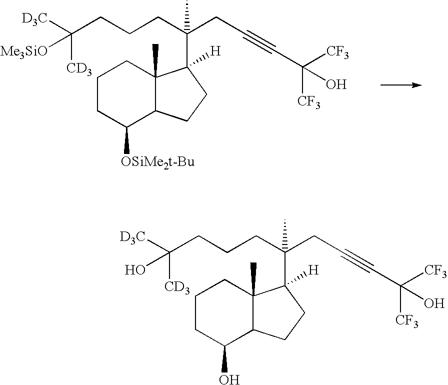

### (6S)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol

A 25 ml round bottom flask equipped with stir bar and Claisen adapter with rubber septum was charged with (6S)-6-[(1R,3aR,4S,7aR)-4-(tert-butyl-dimethyl-silanyloxy)-7a-methyl-octahydro-inden-1-yl]-6-methyl-11,11,11-trideutero-10-trideuteromethyl-1,1,1-trifluoro-2-trifluoromethyl-10-trimethylsilanyloxy-undec-3-yn-2-ol(ca 3.3 mmol) and tetrabutylammonium fluoride (25 ml, 1M/tetrahydrofuran) and reaction was stirred at 70° C. for 113 h. The mixture was dissolved by the addition of ethyl acetate (150 ml) and extracted six times with water-brine (1:1, 50 ml) and dried (Na₂SO₄) and evaporated.

Product was crystallized from hexane (1.996 g, 62%).

[α]_{D}³¹=−6.3 (c=0.46, EtOH)

¹H NMR (DMSO-D6): 8.92 (1H, s), 4.21 (1H, d, J=3.0 Hz), 4.04 (1H, s), 3.87 (1H, s), 2.37 (2H, s), 1.89 (1H, d, J=11.5 Hz), 1.76-1.48 (6H, m), 1.33-1.11 (11H, m), 1.02 (3H, s), 0.96 (3H, m)

¹³C NMR (DMSO-D6): 121.47(q, J=286.8 Hz), 89.70, 70.71, 70.40(sep, J=31.9 Hz), 68.41, 66.86, 56.24, 52.37, 44.45, 42.96, 40.44, 39.38, 33.70, 28.14, 22.43, 22.01, 21.68, 17.73, 17.46, 16.32

| | | | | |
|---|---|---|---|---|
| MS HRES | Calculated for: | C₂₄H₃₀D₆F₆O₃ | [M + Na]⁺ | 515.2837 |
| | Observed: | | [M + Na]⁺ | 515.2838 |

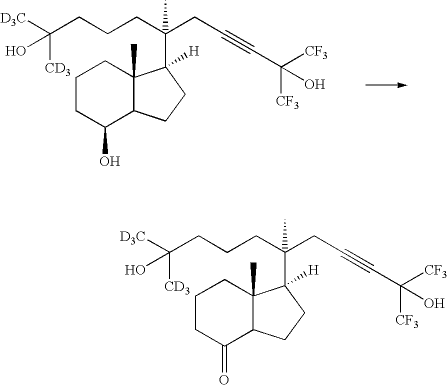

### (1R,3aR,7aR)-7a-Methyl-1-[(1S)-6,6,6-trifluororo-5-hydroxy-1-methyl-1-(5,5,5-trideutero-4-hydroxy-4-trideuteromethyl-pentyl)-5-trifluoromethyl-hex-3-ynyl]-octahydro-inden-4-one

A 25 ml round bottom flask equipped with stir bar and Claisen adapter with rubber septum was charged with pyridinium dirochromate (1.51 g, 4.01 mmol) and dichloromethane (20 ml). The (6S)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol (712 mg, 1.445 mmol) in dichloromethane (5 ml) was added dropwise and mixture was stirred in room temperature for 2 h 45 min.

The reaction mixture was filtrated through column with silica gel (50 cm³) using dichloromethane, dichloromethane:ethyl acetate 4:1. The fractions containing product were pooled and evaporated to give oil. The product was used to the next reaction without purification.

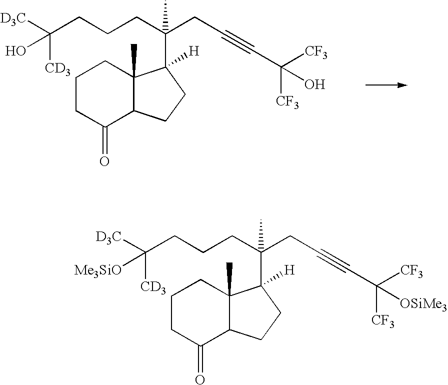

### (1R,3aR,7aR)-7a-Methyl-1-[(1S)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one

A 25 ml round bottom flask equipped with stir bar and Claisen adapter with rubber septum was charged with (1R,3aR,7aR)-7a-methyl-1-[(1S)-6,6,6-trifluororo-5-hydroxy-1-methyl-1-(5,5,5-trideutero-4-hydroxy-4-trideuteromethyl-pentyl)-5-trifluoromethyl-hex-3-ynyl]-octahydro-inden-4-one (ca. 1.445 mmol) and dichloromethane (10 ml). 1-(trimethylsilyl)imidazole (2.00 ml, 13.63 mmol) was added dropwise. The mixture was stirred at room temperature for 2 h. Ethyl acetate (150 ml) was added and the mixture was washed with water (3×50 ml), dried (Na₂SO₄) and evaporated. The oil residue was chromatographed on column (50 cm³) using hexane:ethyl acetate—5:1 as mobile phase. The product is unstable on the silica gel (the monoprotected compound was obtained (246 mg)). Fractions containing product were pooled and evaporated to give product as colorless oil (585 mg, 64%).

¹H NMR (CDCl₃): 2.44-2.37 (3H, m), 2.32-2.16 (2H, m), 2.11-1.99 (2H, m), 1.95-1.84 (2H, m), 1.81-1.52 (5H, m), 1.38-1.20 (6H, m), 1.03 (3H, s), 0.74 (3H, s), 0.28 (9H, s), 0.10 (9H, s)

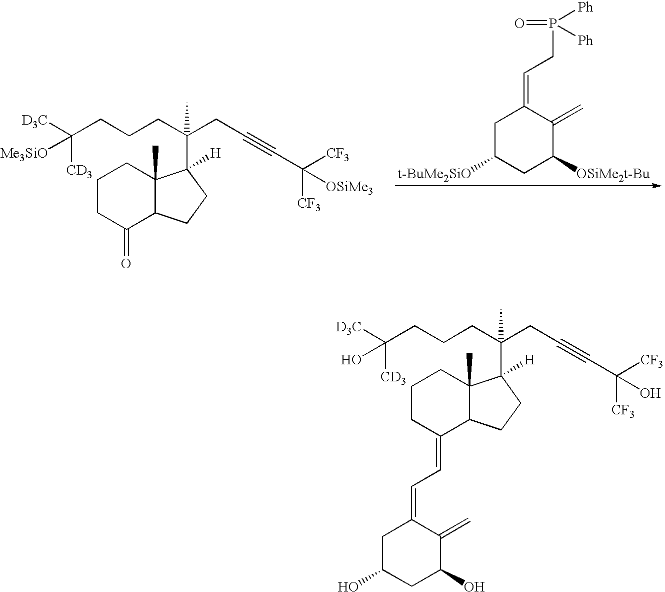

### 1,25-Dihydroxy-20S-20-(4-hydroxy-5,5,5-trideutero-4-trideuteromethyl-pentyl)-23-yne-26,27-hexafluorocholecalciferol

A 25 ml round bottom flask equipped with stir bar and Claisen adapter with rubber septum was charged with (1S,5R)-1,5-bis-((tert-butyldimethyl)silanyloxy)-3-[2-(diphenylfosphinoyl)-eth-(Z)-ylidene]-2-methylene-cyclohexane (532 mg, 0.913 mmol) and tetrahydrofuran (8 ml). The reaction mixture was cooled to −78° C. and n-butyllithium (0.57 ml, 0.912 mmol)) was added dropwise. The resulting deep red solution was stirred at −70° C. for 20 min and (1R,3aR,7aR)-7a-methyl-1-[(1S)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one (281 mg, 0.443 mmol) was added dropwise in tetrahydrofuran (1.5 ml). The reaction mixture was stirred for 5 h (in last hour the temperature was increased from −70 do −55° C.). The bath was removed and the mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product were pooled and evaporated to give colorless oil. The oil residue was used to next reaction. A 25 ml round bottom flask equipped with stir bar and Claisen adapter with rubber septum was charged with substrate and tetrabutylammonium fluoride (15 ml, 1M/tetrahydrofuran). The mixture was stirred for next 25 h. The mixture was dissolved by the addition of ethyl acetate (150 ml) and washed 6 times with water (50 ml) and brine (50 ml), dried (Na₂SO₄) and evaporated. The oil residue was chromatographed on column (50 cm³, protected from light) using ethyl acetate as mobile phase. Fractions containing product were pooled and evaporated to give product as colorless oil. There was an impurity (Bu₃N) in the product (¹H, ¹³C NMR). Material was chromatographed on column (70 cm³, protected from light) using hexane:ethyl acetate 1:1 and ethyl acetate as mobile phase. Oil was dissolved in methyl acetate and evaporated (4 times) to give product as white foam (191 mg, 69%).

[α]_{D}²⁵=+3.6 (c=0.44, EtOH)

UV λmax (EtOH): 213 nm (ε 15402), 264 nm (ε 17663)

¹H NMR (DMSO-D6): 8.95 (1H, br s), 6.18 (1H, d, J=11.1 Hz), 5.97 (1H, d, J=11.1 Hz), 5.23 (1H, d, J=1.1 Hz), 4.88 (1H, d, J=3.4 Hz), 4.75 (1H, d, J=1.7 Hz), 4.56 (1H, s), 4.19 (1H, br s), 4.06 (1H, br s), 3.99 (1H, br s), 2.78 (1H, d, J=12.2 Hz), 2.45-2.29 (2H, m), 2.17 (1H, dd, J=13.2, 5.4 Hz), 1.96-1.91 (2H, m), 1.84-1.73 (2H, m), 1.65-1.18 (17H, m), 0.96 (3H, s), 0.61 (3H, s)

¹³C NMR (DMSO-D6): 149.40, 139.51, 135.95, 122.33, 121.49(q, J=286.0 Hz), 118.02, 109.77, 89.59, 70.84, 70.43(sep, J=31.9 Hz), 68.42, 68.37, 65.09, 56.36, 55.94, 45.97, 44.87, 44.43, 43.12, 39.98, 39.85, 39.43, 28.35, 28.27, 23.11, 22.51, 22.02, 21.42, 17.77, 14.44

| | | | | |
|---|---|---|---|---|
| MS HRES | Calculated for: | C₃₃H₄₀D₆F₆O₄ | [M + Na]⁺ | 649.3569 |
| | Observed: | | [M + Na]⁺ | 649.3572 |

### Example 25

### Synthesis of 1,25-Dihydroxy-20S-20-(4-hydroxy-5,5,5-trideutero-4-trideuteromethyl-pentyl)-23-yne-26,27-hexafluoro-19-nor-cholecalciferol

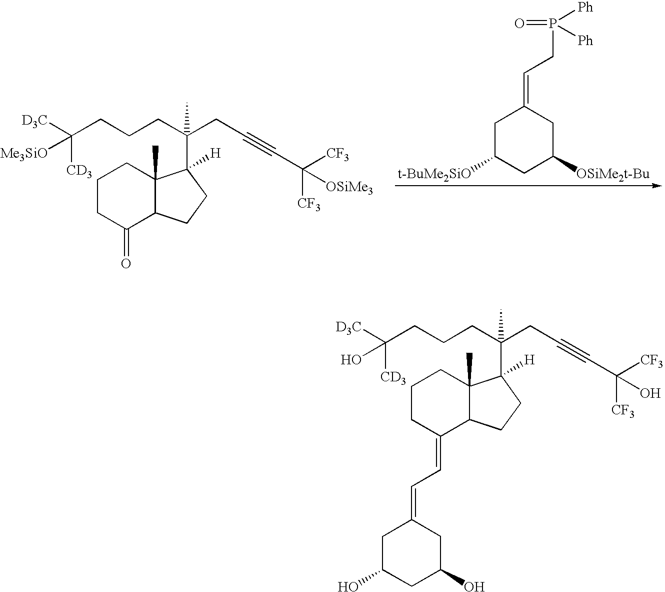

### 1,25-Dihydroxy-20S-20-(4-hydroxy-5,5,5-trideutero-4-trideuteromethyl-pentyl)-23-yne-26,27-hexafluoro-19-nor-cholecalciferol

A 25 ml round bottom flask equipped with stir bar and Claisen adapter with rubber septum was charged with (1R,3R)-1,3-bis-((tert-butyldimethyl)silanyloxy)-5-[2-(diphenylfosphinoyl)ethylidene]-cyclohexane (562 mg, 0.984 mmol) and tetrahydrofuran (8 ml). The reaction mixture was cooled to −70° C. and n-butyllithium (0.61 ml, 0.98 mmol)) was added dropwise. The resulting deep red solution was stirred at −70° C. for 20 min and (1R,3aR,7aR)-7a-methyl-1-[(1S)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one (296 mg, 0.466 mmol) was added dropwise in tetrahydrofuran (1.5 ml). The reaction mixture was stirred for 4 h 40 min (in last hour the temperature was increased from −70 do −55° C.). The bath was removed and the mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil (380 mg). A 25 ml round bottom flask equipped with stir bar and Claisen adapter with rubber septum was charged with substrate and tetrabutylammonium fluoride (15 ml, 1M/tetrahydrofuran). The mixture was stirred for next 49 h. The mixture was dissolved by the addition of ethyl acetate (150 ml) and extracted 6 times with water (50 ml) and brine (50 ml), dried (Na₂SO₄) and evaporated.

The oil residue was chromatographed on column (50 cm³, protected from light) using ethyl acetate as mobile phase. Fractions containing product were pooled and evaporated to give colorless oil. There was an impurity (Bu₃N) in the product (¹H, ¹³C NMR). Material was chromatographed twice on columns (60 cm³, protected from light) using hexane:ethyl acetate 2:1 and ethyl acetate as mobile phase. Oil was dissolved in methyl acetate and evaporated (4 times) to give product as white foam (251 mg, 87%).

[α]_{D}²²=+33.5 (c=0.48, EtOH)

UV λmax (EtOH): 243 nm (ε 29859), 252 nm (ε 34930), 262 nm (ε 23522) ¹H NMR (DMSO-D6): 8.94 (1H, s), 6.07 (1H, d, J=11.0 Hz), 5.78 (1H, d, J=11.0 Hz), 4.48 (1H, d, J=4.0 Hz), 4.38 (1H, d, J=4.0 Hz), 4.04 (1H, s), 3.92-3.76 (2H, m), 2.77 (1H, br d, J=11.0 Hz), 2.49-2.25 (2H, m), 2.05-1.95 (4H, m), 1.76-1.20 (19H, m), 0.97 (3H, s), 0.60 (3H, s)

¹³C NMR (DMSO-D6): 138.95, 134.73, 121.50(q, J=286.0 Hz), 120.80, 116.47, 89.59, 70.84, 70.44(sep, J=31.9 Hz), 68.43, 65.57, 65.45, 65.28, 56.37, 55.91, 45.82, 44.59, 44.45, 42.23, 40.01, 39.43, 36.98, 28.29, 28.19, 22.98, 22.54, 22.08, 21.33, 17.78, 14.55

| | | | | |
|---|---|---|---|---|
| MS HRES | Calculated for: | C₃₂H₄₀D₆F₆O₄ | [M + Na]⁺ | 637.3569 |
| | Observed: | | [M + Na]⁺ | 637.3570 |

### Example 26

### Synthesis of 1α-Fluoro-25-hydroxy-20S-20-(4-hydroxy-5,5,5-trideutero-4-trideuteromethyl-pentyl)-23-yne-26,27-hexafluorocholecalciferol

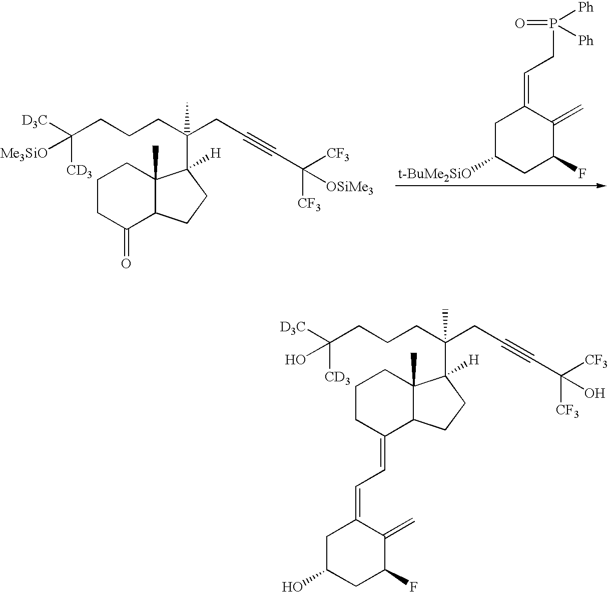

### 1α-Fluoro-25-hydroxy-20S-20-(4-hydroxy-5,5,5-trideutero-4-trideuteromethyl-pentyl)-23-yne-26,27-hexafluorocholecalciferol

A 25 ml round bottom flask equipped with stir bar and Claisen adapter with rubber septum was charged with (1S,5R)-1-((tert-butyldimethyl)silanyloxy)-3-[2-(diphenylfosphinoyl)-eth-(Z)-ylidene]-5-fluoro-2-methylene-cyclohexane (500 mg, 1.062 mmol) and tetrahydrofuran (8 ml). The reaction mixture was cooled to −70° C. and n-butyllithium (0.66 ml, 1.06 mmol)) was added dropwise. The resulting deep red solution was stirred at −70° C. for 20 min and (1R,3aR, 7aR)-7a-methyl-1-[(1S)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one (269 mg, 0.424 mmol) was added dropwise in tetrahydrofuran (1.5 ml). The reaction mixture was stirred for 5 h (in last hour the temperature was increased from −70 do −55° C.). The bath was removed and the mixture was poured into ethyl acetate (50 ml) and saturated solution of ammonium chloride (100 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product were pooled and evaporated to give colorless oil. The oil residue was used to next reaction. A 25 ml round bottom flask equipped with stir bar and Claisen adapter with rubber septum was charged with substrate and tetrabutylammonium fluoride (15 ml, 1M/tetrahydrofuran). The mixture was stirred for 6 h. The mixture was dissolved by the addition of ethyl acetate (150 ml) and washed 6 times with water (50 ml) and brine (50 ml), dried (Na₂SO₄) and evaporated. The oil residue was chromatographed on column (50 cm³, protected from light) using hexane:ethyl acetate—1:1 as mobile phase. Fractions containing product were pooled and evaporated to give product as colorless oil. There was an impurity (Bu₃N) in the product ('H, ¹³C NMR). Material was chromatographed on column (60 cm³, protected from light) using hexane:ethyl acetate 2:1 and 1:1 as mobile phase. Oil was dissolved in methyl acetate and evaporated (4 times) to give product as white foam (229 mg, 86%).

[α]_{D}²⁵=+20.9 (c=0.45, EtOH)

UV λmax (EtOH): 211 nm (ε 15893), 243 nm (ε 16109), 270 nm (ε 16096)

¹H NMR (DMSO-D6): 8.93 (1H, s), 6.36 (1H, d, J=11.1 Hz), 5.93 (1H, d, J=11.3 Hz), 5.38 (1H, s), 5.14 (1H, ddd, J=49.6, 3.4, 2.0 Hz), 4.98 (1H, d, J=1.5 Hz), 4.86 (1H, d, J=4.3 Hz), 4.05 (1H, s), 3.94-3.88 (1H, m), 2.81 (1H, d, J=13.2 Hz), 2.44-2.35 (2H, m), 2.16-2.08 (2H, m), 1.98-1.93 (2H, m), 1.84-1.17 (17H, m), 0.95 (3H, s), 0.59 (3H, s)

¹³C NMR (DMSO-D6): 143.15(d, J=16.7 Hz), 141.49, 133.06, 124.03, 121.49(q, J=286.0 Hz), 117.40, 115.18(d, J=9.9 Hz), 91.97 (d, J=166.9 Hz), 89.61, 70.85, 70.44(sep, J=31.9 Hz), 68.43, 64.55(d, J=4.6 Hz), 56.37, 55.91, 46.06, 44.84, 44.44, 40.70(d, J=20.5 Hz), 39.97, 39.81, 39.43, 28.37, 28.26, 23.06, 22.52, 22.02, 21.32, 17.77, 14.48

| | | | | |
|---|---|---|---|---|
| MS HRES | Calculated for: | C₃₃H₃₉D₆F₇O₃ | [M + Na]⁺ | 651.3526 |
| | Observed: | | [M + Na]⁺ | 651.3528 |

### Example 27

### Synthesis of 1,25-Dihydroxy-20S-20-(4-hydroxy-5,5,5-trideutero-4-trideuteromethyl-pentyl)-23Z-ene-26,27-hexafluorocholecalciferol

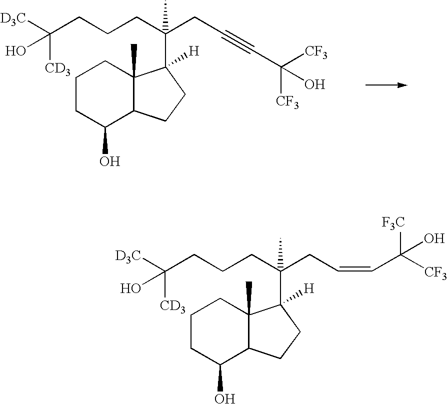

### (6S,3Z)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol

A 50 ml round bottom flask was charged with (6S)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol (722 mg, 1.466 mmol), Pd/CaCO₃ (180 mg, 5%), hexane (16.8 ml), ethyl acetate (6.8 ml) and solution of quinoline in ethanol (0.65 ml, prepared from ethanol (3.1 ml) and quinoline (168 μl)).

The substrate was hydrogenated at ambient temperature and atmospheric pressure of hydrogen. The reaction was monitoring by TLC (dichloromethane:ethyl acetate 4:1, 3×).

After 5 h 10 min the catalyst was filtered off (celite) and solvent evaporated. The residue was purified over silica gel (50 cm³) using dichloromethane:ethyl acetate 4:1. Fractions containing product were pooled and evaporated to give product as colorless oil (720 mg, 99%).

[α]_{D}²¹=+3.3 (c=0.49, EtOH)

¹H NMR (CDCl₃): 6.14-6.05 (1H, m), 5.48 (1H, d, J=12.8 Hz), 4.08 (1H, s), 2.83 (1H, dd, J=15.6, 9.0 Hz), 2.48-2.40 (1H, m), 2.00 (1H, d, J=11.4 Hz), 1.85-1.73 (2H, m), 1.64-1.24 (18H, m), 1.08 (3H, s), 0.99 (3H, s)

¹³C NMR (CDCl₃): 140.29, 117.60, 71.72, 69.91, 56.94, 52.76, 44.28, 43.62, 41.36, 40.39, 39.79, 36.97, 33.53, 22.78, 22.40, 21.88, 17.81, 13.73

| | | | | |
|---|---|---|---|---|
| MS HRES | Calculated for: | C₂₄H₃₂D₆F₆O₃ | [M + Na]⁺ | 517.2994 |
| | Observed: | | [M + Na]⁺ | 517.2997 |

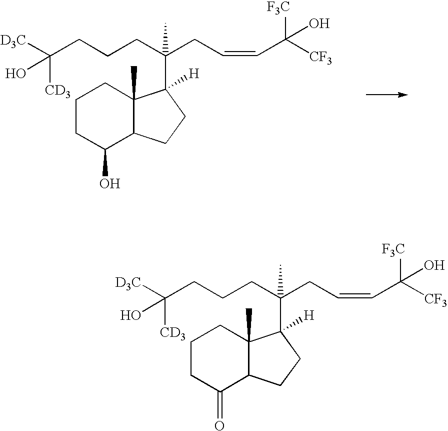

### (1R,3aR,7aR)-7a-Methyl-1-[(1S,3Z)-6,6,6-trifluororo-5-hydroxy-1-methyl-1-(5,5,5-trideutero-4-hydroxy-4-trideuteromethyl-pentyl)-5-trifluoromethyl-hex-3-enyl]-octahydro-inden-4-one

A 25 ml round bottom flask equipped with stir bar and Claisen adapter with rubber septum was charged with pyridinium dichromate (1.50 g, 3.99 mmol) and dichloromethane (15 ml). The (6S,3Z)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol (710 mg, 1.436 mmol) in dichloromethane (5 ml) was added dropwise and mixture was stirred in room temperature for 6 h.

The reaction mixture was filtrated through column with silica gel (50 cm³) using dichloromethane, dichloromethane:ethyl acetate 4:1, 3:1. The fractions containing product were pooled and evaporated to give oil (694 mg, 98%)

¹H NMR (CDCl₃): 6.10(1H, m), 5.52 (1H, d, J=12.4 Hz), 5.07 (1H, br s), 2.92 (1H, dd, J=16.1, 9.9 Hz), 2.48-2.38 (2H, m), 2.91-1.25 (18H, m), 0.99 (3H, s), 0.74 (3H, s)

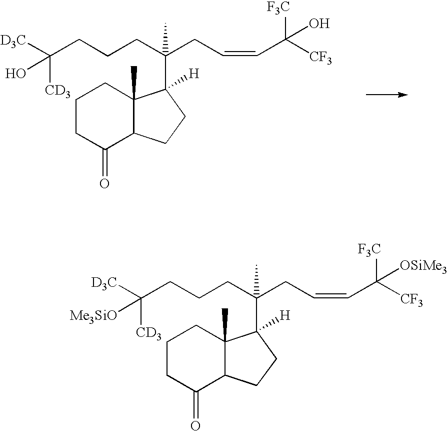

### (1R,3aR,7aR)-7a-Methyl-1-[(1S,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one

A 25 ml round bottom flask equipped with stir bar and Claisen adapter with rubber septum was charged with (1R,3aR,7aR)-7a-methyl-1-[(1S,3Z)-6,6,6-trifluororo-5-hydroxy-1-methyl-1-(5,5,5-trideutero-4-hydroxy-4-trideuteromethyl-pentyl)-5-trifluoromethyl-hex-3-enyl]-octahydro-inden-4-one (690 mg, 1.401 mmol) and dichloromethane (8 ml). 1-(Trimethylsilyl)imidazole (1.8 ml, 12.3 mmol) was added dropwise. The mixture was stirred at room temperature for 1.5 h. Ethyl acetate (150 ml) was added and the mixture was washed three times with water (50 ml), dried (Na₂SO₄) and evaporated. The oil residue was chromatographed on column (50 cm³) using hexane:ethyl acetate—10:1 as mobile phase. Fractions containing product were pooled and evaporated to give product as colorless oil (854 mg, 96%).

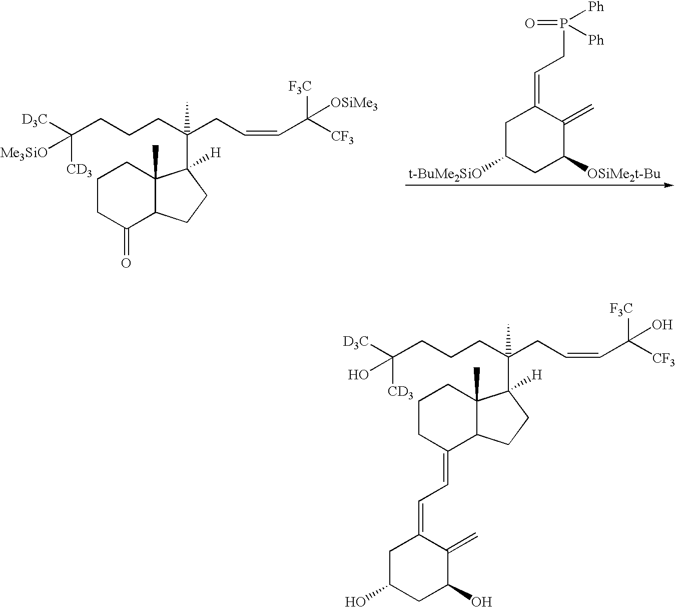

### 1,25-Dihydroxy-20S-20-(4-hydroxy-5,5,5-trideutero-4-trideuteromethyl-pentyl)-23Z-ene-26,27-hexafluorocholecalciferol

A 25 ml round bottom flask equipped with stir bar and Claisen adapter with rubber septum was charged with (1S,5R)-1,5-bis-((tert-butyldimethyl)silanyloxy)-3-[2-(diphenylfosphinoyl)-eth-(Z)-ylidene]-2-methylene-cyclohexane (539 mg, 0.925 mmol) and tetrahydrofuran (8 ml). The reaction mixture was cooled to −78° C. and n-butyllithium (0.58 ml, 0.93 mmol) was added dropwise. The resulting deep red solution was stirred at −78° C. for 20 min and (1R,3aR,7aR)-7a-methyl-1-[(1S,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (270 mg, 0.424 mmol) was added dropwise in tetrahydrofuran (1.5 ml). The reaction mixture was stirred for 4 h 30 min and then the bath was removed and the mixture was poured into ethyl acetate (50 ml) and saturated solution of ammonium chloride (60 ml). The water fraction was extracted three times with ethyl acetate (50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil (350 mg). A 25 ml round bottom flask equipped with stir bar and Claisen adapter with rubber septum was charged with oil and tetrabutylammonium fluoride (15 ml, 1M/tetrahydrofuran). The mixture was stirred for next 24 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give product as white foam (232 mg, 87%).

[α]_{D}²⁷=−5.4 (c=0.46, EtOH)

UV λmax (EtOH): 213 nm (ε 15177), 266 nm (ε 18553)

¹H NMR (DMSO-D6): 8.02 (1H, s), 6.19 (1H, d, J=11.3 Hz), 6.11 (1H, dt, J=12.1, 6.3 Hz), 5.98 (1H, d, J=11.1 Hz), 5.42 (1H, d, J=12.4 Hz), 5.23 (1H, s), 4.87 (1H, d, J=4.7 Hz), 4.76 (1H, s), 4.55 (1H, d, J=3.4 Hz), 4.20-4.17 (1H, m), 4.03 (1H, s), 3.98 (1H, br s), 2.82-2.75 (2H, m), 2.45 (1H, dd, J=16.6, 4.9 Hz), 2.36 (1H, d, J=11.9 Hz), 2.17 (1H, dd, J=13.04, 5.3 Hz), 2.04-1.95 (2H, m), 1.84-1.79 (1H, m), 1.73-1.54 (6H, m), 1.48-1.31 (4H, m), 1.22-1.17 (6H, m), 0.86 (3H, s), 0.61 (3H, s)

¹³C NMR (DMSO-D6): 149.41, 139.79, 139.46, 135.80, 122.95(q, J=186.7 Hz), 122.37, 117.85, 117.01, 109.75, 76.76(sep, J=28.9 Hz), 68.41, 68.37, 65.10, 56.45, 56.02, 51.21, 46.09, 44.87, 44.55, 43.12, 40.31, 39.37, 38.74, 35.68, 28.37, 23.21, 22.88, 21.81, 21.55, 17.60, 14.58

| | | | | |
|---|---|---|---|---|
| MS HRES | Calculated for: | C₃₃H₄₂D₆F₆O₄ | [M + Na]⁺ | 651.3725 |
| | Observed: | | [M + Na]⁺ | 651.3728 |

### Example 28

### Synthesis of 1,25-Dihydroxy-20S-20-(4-hydroxy-5,5,5-trideutero-4-trideuteromethyl-pentyl)-23Z-ene-26,27-hexafluoro-19-nor-cholecalciferol

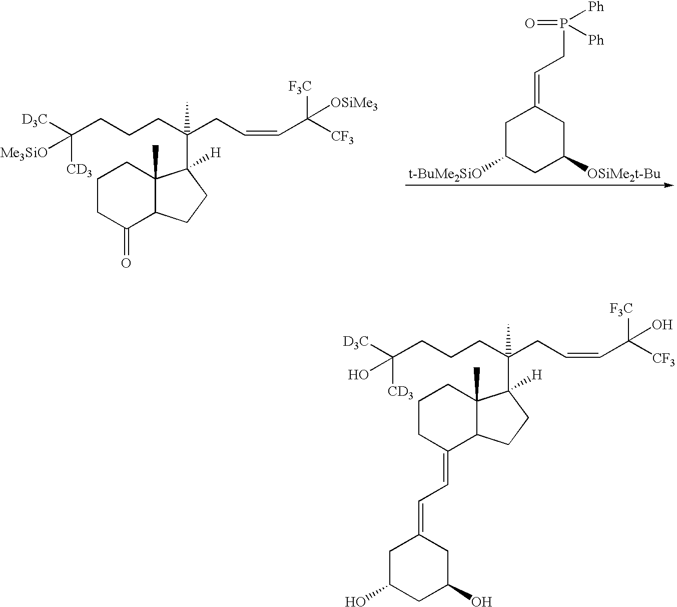

### 1,25-Dihydroxy-20S-20-(4-hydroxy-5,5,5-trideutero-4-trideuteromethyl-pentyl)-23Z-ene-26,27-hexafluoro-19-nor-cholecalciferol

A 25 ml round bottom flask equipped with stir bar and Claisen adapter with rubber septum was charged with (1R,3R)-1,3-bis-((tent-butyldimethyl)silanyloxy)-5-[2-(diphenylfosphinoyl)ethylidene]-cyclohexane (541 mg, 0.948 mmol) and tetrahydrofuran (8 ml). The reaction mixture was cooled to −78° C. and n-butyllithium (0.59 ml, 0.94 mmol) was added dropwise. The resulting deep red solution was stirred at −78° C. for 20 min and (1R,3aR,7aR)-7a-methyl-1-[(1S,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (286 mg, 0.449 mmol) was added dropwise in tetrahydrofuran (1.5 ml). The reaction mixture was stirred for 4 h 10 min and then the bath was removed and the mixture was poured into ethyl acetate (50 ml) and saturated solution of ammonium chloride (60 ml). The water fraction was extracted three times with ethyl acetate (50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil (390 mg).

A 25 ml round bottom flask equipped with stir bar and Claisen adapter with rubber septum was charged with oil and tetrabutylammonium fluoride (15 ml, 1M/ tetrahydrofuran). The mixture was stirred for next 30 h. The mixture was dissolved by the addition of ethyl acetate (150 ml) and extracted 6 times with water and brine (30 ml+20 ml), dried (Na₂SO₄) and evaporated. The oil residue was chromatographed on column (60 cm³, protected from light) using ethyl acetate as mobile phase. Fractions containing product were pooled and evaporated to give product as colorless oil. Oil was dissolved in methyl acetate and evaporated (4 times) to give product as white foam (264 mg, 95%).

[α]_{D}²⁶=+32.0 (c=0.47, EtOH)

UV λmax (EtOH): 244 nm (ε 31469), 252 nm (ε 36060), 262 nm (ε 24658)

¹H NMR (DMSO-D6): 8.02 (1H, s), 6.14-6.08 (1H, m), 6.08 (1H, d, J=11.9 Hz), 5.78 (1H, d, J=11.1 Hz), 5.43 (1H, d, J=12.2 Hz), 4.49 (1H, d, J=4.1 Hz), 4.39 (1H, d, J=4.1 Hz), 4.04 (1H, s), 3.88-3.78 (2H, m), 2.82-2.72 (2H, m), 2.48-2.42 (2H, m), 2.31-2.25 (1H, m), 2.07-1.90 (4H, m), 1.73-1.18 (17H, m), 0.87 (3H, s), 0.61 (3H, s)

¹³C NMR (DMSO-D6): 139.45, 139.19, 134.57, 122.94(q, J=286.8 Hz), 120.84, 117.02, 116.29, 76.75(sep, J=28.8 Hz), 68.41, 65.55, 65.27, 56.43, 55.98, 45.94, 44.60, 44.55, 42.23, 40.32, 39.38, 38.74, 36.97, 35.69, 28.21, 23.07, 22.89, 21.85, 21.44, 17.59, 14.69

| | | | | |
|---|---|---|---|---|
| MS HRES | Calculated for: | C₃₂H₄₂D₆F₆O₄ | [M + Na]⁺ | 639.3725 |
| | Observed: | | [M + Na]⁺ | 639.3724 |

### Example 29

### Synthesis of 1α-Fluoro-25-hydroxy-20S-20-(4-hydroxy-5,5,5-trideutero-4-trideuteromethyl-pentyl)-23Z-ene-26,27-hexafluorocholecalciferol

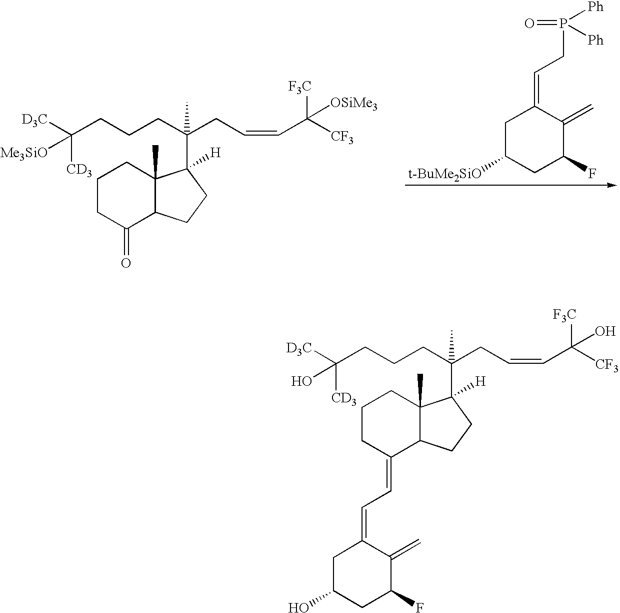

### 1α-Fluoro-25-hydroxy-20S-20-(4-hydroxy-5,5,5-trideutero-4-trideuteromethyl-pentyl)-23Z-ene-26,27-hexafluorocholecalciferol

A 25 ml round bottom flask equipped with stir bar and Claisen adapter with rubber septum was charged with (1S,5R)-1-((tert-butyldimethyl)silanyloxy)-3-[2-(diphenylfosphinoyl)-eth-(Z)-ylidene]-5-fluoro-2-methylene-cyclohexane (462 mg, 0.982 mmol) and tetrahydrofuran (8 ml). The reaction mixture was cooled to −78° C. and n-butyllithium (0.61 ml, 0.98 mmol)) was added dropwise. The resulting deep red solution was stirred at −78° C. for 20 min and (1R,3aR, 7aR)-7a-methyl-1-[(1S,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (267 mg, 0.419 mmol) was added dropwise in tetrahydrofuran (1.5 ml). The reaction mixture was stirred for 5 h and then the bath was removed and the mixture was poured into ethyl acetate (50 ml) and saturated solution of ammonium chloride (60 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil.

A 25 ml round bottom flask equipped with stir bar and Claisen adapter with rubber septum was charged with substrate and tetrabutylammonium fluoride (15 ml, 1M/tetrahydrofuran). The mixture was stirred for next 5 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using hexane:ethyl acetate (1:1) as mobile phase.

Product contained some impurities and was rechromatographed on column (VersaPak, 40×75 mm) using hexane:ethyl acetate (1:1) s mobile phase. Fractions containing product were pooled and evaporated to give product as colorless oil. Oil was dissolved in methyl acetate and evaporated (4 times) to give product as white foam (244 mg, 92%).

[α]_{D}²=+11.8 (c=0.51, EtOH)

UV λmax (EtOH): 244 nm (ε 15004), 270 nm (ε 15084)

¹H NMR (DMSO-D6): 8.02 (1H, s), 6.36 (1H, d, J=11.3 Hz), 6.14-6.07 (1H, m), 5.39 (1H, d, J=11.3 Hz), 5.42 (1H, d, J=11.9 Hz), 5.39 (1H, s), 5.14 (1H, br d, J=49.7 Hz), 4.99 (1H, d, J=1.7 Hz), 4.86 (1H, d, J=4.3 Hz), 4.03 (1H, s), 3.93-3.88 (1H, m), 2.82-2.74 (2H, m), 2.48-2.43 (2H, m), 2.17-1.97 (4H, m), 1.84-1.55 (6H, m), 1.46-1.32 (4H, m), 1.29-1.16 (7H, m), 0.86 (3H, s), 0.60 (3H, s)

¹³C NMR (DMSO-D6): 143.18(d, J=16.7 Hz), 141.74, 139.43, 132.93, 124.08, 122.95(q, J=286.7 Hz), 117.22, 117.01, 115.08(d, J=9.1 Hz), 91.93 (d, J=166.9 Hz), 76.76 (sep, J=28.0 Hz), 68.41, 64.56, 56.43, 55.96, 46.18, 44.82, 44.54, 40.69(d, J=20.5 Hz), 40.27, 38.73, 35.68, 28.38, 23.15, 22.85, 21.80, 21.45, 17.59, 14.61

| | | | | |
|---|---|---|---|---|
| MS HRES | Calculated for: | C₃₃H₄₁D₆F₇O₃ | [M + Na]⁺ | 653.3682 |
| | Observed: | | [M + Na]⁺ | 653.3689 |

### Example 30

### Synthesis of 1,25-Dihydroxy-20S-20-(4-hydroxy-5,5,5-trideutero-4-trideuteromethyl-pentyl)-23E-ene-26,27-hexafluorocholecalciferol

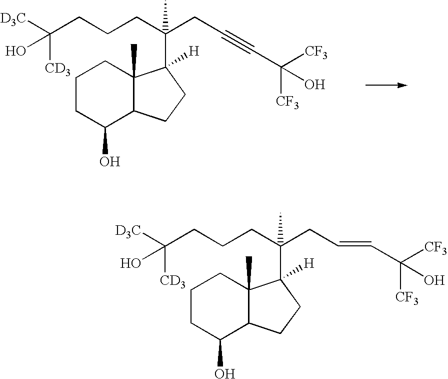

### (6S,3E)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol

A 25 ml round bottom flask equipped with stir bar and condenser with nitrogen sweep was charged with lithium aluminum hydride (12.0 ml, 12.0 mmol, 1M/tetrahydrofuran) and the mixture was cooled to 0° C. Sodium methoxide (648 mg, 12.0 mmol) was added slowly followed by (6S)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol (740 mg, 1.502 mmol) in tetrahydrofuran (8 ml). The reaction mixture was stirred at 80° C. for 4 h and then was cooled to 0° C. Saturated solution of ammonium chloride (5 ml) was added slowly followed by saturated solution of ammonium chloride (60 ml) and 2N HCl (20 ml). The mixture was extracted with ethyl acetate (3×50 ml), dried (Na₂SO₄) and evaporated. The oil residue was chromatographed on columns (50 cm³) using hexane:ethyl acetate—4:1 as mobile phase. Fractions containing product were pooled and evaporated to give colorless oil (727 mg, 98%).

[α]_{D}³⁰=−0.64 (c=0.47, EtOH)

¹H NMR (CDCl₃): 6.32(1H, dt, J=15.4, 7.9), 5.58 (1H, d, J=15.8 Hz), 4.09 (1H, br s), 2.29 (2H, d, J=8.1 Hz), 2.04-1.97 (1H, m), 1.84-1.76 (2H, m), 1.63-1.18 (18H, m), 1.09 (3H, s), 0.98 (3H, s)

¹³C NMR (CDCl₃): 137.23, 120.09, 71.53, 69.83, 57.36, 52.71, 44.27, 43.69, 42.44, 41.61, 40.22, 33.54, 23.20, 22.36, 21.88, 18.02, 17.70, 17.31, 16.77

| | | | | |
|---|---|---|---|---|
| MS HRES | Calculated for: | C₂₄H₃₂D₆F₆O₃ | [M + Na]⁺ | 517.2994 |
| | Observed: | | [M + Na]⁺ | 517.2994 |

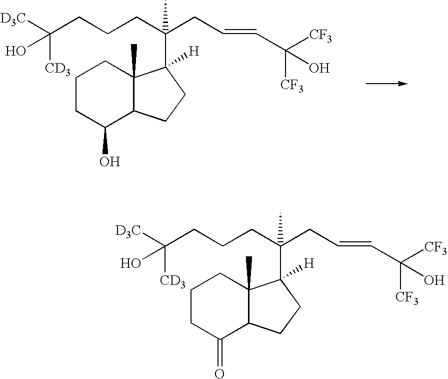

### (1R,3aR,7aR)-7a-Methyl-1-[(1S,3E)-6,6,6-trifluoro-5-hydroxy-1-methyl-1-(5,5,5-trideutero-4-hydroxy-4-trideuteromethyl-pentyl)-5-trifluoromethyl-hex-3-enyl]-octahydro-inden-4-one

A 25 ml round bottom flask equipped with stir bar and Claisen adapter with rubber septum was charged with pyridinium dichromate (1.50 g, 3.99 mmol) and dichloromethane (15 ml). The (6S,3E)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol (730 mg, 1.476 mmol) in dichloromethane (5 ml) was added dropwise and mixture was stirred in room temperature for 4.5 h.

The reaction mixture was filtrated through column with silica gel (50 cm³) using dichloromethane, dichloromethane:ethyl acetate 4:1. The fractions containing product were pooled and evaporated to give oil (706 mg, 97%).

[α]_{D}³⁰=−20.0 (c=0.46, EtOH)

¹H NMR (CDCl₃): 6.33(1H, dt, J=15.3, 7.7 Hz), 5.61 (1H, d, J=15.6 Hz), 2.43 (1H, dd, J=11.2, 7.1 Hz), 2.33-2.19 (4H, m), 2.17-2.12 (1H, m), 2.06-2.00 (1H, m), 1.95-1.84 ((1H, m), 1.80-1.54 (7H, m), 1.40-1.20 (5H, m), 1.15-1.09 (1H, m), 0.98 (3H, s), 0.75 (3H, s)

¹³C NMR (CDCl₃): 211.74, 136.54, 119.96, 71.25, 62.22, 57.49, 50.59, 43.80, 42.54, 40.85, 39.97, 39.80, 24.04, 23.03, 22.10, 18.67, 17.72, 15.71

| | | | | |
|---|---|---|---|---|
| MS HRES | Calculated for: | C₂₄H₃₀D₆F₆O₃ | [M + Na]⁺ | 515.2837 |
| | Observed: | | [M + Na]⁺ | 515.2837 |

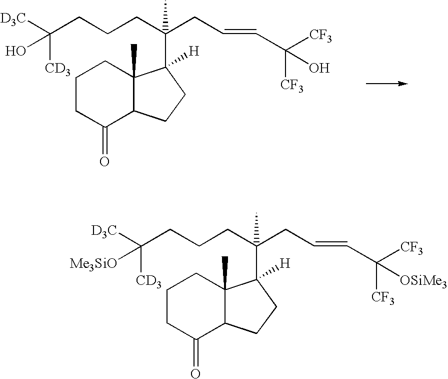

### (1R,3aR,7aR)-7a-Methyl-1-[(1S,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one

A 25 ml round bottom flask equipped with stir bar and Claisen adapter with rubber septum was charged with (1R,3aR,7aR)-7a-methyl-1-[(1S,3E)-6,6,6-trifluoro-5-hydroxy-1-methyl-1-(5,5,5-trideutero-4-hydroxy-4-trideuteromethyl-pentyl)-5-trifluoromethyl-hex-3-enyl]-octahydro-inden-4-one (698 mg, 1.417 mmol) and dichloromethane (8 ml). 1-(trimethylsilyl)imidazole (1.8 ml, 12.3 mmol) was added dropwise. The mixture was stirred at room temperature for 2 h. Ethyl acetate (150 ml) was added and the mixture was washed with water (4×50 ml), dried (Na₂SO₄) and evaporated. The oil residue was chromatographed on column (60 cm³) using hexane:ethyl acetate—10:1 as mobile phase. Fractions containing product were pooled and evaporated to give product as colorless oil (871 mg, 96%).

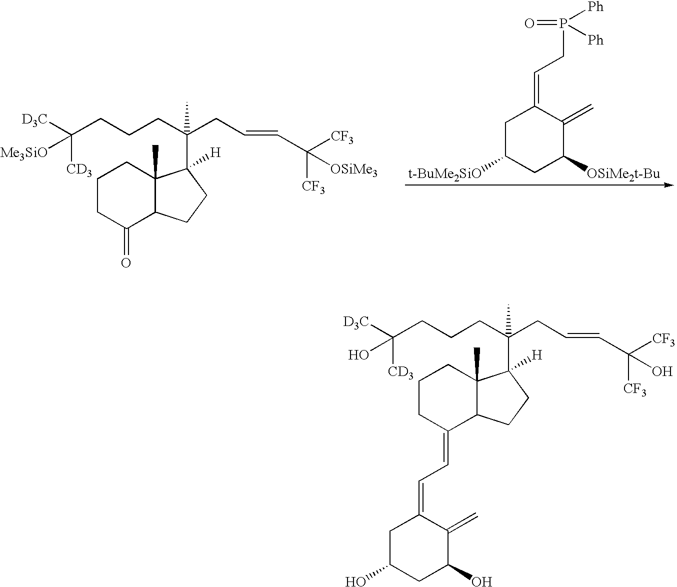

### 1,25-Dihydroxy-20S-20-(4-hydroxy-5,5,5-trideutero-4-trideuteromethyl-pentyl)-23E-ene-26,27-hexafluorocholecalciferol

A 25 ml round bottom flask equipped with stir bar and Claisen adapter with rubber septum was charged with (1S,5R)-1,5-bis-((tert-butyldimethyl)silanyloxy)-3-[2-(diphenylfosphinoyl)-eth-(Z)-ylidene]-2-methylene-cyclohexane (531 mg, 0.911 mmol) and tetrahydrofuran (8 ml). The reaction mixture was cooled to −78° C. and n-butyllithium (0.57 ml, 0.91 mmol)) was added dropwise. The resulting deep red solution was stirred at −78° C. for 20 min and (1R,3aR,7aR)-7a-methyl-1-[(1S,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (260 mg, 0.408 mmol) was added dropwise in tetrahydrofuran (1.5 ml). The reaction mixture was stirred for 5 h 30 min and then the bath was removed and the mixture was poured into ethyl acetate (50 ml) and saturated solution of ammonium chloride (60 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil. A 25 ml round bottom flask equipped with stir bar and Claisen adapter with rubber septum was charged with substrate and tetrahydrofuran (5 ml). Tetrabutylammonium fluoride (2.10 g, 6.66 mmol) was added. The mixture was stirred for next 6 h and tetrabutylammonium fluoride (5 ml, 1M/tetrahydrofuran) was added. The reaction was stirred for next 15 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give product as white foam (186 mg, 73%).

[α]_{D}³⁰=+4.5 (c=0.44, EtOH)

UV λmax (EtOH): 213 nm (ε 13978), 265 nm (ε 16276)

¹H NMR (CDCl₃): 6.37(1H, d, J=11.1 Hz), 6.31 (1H, dd, J=15.6, 7.9 Hz), 6.00 (1H, d, J=11.1 Hz), 5.59 (1H, d, J=15.6 Hz), 5.33 (1H, s), 4.99 (1H, s), 4.43 (1H, br s), 4.23 (1H, br s), 2.81 (1H, dd, J=12.2, 3.4 Hz), 2.59 (1H, br d, J=10.5 Hz), 2.34-2.29 (3H, m), 2.06-1.98 (3H, m), 1.93-1.87 (1H, m), 1.76-1.18 (18H, m), 1.12-1.06 (1H, m), 0.95 (3H, s), 0.66 (3H, s)

¹³C NMR (DMSO-D6): 149.41, 139.75, 136.73, 135.85, 122.63(q, J=285.2 Hz), 122.39, 119.72, 117.94, 109.79, 75.51(sep, J=29.6 Hz), 68.41, 65.11, 56.54, 56.02, 46.13, 44.87, 44.43, 43.11, 41.20, 40.48, 28.37, 23.14, 22.90, 21.72, 21.52, 17.56, 14.70

| | | | | |
|---|---|---|---|---|
| MS HRES | Calculated for: | C₃₃H₄₂D₆F₆O₄ | [M + Na]⁺ | 651.3725 |
| | Observed: | | [M + Na]⁺ | 651.3727 |

### Example 31

### Synthesis of 1,25-Dihydroxy-20S-20-(4-hydroxy-5,5,5-trideutero-4-trideuteromethyl-pentyl)-23E-ene-26,27-hexafluoro-19-nor-cholecalciferol

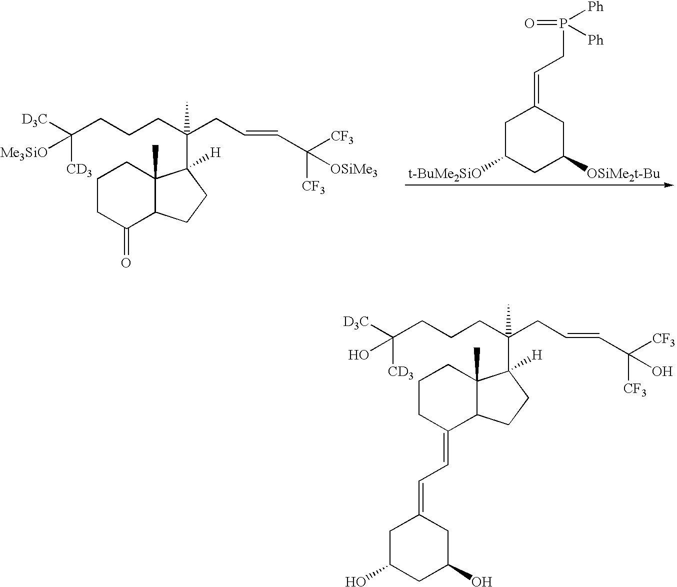

### 1,25-Dihydroxy-20S-20-(4-hydroxy-5,5,5-trideutero-4-trideuteromethyl-pentyl)-23E-ene-26,27-hexafluoro-19-nor-cholecalciferol

A 25 ml round bottom flask equipped with stir bar and Claisen adapter with rubber septum was charged with (1R,3R)-1,3-bis-((tert-butyldimethyl)silanyloxy)-5-[2-(diphenylfosphinoyl)ethylidene]-cyclohexane (546 mg, 0.956 mmol) and tetrahydrofuran (8 ml). The reaction mixture was cooled to −78° C. and n-butyllithium (0.60 ml, 0.96 mmol)) was added dropwise. The resulting deep red solution was stirred at −78° C. for 20 min and (1R,3aR,7aR)-7a-methyl-1-[(1S,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (295 mg, 0.463 mmol) was added dropwise in tetrahydrofuran (1.5 ml). The reaction mixture was stirred for 5 h 30 min and then the bath was removed and the mixture was poured into ethyl acetate (50 ml) and saturated solution of ammonium chloride (60 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil. A 25 ml round bottom flask equipped with stir bar and Claisen adapter with rubber septum was charged with substrate and tetrabutylammonium fluoride (15 ml, 1M/tetrahydrofuran). The mixture was stirred for next 42 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give product as white foam (280 mg, 98%).

[α]_{D}³⁰=+41.1 (c=0.46, EtOH)

UV λmax (EtOH): 244 nm (ε 32355), 252 nm (ε 37697), 262 nm (ε 25353)

¹H NMR (DMSO-D6): 8.04 (1H, s), 6.32 (1H, dt, J=15.6, 7.7 Hz), 6.07 (1H, d, J=11.1 Hz), 5.78 (1H, d, J=11.1 Hz), 5.63 (1H, d, J=15.3 Hz), 4.50 (1H, d, J=3.4 Hz), 4.39 (1H, d, J=3.4 Hz), 4.04 (1H, s), 3.88 (1H, br s), 3.80 (1H, br s), 2.74 (1H, br d, J=13.9 Hz), 2.44 (1H, dd, J=13.0, 3.0 Hz), 2.33-2.21 (2H, m), 2.07-1.95 (2H, m), 1.69-1.04 (17H, m), 0.90 (3H, s), 0.62 (3H, s)

¹³C NMR (DMSO-D6): 139.13, 136.71, 134.63, 122.44(q, J=285.2 Hz), 120.83, 119.71, 116.38, 75.51(sep, J=28.9 Hz), 68.37, 65.57, 65.28, 56.52, 55.97, 45.96, 44.59, 44.44, 42.23, 41.18, 40.48, 39.62, 39.58, 37.00, 28.19, 22.99, 22.91, 21.76, 21.42, 17.55, 14.79

| | | | | |
|---|---|---|---|---|
| MS HRES | Calculated for: | C₃₂H₄₂D₆F₆O₄ | [M + Na]⁺ | 639.3725 |
| | Observed: | | [M + Na]⁺ | 639.3724 |

### Example 32

### Synthesis of 1α-Fluoro-25-hydroxy-20S-20-(4-hydroxy-5,5,5-trideutero-4-trideuteromethyl-pentyl)-23E-ene-26,27-hexafluorocholecalciferol

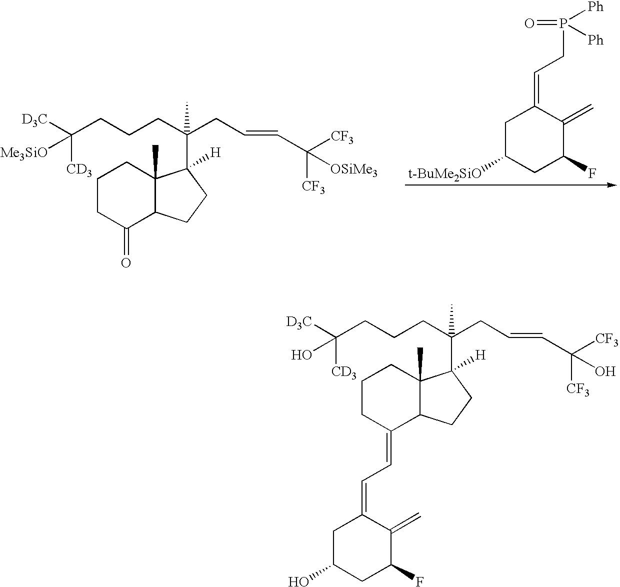

### 1α-Fluoro-25-hydroxy-20S-20-(4-hydroxy-5,5,5-trideutero-4-trideuteromethyl-pentyl)-23E-ene-26,27-hexafluorocholecalciferol

A 25 ml round bottom flask equipped with stir bar and Claisen adapter with rubber septum was charged with (1S,5R)-1-((tert-butyldimethyl)silanyloxy)-3-[2-(diphenylfosphinoyl)-eth-(Z)-ylidene]-5-fluoro-2-methylene-cyclohexane (473 mg, 1.005 mmol) and tetrahydrofuran (8 ml). The reaction mixture was cooled to −78° C. and n-butyllithium (0.63 ml, 1.01 mmol)) was added dropwise. The resulting deep red solution was stirred at −78° C. for 20 min and (1R,3aR, 7aR)-7a-methyl-1-[(1S,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (271 mg, 0.426 mmol) was added dropwise in tetrahydrofuran (1.5 ml). The reaction mixture was stirred for 4.5 h and then the bath was removed and the mixture was poured into ethyl acetate (50 ml) and saturated solution of ammonium chloride (60 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil.

A 25 ml round bottom flask equipped with stir bar and Claisen adapter with rubber septum was charged with substrate and tetrabutylammonium fluoride (10 ml, 1M/tetrahydrofuran). The mixture was stirred for next 17 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using hexane:ethyl acetate (1:1) as mobile phase. Fractions containing product were pooled and evaporated to give product as colorless oil. Oil was dissolved in methyl acetate and evaporated (4 times) to give product as white foam (226 mg, 84%).

[α]_{D}²⁸=+25.3 (c=0.45, EtOH)

UV λmax (EtOH): 243 nm (ε 14182), 269 nm (ε 14044)

¹H NMR (DMSO-D6): 8.03 (1H, s), 6.36 (1H, d, J=10.9 Hz), 6.33-6.27 (1H, m), 5.93 (1H, d, J=11.1 Hz), 5.63 (1H, d, J=15.4 Hz), 5.38 (1H, s), 5.14 (1H, br d, J=49.7 Hz), 4.99 (1H, s), 4.86 (1H, d, J=4.3 Hz), 4.03 (1H, s), 3.94-3.88 (1H, m), 2.81 (1H, br d, J=12.4 Hz), 2.34-2.20 (2H, m), 2.16-2.06 (2H, m), 2.00-1.95 (1H, m), 1.84-1.02 (18H, m), 0.89 (3H, s), 0.61 (3H, s)

¹³C NMR (DMSO-D6): 143.17(d, J=16.7 Hz), 141.68, 136.70, 132.97, 124.05, 122.62(q, J=286.7 Hz), 119.71, 117.29, 115.16, 91.95(d, J=166.9 Hz), 75.50 (sep, J=28.8 Hz), 68.36, 64.56, 56.51, 55.95, 46.19, 44.83, 44.42, 41.15, 40.69(d, J=20.5 Hz), 40.41, 39.61, 28.36, 23.06, 22.88, 21.70, 21.40, 17.54, 14.71

| | | | | |
|---|---|---|---|---|
| MS HRES | Calculated for: | C₃₃H₄₁D₆F₇O₃ | [M + Na]⁺ | 653.3682 |
| | Observed: | | [M + Na]⁺ | 653.3686 |

### Example 33

### Synthesis of 1,25-Dihydroxy-20R-20-(4-hydroxy-5,5,5-trideutero-4-trideuteromethyl-pentyl)-23-yne-26,27-hexafluorocholecalciferol

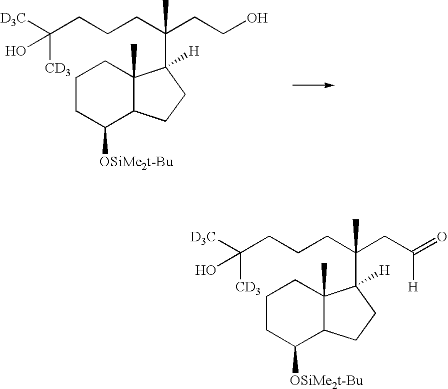

### (3R)-3-[(1R,3aR,4S,7aR)-4-(tert-Butyl-dimethyl-silanyloxy)-7a-methyl-octahydro-inden-1-yl]-8,8,8-trideutero-7-hydroxy-3-methyl-7-trideuteromethyl-octanal

A 250 ml round bottom flask equipped with stir bar and Claisen adapter with rubber septum was charged with pyridinium chlorochromate (3.858 g, 17.898 mmol), celite (3.93 g) and dichloromethane (70 ml). The (3R)-3-[(1R,3aR,4S,7aR)-4-(tert-butyl-dimethyl-silanyloxy)-7a-methyl-octahydro-inden-1-yl]-8,8,8-trideutero-3-methyl-7-trideuteromethyl-octane-1,7-diol (5.00 g, 11.190 mmol) in dichloromethane (10 ml) was added dropwise and mixture was stirred in room temperature for 3 h 45 min. The reaction mixture was filtrated through column with silica gel (250 cm³) and celite (1 cm) and using dichloromethane, dichloromethane:ethyl acetate 4:1. The fractions containing product were pooled and evaporated to give oil (4.42 g, 89%).

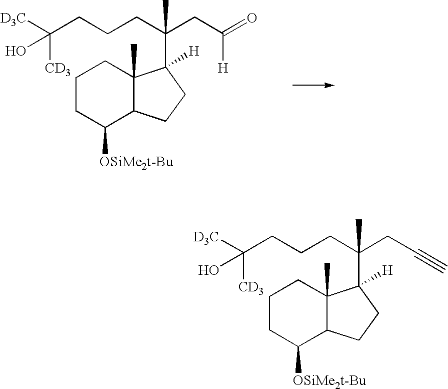

### (6R)-6-[(1R,3aR,4S,7aR)-4-(tert-Butyl-dimethyl-silanyloxy)-7a-methyl-octahydro-inden-1-yl]-1,1,1-trideutero-6-methyl-2-trideuteromethyl-non-8-yn-2-ol

A 250 ml round bottom flask equipped with stir bar and Claisen adapter with rubber septum was charged with (3R)-3-[(1R,3aR,4S,7aR)-4-(tert-butyl-dimethyl-silanyloxy)-7a-methyl-octahydro-inden-1-yl]-8,8,8-trideutero-7-hydroxy-3-methyl-7-trideuteromethyl-octanal (4.42 g, 9.937 mmol) and methanol (65 ml). 1-diazo-2-oxo-propyl)-phosphonic acid dimethyl ester (3.75 g, 19.52 mmol) in methanol (3 ml) was added and the resulting mixture was cooled in an ice bath. Potassium carbonate (3.75 g, 27.13 mmol) was added and the reaction mixture was stirred in the ice bath for 30 min and then at room temperature for 4 h. Water (100 ml) was added and the mixture was extracted with ethyl acetate (4×80 ml), dried (Na₂SO₄) and evaporated. The residue was filtrated through silica gel (50 cm³) using hexane:ethyl acetate—5:1 and evaporated.

The oil residue was chromatographed on column (VersaPak Cartridge 80×150 mm) using hexane:ethyl acetate—5:1 and 4:1 as mobile phase. Fractions containing product were pooled and evaporated to give product as colorless oil (3.83 g, 87%).

¹H NMR (CDCl₃): 3.99 (1H, br s), 2.12-1.92 (4H, m), 1.83-1.75 (1H, m), 1.68-1.22 (17H, m), 1.04 (3H, s), 0.99 (3H, s), 0.88 (9H, s), 0.00 (3H, s), −0.01(3H, s)

¹³C NMR (CDCl₃): 82.90, 70.75, 69.67, 69.60, 60.33, 56.61, 52.99, 44.73, 43.71, 41.35, 39.55, 39.51, 34.34, 29.51, 25.83, 22.77, 22.39, 22.03, 18.49, 18.03, 17.73, 16.48, 14.19, −4.79, −5.14

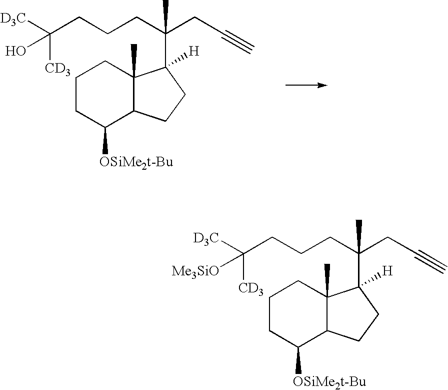

### (1R,3aR,4S,7aR)-4-(tert-Butyl-dimethyl-silanyloxy)-7a-methyl-1-[(1R)-6,6,6-trideutero-1-methyl-1-(prop-2-ynyl)-5-trideuteromethyl-5-trimethylsilanyloxy-hexyl]-octahydro-indene

A 100 ml round bottom flask equipped with stir bar and Claisen adapter with rubber septum was charged with (6R)-6-[(1R,3aR,4S,7aR)-4-(tert-butyl-dimethyl-silanyloxy)-7a-methyl-octahydro-inden-1-yl]-1,1,1-trideutero-6-methyl-2-trideuteromethyl-non-8-yn-2-ol (3.80 g, 8.62 mmol) and dichloromethane (30 ml). 1-(trimethylsilyl)imidazole (3.7 ml, 25.22 mmol) was added dropwise. The mixture was stirred at room temperature for 1 h 35 min. Water (100 ml) was added and the mixture was extracted with hexane (3×70 ml), dried (Na₂SO₄) and evaporated. The oil residue was chromatographed on column (250 cm³) using hexane:ethyl acetate—20:1 as mobile phase. Fractions containing product were pooled and evaporated to give product as colorless oil (4.09 g, 93%).

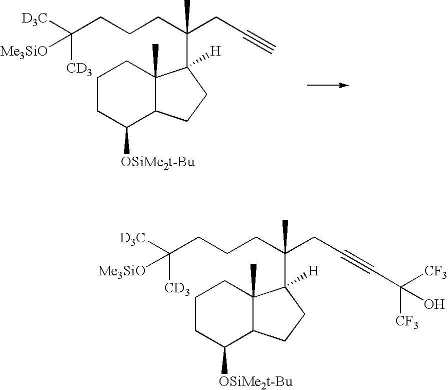

### (6R)-6-[(1R,3aR,4S,7aR)-4-(tert-Butyl-dimethyl-silanyloxy)-7a-methyl-octahydro-inden-1-yl]-6-methyl-11,11,11-trideutero-10-trideuteromethyl-1,1,1-trifluoro-2-trifluoromethyl-10-trimethylsilanyloxy-undec-3-yn-2-ol

A two neck 100 ml round bottom flask equipped with stir bar, Claisen adapter with rubber septum and funnel (with cooling bath) was charged with (1R,3aR,4S,7aR)-4-(tert-butyl-dimethyl-silanyloxy)-7a-methyl-1-[(1R)-6,6,6-trideutero-1-methyl-1-(prop-2-ynyl)-5-trideuteromethyl-5-trimethylsilanyloxy-hexyl]-octahydro-indene (4.09 g, 7.97 mmol) and tetrahydrofuran (50 ml). The funnel was connected to container with hexafluoroacetone and cooled (acetone, dry ice). The reaction mixture was cooled to −70° C. and n-butyllithium (7.5 ml, 12.00 mmol) was added dropwise. After 30 min hexafluoroacetone was added (the container's valve was opened three times). The reaction was steered at −70° C. for 2 h then saturated solution of ammonium chloride (5 ml) was added. The mixture was dissolved by the addition of saturated solution of ammonium chloride (100 ml) and extracted with ethyl acetate (3×80 ml), dried (Na₂SO₄) and evaporated. The residue was chromatographed twice on columns (300 cm³, hexane:ethyl acetate—20:1) to give the mixture of product and polymer (from hexafluoroacetone) (5.56 g). Product was used to the next reaction without purification.

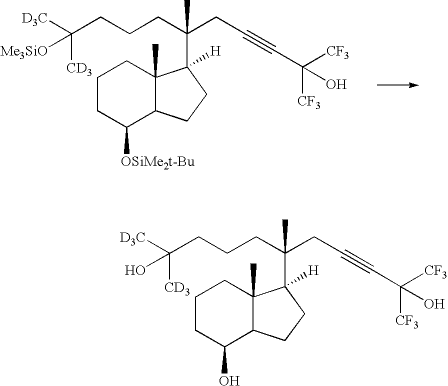

### (6R)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol

A 100 ml round bottom flask equipped with stir bar and rubber septum was charged with (6R)-6-[(1R,3aR,4S,7aR)-4-(tert-butyl-dimethyl-silanyloxy)-7a-methyl-octahydro-inden-1-yl]-6-methyl-11,11,11-trideutero-10-trideuteromethyl-1,1,1-trifluoro-2-trifluoromethyl-10-trimethylsilanyloxy-undec-3-yn-2-ol (5.56 g), acetonitrile (48 ml) and tetrahydrofuran (12 ml). A solution of H₂SiF₆ (35%) was added in small portion: 5 ml, 2 ml (after 1 h 20 min), 4 ml (after 50 min), 5 ml (after 1 h 40 min), 5 ml (after 1 h 30 min), 5 ml (after 16 h). After next 5 h the resulting mixture was diluted with water (50 ml) and poured into a mixture of ethyl acetate (50 ml) and water (50 ml). The organic phase was collected and the aqueous phase was re-extracted with ethyl acetate (2×50 ml). The combined organic layers were dried (Na₂SO₄) and evaporated. The oil residue was chromatographed on column (450 cm³) using dichloromethane:ethyl acetate (5:1) as mobile phase. The mixture fractions were purified on column (VersaPak Cartridge 40×150 mm) using hexane:ethyl acetate—2:1 and 1:1 as mobile phase. Fractions containing product were pooled and evaporated to give product (3.303 g, 84% two steps).

[α]_{D}³⁰=+1.4 (c=0.59, EtOH)

¹H NMR (CDCl₃): 4.09 (1H, br s), 2.16 (1H, AB, J=17.2 Hz), 2.23 (1H, AB, J=17.2 Hz), 2.05-2.01 (1H, m), 1.85-1.76 (2H, m), 1.65-1.21 (18H, m), 1.06 (3H, s), 1.01 (3H, s)

¹³C NMR (CDCl₃): 121.35(q, J=286.0 Hz), 90.34, 72.39, 71.06(sep, J=32.6 Hz), 69.48, 56.99, 52.48, 43.51, 43.13, 40.91, 40.39, 39.97, 33.35, 30.05, 22.54, 22.14, 21.92, 18.09, 17.47, 16.10

| | | | | |
|---|---|---|---|---|
| MS HRES | Calculated for: | C₂₄H₃₀D₆F₆O₃ | [M + Na]⁺ | 515.2837 |
| | Observed: | | [M + Na]⁺ | 515.2836 |

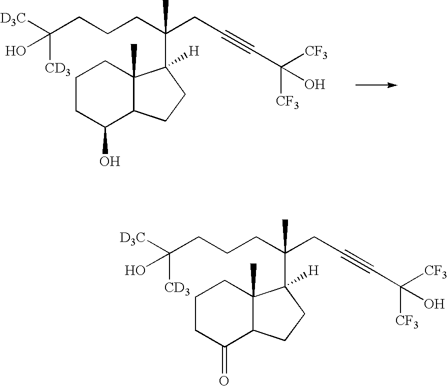

### (1R,3aR,7aR)-7a-Methyl-1-[(1R)-6,6,6-trifluororo-5-hydroxy-1-methyl-1-(5,5,5-trideutero-4-hydroxy-4-trideuteromethyl-pentyl)-5-trifluoromethyl-hex-3-ynyl]-octahydro-inden-4-one

A 25 ml round bottom flask equipped with stir bar and Claisen adapter with rubber septum was charged with pyridinium dichromate (1.620 g, 4.306 mmol) and dichloromethane (15 ml). The (6R)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol (783 mg, 1.583 mmol) in dichloromethane (2 ml) and DMF (0.5 ml) was added dropwise and mixture was stirred in room temperature for 5 h. The reaction mixture was filtrated through column with silica gel (50 cm³) using dichloromethane, dichloromethane:ethyl acetate 4:1. The fractions containing product were pooled and evaporated to give product as yellow oil. The oil residue was used to next reaction.

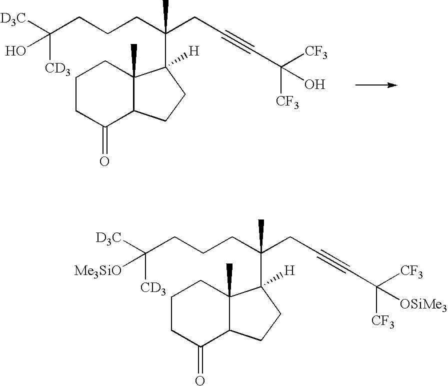

### (1R,3aR,7aR)-7a-Methyl-1-[(1R)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one

A 25 ml round bottom flask equipped with stir bar and Claisen adapter with rubber septum was charged with (1R,3aR,7aR)-7a-methyl-1-[(1R)-6,6,6-trifluororo-5-hydroxy-1-methyl-1-(5,5,5-trideutero-4-hydroxy-4-trideuteromethyl-pentyl)-5-trifluoromethyl-hex-3-ynyl]-octahydro-inden-4-one (ca. 1.58 mmol) and dichloromethane (8 ml). 1-(trimethylsilyl)imidazole (1.90 ml, 12.95 mmol) was added dropwise. The mixture was stirred at room temperature for 1.5 h. Hexane (150 ml) was added and the mixture was washed with water (3×50 ml), dried (Na₂SO₄) and evaporated.

The oil residue was chromatographed on column (50 cm³) using hexane:ethyl acetate—5:1 as mobile phase. Fractions containing product were pooled and evaporated to give product as colorless oil (918 mg, 95%).

[α]_{D}³⁰=−20.8 (c=0.61, DMSO)

¹H NMR (CDCl₃): 2.41 (1H, dd, J=11.3, 7.2 Hz), 2.31-2.12 (4H, m), 2.05-1.24 (15H, m), 1.00 (3H, s), 0.73 (3H, s), 0.27 (9H, s), 0.10 (9H, s)

| | | | | |
|---|---|---|---|---|
| MS HRES | Calculated for: | C₃₀H₄₄D₆F₆O₃Si₂ | [M + Na]⁺ | 657.3471 |
| | Observed: | | [M + Na]⁺ | 657.3467 |

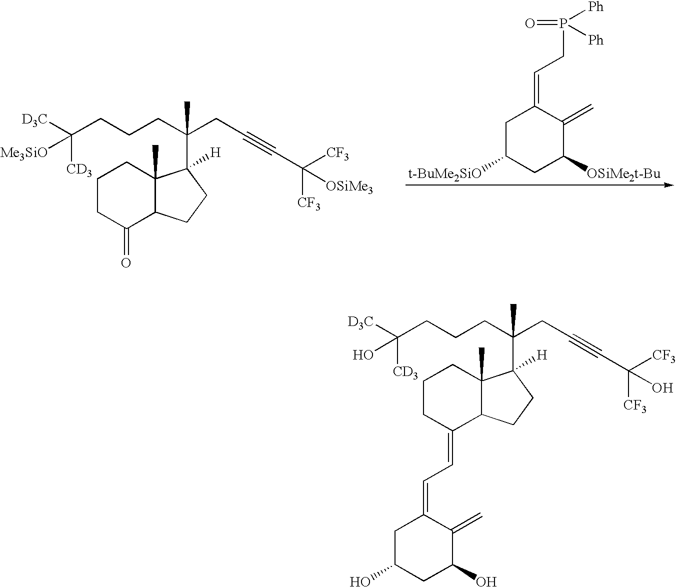

### 1,25-Dihydroxy-20R-20-(4-hydroxy-5,5,5-trideutero-4-trideuteromethyl-pentyl)-23-yne-26,27-hexafluorocholecalciferol

A 25 ml round bottom flask equipped with stir bar and Claisen adapter with rubber septum was charged with (1S,5R)-1,5-bis-((tert-butyldimethyl)silanyloxy)-3-[2-(diphenylfosphinoyl)-eth-(Z)-ylidene]-2-methylene-cyclohexane (500 mg, 0.858 mmol) and tetrahydrofuran (8 ml). The reaction mixture was cooled to −70° C. and n-butyllithium (0.53 ml, 0.85 mmol)) was added dropwise. The resulting deep red solution was stirred at −70° C. for 20 min and (1R,3aR,7aR)-7a-Methyl-1-[(1R)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one (314 mg, 0.495 mmol) was added dropwise in tetrahydrofuran (1.5 ml). The reaction mixture was stirred for 8 h (in last hour the temperature was increased from −70 do −50° C.). Saturated solution of ammonium chloride (1 ml) was added and the bath was removed. The mixture was poured into ethyl acetate (50 ml) and saturated solution of ammonium chloride (50 ml). The water fraction was extracted with ethyl acetate (3×60 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give oil.

A 25 ml round bottom flask equipped with stir bar and Claisen adapter with rubber septum was charged with substrate and tetrabutylammonium fluoride (10 ml, 1M/tetrahydrofuran). The mixture was stirred for next 41 h. The mixture was dissolved by the addition of ethyl acetate (150 ml) and extracted 6 times with water and brine (30 ml+20 ml), dried (Na₂SO₄) and evaporated. The oil residue was chromatographed on column (70 cm³, protected from light) using ethyl acetate as mobile phase. Fraction containing impurity was chromatographed on next column (70 cm³, protected from light) using ethyl acetate as mobile phase. Fractions containing product were pooled and evaporated to give product as colorless oil. Oil was dissolved in methyl acetate and evaporated (4 times) to give product as white foam (198 mg, 64%).

[α]_{D}²³=+11.0 (c=0.50, EtOH)

UV λmax (EtOH): 213 nm (ε 17873), 264 nm (ε 20804)

¹H NMR (DMSO-D6): 8.95 (1H, s), 6.19 (1H, d, J=11.3 Hz), 5.97 (1H, d, J=11.3 Hz), 5.22 (1H, s), 4.86 (1H, d, J=4.9 Hz), 4.75 (1H, d, J=1.9 Hz), 4.55 (1H, d, J=3.8 Hz), 4.20-4.18 (1H, m), 4.04 (1H, s), 4.01-3.98 (1H, m), 2.78 (1H, d, J=13.6 Hz), 2.35 (1H, d, J=13.4 Hz), 2.28-2.14 (3H, m), 1.99-1.92 (2H, m), 1.83-1.78 (2H, m), 1.64-1.57 (5H, m), 1.47-1.21 (10H, m), 0.96 (3H, s), 0.60 (3H, s)

¹³C NMR (DMSO-D6): 149.56, 139.66, 136.09, 122.45, 121.61(q, J=286.7 Hz), 118.13, 109.87, 89.59, 70.67, 70.46(sep, J=31.9 Hz), 68.48, 68.42, 65.13, 56.05, 55.96, 46.09, 44.88, 44.55, 43.13, 40.12, 38.88, 28.77, 28.31, 23.03, 22.37, 21.89, 21.51, 18.21, 14.25

| | | | | |
|---|---|---|---|---|
| MS HRES | Calculated for: | C₃₃H₄₀D₆F₆O₄ | [M + Na]⁺ | 649.3569 |
| | Observed: | | [M + Na]⁺ | 649.3569 |

### Example 34

### Synthesis of 1,25-Dihydroxy-20R-20-(4-hydroxy-5,5,5-trideutero-4-trideuteromethyl-pentyl)-23-yne-26,27-hexafluoro-19-nor-cholecalciferol

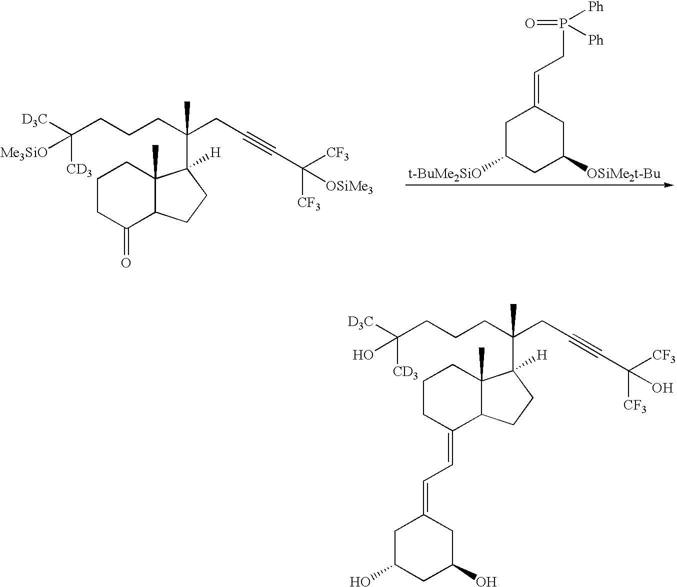

### 1,25-Dihydroxy-20R-20-(4-hydroxy-5,5,5-trideutero-4-trideuteromethyl-pentyl)-23-yne-26,27-hexafluoro-19-nor-cholecalciferol

A 25 ml round bottom flask equipped with stir bar and Claisen adapter with rubber septum was charged with (1R,3R)-1,3-bis-((tert-butyldimethyl)silanyloxy)-5-[2-(diphenylfosphinoyl)ethylidene]-cyclohexane (568 mg, 0.995 mmol) and tetrahydrofuran (8 ml). The reaction mixture was cooled to −70° C. and n-butyllithium (0.62 ml, 0.99 mmol) was added dropwise. The resulting deep red solution was stirred at −70° C. for 20 min and (1R,3aR,7aR)-7a-Methyl-1-[(1R)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one (306 mg, 0.482 mmol) was added dropwise in tetrahydrofuran (1.5 ml). The reaction mixture was stirred for 6 h and then saturated solution of ammonium chloride (1 ml) was added and the bath was removed. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give oil. A 25 ml round bottom flask equipped with stir bar and Claisen adapter with rubber septum was charged with substrate and tetrabutylammonium fluoride (15 ml, 1M/tetrahydrofuran). The mixture was stirred for next 96 h.

The mixture was dissolved by the addition of ethyl acetate (150 ml) and extracted 6 times with water and brine (30 ml+20 ml), dried (Na₂SO₄) and evaporated. The oil residue was chromatographed on column (60 cm³, protected from light) using ethyl acetate as mobile phase. Fractions containing product were pooled and evaporated to give product as colorless oil. Oil was dissolved in methyl acetate and evaporated (4 times) to give product as white foam (223 mg, 75%).

[α]_{D}²⁷=+45.5 (c=0.42, EtOH)

UV λmax (EtOH): 244 nm (ε 36685), 252 nm (ε 42933), 262 nm (ε 28904)

¹H NMR (DMSO-D6): 8.95 (1H, s), 6.07 (1H, d, J=11.1 Hz), 5.78 (1H, d, J=11.1 Hz), 4.48 (1H, d, J=4.3 Hz), 4.38 (1H, d, J=3.8 Hz), 4.04 (1H, s), 3.90-3.76 (2H, m), 2.74 (1H, d, J=13.4 Hz), 2.43 (1H, d, J=14.1 Hz), 2.28-2.19 (3H, m), 2.07-1.93 (3H, m), 1.81 (1H, dd, J=9.6, 9.2 Hz), 1.68-1.22 (17H, m), 0.96 (3H, s), 0.59 (3H, s)

¹³C NMR (DMSO-D6): 139.10, 134.88, 121.61(q, J=286.7 Hz), 120.92, 116.57, 89.60, 70.67, 68.49, 65.60, 65.32, 56.01, 55.94, 45.94, 44.60, 44.55, 42.23, 39.80, 36.96, 28.80, 28.15, 22.89, 22.39, 21.94, 21.42, 18.22, 14.37

| | | | | |
|---|---|---|---|---|
| MS HRES | Calculated for: | C₃₂H₄₀D₆F₆O₄ | [M + Na]⁺ | 637.3569 |
| | Observed: | | [M + Na]⁺ | 637.3565 |

### Example 35

### Synthesis of 1α-Fluoro-25-hydroxy-20R-20-(4-hydroxy-5,5,5-trideutero-4-trideuteromethyl-pentyl)-23-yne-26,27-hexafluorocholecalciferol

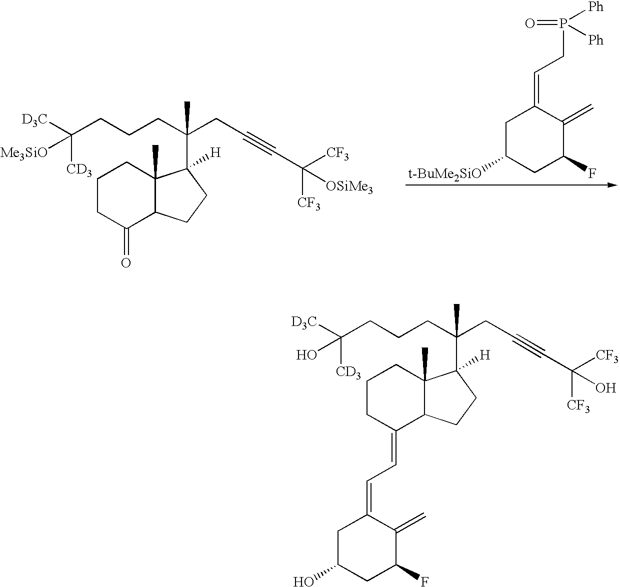

### 1α-Fluoro-25-hydroxy-20R-20-(4-hydroxy-5,5,5-trideutero-4-trideuteromethyl-pentyl)-23-yne-26,27-hexafluorocholecalciferol

A 25 ml round bottom flask equipped with stir bar and Claisen adapter with rubber septum was charged with (1S,5R)-1-((tert-butyldimethyl)silanyloxy)-3-[2-(diphenylfosphinoyl)-eth-(Z)-ylidene]-5-fluoro-2-methylene-cyclohexane] (542 mg, 1.152 mmol) and tetrahydrofuran (8 ml). The reaction mixture was cooled to −70° C. and n-butyllithium (0.71 ml, 1.14 mmol) was added dropwise. The resulting deep red solution was stirred at −70° C. for 20 min and (1R,3aR, 7aR)-7a-Methyl-1-[(1R)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one (292 mg, 0.460 mmol) was added dropwise in tetrahydrofuran (1.5 ml).). The reaction mixture was stirred for 7 h (in last hour the temperature was increased from −70 do −50° C.). The bath was removed and the mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product were pooled and evaporated to give oil. The oil residue was used to next reaction. A 25 ml round bottom flask equipped with stir bar and Claisen adapter with rubber septum was charged with substrate and tetrabutylammonium fluoride (8 ml, 1M/tetrahydrofuran). The mixture was stirred for next 48 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using hexane:ethyl acetate—1:1 as mobile phase. Fractions containing product were pooled and evaporated to give product as colorless oil. Oil was dissolved in methyl acetate and evaporated (4 times) to give product as white foam (278 mg, 96%).

[α]_{D}²⁷=+26.4 (c=0.50, EtOH)

UV λmax (EtOH): 210 nm (ε 14823), 244 nm (ε 14731), 270 nm (ε 14798)

¹H NMR (DMSO-D6): 8.95 (1H, s), 6.36 (1H, d, J=11.1 Hz), 5.93 (1H, d, J=11.3 Hz), 5.38 (1H, s), 5.14 (1H, br d, J=49.6 Hz), 4.98 (1H, d, J=1.9 Hz), 4.86 (1H, d, J=4.5 Hz), 4.04 (1H, s), 3.94-3.87 (1H, m), 2.82 (1H, d, J=10.2 Hz), 2.27-2.05 (4H, m), 2.00-1.93 (2H, m), 1.83-1.55 (7H, m), 1.48-1.21 (10H, m), 0.95 (3H, s), 0.58 (3H, s)

¹³C NMR (DMSO-D6): 143.31(d, J=16.7 Hz), 141.67, 133.23(d, J=1.5 Hz), 124.18, 121.64(q, J=286.0 Hz), 117.53, 115.37(d, J=9.2 Hz), 92.09 (167.6 Hz), 89.59, 70.70, 70.48(sep, J=31.9 Hz), 68.51, 64.61, 64.57, 56.02, 55.96, 46.19, 44.86, 44.56, 40.71(d, J=19.7 Hz), 39.82, 28.80, 28.34, 22.98, 22.35, 21.90, 21.43, 18.24, 14.31

| | | | | |
|---|---|---|---|---|
| MS HRES | Calculated for: | C₃₃H₃₉D₆F₇O₃ | [M + Na]⁺ | 651.3526 |
| | Observed: | | [M + Na]⁺ | 651.3530 |

### Example 36

### Synthesis of 1,25-Dihydroxy-20R-20-(4-hydroxy-5,5,5-trideutero-4-trideuteromethyl-pentyl)-23Z-ene-26,27-hexafluorocholecalciferol

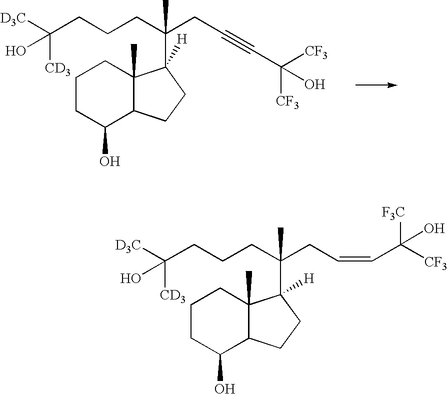

### (6R,3Z)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol

A 50 ml round bottom flask was charged with (6R)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol (800 mg, 1.624 mmol), Pd/CaCO₃ (200 mg, 5%), hexane (18.6 ml), ethyl acetate (7.6 ml) and solution of quinoline in ethanol (0.72 ml, prepared from ethanol (3.1 ml) and quinoline (168 μl)). The substrate was hydrogenated at ambient temperature and atmospheric pressure of hydrogen. The reaction was monitoring by TLC (dichloromethane:ethyl acetate 4:1, 3×). After 5 h 10 min the catalyst was filtered off (silica gel 50 cm³, hexane:ethyl acetate 1:1) and solvent evaporated. Product was crystallized from hexane:ethyl acetate (750 mg, 93%).

[α]_{D}³⁰=−2.34 (c=0.47, EtOH)

¹H NMR (CDCl₃): 6.07(1H, dt, J=12.4, 7.2 Hz), 5.45 (1H, d, J=12.4 Hz), 4.08 (1H, d, J=2.1 Hz), 2.50-2.39 (2H, m), 2.03 (1H, d, J=11.1 Hz), 1.88-1.79 (2H, m), 1.67-1.22 (18H, m), 1.09 (3H, s), 0.98 (3H, s)

¹³C NMR (CDCl₃): 139.98, 122.83(q, J=286.7 Hz), 117.24, 71.45, 69.57, 56.67, 52.55, 44.08, 43.56, 41.21, 39.71, 39.13, 37.19, 33.39, 22.42, 22.15, 21.86, 17.92, 17.54, 16.47

| | | | | |
|---|---|---|---|---|
| MS HRES | Calculated for: | C₂₄H₃₂D₆F₆O₃ | [M + Na]⁺ | 517.2994 |
| | Observed: | | [M + Na]⁺ | 517.2992 |

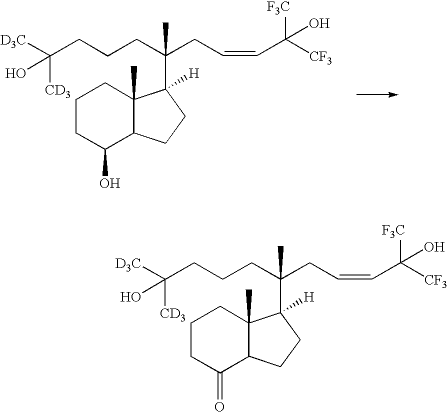

### (1R,3aR,7aR)-7a-Methyl-1-[(1R,3Z)-6,6,6-trifluororo-5-hydroxy-1-methyl-1-(5,5,5-trideutero-4-hydroxy-4-trideuteromethyl-pentyl)-5-trifluoromethyl-hex-3-enyl]-octahydro-inden-4-one

A 50 ml round bottom flask equipped with stir bar and Claisen adapter with rubber septum was charged with pyridinium dichromate (1.520 g, 4.040 mmol) and dichloromethane (20 ml). The (6R,3Z)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol (730 mg, 1.476 mmol) in dichloromethane (5 ml) was added dropwise and mixture was stirred in room temperature for 4 h 20 min.

The reaction mixture was filtrated through column with silica gel (50 cm³) using dichloromethane, dichloromethane:ethyl acetate 4:1. The fractions containing product were pooled and evaporated. The product was used to the next reaction without purification.

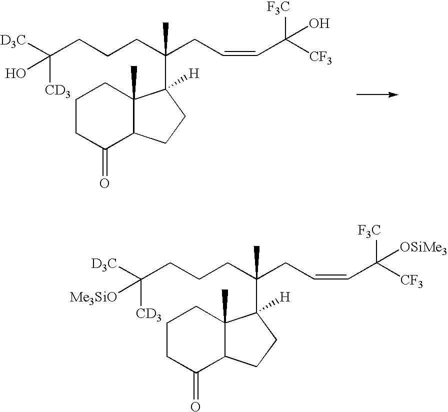

### (1R,3aR,7aR)-7a-Methyl-1-[(1R,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one

A 25 ml round bottom flask equipped with stir bar and Claisen adapter with rubber septum was charged with (1R,3aR,7aR)-7a-methyl-1-[(1R,3Z)-6,6,6-trifluororo-5-hydroxy-1-methyl-1-(5,5,5-trideutero-4-hydroxy-4-trideuteromethyl-pentyl)-5-trifluoromethyl-hex-3-enyl]-octahydro-inden-4-one (ca. 1.47 mmol) and dichloromethane (8 ml). 1-(trimethylsilyl)imidazole (1.80 ml, 12.27 mmol) was added dropwise. The mixture was stirred at room temperature for 3 h. Water (50 ml) was added and the mixture was extracted with ethyl acetate (3×50 ml), dried (Na₂SO₄) and evaporated. The oil residue was chromatographed on column (75 cm³) using hexane:ethyl acetate—5:1 as mobile phase. Fractions containing product were pooled and evaporated to give product as colorless oil (766 mg, 81%)

¹H NMR (CDCl₃): 5.98 (1H, dt, J=12.5, 6.2 Hz), 5.42 (1H, d, J=11.4 Hz), 2.49-2.40 (2H, m), 2.34-2.15 (4H, m), 2.07-1.95 (1H, m), 1.93-1.60 (6H, m), 1.43-1.19 (7H, m), 0.95 (3H, s), 0.74 (3H, s), 0.24 (9H, s), 0.10 (9H, s)

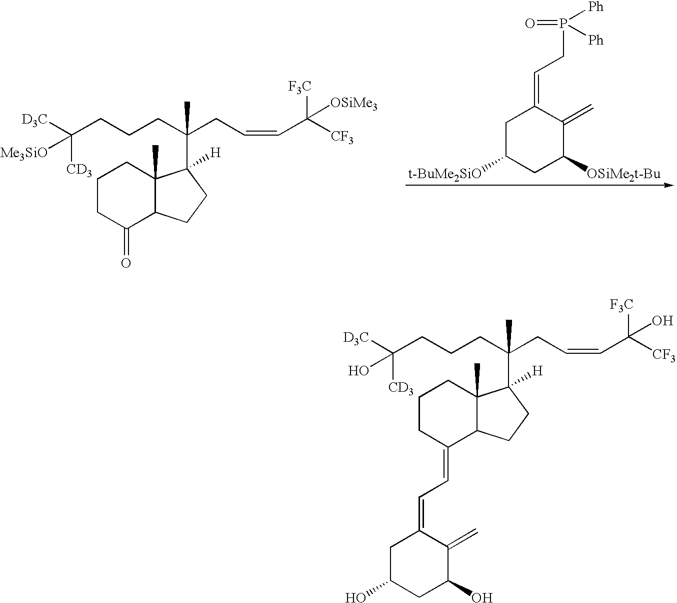

### 1,25-Dihydroxy-20R-20-(4-hydroxy-5,5,5-trideutero-4-trideuteromethyl-pentyl)-23Z-ene-26,27-hexafluorocholecalciferol

A 25 ml round bottom flask equipped with stir bar and Claisen adapter with rubber septum was charged with (1S,5R)-1,5-bis-((tert-butyldimethyl)silanyloxy)-3-[2-(diphenylfosphinoyl)-eth-(Z)-ylidene]-2-methylene-cyclohexane (473 mg, 0.811 mmol) and tetrahydrofuran (8 ml). The reaction mixture was cooled to −70° C. and n-butyllithium (0.50 ml, 0.80 mmol)) was added dropwise. The resulting deep red solution was stirred at −70° C. for 20 min and (1R,3aR,7aR)-7a-methyl-1-[(1R,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (280 mg, 0.440 mmol) was added dropwise in tetrahydrofuran (1.5 ml). The reaction mixture was stirred for 6 h (in last hour the temperature was increased from −70 do −50° C.). Saturated solution of ammonium chloride (1 ml) was added and the bath was removed. The mixture was poured into ethyl acetate (50 ml) and saturated solution of ammonium chloride (100 ml). The water fraction was extracted with ethyl acetate (3×70 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil.

A 25 ml round bottom flask equipped with stir bar and Claisen adapter with rubber septum was charged with substrate and tetrabutylammonium fluoride (15 ml, 1M/tetrahydrofuran). The mixture was stirred for next 29 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using hexane:ethyl acetate—1:2 as mobile phase. Fractions containing product were pooled and evaporated to give product as colorless oil. Oil was dissolved in methyl acetate and evaporated (4 times) to give product as white foam (224 mg, 81%).

[α]_{D}²⁹=+7.5 (c=0.48, EtOH)

UV λmax (EtOH): 213 nm (ε 15024), 265 nm (ε 17330)

¹H NMR (DMSO-D6): 7.98 (1H, s), 6.18 (1H, d, J=11.1 Hz), 6.10 (1H, dt, J=12.8, 6.4 Hz), 5.97 (1H, d, J=11.3 Hz), 5.43 (1H, d, J=11.9 Hz), 5.23 (1H, s), 4.86 (1H, d, J=4.7 Hz), 4.75 (1H, d, J=1.7 Hz), 4.54 (1H, d, J=3.6 Hz), 4.21-4.16 (1H, m), 4.02 (1H, s), 4.05-3.95 (1H, m), 2.77 (1H, d, J=11.7 Hz), 2.50-2.29 (2H, m), 2.16 (1H, dd, J=13.5, 5.2 Hz), 2.00-1.94 (2H, m), 1.82-1.78 (1H, m), 1.71-1.25 (17H, m), 0.90 (3H, s), 0.61 (3H, s)

¹³C NMR (DMSO-D6): 149.40, 139.76, 139.25, 135.81, 122.93(q, J=287.5 Hz), 122.35, 117.88, 117.11, 109.75, 76.78(sep, J=29.6 Hz), 68.41, 68.35, 65.07, 56.55, 55.98, 46.15, 44.86, 44.59, 43.11, 40.34, 38.76, 36.05, 28.98, 23.13, 22.80, 21.83, 29.50, 20.07, 17.93, 14.57

| | | | | |
|---|---|---|---|---|
| MS HRES | Calculated for: | C₃₃H₄₂D₆F₆O₄ | [M + Na]⁺ | 651.3725 |
| | Observed: | | [M + Na]⁺ | 651.3726 |

### Example 37

### Synthesis of 1,25-Dihydroxy-20R-20-(4-hydroxy-5,5,5-trideutero-4-trideuteromethyl-pentyl)-23Z-ene-26,27-hexafluoro-19-nor-cholecalciferol

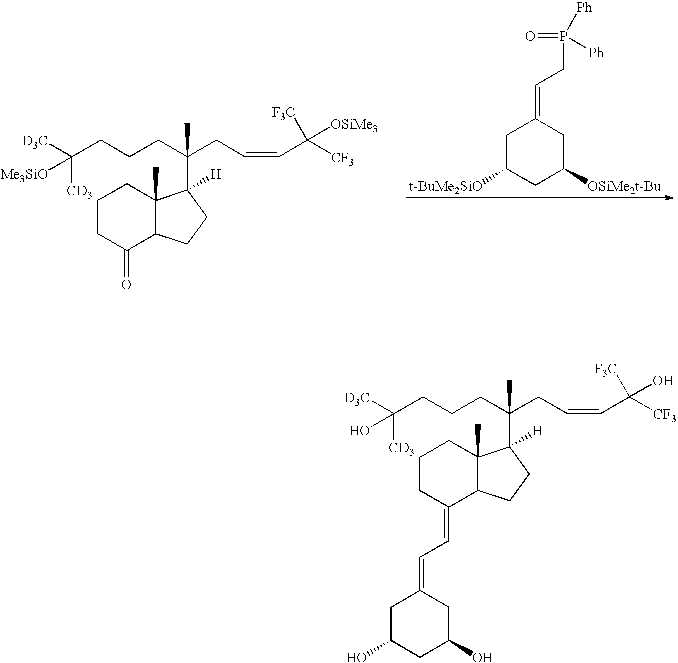

### 1,25-Dihydroxy-20R-20-(4-hydroxy-5,5,5-trideutero-4-trideuteromethyl-pentyl)-23Z-ene-26,27-hexafluoro-19-nor-cholecalciferol

A 25 ml round bottom flask equipped with stir bar and Claisen adapter with rubber septum was charged with (1R,3R)-1,3-bis-((tert-butyldimethyl)silanyloxy)-5-[2-(diphenylfosphinoyl)ethylidene]-cyclohexane (575 mg, 1.007 mmol) and tetrahydrofuran (8 ml). The reaction mixture was cooled to −70° C. and n-butyllithium (0.61 ml, 0.98 mmol)) was added dropwise. The resulting deep red solution was stirred at −70° C. for 20 min and (1R,3aR,7aR)-7a-methyl-1-[(1R,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (303 mg, 0.476 mmol) was added dropwise in tetrahydrofuran (1.5 ml). The reaction mixture was stirred for 5 h and then saturated solution of ammonium chloride (1 ml) was added and the bath was removed. The mixture was poured into ethyl acetate (50 ml) and saturated solution of ammonium chloride (100 ml). The water fraction was extracted with ethyl acetate (3×70 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil. A 25 ml round bottom flask equipped with stir bar and Claisen adapter with rubber septum was charged with substrate and tetrabutylammonium fluoride (15 ml, 1M/tetrahydrofuran). The mixture was stirred for next 64 h. The mixture was dissolved by the addition of ethyl acetate (150 ml) and extracted 6 times with water and brine (30 ml+20 ml), dried (Na₂SO₄) and evaporated. The oil residue was chromatographed on column (60 cm³, protected from light) using ethyl acetate as mobile phase. Fractions containing product were pooled and evaporated to give product as colorless oil. Oil was dissolved in methyl acetate and evaporated (4 times) to give product as white foam (251 mg, 85%).

[α]_{D}²⁹=+44.3 (c=0.42, EtOH)

UV λmax (EtOH): 244 nm (ε 36100), 252 nm (ε 42319), 262 nm (ε 28518)

¹H NMR (DMSO-D6): 7.99 (1H, s), 6.14-6.06 (1H, m), 6.07 (1H, d, J=12.4 Hz), 5.78 (1H, d, J=11.3 Hz), 5.43 (1H, d, J=12.2 Hz), 4.48 (1H, d, J=4.0 Hz), 4.38 (1H, d, J=4.1 Hz), 4.02 (1H, s), 3.90-3.84 (1H, m), 3.84-3.76 (1H, m), 2.73 (1H, d, J=13.6 Hz), 2.54-2.41 (2H, m), 2.26 (1H, br d, J=10.4 Hz), 2.07-1.97 (3H, m), 1.72-1.18 (19H, m), 0.90 (3H, s), 0.60 (3H, s)

¹³C NMR (DMSO-D6): 139.25, 139.18, 134.60, 122.94(q, J=286.8 Hz), 120.82, 117.13, 116.33, 76.77(sep, J=28.0 Hz), 68.41, 65.54, 65.26, 56.53, 55.95, 46.00, 44.59, 42.22, 40.34, 38.78, 36.96, 36.07, 28.17, 22.99, 22.80, 21.89, 21.40, 17.94, 14.67

| | | | | |
|---|---|---|---|---|
| MS HRES | Calculated for: | C₃₂H₄₂D₆F₆O₄ | [M + Na]⁺ | 639.3725 |
| | Observed: | | [M + Na]⁺ | 639.3717 |

### Example 38

### Synthesis of 1α-Fluoro-25-hydroxy-20R-20-(4-hydroxy-5,5,5-trideutero-4-trideuteromethyl-pentyl)-23Z-ene-26,27-hexafluorocholecalciferol

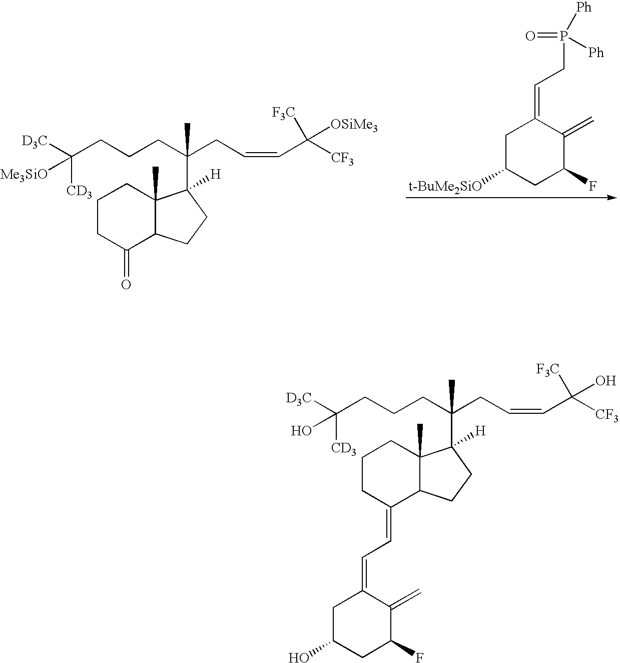

### 1α-Fluoro-25-hydroxy-20R-20-(4-hydroxy-5,5,5-trideutero-4-trideuteromethyl-pentyl)-23Z-ene-26,27-hexafluorocholecalciferol

A 25 ml round bottom flask equipped with stir bar and Claisen adapter with rubber septum was charged with (1S,5R)-1-((tert-butyldimethyl)silanyloxy)-3-[2-(diphenylfosphinoyl)-eth-(Z)-ylidene]-5-fluoro-2-methylene-cyclohexane (520 mg, 1.105 mmol) and tetrahydrofuran (8 ml). The reaction mixture was cooled to −70° C. and n-butyllithium (0.69 ml, 1.10 mmol)) was added dropwise. The resulting deep red solution was stirred at −70° C. for 20 min and (1R,3aR, 7aR)-7a-Methyl-1-[(1R,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (314 mg, 0.493 mmol) was added dropwise in tetrahydrofuran (1.5 ml).). The reaction mixture was stirred for 5 h 30 min (in last hour the temperature was increased from −70 do −50° C.). The bath was removed and the mixture was poured into ethyl acetate (50 ml) and saturated solution of ammonium chloride (100 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product were pooled and evaporated to give colorless oil. The oil residue was used to next reaction. A 25 ml round bottom flask equipped with stir bar and Claisen adapter with rubber septum was charged with substrate and tetrabutylammonium fluoride (10 ml, 1M/tetrahydrofuran). The mixture was stirred for next 22 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using hexane:ethyl acetate—1:1 as mobile phase. Fractions containing product and impurity were purified on column (50 cm³, protected from light) using hexane:ethyl acetate—2:1 and 1:1 as mobile phase. Fractions containing product were pooled and evaporated to give product as colorless oil. Oil was dissolved in methyl acetate and evaporated (4 times) to give product as white foam (258 mg, 83%).

[α]_{D}²⁸=+25.0 (c=0.44, EtOH)

UV λmax (EtOH): 210 nm (ε 15800), 245 nm (ε 15638), 269 nm (ε 15445)

¹H NMR (DMSO-D6): 7.99 (1H, s), 6.36 (1H, d, J=11.3 Hz), 6.10 (1H, dt, J=11.9, 6.3 Hz), 5.92 (1H, d, J=11.3 Hz), 5.43 (1H, d, J=12.4 Hz), 5.39 (1H, s), 5.14 (1H, ddd, J=49.4, 5.5, 3.7 Hz), 4.98 (1H, d, J=1.7 Hz), 4.85 (1H, d, J=4.5 Hz), 4.02 (1H, s), 3.93-3.87 (1H, m), 2.81 (1H, d, J=12.8 Hz), 2.54-2.40 (2H, m), 2.16-1.97 (4H, m), 1.82-1.17 (17H, m), 0.89 (3H, s), 0.59 (3H, s)

¹³C NMR (DMSO-D6): 143.13(d, J=16.7 Hz), 141.74, 139.20, 132.94, 124.06, 122.93(q, J=286.0 Hz), 117.26, 117.12, 115.18(d, J=9.1 Hz), 91.95 (d, J=166.9 Hz), 76.78 (sep, J=28.8 Hz), 68.41, 64.54, 65.50, 56.51, 55.92, 46.24, 44.81, 44.58, 40.68(d, J=20.5 Hz), 40.28, 38.97, 38.78, 36.07, 28.33, 23.06, 22.74, 21.83, 21.40, 17.93, 14.59

| | | | | |
|---|---|---|---|---|
| MS HRES | Calculated for: | C₃₃H₄₁D₆F₇O₃ | [M + Na]⁺ | 653.3682 |
| | Observed: | | [M + Na]⁺ | 653.3686 |

### Example 39

### Synthesis of 1,25-Dihydroxy-20R-20-(4-hydroxy-5,5,5-trideutero-4-trideuteromethyl-pentyl)-23E-ene-26,27-hexafluorocholecalciferol

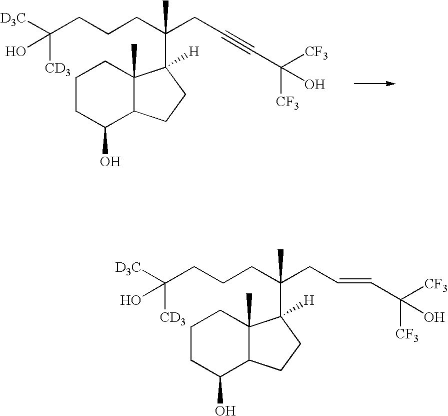

### (6R,3E)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol

A 25 ml round bottom flask equipped with stir bar and condenser with nitrogen sweep was charged with lithium aluminum hydride (13.00 ml, 13.00 mmol, 1M/tetrahydrofuran) and the mixture was cooled to 0° C. Sodium methoxide (702 mg, 13.00 mmol) was added slowly followed by (6R)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol (810 mg, 1.665 mmol) in tetrahydrofuran (8 ml). The reaction mixture was stirred at 80° C. for 6.5 h and then was cooled to 0° C. Saturated solution of ammonium chloride (5 ml) was added slowly followed by saturated solution of ammonium chloride (60 ml) and 2N HCl (20 ml). The mixture was extracted with ethyl acetate (3×50 ml), dried (Na₂SO₄) and evaporated.

The oil residue was chromatographed on columns (75 cm³) using hexane:ethyl acetate 2:1 and 1:1 as mobile phase. Fractions containing product were pooled and evaporated to give colorless oil (806 mg, 98%).

¹H NMR (CDCl₃): 6.28(1H, dt, J=15.4, 7.7 Hz), 5.59 (1H, d, J=15.7 Hz), 4.08 (1H, br s), 2.13-2.00 (3H, m), 1.83-1.79 (2H, m), 1.63-1.24 (18H, m), 1.08 (3H, s), 0.97 (3H, s)

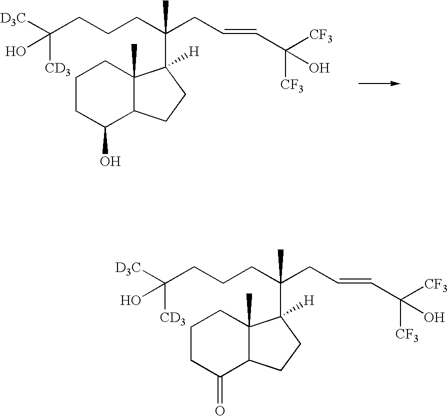

### (1R,3aR,7aR)-7a-Methyl-1-[(1R,3E)-6,6,6-trifluoro-5-hydroxy-1-methyl-1-(5,5,5-trideutero-4-hydroxy-4-trideuteromethyl-pentyl)-5-trifluoromethyl-hex-3-enyl]-octahydro-inden-4-one

A 25 ml round bottom flask equipped with stir bar and Claisen adapter with rubber septum was charged with pyridinium dichromate (1.600 g, 4.253 mmol) and dichloromethane (15 ml). The (6R,3E)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol (782 mg, 1.581 mmol) in dichloromethane (2 ml) was added dropwise and mixture was stirred in room temperature for 4 h 30 min.

The reaction mixture was filtrated through column with silica gel (25 cm³) using dichloromethane, dichloromethane:ethyl acetate 4:1. The fractions containing product were pooled and evaporated to give product as colorless oil (746 mg, 96%).

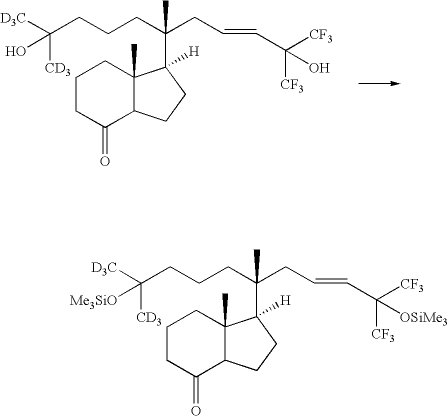

### (1R,3aR,7aR)-7a-Methyl-1-[(1R,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one

A 25 ml round bottom flask equipped with stir bar and Claisen adapter with rubber septum was charged with (1R,3aR,7aR)-7a-methyl-1-[(1R,3E)-6,6,6-trifluoro-5-hydroxy-1-methyl-1-(5,5,5-trideutero-4-hydroxy-4-trideuteromethyl-pentyl)-5-trifluoromethyl-hex-3-enyl]-octahydro-inden-4-one (746 mg, 1.515 mmol) and dichloromethane (10 ml). 1-(trimethylsilyl)imidazole (1.90 ml, 12.95 mmol) was added dropwise. The mixture was stirred at room temperature for 3 h. Hexane (150 ml) was added and the mixture was washed with water (3×50 ml), dried (Na₂SO₄) and evaporated. The oil residue was chromatographed on column (50 cm³) using hexane:ethyl acetate—5:1 as mobile phase. Fractions containing product were pooled and evaporated to give product as colorless oil (917 mg, 95%).

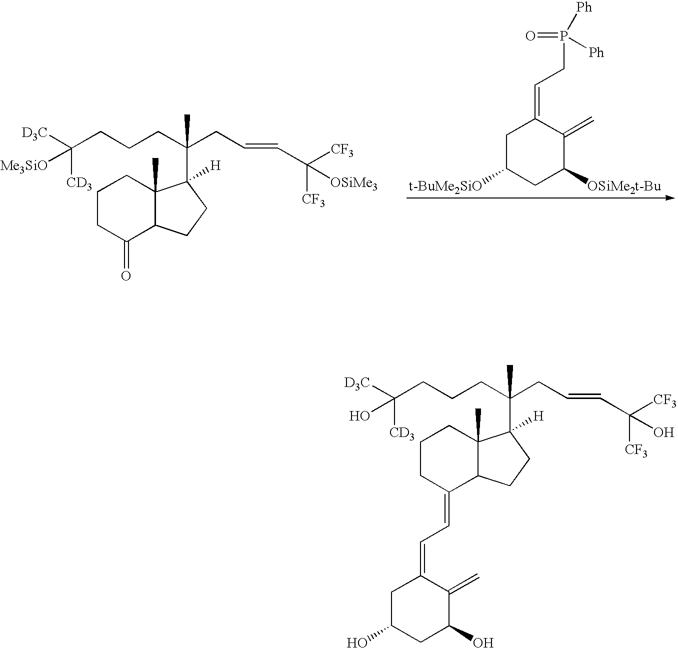

### 1,25-Dihydroxy-20R-20-(4-hydroxy-5,5,5-trideutero-4-trideuteromethyl-pentyl)-23E-ene-26,27-hexafluorocholecalciferol

A 25 ml round bottom flask equipped with stir bar and Claisen adapter with rubber septum was charged with (1S,5R)-1,5-bis-((tert-butyldimethyl)silanyloxy)-3-[2-(diphenylfosphinoyl)-eth-(Z)-ylidene]-2-methylene-cyclohexane (460 mg, 0.789 mmol) and tetrahydrofuran (8 ml). The reaction mixture was cooled to −70° C. and n-butyllithium (0.49 ml, 0.78 mmol)) was added dropwise. The resulting deep red solution was stirred at −70° C. for 20 min and (1R,3aR,7aR)-7a-Methyl-1-[(1R,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (302 mg, 0.474 mmol) was added dropwise in tetrahydrofuran (1.5 ml). The reaction mixture was stirred for 5.5 h (in last hour the temperature was increased from −70 do −50° C.). Saturated solution of ammonium chloride (1 ml) was added and the bath was removed. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil.

A 25 ml round bottom flask equipped with stir bar and Claisen adapter with rubber septum was charged with substrate and tetrabutylammonium fluoride (15 ml, 1M/tetrahydrofuran). The mixture was stirred for next 18 h. The mixture was dissolved by the addition of ethyl acetate (150 ml) and washed 6 times with water (50 ml) and brine (50 ml), dried (Na₂SO₄) and evaporated. The oil residue was chromatographed on column (50 cm³, protected from light) using ethyl acetate as mobile phase (tetrahydrofuran was used to transfer material on kolumn). Fractions with product contained some impurity. Fractions containing product were pooled and evaporated to give a white solid. The solid phase was transferred to Buchner funnel (10-15 μm) with hexane and washed with hexane (20 ml) to remove impurity. Then product was removed from funnel with ethanol (25 ml) and solution was evaporated to give product as white solid (215 mg, 71%).

[α]_{D}²⁷=+16.1 (c=0.44, EtOH)

UV λmax (EtOH): 214 nm (ε 1377), 265 nm (ε 1675)

¹H NMR (DMSO-D6): 8.05 (1H, s), 6.28 (1H, dt, J=15.3, 7.7 Hz), 6.18 (1H, d, J=11.1 Hz), 5.97 (1H, d, J=11.3 Hz), 5.62 (1H, d, J=15.3 Hz), 5.22 (1H, s), 4.87 (1H, d, J=4.7 Hz), 4.75 (1H, d, J=2.1 Hz), 4.55 (1H, d, J=3.6 Hz), 4.21-4.16 (1H, m), 4.04 (1H, s), 4.05-3.95 (1H, m), 2.79-2.76 (1H, m), 2.35 (1H, d, J=13.9 Hz), 2.16 (1H, dd, J=13.3, 5.2 Hz), 2.07 (2H, d, J=7.5 Hz), 2.00-1.90 (2H, m), 1.82-1.78 (1H, m), 1.65-1.55 (6H, m), 1.43-1.24 (10H, m), 0.90 (3H, s), 0.61 (3H, s)

¹³C NMR (DMSO-D6): 149.37, 139.67, 136.44, 135.84, 122.60(q, J=286.8 Hz), 122.35, 119.82, 117.93, 109.79, 75.49(sep, J=28.8 Hz), 68.39, 65.06, 56.36, 56.01, 46.20, 44.87, 44.56, 43.11, 41.06, 40.43, 28.33, 23.09, 22.49, 21.80, 21.60, 17.90, 14.59

| | | | | |
|---|---|---|---|---|
| MS HRES | Calculated for: | C₃₃H₄₂D₆F₆O₄ | [M + Na]⁺ | 651.3725 |
| | Observed: | | [M + Na]⁺ | 651.3729 |

### Example 40

### Synthesis of 1,25-Dihydroxy-20R-20-(4-hydroxy-5,5,5-trideutero-4-trideuteromethyl-pentyl)-23E-ene-26,27-hexafluoro-19-nor-cholecalciferol

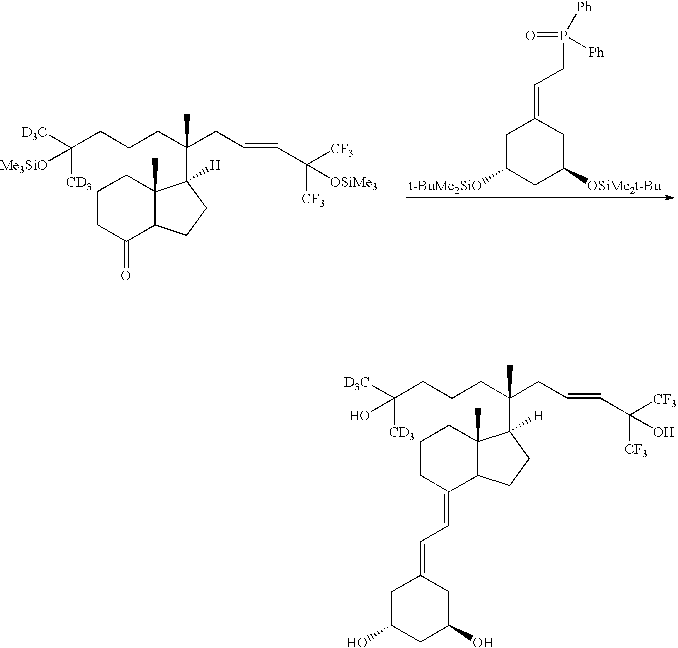

### 1,25-Dihydroxy-20R-20-(4-hydroxy-5,5,5-trideutero-4-trideuteromethyl-pentyl)-23E-ene-26,27-hexafluoro-19-nor-cholecalciferol

A 25 ml round bottom flask equipped with stir bar and Claisen adapter with rubber septum was charged with (1R,3R)-1,3-bis-((tert-butyldimethyl)silanyloxy)-5-[2-(diphenylfosphinoyl)ethylidene]-cyclohexane (584 mg, 1.023 mmol) and tetrahydrofuran (8 ml). The reaction mixture was cooled to −70° C. and n-butyllithium (0.63 ml, 1.01 mmol)) was added dropwise. The resulting deep red solution was stirred at −70° C. for 20 min and (1R,3aR,7aR)-7a-Methyl-1-[(1R,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (308 mg, 0.484 mmol) was added dropwise in tetrahydrofuran (1.5 ml). The reaction mixture was stirred for 6 h and then saturated solution of ammonium chloride (1 ml) was added and the bath was removed. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil. A 25 ml round bottom flask equipped with stir bar and Claisen adapter with rubber septum was charged with substrate and tetrabutylammonium fluoride (15 ml, 1M/tetrahydrofuran). The mixture was stirred for next 96 h. The mixture was dissolved by the addition of ethyl acetate (150 ml) and washed 6 times with water (50 ml) and brine (50 ml), dried (Na₂SO₄) and evaporated. The oil residue was chromatographed on column (50 cm³, protected from light) using hexane:tetrahydrofuran—1:1, 1:2 as mobile phase. (tetrahydrofuran contained some impurity). Fractions containing product were pooled and evaporated to give a white solid. The solid phase was transferred to Buchner funnel (10-15 μm) with hexane and washed with hexane (20 ml) to remove impurity. Then product was removed from funnel with ethanol (25 ml) and solution was evaporated to give product as white solid (274 mg, 92%).

[α]_{D}²⁷=+48.2 (c=0.44, EtOH)

UV λmax (EtOH): 244 nm (ε 35585), 252 nm (ε 41634), 262 nm (ε 28023)

¹H NMR (DMSO-D6): 8.05 (1H, s), 6.29 (1H, dt, J=15.6, 7.7 Hz), 6.07 (1H, d, J=11.3 Hz), 5.78 (1H, d, J=11.3 Hz), 5.62 (1H, d, J=15.6 Hz), 4.48 (1H, d, J=4.1 Hz), 4.38 (1H, d, J=3.8 Hz), 4.04 (1H, s), 3.90-3.84 (1H, m), 3.83-3.76 (1H, m), 2.73 (1H, d, J=13.2 Hz), 2.43 (1H, dd, J=12.9, 3.3 Hz), 2.26 (1H, d, J=10.4 Hz), 2.09-1.91 (6H, m), 1.69-1.24 (17H, m), 0.91 (3H, s), 0.60 (3H, s)

¹³C NMR (DMSO-D6): 139.10, 136.46, 134.64, 122.59(q, J=286.0 Hz), 120.80, 119.84, 116.38, 75.50(sep, J=28.8 Hz), 68.40, 65.54, 65.25, 56.36, 55.98, 46.04, 44.56, 42.22, 41.07, 40.43, 36.96, 28.16, 22.95, 22.50, 21.85, 21.50, 17.90, 14.70

| | | | | |
|---|---|---|---|---|
| MS HRES | Calculated for: | C₃₂H₄₂D₆F₆O₄ | [M + Na]⁺ | 639.3725 |
| | Observed: | | [M + Na]⁺ | 639.3725 |

### Example 41

### Synthesis of 1α-Fluoro-25-hydroxy-20R-20-(4-hydroxy-5,5,5-trideutero-4-trideuteromethyl-pentyl)-23E-ene-26,27-hexafluorocholecalciferol

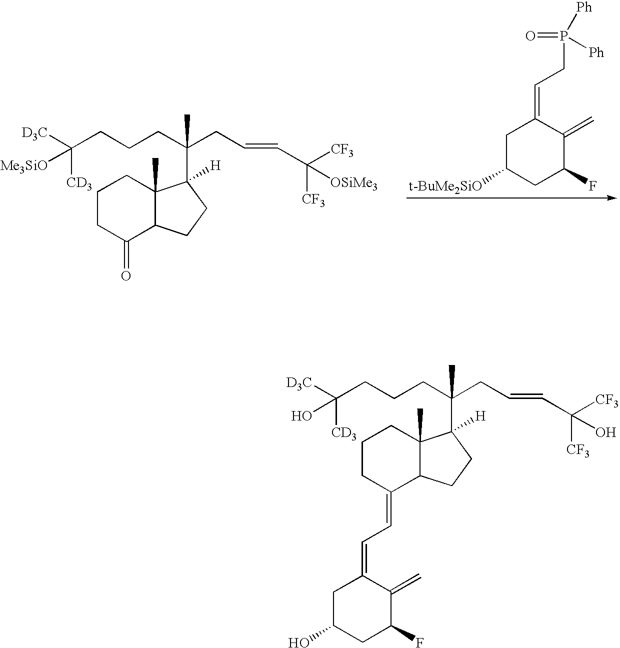

### 1α-Fluoro-25-hydroxy-20R-20-(4-hydroxy-5,5,5-trideutero-4-trideuteromethyl-pentyl)-23E-ene-26,27-hexylluorocholecalciferol

A 25 ml round bottom flask equipped with stir bar and Claisen adapter with rubber septum was charged with (1S,5R)-1-((tert-butyldimethyl)silanyloxy)-3-[2-(diphenylfosphinoyl)-eth-(Z)-ylidene]-5-fluoro-2-methylene-cyclohexane (543 mg, 1.154 mmol) and tetrahydrofuran (8 ml). The reaction mixture was cooled to −70° C. and n-butyllithium (0.72 ml, 1.15 mmol)) was added dropwise. The resulting deep red solution was stirred at −70° C. for 20 min and (1R,3aR, 7aR)-7a-Methyl-1-[(1R,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (279 mg, 0.438 mmol) was added dropwise in tetrahydrofuran (1.5 ml).). The reaction mixture was stirred for 8 h (in last hour the temperature was increased from −70 do −50° C.). The bath was removed and the mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product were pooled and evaporated to give oil. The oil residue was used to next reaction. A 25 ml round bottom flask equipped with stir bar and Claisen adapter with rubber septum was charged with substrate and tetrabutylammonium fluoride (8 ml, 1M/tetrahydrofuran). The mixture was stirred for next 25 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using hexane:ethyl acetate—2:1, 1:1 as mobile phase. Fractions containing product were pooled and evaporated to give product as colorless oil. Oil was dissolved in methyl acetate and evaporated (4 times) to give product as white foam (216 mg, 78%).

[α]_{D}²⁸=+32.5 (c=0.48, EtOH)

UV λmax (EtOH): 211 nm (ε 16931), 243 nm (ε 17696), 269 nm (ε 17736)

¹H NMR (DMSO-D6): 8.05 (1H, s), 6.36 (1H, d, J=11.3 Hz), 6.28 (1H, dt, J=15.6, 7.6 Hz), 5.92 (1H, d, J=11.3 Hz), 5.62 (1H, d, J=15.3 Hz), 5.39 (1H, s), 5.14 (1H, br d, J=49.7 Hz), 4.99 (1H, d, J=1.7 Hz), 4.86 (1H, d, J=4.3 Hz), 4.04 (1H, s), 3.94-3.86 (1H, m), 2.81 (1H, d, J=12.4 Hz), 2.15-2.06 (4H, m), 1.99-1.91 (3H, m), 1.82-1.55 (6H, m), 1.46-1.20 (10H, m), 0.90 (3H, s), 0.59 (3H, s)

¹³C NMR (DMSO-D6): 143.29(d, J=17.4 Hz), 141.83, 136.58, 133.13(d, J=1.5 Hz), 124.20, 122.76(q, J=287.5 Hz), 119.99, 117.46, 115.39(d, J=9.9 Hz), 92.09 (d, J=166.8 Hz), 75.57 (sep, J=28.8 Hz), 68.48, 64.60, 64.56, 56.40, 56.02, 46.31, 44.86, 44.58, 41.11, 40.71(d, J=20.4 Hz), 40.43, 39.36, 28.34, 23.02, 22.44, 21.79, 21.50, 17.90, 14.60

| | | | | |
|---|---|---|---|---|
| MS HRES | Calculated for: | C₃₃H₄₁D₆F₇O₃ | [M + Na]⁺ | 653.3682 |
| | Observed: | | [M + Na]⁺ | 653.3684 |

### Example 42

### Synthesis of 1,25-Dihydroxy-20-cyclopropyl-26,27-hexadeutero-19-nor-cholecalciferol

### (1R,3aR,4S,7aR)-2-{1-[4-(tert-butyl-dimethyl-silanyloxy)-7a-methyl-octahydro-inden-1-yl]-cyclopropyl}ethyl toluene-4-sulfonic acid ester

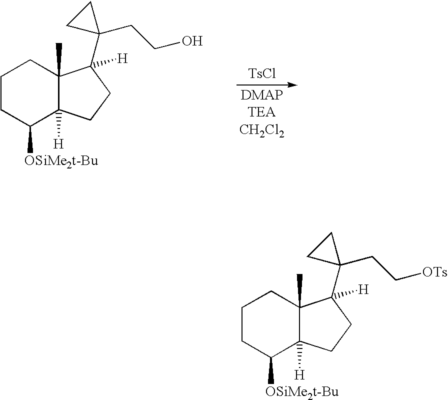

A 100 ml round bottom flask equipped with stir bar and nitrogen sweep was charged with 5.98 g (16.958 mmol) of (1R,3aR,4S,7aR)-2-{1-[4-(tert-Butyl-dimethyl-silanyloxy)-7a-methyl-octahydro-inden-1-yl]-cyclopropyl}-ethanol, 50 ml of dichloromethane, 6 ml of triethylamine and 230 mg (1.883 mmol) of 4-dimethylamino pyridine. A 4.83 g (25.334 mmol) of tosyl chloride was added in one portion. The mixture was stirred at room temperature for 2 h. The suspension was poured into a mixture of 40 g of ice, 100 ml of saturated sodium hydrogen carbonate solution and 100 ml of hexane. The aqueous layer was re-extracted three times with 50 ml of dichloromethane. These combined extracts were washed with 100 ml of brine, dried over Na₂SO₄ and evaporated. The residue was purified on a short flash chromatography column using hexane:ethyl acetate (20:1) as mobile phase to give 9.0 g of crude product as colorless oil. Product was used to the next reaction without farther purification.

### (1R,3aR,4S,7aR)-2-(2-{1-[4-(tert-Butyl-dimethyl-silanyloxy)-7a-methyl-octahydro-inden-1-yl]-cyclopropyl}-ethyl)-malonic acid dimethyl ester

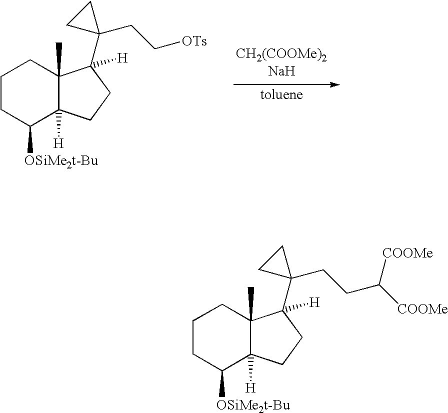

A 500 ml 3-neck round bottom flask equipped with mechanical stirrer, additional funnel with nitrogen sweep and condenser was charged with 160 ml of toluene. A 5.20 g (130 mmol) of sodium hydride (60% dispersion in mineral oil) was added in one portion. To the stirred suspension was added dropwise a solution of 19.36 g (146.5 mmol) of dimethyl malonate in 50 ml of toluene. The gel was heated in 120° C. oil bath for 10 min and then a solution of 9.0 g (ca. 16.958 mmol) of crude (1R,3aR,4S,7aR)-2-{1-[4-(tert-butyl-dimethyl-silanyloxy)-7a-methyl-octahydro-inden-1-yl]-cyclopropyl}ethyl toluene-4-sulfonic acid ester in 100 ml of toluene was added dropwise. The reaction was stirred at this temperature for 6 h. The flask was placed into an ice bath and 100 ml of cold water was added to dissolve the voluminous precipitate. The mixture was equilibrated with 100 ml of hexane. The resulting aqueous phase was re-extracted three times with 50 ml of toluene. The combined extracts were washed with 100 ml of water and 50 ml of brine, then dried over Na₂SO₄ and evaporated. The oil residue was chromatographed on column (500 cm³) using hexane:ethyl acetate (20:1; 15:1) as mobile phase and collecting ca. 50 ml fractions. Fractions containing product were pooled and evaporated. The fractions which were mixtures were pooled, evaporated separately and was re-chromatographed on column (300 cm³) using hexane:ethyl acetate (20:1) as mobile phase and collecting ca. 25 ml fractions. Fractions containing product were pooled and evaporated to give 6.148 g (78% for two steps) of product as colorless oil.

### (1R,3aR,4S,7aR)-4-{1-[4-(tert-Butyl-dimethyl-silanyloxy)-7a-methyl-octahydro-inden-1-yl]-cyclopropyl}-butyric acid methyl ester

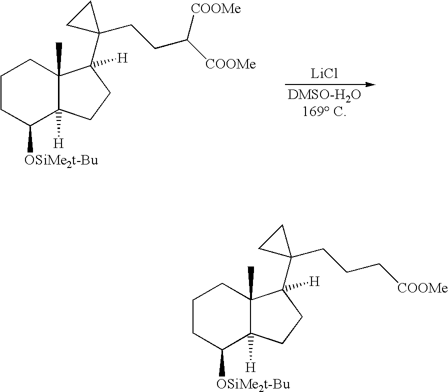

A 100 ml round bottom flask equipped with stir bar and condenser with nitrogen sweep was charged with 6.11 g (13.091 mmol) of (1R,3aR,4S,7aR)-2-(2-{1-[4-(tert-Butyl-dimethyl-silanyloxy)-7a-methyl-octahydro-inden-1-yl]-cyclopropyl}-ethyl)-malonic acid dimethyl ester, 25 ml of mixture of dimethylsulfoxide and water (100:1) and 1.11 g (26.185 mmol) of lithium chloride. The mixture was stirred and heated under nitrogen at 160° C. for 3 h. Then the solution was allowed to cool and distributed between 100 ml of water and 200 ml of hexane The aqueous layer was extracted three times with 50 ml of hexane. The combined organic layers were washed five times with 50 ml of water and 50 ml of brine then dried over Na₂SO₄ and evaporated. The oil residue was chromatographed on column (500 cm³) using hexane:ethyl acetate (50:1) as mobile phase and collecting ca. 50 ml fractions. Fractions containing product were pooled and evaporated to give of colorless oil. The fractions which were mixtures were pooled, evaporated separately and re-chromatographed on column (160 cm³) using hexane:ethyl acetate (50:1). It gave 4.19 g (78%) of product.

¹H NMR (CDCl₃): 3.98 (1H, br s), 3.66 (3H, s), 2.29-2.23 (2H, m), 2.10-1.75 (5H, m), 1.68-1.22 (10H, m), 0.94 (3H, s), 0.88 (9H, s), 0.71-0.65 (1H, m), 0.61-0.50 (1H, m), 0.21-0.14 (2H, m), 0.00 (3H, s), −0.02(3H, s), −0.05-0.12 (1H, m).

### (1R,3aR,4S,7aR)-5-{1-[4-(tert-Butyl-dimethyl-silanyloxy)-7a-methyl-octahydro-inden-1-yl]-cyclopropyl}-1,1,1-trideutero-2-trideuteromethyl-pentan-2-ol

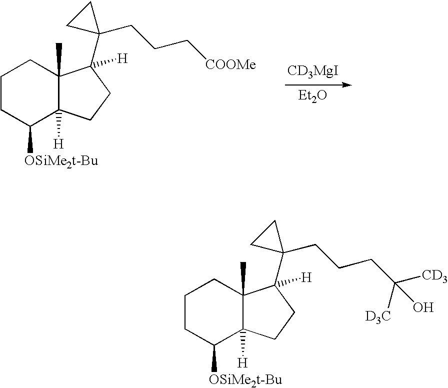

A 250 ml round bottom flask equipped with stir bar, Claisen adapter with rubber septum was charged with 3.012 g (7.370 mmol) of (1R,3aR,4S,7aR)-4-{1-[4-(tert-Butyl-dimethyl-silanyloxy)-7a-methyl-octahydro-inden-1-yl]-cyclopropyl}-butyric acid methyl ester and 75 ml of anhydrous diethyl ether. The solution was cooled in ace-water bath and 20 ml (20 mmol) of 1M methyl-d₃-magnesium iodide in diethyl ether was added dropwise. After completion of the addition the mixture was stirred at room temperature for 1.5 h then cooled again in an ice bath. A 25 ml of saturated solution of ammonium chloride was added dropwise. The resulting precipitate was dissolved by the addition of 100 ml of water. The aqueous layer was re-extracted three times with 50 ml of diethyl ether. The combined ether layers were dried over Na₂SO₄ and evaporated. The oil residue was chromatographed on column (350 cm³) using hexane:ethyl acetate (9:1) as mobile phase and collecting ca. 50 ml fractions. Fractions containing product were pooled and evaporated to give of colorless oil. The fractions which were mixtures were pooled, evaporated separately and re-chromatographed on column (100 cm³) using hexane:ethyl acetate (9:1). It gave 2.95 g (96%) of product.

¹H NMR (CDCl₃): 3.99 (1H, br s), 2.05-1.76 (4H, m), 1.68-1.17 (14H, m), 0.95 (3H, s), 0.88 (9H, s), 0.70-0.52 (2H, m), 0.22-0.12 (2H, m), 0.01 (3H, s), −0.01(3H, s), −0.05-−0.11 (1H, m).

### (1R,3aR,4S,7aR)-1-[1-(4-Hydroxy-5,5,5-tridutero-4-trideuteromethyl-pentyl)-cyclopropyl]-7a-methyl-octahydro-inden-4-ol

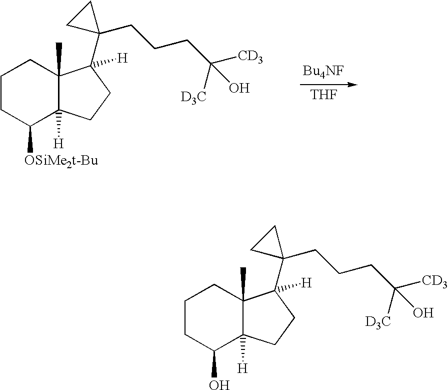

A 50 ml round bottom flask equipped with stir bar and Claisen adapter with rubber septum was charged with 2.940 g, (7.088 mmol) of (1R,3aR,4S,7aR)-5-{1-[4-(tert-Butyl-dimethyl-silanyloxy)-7a-methyl-octahydro-inden-1-yl]-cyclopropyl}-1,1,1-trideutero-2-trideuteromethyl-pentan-2-ol and 25 ml (25.0 mmol) of 1.0M tetrabutyl ammonium fluoride in tetrahydrofuran. The reaction mixture was stirred at 70° C. for 22 h and the new portion 10 ml (10.0 mmol) of tetrabutylammonium fluoride was added. The reaction was stirred at 70° C. for next 26 h. The mixture was dissolved by the addition of 150 ml of ethyl acetate and washed six times with 40 ml of water and 20 ml of brine, dried over Na₂SO₄ and evaporated. The oil residue was chromatographed on column (250 cm³) using hexane:ethyl acetate (3:1) as mobile phase. Fractions containing product were pooled and evaporated to give 2.00 g (94%) of product.

### (1R,3aR,7aR)-1-[1-(4-Hydroxy-5,5,5-trideutero-4-trideuteromethyl-pentyl)-cyclopropyl]-7a-methyl-octahydro-inden-4-one

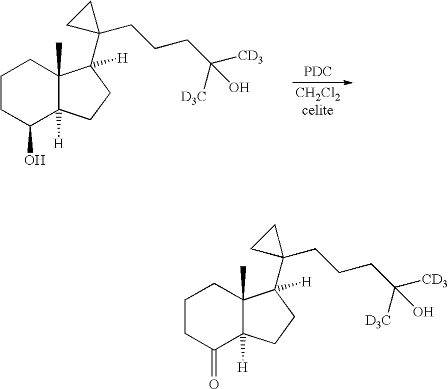

A 250 ml round bottom flask equipped with stir bar and Claisen adapter with rubber septum was charged with 7.42 g (19.723 mmol) of pyridinium dichromate, 7.28 g of celite and 75 ml of dichloromethane. A 1.96 g (6.522 mmol) of (1R,3aR,4S,7aR)-1-[1-(4-Hydroxy-5,5,5-tridutero-4-trideuteromethyl-pentyl)-cyclopropyl]-7a-methyl-octahydro-inden-4-ol in 5 ml of dichloromethane was added dropwise and mixture was stirred in room temperature for 6 h. The reaction mixture was filtrated through column with 100 cm³ of silica gel using dichloromethane and dichloromethane:ethyl acetate (4:1, 3:1, 2:1) as mobile phases. The fractions containing product were pooled and evaporated to give 1.92 g (98%) of ketone.

¹H NMR (CDCl₃): 2.50 (1H, dd, J=11.4, 7.0 Hz), 2.29-2.12 (4H, m), 2.05-1.86 (3H, m), 1.75-1.17 (9H, m), 1.08-0.98 (1H, m), 0.73-0.60 (2H, m), 0.69 (3H, s), 0.26-0.19 (2H, m), 0.06-−0.01(1H, m).

### (1R,3aR,7aR)-7a-Methyl-1-[1-(5,5,5-trideutero-4-trideuteromethyl-4-trimethylsilanyloxy-pentyl)-cyclopropyl]-octahydro-inden-4-one

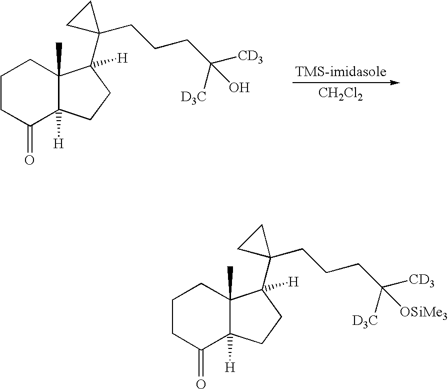

A 100 ml round bottom flask equipped with stir bar and Claisen adapter with rubber septum was charged with 1.91 g (6.399 mmol) of (1R,3aR,7aR)-1-[1-(4-Hydroxy-5,5,5-trideutero-4-trideuteromethyl-pentyl)-cyclopropyl]-7a-methyl-octahydro-inden-4-one and 60 ml of dichloromethane. A 3.8 ml (25.90 mmol) of 1-(trimethylsilyl)imidasole was added dropwise. The mixture was stirred at room temperature for 1 h 45 min. A 25 ml of water was added and the mixture was stirred for 10 min. The resulting mixture was dissolved by the addition of 200 ml of water. The aqueous layer was extracted five times with 50 ml of ethyl acetate. The combined organic layers were washed with 50 ml of brine, dried over Na₂SO₄ and evaporated. The oil residue was chromatographed on column (200 cm³) using hexane:dichloromethane (2:1, 1:1) and dichloromethane as mobile phases. Fractions containing product were pooled and evaporated to give 2.10 g (89%) of product as colorless oil.

### 1α,25-Dihydroxy-20-cyclopropyl-26,27-hexadeutero-19-nor-cholecalciferol

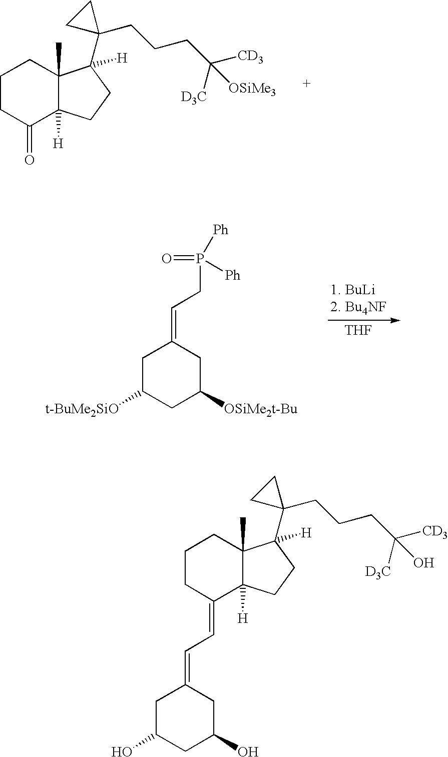

A 50 ml round bottom flask equipped with stir bar and Claisen adapter with rubber septum was charged with 2.155 g (3.776 mmol) of (1R,3R)-1,3-bis-((tert-butyldimethyl)silanyloxy)-5-[2-(diphenylfosphinoyl)ethylidene]-cyclohexane and 15 ml of anhydrous tetrahydrofurane. The reaction mixture was cooled to −78° C. and 2.3 ml (3.68 mmol) 1.6M n-butyllithium in hexane was added dropwise. The resulting deep red solution was stirred at −78° C. for 20 min and 700 mg (1.888 mmol) of (1R,3aR,7aR)-7a-Methyl-1-[1-(5,5,5-trideutero-4-trideuteromethyl-4-trimethylsilanyloxy-pentyl)-cyclopropyl]-octahydro-inden-4-one was added dropwise in 2 ml of anhydrous tetrahydrofurane. The reaction mixture was stirred for 4 h and then the bath was removed and the mixture was poured into 50 ml of ethyl acetate and 50 ml brine. The water fraction was extracted three times with 75 ml of ethyl acetate. All organic layers were combined, dried over Na₂SO₄ and evaporated. The oil residue was chromatographed on column (100 cm³, protected from light) using hexane:ethyl acetate (20:1) as mobile phase. Fractions containing product were pooled and evaporated to give colorless oil which was treated with 20 ml 1.0M tetrabutylammonium fluoride in tetrahydrofurane. The reaction mixture was stirred at room temperature for 24 h. The mixture was dissolved by the addition of 150 ml of ethyl acetate. The organic layer was washed five times with 50 ml of water and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give 525 mg (66%) of white foam.

[α]³⁰_{D}=+47.8 c 0.46, CHCl₃

UV λmax (EtOH): 243 nm (ε 32133), 251 nm (ε 37757), 261 nm (ε 25993)

¹H NMR (CDCl₃): 6.30(1H, d, J=11.3 Hz), 5.82 (1H, d, J=11.3), 4.15-4.08 (1H, m), 4.07-4.00 (1H, m), 2.82-2.78 (1H, m), 2.73 (1H, dd, J=13.1, 3.7 Hz), 2.48 (1H, dd, J=13.3, 3.3 Hz), 2.24-1.24 (21H, m), 1.19 (1H, s), 1.00-0.91 (1, m), 0.68-0.61 (2H, m), 0.59 (3H, s), 0.23-0.17(2H, m), 0.05-−0.05 (1H, m)

| | | | | |
|---|---|---|---|---|
| MS HRES | Calculated for: | C₂₇H₃₈D₆FO₃ | [M + Na]⁺ | 445.3559 |
| | Observed: | | [M + Na]⁺ | 445.3561 |

### Example 43

### Synthesis of Acetic acid 1α-acetoxy-25-hydroxy-20-cyclopropyl-26,27-hexadeutero-19-nor-cholecalciferyl ester

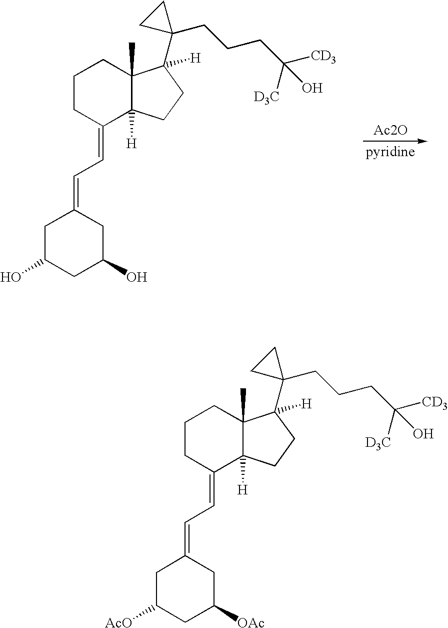

A 25 ml round bottom flask equipped with stir bar and Claisen adapter with rubber septum was charged with 245 mg (0.579 mmol) of 1α,25-Dihydroxy-20-cyclopropyl-26,27-hexadeutero-19-nor-cholecalciferol and 6 ml of pyridine. The mixture was stirred at 0-5° C. and 1 ml (10.6 mmol) of acetic anhydride was added dropwise. The reaction mixture was stirred at 0-5° C. for 17 h and new portion 0.75 ml (7.9 mmol) of acetic anhydride was added dropwise. The reaction mixture was stirred for next 24 h. The mixture was dissolved by the addition of 10 ml of water, stirred for 15 min and poured into 100 ml of ethyl acetate. The mixture was extracted five times with 50 ml of water and 50 ml of brine, dried over Na₂SO₄ and evaporated. The oil residue was chromatographed on column (50 cm³, protected from light) using hexane:ethyl acetate (2:1) as mobile phase. Fractions containing product were pooled and evaporated to give product as colorless oil. The product was dissolved in methyl acetate and evaporated (4 times) to give 259 mg (88%) of white foam.

[α]³⁰_{D}=+8.2 c 0.45, CHCl₃

UV λmax (EtOH): 243 nm (ε 34931), 251 nm (ε 40870), 260 nm (ε 27807)

¹H NMR (CDCl₃): 6.25(1H, d, J=11.1 Hz), 5.72 (1H, d, J=11.5 Hz), 5.12-5.06 (2H, m), 2.80-2.76 (1H, m), 2.60-2.44 (3H, m), 2.27 (1H, dd, J=13.5, 7.7 Hz), 2.14-1.87 (6H, m), 2.03 (3H, s), 2.00 (3H, s), 1.70-1.25 (11H, m), 1.18 (1H, s), 1.00-0.91 (1H, m), 0.68-0.60 (2H, m), 0.57 (3H, s), 0.23-0.16 (2H, m), 0.00-−0.06 (1H, m)

| | | | | |
|---|---|---|---|---|
| MS HRES | Calculated for: | C₃₁H₄₂D₆FO₅ | [M + Na]⁺ | 529.3770 |
| | Observed: | | [M + Na]⁺ | 529.3782 |

### Example 44

### 1α,25-Dihydroxy-20-cyclopropyl-26,27-hexadeutero-cholecalciferol

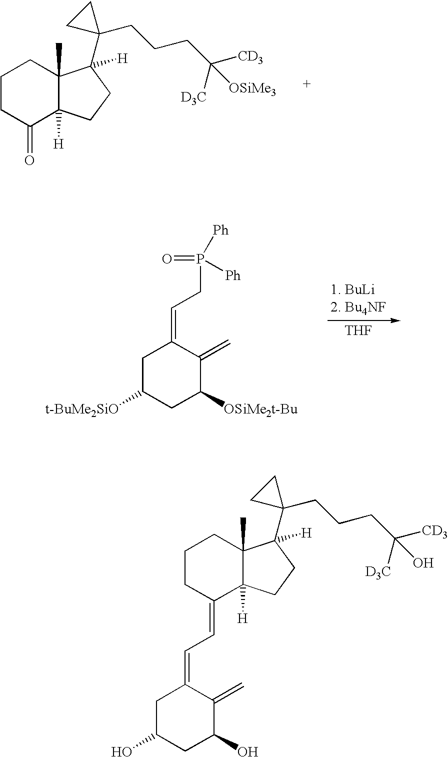

A 50 ml round bottom flask equipped with stir bar and Claisen adapter with rubber septum was charged with 2.201 g (3.774 mmol) of (1S,5R)-1,5-bis-((tert-butyldimethyl)silanyloxy)-3-[2-(diphenylfosphinoyl)-eth-(Z)-ylidene]-2-methylene-cyclohexane and 15 ml of anhydrous tetrahydrofurane. The reaction mixture was cooled to −78° C. and 2.3 ml (3.68 mmol) 1.6M n-butyllithium in hexane was added dropwise. The resulting deep red solution was stirred at −78° C. for 20 min and 700 mg (1.888 mmol) of (1R,3aR,7aR)-7a-Methyl-1-[1-(5,5,5-trideutero-4-trideuteromethyl-4-trimethylsilanyloxy-pentyl)-cyclopropyl]-octahydro-inden-4-one was added dropwise in 2 ml of anhydrous tetrahydrofurane. The reaction mixture was stirred for 4 h and then the bath was removed and the mixture was poured into 60 ml of ethyl acetate and 50 ml of brine. The water fraction was extracted four times with 75 ml of ethyl acetate, dried over Na₂SO₄ and evaporated. The oil residue was chromatographed on column (100 cm³, protected from light) using hexane:ethyl acetate (20:1) as mobile phase. Fractions containing product were pooled and evaporated to give colorless oil which was treated with 20 ml 1.0M tetrabutylammonium fluoride in tetrahydrofurane. The reaction mixture was stirred at room temperature for 24 h. The mixture was dissolved by the addition of 150 ml of ethyl acetate and washed five times with 50 ml of water and 50 ml of brine, dried over Na₂SO₄ and evaporated. The oil residue was chromatographed on column (75 cm³, protected from light) using ethyl acetate as mobile phase. Fractions containing product were pooled and evaporated to give product as colorless oil. Some fractions contain impurity were purified on column (50 cm³, protected from light) using ethyl acetate:hexane (2:1) as mobile phase. The product was dissolved in methyl acetate and evaporated (4 times) to give 749 mg (91%) of white foam.

[α]³⁰_{D}=+3.3 c 0.46, CHCl₃

UV λmax (EtOH): 213 nm (ε 12528), 264 nm (ε 14832)

¹H NMR (CDCl₃): 6.37(1H, d, J=11.5 Hz), 5.99 (1H, d, J=11.1), 5.32 (1H, s), 4.99 (1H, s), 4.44-4.42 (1H, m), 4.23 (1H, br s), 2.84-2.80 (1H, m), 2.59 (1H, dd, J=13.5, 3.5 Hz), 2.31 (1H, dd, J=13.4, 6.4 Hz), 2.13-2.09 (1H, m), 2.06-1.88 (5H, m), 1.73-1.26 (13H, m), 1.18 (1H, br s), 0.99-0.90 (1H, m), 0.68-0.61 (2H, m), 0.59 (3H, s), 0.21-0.16 (2H, m), 0.00-−0.06 (1H, m)

| | | | | |
|---|---|---|---|---|
| MS HRES | Calculated for: | C₂₈H₃₈D₆FO₃ | [M + Na]⁺ | 457.3559 |
| | Observed: | | [M + Na]⁺ | 457.3563 |

### Example 45

### Acetic acid 1α-acetoxy-25-hydroxy-20-cyclopropyl-26,27-hexadeutero-cholecalciferyl ester

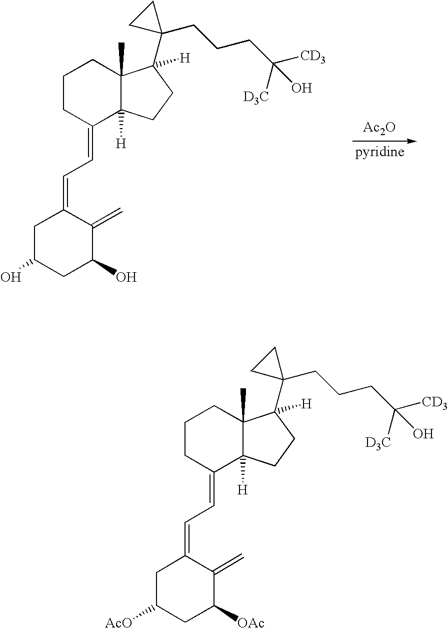

A 25 ml round bottom flask equipped with stir bar and Claisen adapter with rubber septum was charged with 345 mg (0.794 mmol) of 1α, 25-Dihydroxy-20-cyclopropyl-26,27-hexadeutero-cholecalciferol and 7 ml of pyridine. The mixture was stirred at 0-5° C. and 1.5 ml (15.9 mmol) of acetic anhydride was added dropwise. The reaction mixture was stirred at 0-5° C. for 17 h and new portion 0.5 ml (5.3 mmol) of acetic anhydride was added. The next portion 1 ml (10.6 mmol) of acetic anhydride was added after next 25 h. The reaction mixture was stirred for additional 16 h. The mixture was dissolved by the addition of 15 ml of water, stirred for 15 min and poured into 120 ml of ethyl acetate. The mixture was extracted five times with 50 ml of water and 50 ml of brine, dried over Na₂SO₄ and evaporated. The oil residue was chromatographed on column (75 cm³, protected from light) using hexane:ethyl acetate (2:1) as mobile phase. Fractions containing product were pooled and evaporated to give product as colorless oil. The product was dissolved in methyl acetate and evaporated (4 times) to give 364 mg (88%) of white foam.

[α]³⁰_{D}=−20.2 c 0.46, CHCl₃

UV λmax (EtOH): 207 nm (ε 14863), 250 nm (ε 15225), 265 nm (ε 15985)

¹H NMR (CDCl₃): 6.34(1H, d, J=11.3 Hz), 5.89 (1H, d, J=11.5 Hz), 5.47 (1H, dd, J=6.2, 4.0 Hz), 5.30 (1H, s), 5.21-5.15 (1H, m), 5.03 (1H, d, J=1.7 Hz), 2.82-2.78 (1H, m), 2.64 (1H, dd, J=13.2, 4.3 Hz), 2.38-2.33 (1H, m), 2.13-1.92 (6H, m), 2.05 (3H, s), 2.03 (3H, s), 1.72-1.28 (11H, m), 1.19 (1H, s), 0.98-0.88 (1H, m), 0.68-0.59 (2H, m), 0.56 (3H, s), 0.22-0.16 (2H, m), 0.01-−0.06(1H, m)

| | | | | |
|---|---|---|---|---|
| MS HRES | Calculated for: | C₃₂H₄₂D₆FO₅ | [M + Na]⁺ | 541.3770 |
| | Observed: | | [M + Na]⁺ | 541.3764 |

### Example 46

### 1α-Fluoro-25-hydroxy-20-cyclopropyl-26,27-hexadeutero-cholecalciferol

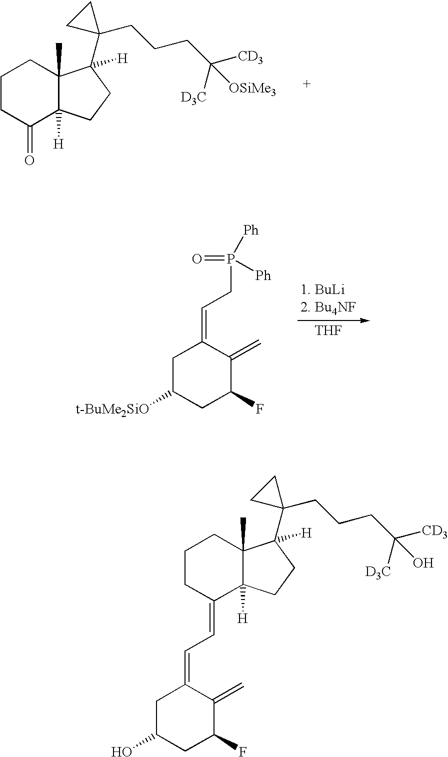

A 50 ml round bottom flask equipped with stir bar and Claisen adapter with rubber septum was charged with 1.907 g (4.052 mmol) of (1S,5R)-1-((tert-butyldimethyl)silanyloxy)-3-[2-(diphenylfosphinoyl)-eth-(Z)-ylidene]-5-fluoro-2-methylene-cyclohexane and 15 ml of anhydrous tetrahydrofurane. The reaction mixture was cooled to −78° C. and 2.5 ml (4.00 mmol) 1.6M n-butyllithium in hexane was added dropwise. The resulting deep red solution was stirred at −78° C. for 20 min and 650 mg (1.754 mmol) of (1R,3aR,7aR)-7a-Methyl-1-[1-(5,5,5-trideutero-4-trideuteromethyl-4-trimethylsilanyloxy-pentyl)-cyclopropyl]-octahydro-inden-4-one was added dropwise in 2 ml of anhydrous tetrahydrofurane. The reaction mixture was stirred for 3.5 h and then the bath was removed and the mixture was poured into 60 ml of ethyl acetate and 50 ml of brine. The water fraction was extracted four times with 60 ml of ethyl acetate dried (Na₂SO₄) and evaporated. The oil residue was chromatographed on column (75 cm³, protected from light) using hexane:ethyl acetate—20:1 as mobile phase. Fractions containing product were pooled and evaporated to give colorless oil which was treated with 20 ml 1.0M tetrabutylammonium fluoride in tetrahydrofurane. The reaction mixture was stirred at room temperature for 7.5 h. The mixture was dissolved by the addition of 150 ml of ethyl acetate and washed five times with 50 ml of water and 50 ml of brine, dried over Na₂SO₄ and evaporated. The oil residue was chromatographed on column (75 cm³, protected from light) using hexane:ethyl:acetate (1:1) as mobile phase. Fractions containing product were pooled and evaporated to give product as colorless oil. Some fractions contain impurity were purified on column (50 cm³, protected from light) using hexane:ethyl acetate (2:1) as mobile phase. The product was dissolved in methyl acetate and evaporated (4 times) to give 629 mg (82%) of white foam.

[α]³⁰_{D}=+22.1 c 0.43, CHCl₃

UV λmax (EtOH): 209 nm (ε 14376), 243 nm (ε 13949), 269 nm (ε 14083)

¹H NMR (CDCl₃): 6.39(1H, d, J=11.1 Hz), 6.00 (1H, d, J=11.1 Hz), 5.38 (1H, s), 5.13 (1H, ddd, J=49.5, 6.9, 3.7 Hz), 5.09 (1H, s), 4.22 (1H, br s), 2.84-2.80 (1H, m), 2.62 (1H, dd, J=13.3, 3.7 Hz), 2.30 (1H, dd, J=13.3, 7.5 Hz), 2.23-1.92 (6H, m), 1.74-1.26 (12H, m), 1.18 (1H, s), 0.98-0.91(1H, m), 0.68-0.61 (2H, m), 0.59 (3H, s), 0.21-0.16 (2H, m), 0.00-−0.06 (1H, m)

| | | | | |
|---|---|---|---|---|
| MS HRES | Calculated for: | C₂₈H₃₇D₆FO₂ | [M + Na]⁺ | 459.3516 |
| | Observed: | | [M + Na]⁺ | 459.3521 |

### Example 47

### Acetic acid 1α-fluoro-25-hydroxy-20-cyclopropyl-26,27-hexadeutero-cholecalciferyl ester

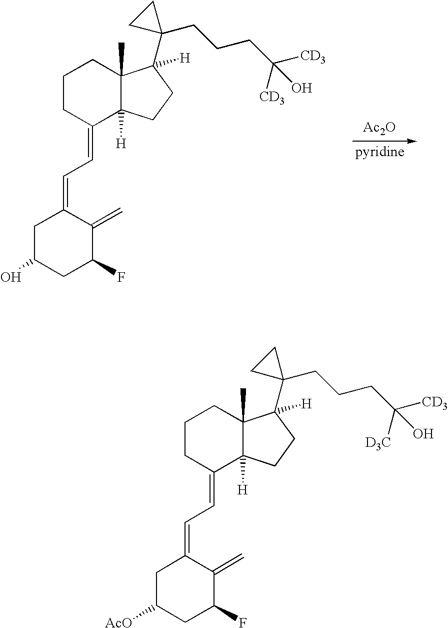

A 25 ml round bottom flask equipped with stir bar and Claisen adapter with rubber septum was charged with 300 mg (0.687 mmol) of 1α-Fluoro-25-hydroxy-20-cyclopropyl-26,27-hexadeutero-cholecalciferol and 6 ml of pyridine. The mixture was stirred at 0-5° C. and 1 ml (10.6 mmol) of acetic anhydride was added dropwise. The reaction mixture was stirred at 0-5° C. for 16 h and new portion 0.5 ml (5.3 mmol) of acetic anhydride was added. The reaction mixture was stirred for next 3 h. The mixture was dissolved by the addition of 15 ml of water, stirred for 15 min and poured into 120 ml of ethyl acetate. The mixture was extracted five times with 50 ml of water and 50 ml of brine, dried over Na₂SO₄ and evaporated. The oil residue was chromatographed on column (50 cm³, protected from light) using hexane:ethyl acetate (3:1) as mobile phase. Fractions containing product were pooled and evaporated to give product as colorless oil. The product was dissolved in methyl acetate and evaporated (4 times) to give 292 mg (89%) of white foam.

[α]³⁰_{D}=−15.9 c 0.46, CHCl₃

UV λmax (EtOH): 210 nm (ε 11176), 245 nm (ε 10496), 264 nm (ε 10387)

¹H NMR (CDCl₃): 6.36(1H, d, J=11.3 Hz), 6.00 (1H, d, J=11.3 Hz), 5.40 (1H, s), 5.23-5.16 (1H, m), 5.10 (1H, dm, J=49.7 Hz), 5.10 (1H, s), 2.82-2.79 (1H, m), 2.64 (1H, dd, J=13.7, 3.7 Hz), 2.41-2.36 (1H, m), 2.23-1.93 (6H, m), 2.04 (3H, s), 1.73-1.26 (12H, m), 0.99-0.92 (1H, m), 0.68-0.61(2H, m), 0.60 (3H, s), 0.22-0.17 (2H, m), 0.00-−0.06 (1H, m)

| | | | | |
|---|---|---|---|---|
| MS HRES | Calculated for: | C₃₀H₃₉D₆FO₃ | [M + Na]⁺ | 501.3621 |
| | Observed: | | [M + Na]⁺ | 501.3619 |

### Example 48

### Synthesis of 1,25-Dihydroxy-16-ene-20 cyclopropyl-26,27-hexadeutero-19-nor-cholecalciferol

### (3aR,4S,7aR)-1-E/Z-{1-[4-(tert-Butyl-dimethyl-silanyloxy)-7a-methyl-3a,4,5,6,7,7a-hexahydro-3H-inden-1-yl])-cyclopropyl}-2-methoxy-vinyl

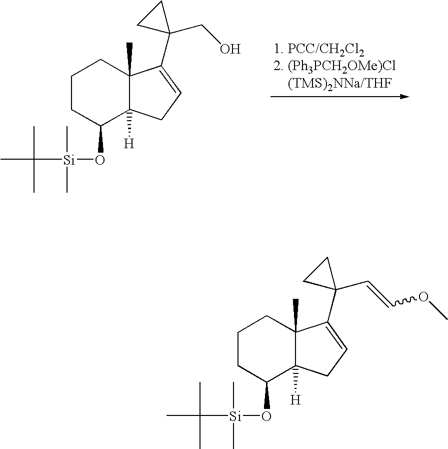

To a stirred suspension of pyridinium chlorochromate (10.3 g, 47.7 mmol) in dichloromethane (100 mL) at room temperature was added dropwise a solution of (3aR,4S,7aR)-{1-[4-(tert-Butyl-dimethyl-silanyloxy)-7a-methyl-3a,4,5,6,7,7a-hexahydro-3H-inden-1-yl]-cyclopropyl}-methanol (6.5 g, 19.31 mmol) in dichloromethene (10.0 mL). The reaction mixture was stirred for 1.0 h and filtered through Celite/Silca gel column (20 g+50 g), which was then washed with 10% AcOEt in hexane to give crude (3aR,4S,7aR)-1-[4-(tert-Butyl-dimethyl-silanyloxy)-7a-methyl-3a,4,5,6,7,7a-hexahydro-3H-inden-1-yl]-cyclopropanecarbaldehyde (5.6 g). To a stirred suspension of (methoxymethyl)triphenylphosphonium chloride (7.5 g, 21.88 mmol) in tetrahydrofurane (150 mL) at 0° C. was added dropwise sodium bis(trimethysilyl)amide (22 mL, 22 mmol, 1.0 M in THF). After 30 min. at 0° C. the solution of (3aR,4S,7aR)-1-[4-(tert-Butyl-dimethyl-silanyloxy)-7a-methyl-3a,4,5,6,7,7a-hexahydro-3H-inden-1-yl]-cyclopropanecarbaldehyde (5.6 g, 16.74 mmol) in tetrahydrofurane (20 mL) was added dropwise. The reaction mixture was stirred for 1 h at 0° C., then water (150 mL) was added and the reaction was extracted with hexane (2×150 mL) and dried over Na₂SO₄. The residue (12.5 g) after evaporationof the solvent was purified by FC (200 g, hexane, 5% AcOEt in hexane) to give the titled compound (5.41 g, 14.92 mmol, 77%)

### (3aR,4S,7aR)-1-{1-[4-(tert-Butyl-dimethyl-silanyloxy)-7a-methyl-3a,4,5,6,7,7a-hexahydro-3H-inden-1-yl])-cyclopropyl}-ethynyl

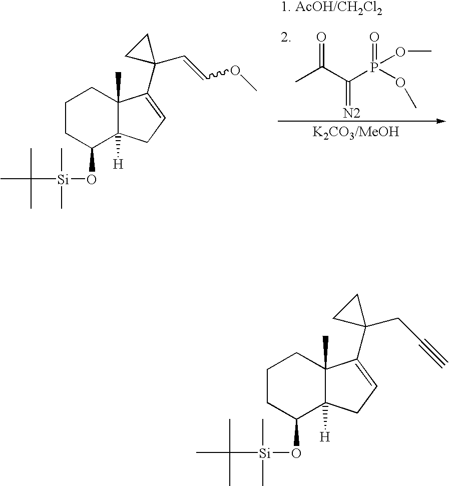

To a stirred solution of (3aR,4S,7aR)-1-E/Z-{1-[4-(tert-Butyl-dimethyl-silanyloxy)-7a-methyl-3a,4,5,6,7,7a-hexahydro-3H-inden-1-yl])-cyclopropyl}-2-methoxy-vinyl (5.41 g, 14.92 mmol) in dichloromethene (50 mL) at room temperature was added acidic acid (25 mL) and the reaction mixture was heated at reflux for 72 hours. NaHCO_{3aq} (350 mL) was added and the reaction mixture was extracted with dichloromethane (2×200 mL), washed with brine (200 mL) and dried over Na₂SO₄) The residue after evaporation of the solvent (1.2 g) was purified by FC (150 g, hexane, 2% AcOEt in hexane) to give (3aR,4S,7aR)-{1-[4-(tert-Butyl-dimethyl-silanyloxy)-7a-methyl-3a,4,5,6,7,7a-hexahydro-3H-inden-1-yl]-cyclopropyl}-acetaldehyde (3.65 g, 10.47 mmol). To a stirred solution of (3aR,4S,7aR)-{1-[4-(tert-Butyl-dimethyl-silanyloxy)-7a-methyl-3a,4,5,6,7,7a-hexahydro-3H-inden-1-yl]-cyclopropyl}-acetaldehyde (3.65 g, 10.47 mmol) in methanol (15 mL) at room temperature was added (1-diazo-2-oxo-propyl)-phosphonic acid dimethyl ester (3.0 g, 15.61 mmol) in methanol (5 mL). The resulting mixture was cooled in an ice bath and potassium carbonate (3.07 g, 22.21 mmol, powdered) was added. The reaction mixture was stirred in the ice bath for 30 min and then at room temperature for 45 min. Water was added (100 mL) and the mixture was extracted with hexane (2×150 mL). The combined extracts were washed with brine (100 mL) and dried over Na₂SO₄. The residue after evaporation of the solvent (3.9 g) was purified by FC (100 g, hexane, 2% AcOEt in hexane) to give the titled compound (2.6 g, 7.54 mmol, 72%)

[α]²⁸_{D}=+29.8 c 0.8, CHCl₃

¹H NMR (CDCl₃): 5.45 (1H, br. s), 4.04 (1H, br. s), 2.40 (2H, m), 2.24 (1H, m), 1.96-1.38 (9H, m), 1.17 (3H, s), 0.88 (9H, s), 0.74-0.54 (4H, m), 0.01 (6H, s);

¹³C NMR (CDCl₃): 156.44(0), 125.39 (1), 82.65 (1), 69.39 (0), 69.23 (1), 55.92 (1), 47.60 (0), 36.42 (2), 34.65 (2), 30.76 (2), 26.04 (2), 20.34 (3), 19.35 (0), 18.30 (2), 11.516 (2), 10.97 (2), −4.55(3), −4.87(3);

| | | | | |
|---|---|---|---|---|
| MS HREI | Calculated for | C₂₂H₃₆OSi | M+ | 344.2535 |
| | Observed | | M+ | 344.2539 |

### (3aR,4S,7aR)-7a-Methyl-1-[1-(5,5,5-trideutero-4-hydroxy-4-trideuteromethyl-pent-2-ynyl)-cyclopropyl]-3a,4,5,6,7,7a-hexahydro-3H-inden-4-ol

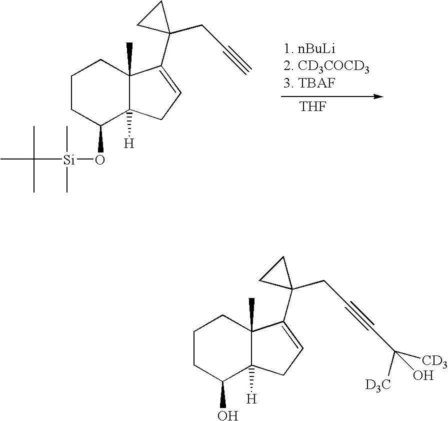

To a stirred solution of (3aR,4S,7aR)-1-{1-[4-(tert-Butyl-dimethyl-silanyloxy)-7a-methyl-3a,4,5,6,7,7a-hexahydro-3H-inden-1-yl])-cyclopropyl}-ethynyl (1.6 g, 4.64 mmol) in tetrahydrofurane (22 mL) at −78° C. was added n-BuLi (4.35 mL, 6.96 mmol, 1.6M in hexane). After stirring at −78° C. for 1 h., acetone-d₆ (1.0 mL, 13.6 mmol, (D,99,96) was added and the stirring was continued for 2.5 h. NH₄Cl_{aq} was added (15 mL) and the mixture was stirred for 15 min at room temperature then extracted with AcOEt (2×50 mL). The combined extracts were washed with brine (50 mL) and dried over Na₂SO₄. The residue after evaporation of the solvent (2.4 g) was purified by FC (50 g, 10% AcOEt in hexane) to give (3aR, 4S,7aR)-5-{1-[4-(tert-Butyl-dimethyl-silanyloxy)-7a-methyl-3a,4,5,6,7,7a-hexahydro-3H-inden-1-yl]-cyclopropyl}-1,1,1-trideutero-2-trideuteromethyl-pent-3-yn-2-ol (1.81 g, 4.43 mmol) which was treated with tetrabutylammonium fluoride (12 mL, 12 mmol, 1.0M in THF) and stirred at 65-75° C. for 48 h. The mixture was diluted with AcOEt (25 mL) and washed with water (5×25 mL), brine (25 mL). The combined aqueous washes were extracted with AcOEt (25 mL) and the combined organic extracts were dried over Na₂SO₄). The residue after evaporation of the solvent (2.5 g) was purified by FC (100 g, 20% AcOEt in hexane) to give the titled compound (1.21 g, 4.11 mmol, 89%)

[α]³⁰_{D}=+2.0 c 0.35, CHCl₃

¹H NMR (CDCl₃): 5.47 (1H, m), 4.15 (1H, m), 2.40 (2H, s), 2.28 (1H, ddd, J=13.4, 11.9, 1.5 Hz), 1.98-1.36 (10H, m), 1.19 (3H, s), 0.70-0.52 (4H, m);

¹³C NMR (CDCl₃): 156.32(0), 125.22 (1), 86.36 (0), 80.33 (0), 69.31 (1), 69.14 (0), 55.20 (1), 47.01 (0), 35.87 (2), 33.70 (2), 29.99 (2), 27.34 (2), 19.39 (2), 19.29 (0), 17.83 (3), 11.05 (2), 10.50 (2);

| | | | | |
|---|---|---|---|---|
| MS HREI | Calculated for | C₁₉H₂₂O₂D₆ | M+ | 294.2466 |
| | Observed | | M+ | 294.2474 |

### (3aR,4S,7aR)-7a-Methyl-1-[1-(5,5,5-trideutero-4-hydroxy-4-trideuteromethyl-pent-2Z-enyl)-cyclopropyl]-3a,4,5,6,7,7a-hexahydro-3H-inden-4-ol

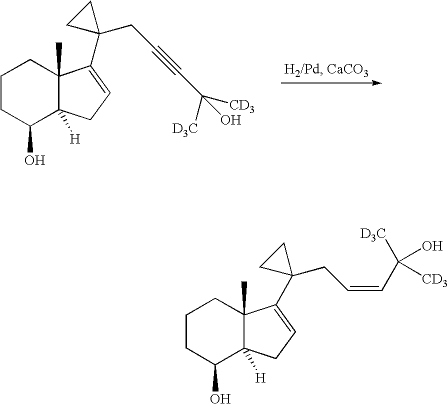

The mixture of (3aR,4S,7aR)-7a-Methyl-1-[1-(5,5,5-trideutero-4-hydroxy-4-trideuteromethyl-pent-2-ynyl)-cyclopropyl]-3a,4,5,6,7,7a-hexahydro-3H-inden-4-ol (1.02 g, 3.46 mmol), ethyl acetate (14 mL), hexane (31 mL), absolute ethanol (1.25 mL), quinoline (66 μl) and Lindlar catalyst (222 mg, 5% Pd on CaCO₃) was hydrogenated at room temperature for 2 h. The reaction mixture was filtered through a celite pad and the pad was washed with AcOEt. The filtrates and the washes were combined and washed with 1M HCl, NaHCO₃ and brine. After drying over Na₂SO₄ the solvent was evaporated and the residue (1.2 g) was purified by FC (75 g, 20% AcOEt in hexane) to give the titled compound (890 mg, 3.0 mmol, 87%)

[α]²⁸_{D}=+1.7 c 0.48, CHCl₃

¹H NMR (CDCl₃): 5.45 (1H, dt, J=11.9, 1.8 Hz), 5.42 (1H, m,), 5.36 (1H, dt, J=12.1, 6.3 Hz), 4.14 (1H, m), 2.43 (1H, m), 2.27 (1H, ddd, J=13.6, 12.2, 1.7 Hz), 2.00-1.24 (11H, m), 1.18 (3H, s), 0.70-0.36 (4H, m);

¹³C NMR (CDCl₃): 156.67(0), 136.58 (1), 128.65 (1), 125.21 (1), 71.48 (0), 69.37 (1), 55.28 (1), 47.07 (0), 35.89 (2), 35.57 (2), 33.68 (2), 30.04 (2), 21.14 (0), 19.37 (3), 17.84 (2), 11.85 (2), 11.06 (2);

| | | | | |
|---|---|---|---|---|
| MS HRES | Calculated for | C₁₉H₂₄O₂D₆ | M + H | 296.2622 |
| | Observed | | M + H | 296.2619 |

### (3aR,4S,7aR)-7a-Methyl-1-[1-(5,5,5-trideutero-4-hydroxy-4-trideuteromethyl-pentyl)-cyclopropyl]-3a,4,5,6,7,7a-hexahydro-3H-inden-4-ol

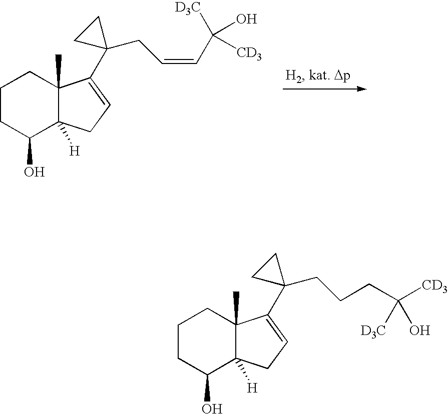

The mixture of (3aR,4S,7aR)-7a-Methyl-1-[1-(5,5,5-trideutero-4-hydroxy-4-trideuteromethyl-pent-2Z-enyl)-cyclopropyl]-3a,4,5,6,7,7a-hexahydro-3H-inden-4-ol (860 mg, 2.9 mmol), 1,4-bis(diphenyl-phosphino)butane 1,5 cyclooctadiene rhodium tetrafluoroborate (200 mg, 0.28 mmol), dichloromethane (35 mL) and one drop of mercury was hydrogenated using Paar apparatus at room temperature and 50 p.s.i. pressure for 2 h. The reaction mixture was filtered through Celite pad, which was then washed with ethyl acetate. The combine filtrates and washes were evaporated to dryness (950 mg) and purified three times by FC (100 g, 20% AcOEt in hexane) to give the titled compound (600 mg, 2.01 mmol, 69%)

[α]³⁰_{D}=−5.3 c 0.45, CHCl₃

¹H NMR (CDCl₃): 5.37 (1H, m,), 4.14 (1H, m), 2.32-1.20 (17H, m), 1.18 (3H, s), 0.64-0.26 (4H, m);

¹³C NMR (CDCl₃): 156.84(0), 124.87(1), 70.79 (0), 69.39 (1), 55.42 (1), 47.19 (0), 43.75 (2), 38.31 (2), 35.86 (2), 33.69 (2), 29.97 (2), 22.35 (2), 21.14 (0), 19.46 (3), 17.88 (2), 12.19 (2), 11.28 (2);

| | | | | |
|---|---|---|---|---|
| MS HREI | Calculated for | C₁₉H₂₆O₂D₆ | M + H | 298.2779 |
| | Observed | | M + H | 298.2787 |

### (3aR,7aR)-7a-Methyl-1-[1-(5,5,5-trideutero-4-trideuteromethyl-4-trimethylsilanyloxy-pentyl)-cyclopropyl]-3a,4,5,6,7,7a-hexahydro-3H-inden-4-one

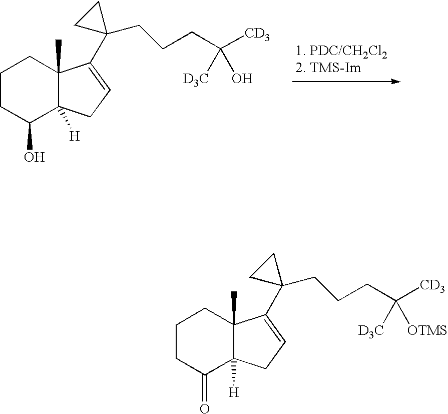

To a stirred suspension of (3aR,4S,7aR)-7a-Methyl-1-[1-(5,5,5-trideutero-4-hydroxy-4-trideuteromethyl-pentenyl)-cyclopropyl]-3a,4,5,6,7,7a-hexahydro-3H-inden-4-ol (450 mg, 1.51 mmol) and Celite (2.0 g) in dichloromethane (10 mL) at room temperature wad added pyridinium dichromate (1.13 g, 3.0 mmol). The resulting mixture was stirred for 3.5 h filtered through silica gel (10 g), and then silica gel pad was washed with 25% AcOEt in hexane. The combined filtrate and washes were evaporated, to give a crude (3aR,7aR)-7a-Methyl-1-[1-(5,5,5-trideutero-4-hydroxy-4-trideuteromethyl-pentenyl)-cyclopropyl]-3a,4,5,6,7,7a-hexahydro-3H-inden-4-one (425 mg, 1.44 mmol, 95%). To a stirred solution of (3aR,7aR)-7a-Methyl-1-[1-(5,5,5-trideutero-4-hydroxy-4-trideuteromethyl-pentenyl)-cyclopropyl]-3a,4,5,6,7,7a-hexahydro-3H-inden-4-one (425 mg, 1.44 mmol) in dichloromethane (10 mL) at room temperature was added trimethylsilyl-imidazole (0.44 mL, 3.0 mmol). The resulting mixture was stirred for 1.0 h filtered through silica gel (15 g) and the silica gel pad was washed with 10% AcOEt in hexane. Combined filtered and washes were evaporated to give the titled compound (450 mg, 1.22 mmol, 85%)

[α]²⁹_{D}=−14.2 c 0.43, CHCl₃

¹H NMR (CDCl₃): 5.33 (1H, dd, J=3.2, 1.5 Hz), 2.81 (1H, dd, J=10.7, 6.4 Hz), 2.44 (1H, ddd, J=15.8, 10.7, 1.6 Hz), 2.30-1.12 (13H, m) overlapping 2.03 (ddd, J=15.9, 6.4, 3.2 Hz), 0.92 (3H, s), 0.66-0.28 (4H, m), 0.08 (9H, s);

¹³C NMR (CDCl₃): 210.74 (0), 155.41 (0), 124.81(1), 73.71 (0), 64.37 (1), 53.92 (0), 44.67 (2), 40.46 (2), 38.21 (2), 34.80 (2), 26.86 (2), 24.06 (2), 22.28 (2), 21.28 (0), 18.40 (3), 12.59 (2), 10.69 (2), 2.62 (3);

| | | | | |
|---|---|---|---|---|
| MS HRES | Calculated for | C₂₂H₃₂O₂SiD₆ | M+ | 368.0318 |
| | Observed | | M+ | 368.3029 |

### 1α,25-Dihydroxy-16-ene-20-cyclopropyl-26,27-hexadeutero-19-nor-cholecalciferol

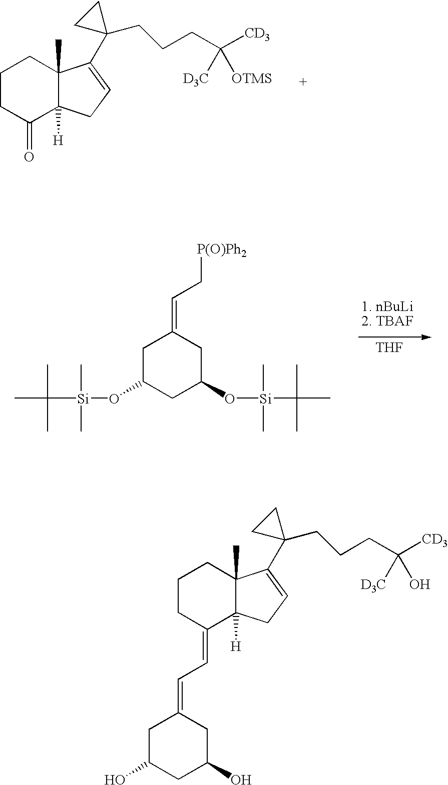

To a stirred solution of a (1R,3R)-1,3-bis-((tert-butyldimethyl)silanyloxy)-5-[2-(diphenylphosphinoyl)ethylidene]-cyclohexane (536 mg, 0.92 mmol) in tetrahydrofurane (7 mL) at −78° C. was added n-BuLi (0.58 mL, 0.93 mmol). The resulting mixture was stirred for 15 min and solution of (3aR,7aR)-7a-Methyl-1-[1-(5,5,5-trideutero-4-trideuteromethyl-4-trimethylsilanyloxy-pentyl)-cyclopropyl]-3a,4,5,6,7,7a-hexahydro-3H-inden-4-one (170 mg, 0.46 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (725 mg) after evaporation of the solvent was purified by FC (15 g, 5% AcOEt in hexane) to give 1α,3β-Di(tert-Butyl-dimethyl-silanyloxy)-25-trimethylsilanyloxy-16-ene-20-cyclopropyl-26,27-hexadeutero-19-nor-cholecalciferol (293 mg, 041 mmol).

To the 1α,3β-Di(tert-Butyl-dimethyl-silanyloxy)-25-trimethylsilanyloxy-16-ene-20-cyclopropyl-26,27-hexadeutero-19-nor-cholecalciferol (293 mg, 0.41 mmol) tetrabutylammonium fluoride (4 mL, 4 mmol, 1M solution in THF) was added, at room temperature. The mixture was stirred for 40 h. diluted with AcOEt (25 mL) and washed with water (5×20 mL), brine (20 mL) and dried over Na₂SO₄. The residue (280 mg) after evaporation of the solvent was purified by FC (15 g, 50% AcOEt in hexane and AcOEt) to give the titled compound (163 mg, 0.39 mmol, 84%)

[α]²⁹_{D}=+65.8 c 0.40, EtOH

UV λmax (EtOH): 243 nm (ε32702251 nm (ε 39060), 261 nm (ε 26595);

¹H NMR (CDCl₃): 6.30 (1H, d, J=11.3 Hz), 5.93 (1H, d, J=11.3 Hz), 5.36 (1H, m), 4.13 (1H, m), 4.05 (1H, m), 2.76 (2H, m), 2.52-1.10 (22H, m), 0.79 (3H, s),0.66-0.24 (4H, m);

¹³C NMR (CDCl₃): 157.05(0), 142.25 (0), 131.15 (0), 124.66(1), 123.70 (1), 115.44 (1), 70.82 (0), 67.42 (1), 67.22 (1), 59.51 (1), 50.17 (0), 44.66 (2), 43.79 (2), 42.22 (2), 38.18 (2), 37.25 (2), 35.64 (2), 29.19 (2), 28.56 (2), 23.55 (2), 22.31 (2), 21.37 (0), 18.04 (3), 12.81 (2), 10.38 (2);

| | | | | |
|---|---|---|---|---|
| MS HRES | Calculated for | C₂₇H₃₆O₃D₆ | M+ | 420.3511 |
| | Observed | | M+ | 420.3524 |

### Example 49

### 1α,25-Dihydroxy-16-ene-20-cyclopropyl-26,27-hexadeutero-cholecalciferol

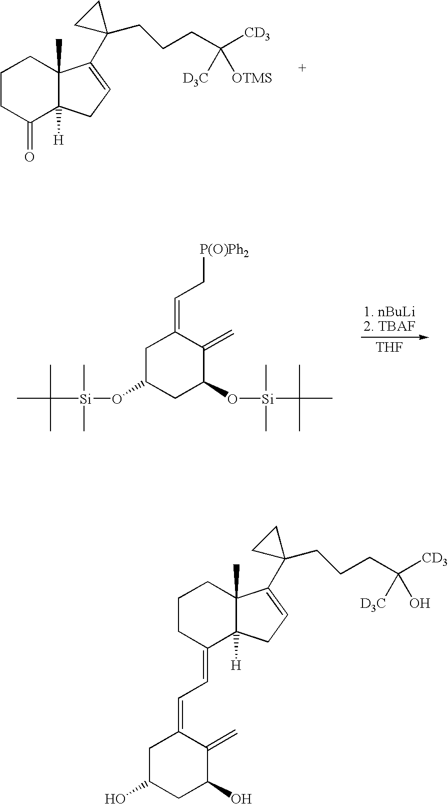

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (536 mg, 0.92 mmol) in tetrahydrofurane (7 mL) at −78° C. was added n-BuLi (0.58 mL, 0.93 mmol). The resulting mixture was stirred for 15 min and solution of (3aR,7aR)-7a-Methyl-1-[1-(5,5,5-trideutero-4-trideuteromethyl-4-trimethylsilanyloxy-pentyl)-cyclopropyl]-3a,4,5,6,7,7a-hexahydro-3H-inden-4-one (170 mg, 0.46 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (725 mg) after evaporation of the solvent was purified by FC (15 g, 5% AcOEt in hexane) to give 1α,3β-Di(tert-Butyl-dimethyl-silanyloxy)-25-trimethylsilanyloxy-16-ene-20-cyclopropyl-26,27-hexadeutero-cholecalciferol (302 mg, 041 mmol). To the 1α,3β-Di(tert-Butyl-dimethyl-silanyloxy)-25-trimethylsilanyloxy-16-ene-20-cyclopropyl-26,27-hexadeutero-cholecalciferol (302 mg, 0.41 mmol) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (280 mg) after evaporation of the solvent was purified by FC (15 g, 50% AcOEt in hexane and AcOEt) to give the titled compound (160 mg, 0.37 mmol, 80%)

[α]²⁹_{D}=+15.3 c 0.34, EtOH

UV λmax (EtOH): 207 nm (ε17011), 264 nm (ε 15067);

¹H NMR (CDCl₃): 6.37 (1H, d, J=11.3 Hz), 6.09 (1H, d, J=11.3 Hz), 5.33 (2H, m), 5.01 (1H, s), 4.44 (1H, m), 4.23 (1H, m), 2.80 (1H, dd, J=11.9, 3.2 Hz), 2.60 (1H, dd, J=13.2, 3.2 Hz), 2.38-1.08 (20H, m), 0.79 (3H, s),0.66-0.24 (4H, m);

¹³C NMR (CDCl₃): 156.97(0), 147.53 (0), 142.41 (0), 132.94 (0), 124.83(1), 124.68(1), 117.14 (1), 111.60 (2), 70.82 (0), 70.71 (1), 66.88 (1), 59.55 (1), 50.30 (0), 45.23 (2), 43.79 (2), 42.90 (2), 38.18 (2), 35.64 (2), 29.19 (2), 28.71 (2), 23.63 (2), 22.30 (2), 21.36 (0), 17.91 (3), 12.82 (2), 10.39 (2);

| | | | | |
|---|---|---|---|---|
| MS HRES | Calculated for | C₂₈H₃₆O₃D₆ | M+ | 432.3510 |
| | Observed | | M+ | 432.3517 |

### Example 50

### 1α-fluoro-25-hydroxy-16-ene-20-cyclopropyl-26,27-hexadeutero-cholecalciferol

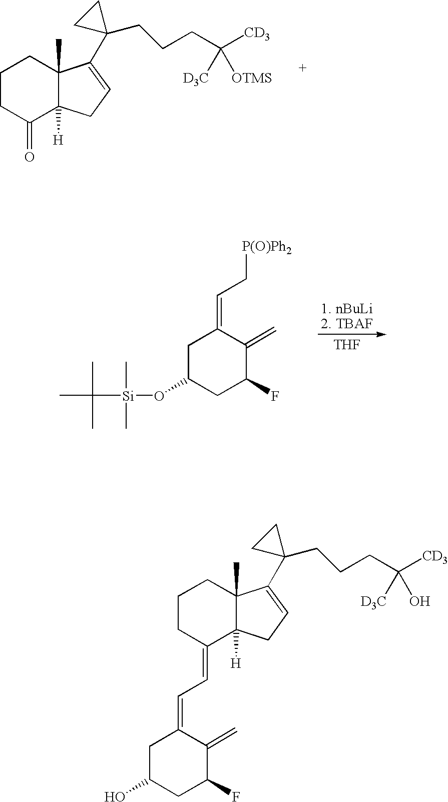

To a stirred solution of a (1S,5R)-1-((tert-butyldimethyl)silanyloxy)-3-[2-(diphenylphosphinoyl)-eth-(Z)-ylidene]-5-fluoro-2-methylene-cyclohexane (433 mg, 0.92 mmol) in tetrahydrofurane (7 mL) at −78° C. was added n-BuLi (0.58 mL, 0.93 mmol). The resulting mixture was stirred for 15 min and solution of (3aR,7aR)-7a-Methyl-1-[1-(5,5,5-trideutero-4-trideuteromethyl-4-trimethylsilanyloxy-pentyl)-cyclopropyl]-3a,4,5,6,7,7a-hexahydro-3H-inden-4-one (170 mg, 0.46 mmol, in tetrahydrofurane (2 mL) was added dropwise. The reaction mixture was stirred at −72° C. for 3.5 h diluted with hexane (25 mL) washed brine (20 mL) and dried over Na₂SO₄. The residue (580 mg) after evaporation of the solvent was purified by FC (15 g, 10% AcOEt in hexane) to give 1α-tert-Butyl-dimethyl-silanyloxy-3,3-fluoro-25-trimethylsilanyloxy-16-ene-20-cyclopropyl-26,27-hexadeutero-cholecalciferol (260 mg, 0.42 mmol). To the give 1α-tert-Butyl-dimethyl-silanyloxy-3,3-fluoro-25-trimethylsilanyloxy-16-ene-20-cyclopropyl-26,27-hexadeutero-cholecalciferol (260 mg, 0.42 mmol) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (260 mg) after evaporation of the solvent was purified by FC (10 g, 30%, 50% AcOEt in hexane) to give the titled compound (140 mg, 0.32 mmol, 70%)

[α]^{\tilde{3}}_{D}=+30.0 c 0.30, EtOH

UV λmax (EtOH): 243 nm (ε12254), 265 nm (ε 12144);

¹H NMR (CDCl₃): 6.40 (1H, d, J=11.3 Hz), 6.10 (1H, d, J=11.1 Hz), 5.39 (1H, s), 5.34 (1H, m), 5.13 (1H, dm, J=50 Hz), 5.11 (1H, s), 4.23 (1H, m), 2.80 (1H, m), 2.63 (1H, m), 2.38-1.08 (19H, m), 0.80 (3H, s),0.66-0.24 (4H, m);

¹³C NMR (CDCl₃): 156.92(0), 143.06 (0, d, J=17 Hz), 142.78 (0), 131.49 (0), 125.48 (1), 124.71(1), 117.16 (1), 114.67(2, d, J=10 Hz), 91.47 (1, d, J=172 Hz), 70.83 (0), 66.58 (1, d, J=6 Hz), 59.55 (1), 50.35 (0), 45.00 (2), 43.80 (2), 40.79 (2, d, J=20 Hz), 38.20 (2), 35.68 (2), 29.15 (2), 28.74 (2), 23.64 (2), 22.32 (2), 20.79 (0), 17.96 (3), 12.81 (2), 10.41 (2);

| | | | | |
|---|---|---|---|---|
| MS HRES | Calculated for | C₂₈H₃₅FO₂D₆ | M + Na | 457.3359 |
| | Observed | | M + Na | 457.3360 |

### BIOLOGICAL EXAMPLES

### Biological Example 1

### Methods

Animals.

Female BALB/c mice (20 g; Charles River Breeding Laboratories) were used for all experiments. All mice were maintained under standard conditions of food and water ad libitum on a 12-hour day-night cycle according to Home Office regulations.

### Mouse Experimental Models of Adhesion Formation

Adhesions were induced in mice with a protocol based on a procedure developed by Sulaiman and colleagues (Sulaiman H, Gabella G, Davis C, Mutsaers S E, Boulos P, Laurent G J, Herrick S E: Growth of nerve fibres into murine peritoneal adhesions. J Pathol 2000, 192:396-403). Briefly, mice were anesthetized with a mixture of inhaled isoflurane and oxygen, and a midline incision was made through the abdominal wall and peritoneum. A standard site (6 mm diameter and 1 mm depth), midway and 0.5 cm lateral to the midline incision on the left abdominal wall, was injured scraping 30 times with a scalpel blade. The cecum was isolated and scraped 30 times on its lateral aspect with a scalpel blade, after first irrigating with 0.9% sterile saline. The two injured surfaces were then apposed by placing two horizontal mattress sutures (8/0 Ethilon; Ethicon, Berkshire, UK) 1 cm apart. The midline incision was closed in two layers; the linea-alba with a continuous suture, and then the skin using michel suture clips (Aesculap, Tuttlingen Germany). This model was validated using leuprolide acetate, a GnRH agonist (Enantone, Takeida) injected at 1 mg/kg one week before surgery. The animals were sacrified at day 7 as usual and adhesion score reduction was evaluated.

To assess the effects of vitamin D compounds on adhesion formation, peritoneal adhesions were induced in 10 animals that were randomized to experimental groups receiving the vitamin D compound or vehicle. In the initial study, animals in the experimental group (n=5) received 0.1-ml i.p. injection of 200 ug/kg of Compound X in saline solution or vehicle (saline solution) only (n=5), at the surgery day then 4 days of daily oral administration (Compound X 100 ug/kg in miglyol or miglyol vehicle only). A second experiment was done with one group of animals (n=5) receiving 0.1-ml i.p. injection of 6 ug/kg of Compound Y in saline solution at the surgery day and then 4 days of daily oral administration (3 ug/kg in miglyol). Control animals (n=5) were similarly administered with vehicle alone (miglyol or saline solution according to the route of administration). The third experiment was designed to compare the effect of calcitriol. Briefly, 28 animals were operated and randomized four different groups: vehicle (miglyol or saline solution according to the route of administration) (n=13), Compound X i.p. 200 ug/kg plus three days oral treatment at MTD (n=5), Compound Y i.p. 30 ug/kg plus three days oral administration at MTD (n=5), calcitriol i.p. 0.6 ug/kg plus three days oral treatment at MTD (n=5). All animals were killed at 7 days, and the adhesions were quantified in a blinded fashion. Each animal received an adhesion score based on the number, length and strength of adhesions formed. Millimetres of caecum adherent to peritoneum was also measured.

### Colorimetric Determination of Serum Calcium Level

Serum calcium was measured with Calcium Dry-fast kit (Sentinel diagnostics, Milan, Italy) with minor modification over the standard method: 10 ul serum, standard solution, or water are added to 100 ul of reconstituted assay reagent in a 96 well plate and incubated 5 min before reading at 550 nm, RT. Standards are run in quadruplicate, blank (water) in duplicate, and samples in triplicate.

### Results

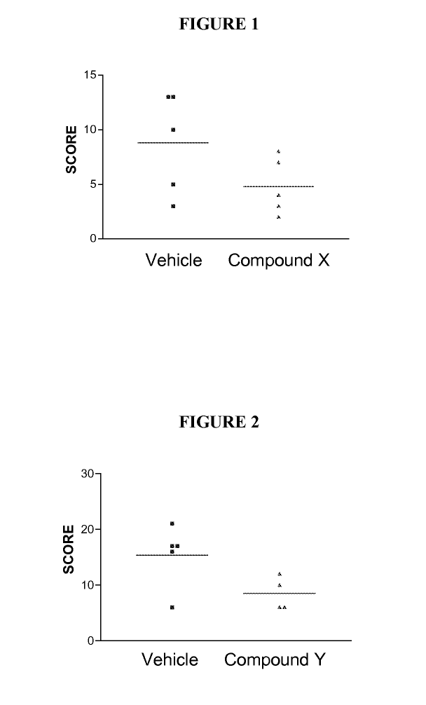

Balb/c mice were used to induce post surgical adhesions in a model of caecum abrasion. Four hours after surgery, mice were administered once with Compound X 200 ug/kg i.p. or vehicle alone, and then oral, once a day, for 4 days with 100 ug/kg. At day seven, mice were sacrificed and their abdomen was open to check for adhesion presence. The length and strength of each adhesion were measured and represented as a score for every single animals. FIG. 1 shows that Compound X administration significantly reduces total adhesions score.

Balb/c mice were used to induce post surgical adhesions in a model of caecum abrasion. Four hours after surgery, mice were administered once with Compound Y 6 ug/kg i.p. or vehicle alone, and then orally 3 ug/kg, once a day, for 4 days. At day seven, mice were sacrificed and their abdomen was open to check for adhesion presence. The length and strength of each adhesion were measured and represented as a score for every single animals. FIG. 2 shows that Compound Y administration significantly reduces total adhesions score.

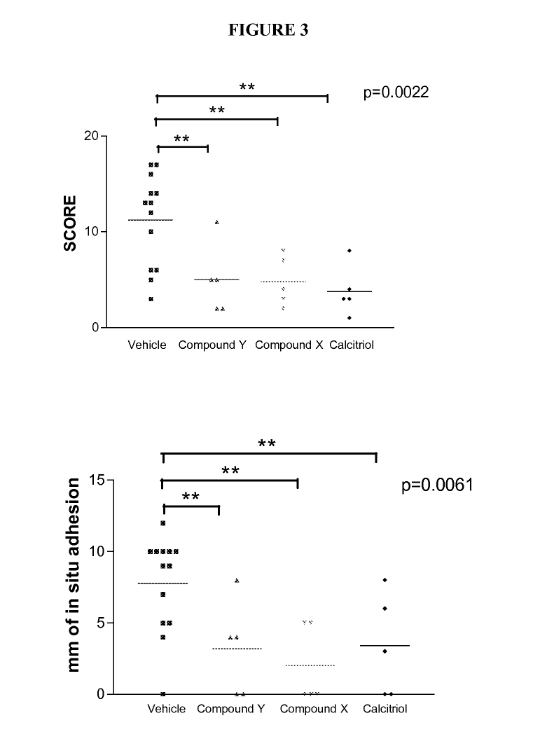

Balb/c mice were used to induce post surgical adhesions in a model of caecum abrasion. Four hours after surgery, mice were administered once with vehicle or Compound X 200 ug/kg i.p., Compound Y 30 ug/kg i.p., calcitriol 0.6 ug/kg i.p., and then orally at MTD (100, 3, 0.3 ug/kg respectively), once a day, for 4 days. At day seven, mice were sacrificed and their abdomen was opened to check for adhesion presence. The length and strength of each adhesion were measured and represented as a score for every single animal. Alternatively length (mm) of caecum adherent to peritoneum were measured. Statistical analysis was performed by Anova with Bonferroni's Multiple Comparison Test. FIG. 3 shows the effect of Compound X and Compound Y and calcitriol compared to miglyol control. The difference between animals receiving vitamin D compound and those in vehicle groups was statistically significant.

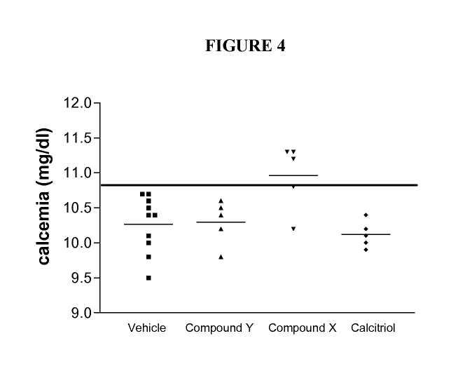

Blood serum was obtained from animals of the experiment described in FIG. 3 and calcium level was measured. FIG. 4 shows the serum calcium levels in these experiments. Except for Compound X administration, that is slightly hypercalcemic in this setting, the other animal groups had normal serum calcium levels.

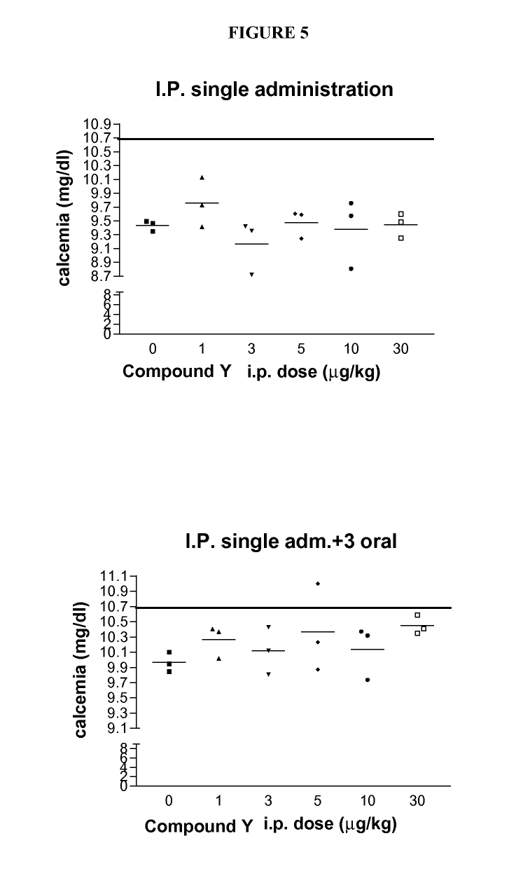

CD1 mice were used to test serum calcemia after different doses of Compound Y administered i.p. A second group of animals received both i.p. (different doses) and oral administration for 3 days at MTD (3 ug/kg). Serum calcemia was measured 24 h after the last administration. FIG. 5 shows that no hypercalcemic effect was observed with this regimen.

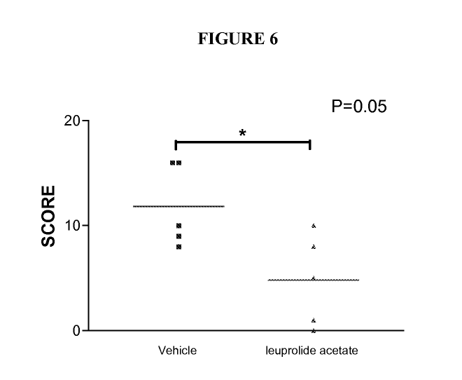

The caecum abration mouse model used in the experiments of FIGS. 1, 2 and 3 was validated using leuprolide acetate, a GnRH agonist (Enantone, Takeda) injected at 1 mg/kg one week before surgery. The animals were sacrificed at day 7 as usual, and total adhesion score was measured, as shown in FIG. 6. A statistically significant reduction of score was observed.

In summary, the results show that vitamin D compounds, as exemplified by Compound X, Compound Y and calcitriol, are effective in reducing the incidence and/or severity of peritoneal adhesions in a mouse model.

### Biological Example 2

### Effect of Compound Y on the Fibrinolysis Pathway

### Methods:

Confluent 3T3-L1 cells were incubated overnight with the following compounds: TGF-β1 5 ng/ml, Compound Y 10⁻⁶, 10⁻⁷, 10⁻⁸ and 10⁻⁹ M. After removing the supernatants, Fibrinolysis Buffer containing 3 μM fibrinogen and 100 nM plasminogen (“BM”) was added to each well. A fibrin clot was induced by adding 20 nM thrombin. Plates were incubated at 37° C. and clot density was determined by measuring optical density at 405 nm. Results were shown as relative fibrin clot density at different timepoints normalized to untreated cells.

### Results:

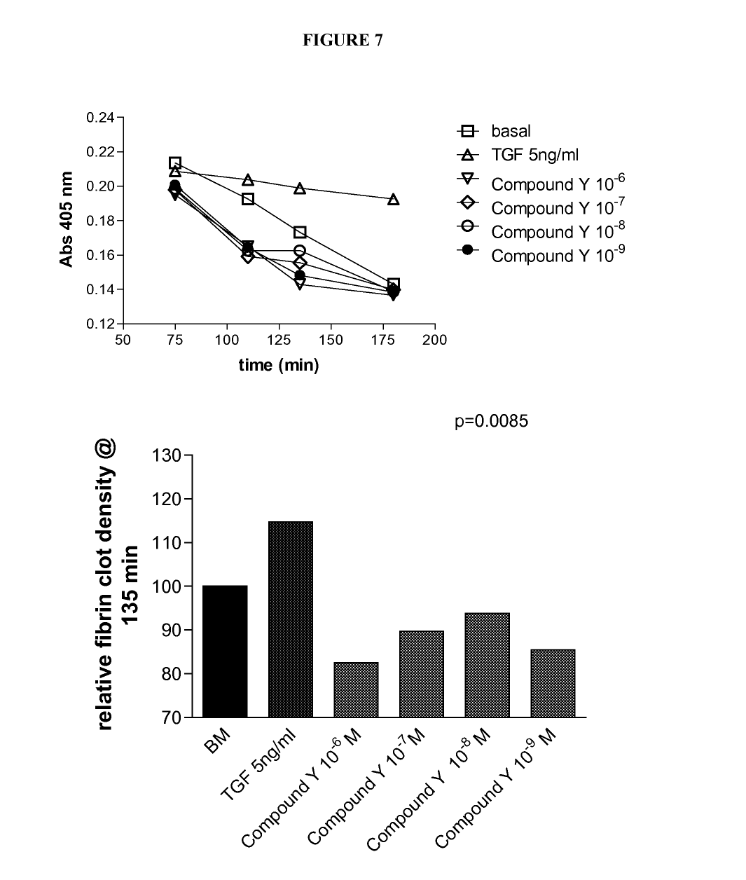

The results are shown in FIG. 7. Compound Y accelerates fibrin clot lysis by mouse fibroblasts in a dose dependent manner.

### Biological Example 3

### Effect of Compound Y on the Fibrinolysis Pathway

### Methods:

Human mesothelial cells (LP9, Cornell University) were plated in a 96 well plate in the presence of different doses of Compound Y. Supernatants were harvested at 48 h and tPA and PAI-1 production was measured by ELISA. tPA/PAI-1 ratio was calculated. Statistical analysis was performed by ANOVA test.

### Results:

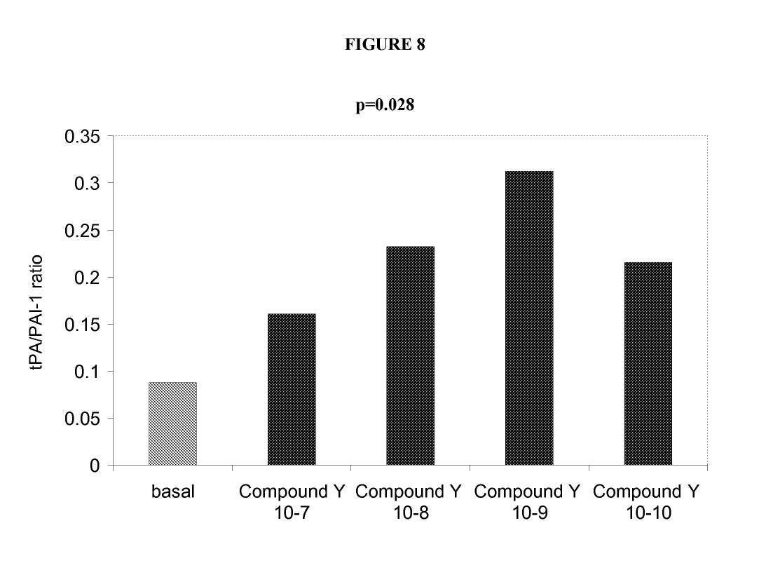

The results are shown in FIG. 8. tPA/PAI-1 ratio is increased by Compound Y treatment on human mesothelial cells, indicating an upregulation of fibrinolytic activity.

### Biological Example 4

### Intraperitoneal Single Administration of Compound Y—Serum Calcium Levels

### Methods:

Female BALB/c mice were injected i.p. with 100 ml of Compound Y or calcitriol at different doses in sterile saline solution with 0.1% Tween-20 0.1% ethanol. Blood was collected at two timepoints and serum calcemia was measured.

### Results:

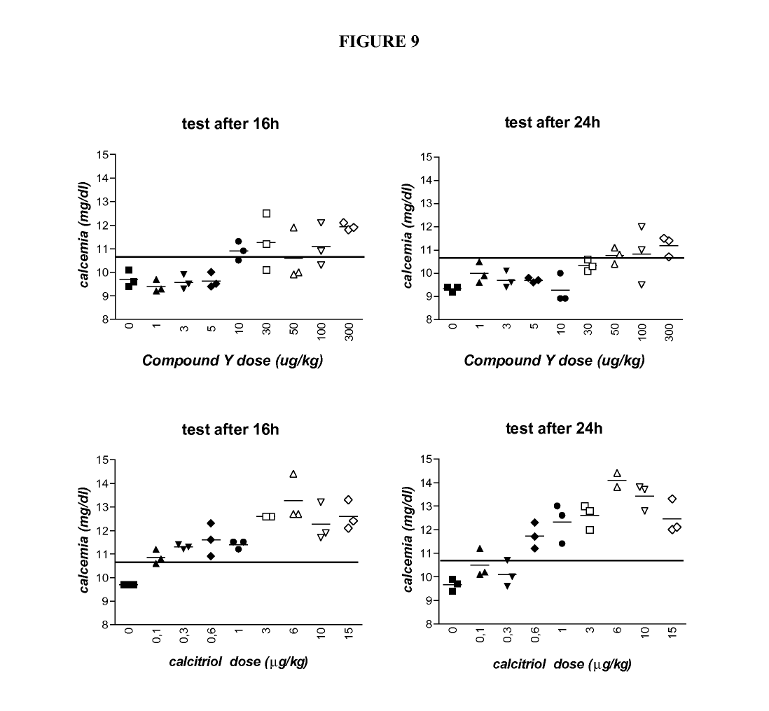

The results are shown in FIG. 9. Transient hypercalcemia was observed with Compound Y at high dosage (300 mg/kg) whereas calcitriol induces more pronounced and sustained hypercalcemia at both 16 and 24 hours after i.p. administration.

### Biological Example 5

### Dose Response Efficacy Study—Cecal Peritoneal Scraping and Sewing Mouse Model of Post-Surgical Adhesions

### Methods:

Different doses of Compound Y were tested in the CPSS (cecal peritoneal scraping and sewing) mouse model of post-surgical adhesion. Animals were treated with 30-100-300 ug/kg of Compound Y in 100 ml of sterile saline with 0.1% Tween-20, 0.1% ethanol, i.p. at the end of surgery. Animals were sacrificed after two weeks and peritoneral adhesion evaluated. Means of in situ score of the four groups were compared and percentage of reduction of the mean in situ score of treated animals compared with vehicle group was considered.

### Results:

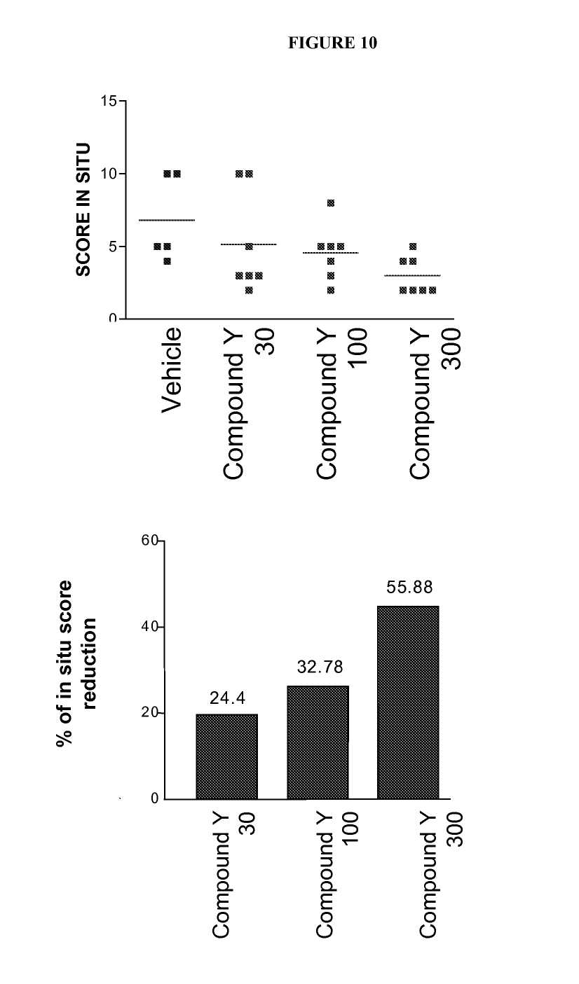

The results are shown in FIG. 10. Compound Y reduces cecal-peritoneal adhesion formation in a dose dependent manner.

### Biological Example 6

### Dose Response Efficacy Study—Double Uterine Horn Rabbit Model of Post-Surgical Adhesions

Methods:

### General Description of the Double Uterine Horn Rabbit Model (DUH).

### Protocol:

Animals: Twenty eight, female New Zealand White rabbits, 2.4-2.7 kg, were purchased from Irish Farms (Norco, Calif.) and quarantined for at least 2 days prior to use. Seven rabbits were randomized into four treatment groups prior to initiation of surgery. The rabbits were housed on a 12:12 light:dark cycle with food and water available ad libitum.

Double Uterine Horn Model: Rabbits were anesthetized with a mixture of 55 mg/kg ketamine hydrochloride and 5 mg/kg Rompum intramuscularly. Following preparation for sterile surgery, a midline laparotomy was performed. The uterine horns were exteriorized and traumatized by abrasion of the serosal surface with gauze until punctate bleeding developed. Ischemia of both uterine horns was induced by removal of the collateral blood supply. The remaining blood supply to the uterine horns was the ascending branches of the utero-vaginal arterial supply of the myometrium. Treated animals received 25 mL of either vehicle (lactated Ringers' solution with 0.1% Tween 20, 0.1% isopropyl alcohol), or vehicle with one of two doses of Compound Y (30 or 300 μg/kg). An additional group of animals received surgery only. The horns were then returned to their normal anatomic position and the midline sutured with 3-0 Vicryl. The animals were weighed before surgery and necropsy.

After 7 days, the rabbits were terminated and the percentage of the area of the horns adherent to various organs determined. In addition, the tenacity of the adhesions was scored using the following system:
0=No Adhesions
1=mild, easily dissectable adhesions
2=moderate adhesions; non-dissectable, does not tear the organ
3=dense adhesions; non-dissectable, tears organ when removed

In addition an overall score which took into account all of the above data was given to each rabbit. The following scoring system was used:

| Adhesion | |
|---|---|
| Score | Description |
| 0 | No adhesions |
| 0.5 | Light, filmy pelvic adhesions involving only one organ, |
| | typically only 1 or 2 small adhesions |
| 1.0 | Light, filmy adhesions, not extensive although slightly more |
| | extensive than 0.5 |
| 1.5 | Adhesions slightly tougher and more extensive than a 1 rating |
| 2.0 | Tougher adhesions, a little more extensive, uterine horns |
| | usually have adhesions to both bowel and bladder |
| 2.5 | Same as 2, except the adhesions are usually not filmy at any |
| | site and more extensive |
| 3.0 | Tougher adhesions than 2, more extensive, both horns are |
| | attached to the bowel and bladder, some movement of the |
| | uterus possible |
| 3.5 | Same as 3, but adhesions slightly more extensive and tougher |
| 4.0 | Severe adhesions, both horns attached to the bowel and |
| | bladder, unable to move the uterus without tearing the |
| | adhesions |

The rabbits were scored by two independent observers that were blinded to the prior treatment of the animal. If there was disagreement as to the score to be assigned to an individual animal, the higher score was given.

### Results:

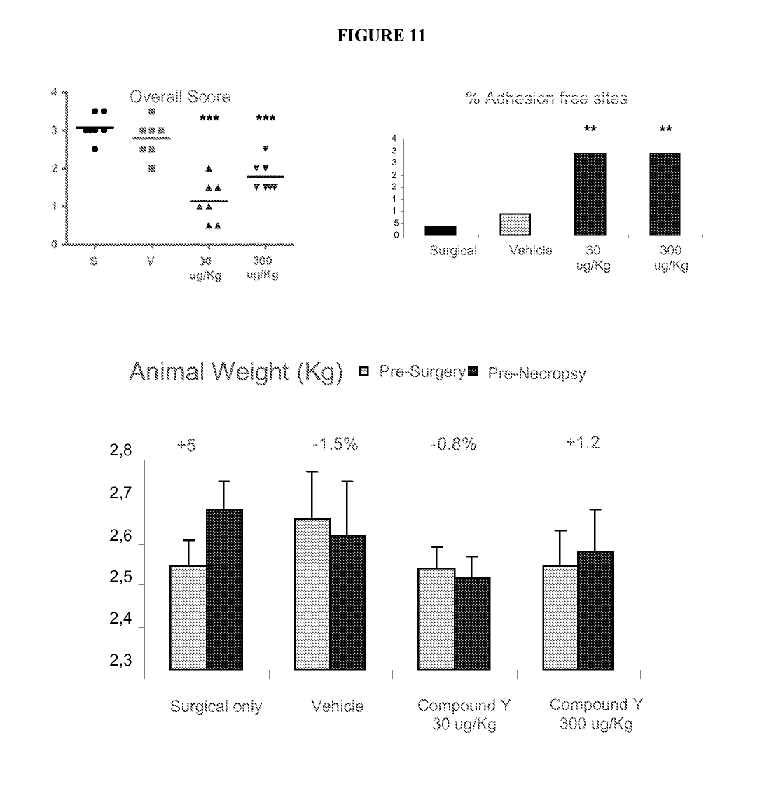

The results are shown in FIG. 11. Compound Y significantly reduced adhesion formation at both doses compared to the vehicle group (P<0.001 for both doses compared with both controls). No peritoneal inflammation, weight loss or hypercalcemia were observed at necropsy.

### Biological Example 7

### Effect of Compound Y on Serum Calcium Levels in DUH Rabbit Model Animals

### Methods:

One ml of blood was drawn into red top tubes prior to surgery, 24 hours after surgery and prior to necropsy to allow the generation of serum to measure serum calcium levels. The blood was allowed to clot, the serum harvested by centrifugation and the serum calcium measured.

### Results:

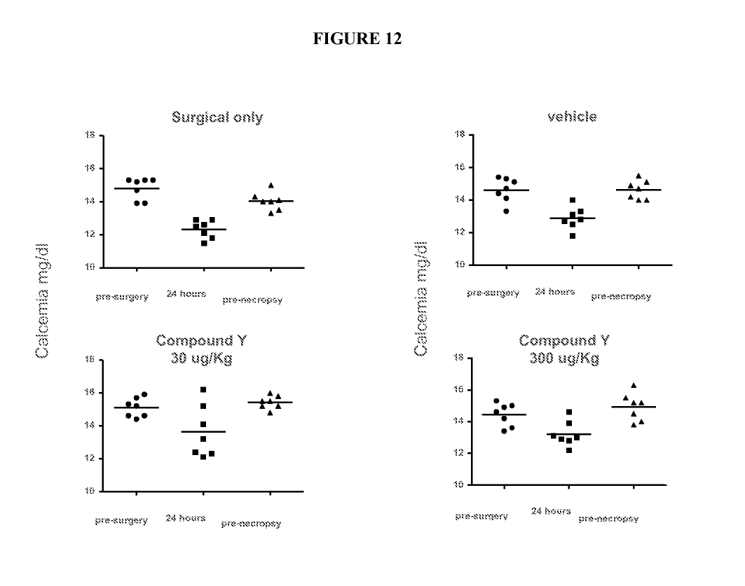

The results are shown in FIG. 12. No signs of hypercalcemia were observed at the three timepoints at both doses.

### Biological Example 8

### Investigation of Possible Impairment of Tissue Healing in Mouse Model

### Methods:

One full thickness wound of 1 cm in diameter was marked using a template and the tissue was excised to the level of the panniculus carnosus using dissecting scissors and forceps. The wound healing was determined by drawing the wound margin with tracing film. The rate of wound healing is expressed as the percentage area remaining 5 hairless mice per group, daily oral treatment with 3 mg/kg of Compound Y, 5 days a week. Wound healing curve using the Boltzman equation and V50 (half time of wound healing) were calculated. No statistically significant difference between the two curves was observed.

Human mesothelial cells (Cornell Cell Repositories) were plated in a 96 well plate in the presence of different doses of Compound Y. Supernatants were harvested at 48 h and VEGF and TGF-b production was measured by ELISA. Total number of live cells were quantified by fluorimetric assay.

### Results:

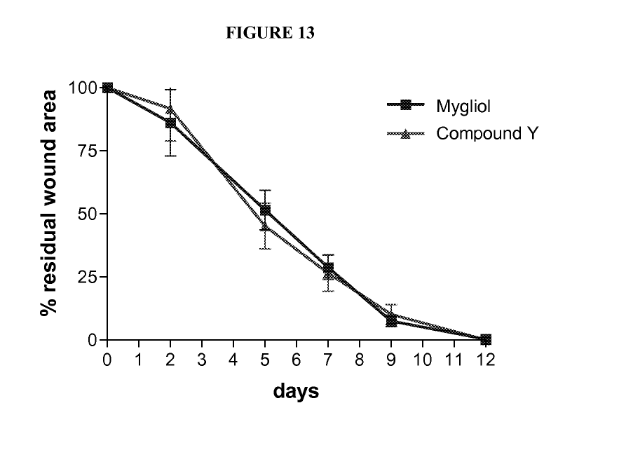

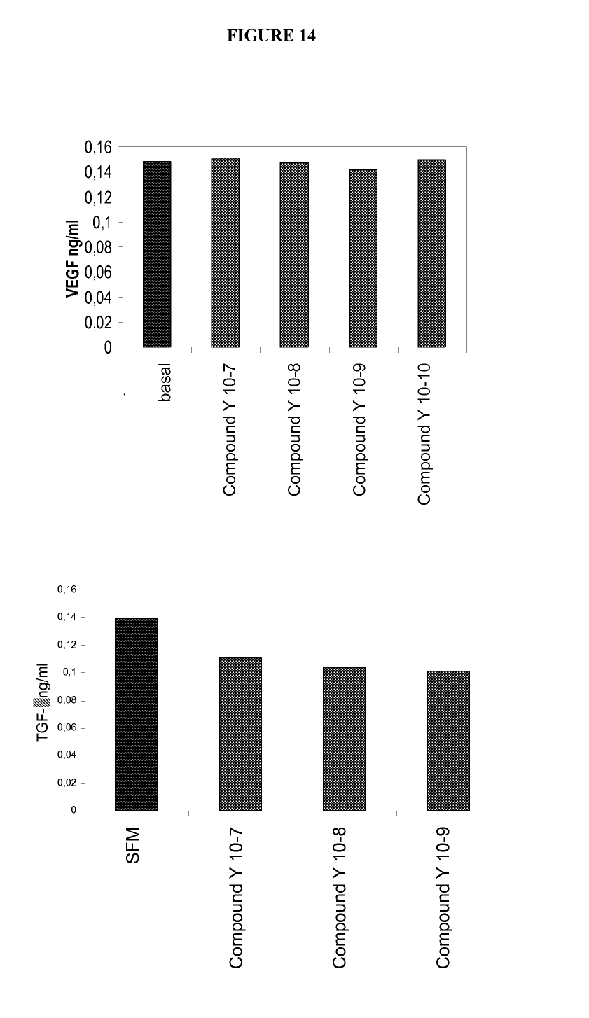

The results are shown in FIGS. 13 and 14.

The results show that Compound Y does not delay wound healing in the full thickness mouse model (FIG. 13). Compound Y treatment of human mesothelial cells does not affect VEGF and TGF-b production, these factors being important in promoting wound healing (FIG. 14).

### Biological Example 9

### Investigation of Risk of Mortality Due to Infection Potentiation in Mice

### Methods:

CLP (cecal ligation and puncture) was performed with 23G needle and 50-70% of cecal ligation. Compound Y 30 mg/kg was administered IP on the day of surgery. The mortality was checked twice a day. Ten C57/BL6 mice per group were used.

### Results:

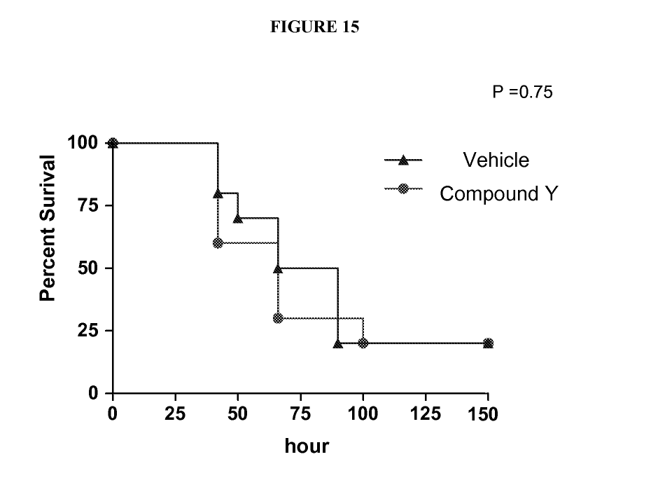

The results are shown in FIG. 15. Comparison of survival curves with log rank test revealed no statistically significant difference between vehicle and Compound Y treated mice. This confirms that Compound Y is not associated with risk of increase in mortality due to potentiation of bacterial infection.

### Biological Example 10

### Efficacy as Compared with Icodextrin and Lazaroids

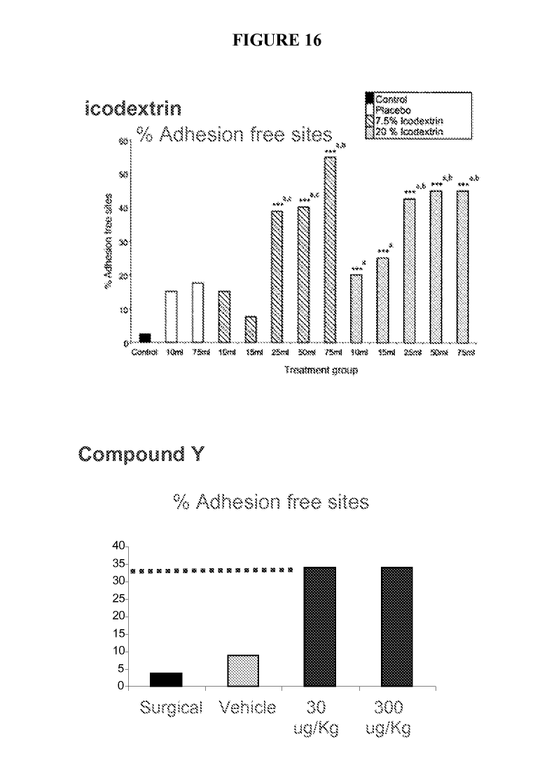

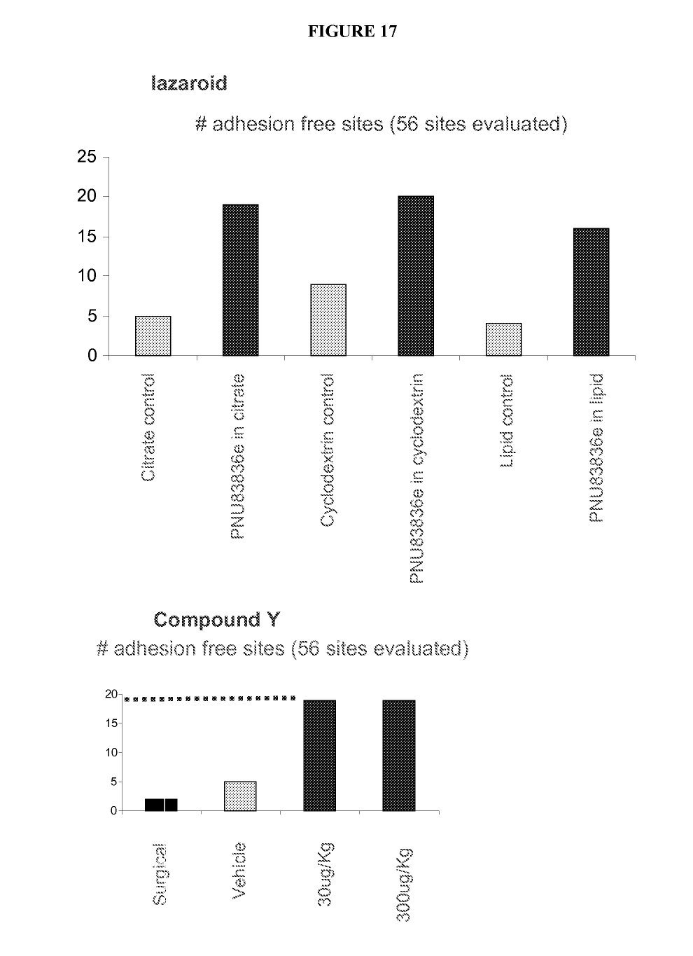

Results obtained using Compound Y have been compared with those obtained using icodextrin and lazaroids as reported in literature publications. The results are shown in FIGS. 16 and 17. The results show that Compound Y has comparable efficacy in preventing adhesion formation in the DUH rabbit model to prior studies of icodextrin (FIG. 16) and lazaroids (FIG. 17). Data on icodextrin is taken from SJS Verco et al. Human Reproduction, 15(8) 1764-1772 (2000). Data on lazaroid is taken from K E Rodgers et al. Human Reproduction, 13(9) 2443-2451 (1998). The comparator Compound Y experiments were performed in the same laboratory as the Rodgers et al experiments.

### Summary of Biological Results

It may be concluded from the results that vitamin D compounds, as exemplified by calcitriol, Compound X and particularly Compound Y, are efficacious in reducing adhesion formation in animal models without adverse effect on wound healing and without increasing mortality due to potentiation of infection.

### ABBREVIATIONS

SFM=serum free medium
i.p.=intraperitoneal

### “Comprising”

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. (canceled)

2. A method for preventing adhesions in a subject, comprising administering to a subject in need thereof an effective amount of a vitamin D compound, such that adhesions are prevented in said subject.

3. The method of claim 2, further comprising identifying a subject in need of prevention of adhesions.

4. The method according to claim 2, further comprising the step of obtaining the vitamin D compound.

5. The method of claim 2, wherein the subject is a mammal.

6. The method of claim 5, wherein the subject is a human.

7. The method according to claim 1, wherein the vitamin D compound is formulated in a pharmaceutical composition together with a pharmaceutically acceptable diluent or carrier.

8. (canceled)

9. A pharmaceutical formulation comprising a vitamin D compound and a pharmaceutically acceptable carrier for use in the prevention of adhesions.

10. The pharmaceutical formulation according to claim 9, packaged with instructions directing administration of said formulation to a patient in need of the prevention of adhesions to prevent adhesions in said patient.

11. (canceled)

12. (canceled)

13. The method according to claim 2, wherein the vitamin D compound is administered separately, sequentially or simultaneously in separate or combined pharmaceutical formulations with a second medicament for the prevention and/or treatment of adhesions.

14. The method according to claim 2 wherein the adhesions are post-surgical adhesions.

15. The method according to claim 2 wherein the adhesions are peritoneal adhesions.

16. The method of claim 2, wherein said vitamin D compound is a compound of the formula: 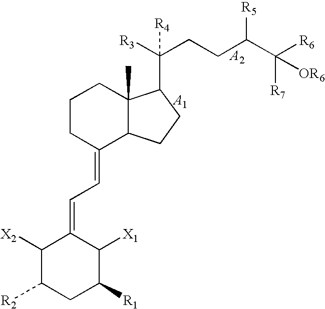
wherein:
A₁ is single or double bond;
A₂ is a single, double or triple bond;
X₁ and X₂ are each independently H or ═CH₂, provided X₁ and X₂ are not both ═CH₂;
R₁ and R₂ are each independently OH, OC(O)C₁-C₄ alkyl, OC(O)hydroxyalkyl, OROC(O)haloalkyl, OAc;
R₃, R₄ and R₅ are each independently hydrogen, C₁-C₄ alkyl, hydroxyalkyl, or haloalkyl, or R₃ and R₄ taken together with C₂₀ form C₃-C₆ cycloalkyl; and
R₆ and R₇ are each independently C₁₋₄alkyl or haloalkyl; and
R₈ is H, —COhydroxyalkyl or —COhaloalkyl; and
pharmaceutically acceptable esters, salts, and prodrugs thereof.

17. The method of claim 16, wherein R₁ and R₂ are OH or OC(O)C₁-C₄ alkyl.

18. The method of claim 17, wherein R₁ and R₂ are OAc.

19. The method of claim 17, wherein R₁ and R₂ are OH.

20. The method of claim 16, wherein X₁ is ═CH₂ and X₂ is H.

21. The method of claim 16, wherein A₁ is single bond and A₂ is a single bond.

22. The method of claim 16, wherein R₃ and R₄ taken together with C₂₀ form C₃-C₆ cycloalkyl.

23. The method of claim 22, wherein R₃ and R₄ taken together with C₂₀ form cyclopropyl.

24. The method of claim 16, wherein R₅ is hydrogen.

25. The method of claim 16, wherein R₆ and R₇ are each independently C₁₋₄alkyl.

26. The method of claim 25, wherein R₆ and R₇ are each independently methyl.

27. The method of claim 16, wherein R₈ is H.

28. The method of claim 16, wherein R₁ and R₂ are OH or OC(O)C₁-C₄ alkyl, X₁ is ═CH₂ and X₂ is H, A₁ is single bond, A₂ is a single bond, R₃ and R₄ taken together with C₂₀ form C₃-C₆ cycloalkyl, R₅ is hydrogen, R₆ and R₇ are each independently C₁₋₄alkyl, and R₈ is H.

29. The method of claim 28, wherein R₁ and R₂ are OH or OAc, R₃ and R₄ taken together with C₂₀ form cyclopropyl, and R₆ and R₇ are each methyl.

30. The method of claim 2, wherein said vitamin D compound is a compound selected from the group consisting of:
(a) a compound of the formula (IV): 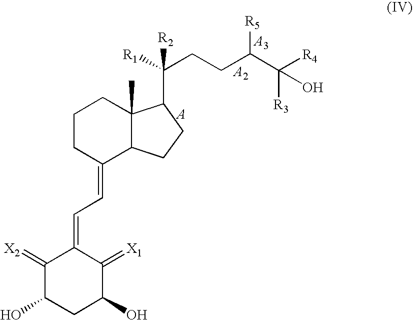
wherein:
X₁ and X₂ are H₂ or CHₐ, wherein X₁, and X₂ are not CH₂ at the same time;
A is a single or double bond;
A₂ is a single, double or triple bond;
A₃ is a single or double bond;
R₁ and R₂ are hydrogen, C₁-C₄ alkyl or 4-hydroxy-4-methylpentyl, wherein R₁ and R₂ are not both hydrogen;
R₅ is H₉ or oxygen, R₅ may also represent hydrogen or may be absent;
R₃ is C₁-C₄ alkyl, hydroxyalkyl or haloalkyl, and
R₄ is C₁-C₄ alkyl, hydroxyalkyl or haloalkyl;
(b) a compound of the formula (V): 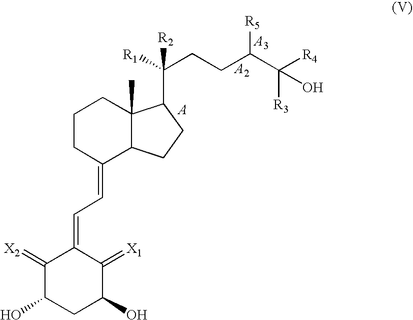
wherein:
X₁ and X₂ are H₂ or CH₂, wherein X₁ and X₂ are not CH₂ at the same time;
A is a single or double bond;
A₂ is a single, double or triple bond;
A₃ is a single or double bond;
R₁ and R₂ are alkyl, wherein R₁ and R₂ are not both hydrogen;
R₅ is H₂ or oxygen, R₅ may also represent hydrogen or may be absent;
R₃ is C₁-C₄ alkyl, hydroxyalkyl or haloalkyl; and
R₄ is C₁-C₄ alkyl, hydroxyalkyl haloalkyl;
c) a compound of the formula (VI): 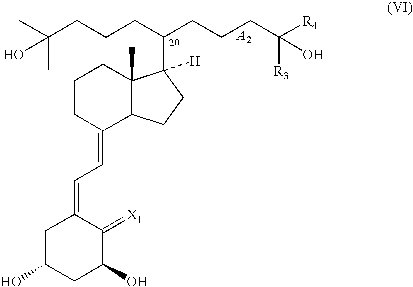
wherein:
X₁ is H₂ or CH₂;
A₂ is a single, a double or a triple bond;
R₃ is C₁-C₄ alkyl, hydroxyalkyl, or haloalkyl;
R₄ is C₁-C₄ alkyl, hydroxyalkyl or haloalkyl;
and the configuration at C₂₀ is R or S;
(d) a compound of the formula (VII): 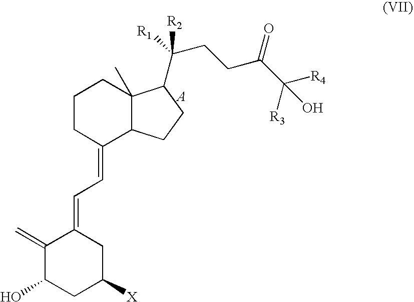
wherein:
A is a single or double bond;
R₁ and R₂ are each, independently, hydrogen, alkyl;
R₃, and R₄, are each independently alkyl, and
X is hydroxyl or fluoro;
(e) a compound of the formula (VIII): 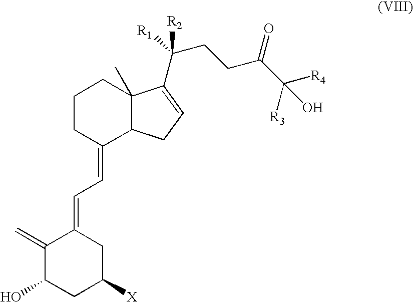
wherein:
R₁ and R₂, are each, independently, hydrogen, or alkyl;
R₃ is alkyl,
R₄ is alkyl; and
X is hydroxyl or fluoro;
(f) a compound of the formula (IX): 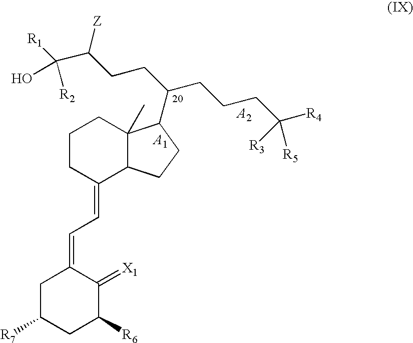
wherein:
A₁ is a single or double bond;
A₂ is a single, a double or a triple bond;
R₁, R₂, R₃ and R₄ are each independent C₁-C₄ alkyl, C₁-C₄ deuteroalkyl, hydroxyalkyl, or haloalkyl;
R₅, R₆ and R₇ are each independently hydroxyl, OC(O)C₁-C₄ alkyl, OC(O)hydroxyalkyl, or OC(O)haloalkyl;
the configuration at C₂₀ is R or S;
X₁ is H₂ or CH₂;
Z is hydrogen when at least one of R₁ and R₂ is C₁-C₄ deuteroalkyl and at least one of R₃ and R₄ is haloalkyl or when at least one of R₁ and R₂ is haloalkyl and at least one of R₃ and R₄ is C₁-C₄ deuteroalkyl; or Z is —OH, ═O, —SH, or —NH₂;
and pharmaceutically acceptable esters, salts, and prodrugs thereof;
(f) a compound of the formula (X): 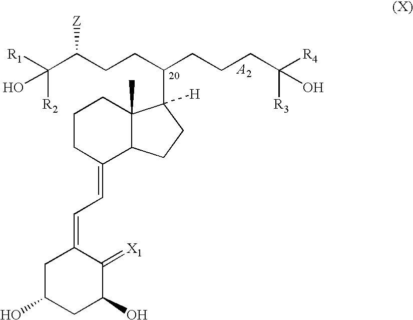
wherein:
X₁ is H₂ or CH₂;
A₂ is a single, a double or a triple bond;
R₁, R₂, R₃ and R₄ are each independently C₁-C₄ alkyl, hydroxyalkyl, or haloalkyl;
Z is —OH, ═O, —NH₂ or —SH; and
the configuration at C₂₀ is R or S,
and pharmaceutically acceptable esters, salts, and prodrugs thereof;
(g) a compound of the formula (XI): 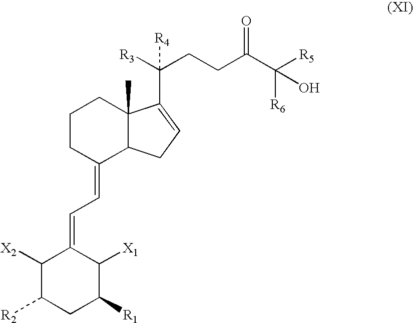
wherein:
X₁ and X₁ are each independently H₂ or ═CH₂, provided X₁ and X₁ are not both ═CH₂;
R₁ and R₂ are each independently, hydroxyl, OC(O)C₁-C₄ alkyl, OC(O)hydroxyalkyl, OC(O)fluoroalkyl;
R₃ and R₄ are each independently hydrogen, C₁-C₄ alkyl, hydroxyalkyl or haloalkyl, or R₃ and R₄ taken to ether with C₂₀ form C₃-C₆ cylcoalkyl; and
R₅ and R₆ are each independently C₁-C₄ alkyl or haloalkyl;
and pharmaceutically acceptable esters, salts, and prodrugs thereof;
(h) a compound of the formula (XII): 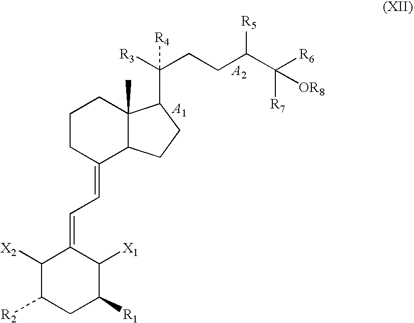
wherein:
A₁ is single or double bond;
A₂ is a single, double or triple bond;
X₁ and X₂ are each independently H or ═CH₂, provided X₁ and X₂ are not both ═CH₂;
R₁ and R₂ are each independently H, OC(O)C₁-C₄ alkyl, OC(O)hydroxyalkyl, OC(O)haloalkyl;
R₃, R₄, and R₅ are each independently hydrogen, C₁-C₄ alkyl, hydroxyalkyl, or haloalkyl, or R₃ and R₄ taken together with C₂₀ form C₃-C₆ cycloalkyl; and
R₆ and R₇ are each independently C₁₋₄alkyl or haloalkyl; and
R₈ is H, —COC₁-C₄alkyl, —COhydroxyalkyl or —COhaloalkyl; and
pharmaceutically acceptable esters, salts, and prodrugs thereof;
(i) a compound of the formula (XIV): 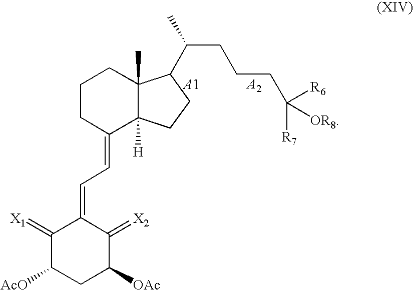
(i) a compound of the formula (XV): 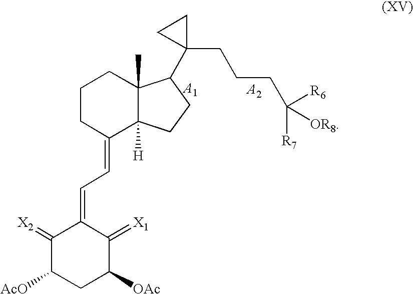
(k) a compound of the formula (XVI): 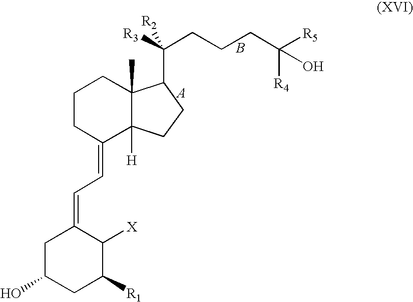
wherein:
X is H₂ or CH₂
R₁ is hydrogen, hydroxy or fluorine
R₂ is hydrogen or methyl
R₃ is hydrogen or methyl provided that when R₂ or R₃ is methyl, R₃ or R₂ must be hydrogen
R₄ is methyl, ethyl or trifluoromethyl
R₅ is methyl, ethyl or trifluoromethyl
A is a single or double bond
B is a single, E-double, Z-double or triple bond;
(I) a compound of the formula (XVII): 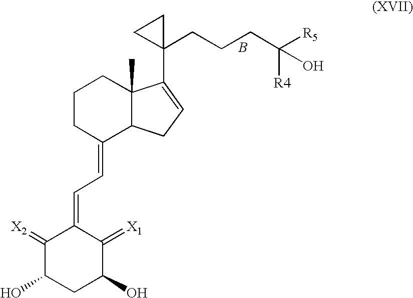
wherein:
B is single, double, or triple bond;
X₁ and X₂ are each independently H₂ or CH₂, provided X₁ and X₂ are not both CH₁; and
R₄ and R₅ are each independently alkyl or haloalkyl;
(l) a compound of the formula (XVIII): 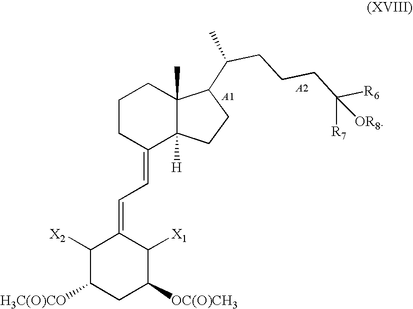
wherein A₁ is a double bond, or single bond;
A₂ is a triple bond, double bond, or single bond;
X₁ is ═CH₂ or H₂.
X₂ is H₂:
R₆ and R₇ are each independently alkyl or haloalkyl; and
R₈ is H or C(O)CH₃;
(m) a compound of the formula (XIX): 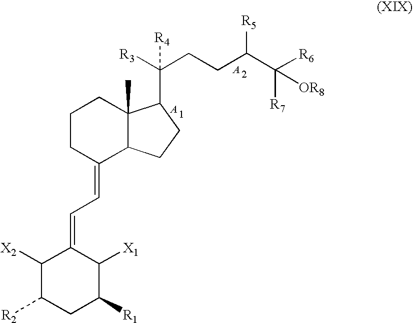
wherein:
A₁ is single or double bond;
A₂ is a single, double or triple bond,
X₁ and X₇ are each independently H₂ or CH₂, provided X₁ and X₂ are not both CH₁;
R₁ and R₂ are each independently OC(O)C₁-C₄ alkyl, OC(O)hydroxyalkyl, or OC(O)haloalkyl;
R₃, R₄ and R₅ are each independently hydrogen C₁-C₄ alkyl, hydroxyalkyl, or haloalkyl, or R₃ and R₄ taken to ether with C form C₃-C₆ cylcoalkyl;
R₆ and R₇ are each independently haloalkyl; and
R₈ is H, OC(O)C₁-C₄ alkyl OC(O)hydroxyalkyl, or OC(O)haloalkyl; and
pharmaceutically acceptable esters, salts, and prodrugs thereof;
(n) a compound of the formula (XX): 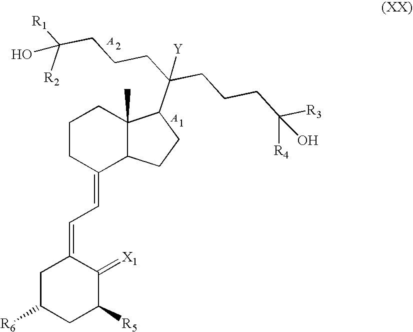
wherein:
A₁ is a single or double bond;
A₂ is a single, a double or a triple bond;
R₁, R₂, R₃ and R₄ are each independently alkyl, deuteroalkyl, hydroxyalkyl, or haloalkyl;
R₅ is halogen, hydroxyl, OC(O)alkyl, OC(O)hydroxyalkyl, or OC(O)haloalkyl;
R₆ is halogen, hydroxyl, OC(O)alkyl, OC(O)hydroxyalkyl, or OC(O)haloalkyl;
X₁ is H₇ or CH₂;
Y is alkyl;
and pharmaceutically acceptable esters, salts, and prodrugs thereof;
(o) a compound of the formula (XX-a): 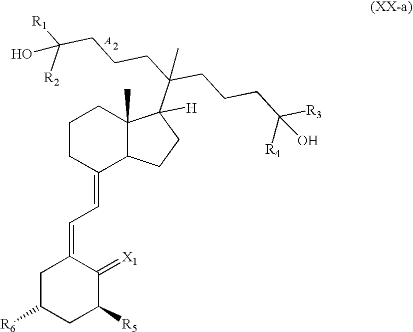
wherein:
A₂ is a single, a double or a triple bond;
R₁, R₂, R₃ and R₄ are each independently alkyl, hydroxyalkyl, or haloalkyl;
R₅ is halogen, hydroxyl, OC(O)alkyl, OC(O)hydroxyalkyl, or OC(O)haloalkyl;
R₆ is hydroxyl, OC(O)alkyl, OC(O)hydroxyalkyl, or OC(O)haloalkyl;
X₁ is H₂ or CH₂;
and pharmaceutically acceptable esters, salts, and prodrugs thereof;
(p) a compound of the formula (XX-b): 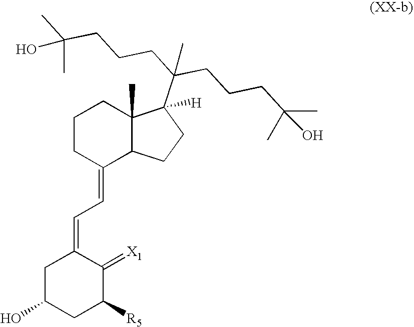
wherein:
R₅ is fluoro or hydroxyl;
X₁ is H₂ or CH₂;
and pharmaceutically acceptable esters, salts, and prodrugs thereof;
(q) a compound of the formula (XX-c): 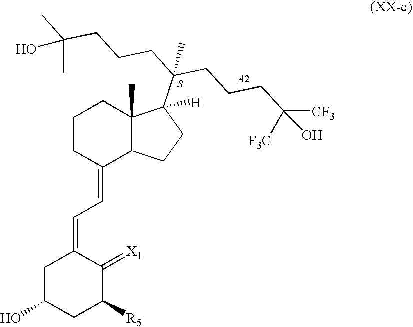
wherein:
A₂ is a single, a double or a triple bond;
R₅ is fluoro or hydroxyl;
X₁ is H₂ or CH₂;
and pharmaceutically acceptable esters, salts, and prodrugs thereof.
(r) a compound of the formula (XX-d): 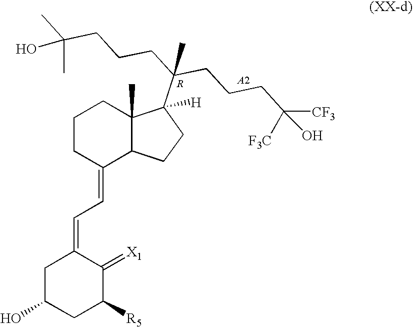
wherein:
A₂ is a single, a double or a triple bond;
R₅ is fluoro or hydroxyl;
X₁ is H₂ or C₂;
and pharmaceutically acceptable esters, salts, and prodrugs thereof;
(s) a compound of the formula (XX-e): 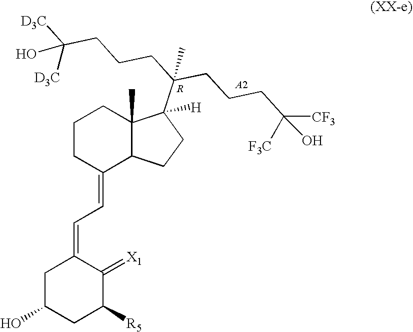
wherein:
A₂ is a single a double or a triple bond;
R₅ is fluoro or hydroxyl;
X₁ is H₂ or C₂;
and pharmaceutically acceptable esters, salts, and prodrugs thereof;
(t) a compound of the formula (XX-f): 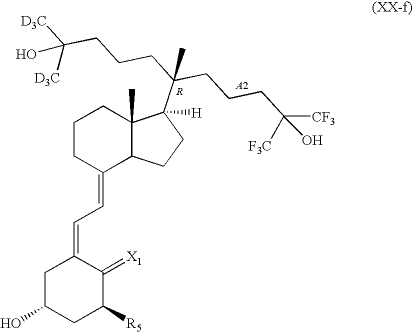
wherein:
A₂ is a single, a double or a triple bond;
R₅ is fluoro or hydroxyl;
X₁ is H₂ or CH₂;
and pharmaceutically acceptable esters, salts, and prodrugs thereof; and
(u) a compound of the formula (XXII): 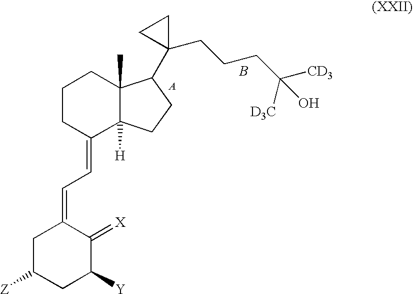
wherein:
A is single or double bond; B is a single, double, or triple bond; X is H₂ or CH₂;
Y is hydroxyl, OC(O) C₁-C₄ alkyl, OC(O)hydroxyalkyl, OC(O)haloalkyl; or halogen;
Z is hydroxyl, OC(O)C₁-C₄ alkyl, OC(O)hydroxyalkyl, or OC(O)haloalkyl; and
pharmaceutically acceptable esters, salts, and prodrugs thereof.

31. (canceled)

32. The method of claim 30, wherein said compound of formula (XVI) is 1-alpha-fluoro-25-hydroxy-16,23E-diene-26,27-bishomo-20-epi-cholecalciferol, having the formula:

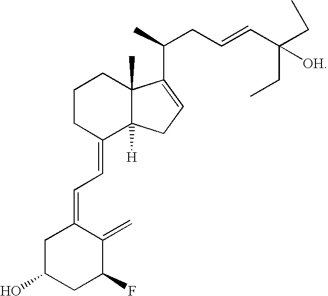

33-35. (canceled)

36. The method of claim 30, wherein said compound of formula (VI) is 1,25-dihydroxy-21-(3-hydroxy-3-methylbutyl)-19-nor-cholecalciferol having the formula:

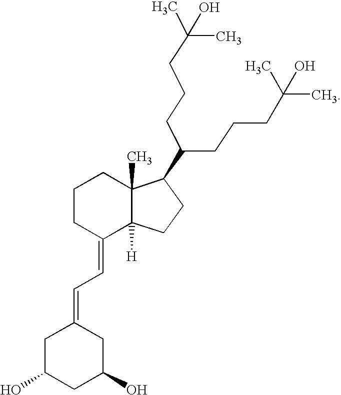

37-75. (canceled)

76. The method of claim 2, wherein said vitamin D compound is 2-methylene-19-nor-20(S)-1-alpha,25-hydroxyvitamin D₃:

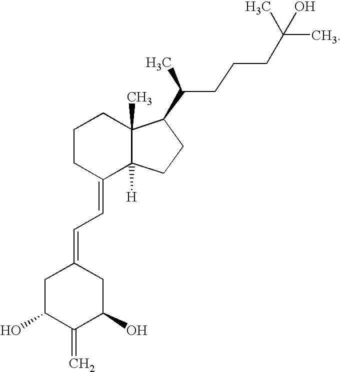

77-109. (canceled)

110. The method of claim 30, wherein said compound of formula (XIX) is 1,3-Di-O-acetyl-1,25-dihydroxy-20-cyclopropyl-cholecalciferol having the formula: 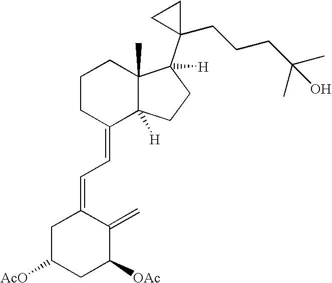
or is 1,25-dihydroxy-20,21,28-cyclopropyl-cholecalciferol having the formula:

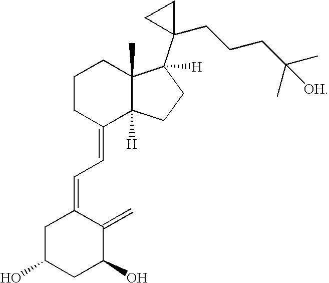

111. (canceled)

112. The method of claim 2 wherein said compound is calcitriol.

113. The method of claim 2 wherein the vitamin D compound is not co-administered or co-formulated with crosslinked compositions or crosslinkable compositions which inter-react in an aqueous environment to form a three-dimensional matrix.
